(19) United States
(12) Patent Application Publication
(10) Pub. No.: US 2022/0290139 A1
(43) Pub. Date: Sep. 15, 2022

(54) METHODS AND COMPOSITIONS FOR GENE SPECIFIC DEMETHYLATION AND ACTIVATION

(71) Applicants: NATIONAL UNIVERSITY OF SINGAPORE, Singapore (SG); BETH ISRAEL DEACONESS MEDICAL CENTER, INC., Boston, MA (US)

(72) Inventors: Yanjing LIU, Singapore (SG); Daniel G. TENEN, Singapore (SG); Annalisa DI RUSCIO, Boston, MA (US); Alexander K. EBRALIDZE, Boston, MA (US)

(21) Appl. No.: 17/627,966

(22) PCT Filed: Jul. 15, 2020
(86) PCT No.: PCT/US2020/042132

§ 371 Date: Jan. 18, 2022

Related U.S. Application Data
(60) Provisional application No. 62/874,160, filed on Jul. 15, 2019.

(51) Int. Cl. C12N 15/113; C12N 15/10; C12N 9/22; A61K 38/46; A61K 31/7088

(52) U.S. Cl. CPC C12N 15/113; C12N 15/10; C12N 9/22; A61K 38/465; A61K 31/7088; C12N 2310/531; C12N 2310/20

(57) ABSTRACT

Provided herein are methods and agents for gene specific demethylation and/or activation. Oligonucleotide constructs are provided, the oligonucleotide constructs including: [1] a targeting portion having sequence complementarity and binding affinity with a region of genomic DNA within a gene, near a gene, or both; and [2] a single guide RNA (sgRNA) scaffold portion, wherein a tetra-loop portion of the sgRNA is modified and includes an R2 stem loop of DNMT1-interacting RNA (DiR), and wherein a stem loop 2 portion of the sgRNA is modified and includes an R5 step loop of DiR. The oligonucleotide constructs may be used, together with deactivated (dead) Cas9 (dCas9) for providing gene specific demethylation and/or activation of gene(s) of interest in a cell or subject in need thereof.

### CROSS-REFERENCE TO RELATED APPLICATIONS

This application claims priority to PCT Application No. PCT/US2020/042132, having a filing date of Jul. 15, 2020, based on U.S. Provisional Application No. 62/874,160, having a filing date of Jul. 15, 2019, the entire contents both of which are hereby incorporated by reference.

### SEQUENCE LISTING

This application includes a separate sequence listing in compliance with the requirements of 37 C.F.R. §§ 1.824(a)(2)-1.824(a)(6) and 1.824(b), submitted under the file name “0016WO01_Sequence_Listing_942729WO_ST25”, created on Jan. 6, 2022, having a file size of 32 KB, the contents of which are hereby incorporated by reference.

### FIELD OF INVENTION

The present invention relates generally to gene demethylation and/or activation. More specifically, the present invention relates to methods and compositions for gene specific demethylation and/or activation using oligonucleotide constructs and deactivated Cas9.

### BACKGROUND

Epigenetics and DNA methylation abnormalities play a significant role in a number of important diseases, particularly cancer. Suppression of gene expression by methylation, particularly at CpG-rich promoters, has been associated with several tumor suppressor genes (TSG), and may be associated with long-term gene silencing in malignant cells. Reactivation of one or more tumor suppressor genes, in a targeted or specific manner, is highly desirable in the therapeutic field.

Unfortunately, development of approaches and treatments for reverting gene methylation and re-activating expression of aberrantly methylated genes has proven difficult. Development of broad demethylating agents (i.e. azacitidine, decitabine) to treat hypermethylation-associated diseases has been actively investigated, but the lack of specificity for the genetic loci and the high toxicity has presented challenges for such approaches.

Indeed, methods and agents providing for gene-specific demethylation and activation remain highly sought after in the field, particularly for anti-cancer applications. Approaches providing for demethylation and/or activation in a manner which more closely mimics natural processes are especially desirable.

Traditional approaches for gene demethylation are non-specific and often utilize small-molecule agents such as azacitidine or decitabine. Non-specific approaches can create a variety of unintended or undesirable effects. The discovery of Crispr and Cas9 has led to approaches for targeting particular sequences or regions within the genomic DNA based on sequence complementarity with a guide RNA targeting sequence; however, Crispr/Cas9 has traditionally been utilized as a gene editing system, and gene editing does not readily address gene deactivation by methylation.

Alternative, additional, and/or improved methods and agents for providing gene-specific demethylation and/or activation are desirable.

### SUMMARY OF INVENTION

Provided herein are methods and compositions for gene specific demethylation and/or activation. As described in detail herein below, oligonucleotides and methods have now been developed for providing targeted demethylation of one or more genes of interest, leading to activation and increased expression thereof. Using targeted oligonucleotide constructs designed for inhibiting DNA methyltransferase 1 (DNMT1) activity, and deactivated (dead) Cas9 (dCas9), it is shown herein that DNA methylation may be decreased in target methylated genomic regions, leading to increased gene expression of target gene(s) of interest. In certain embodiments, methods described herein may provide a more natural and targeted demethylation effect as compared with traditional non-specific demethylating agents, and results provided herein observed demethylation and activation over extended periods of time. Remarkably, as described herein it is found that targeting the non-template strand of the genomic DNA with the oligonucleotide(s) provided notably better gene demethylation/activation as compared with targeting the template strand of the genomic DNA.

In an embodiment, there is provided herein an oligonucleotide comprising:
a targeting portion having sequence complementarity and binding affinity with a region of genomic DNA within a gene, near a gene, or both; and
a single guide RNA (sgRNA) scaffold portion, wherein a tetra-loop portion of the sgRNA is modified and comprises an R2 stem loop of DNMT1-interacting RNA (DiR), and wherein a stem loop 2 portion of the sgRNA is modified and comprises an R5 step loop of DiR.

In another embodiment of the above oligonucleotide, the targeting portion may have sequence complementarity and binding affinity with a non-template strand of the genomic DNA within the gene, near the gene, or both.

In still another embodiment of any of the above oligonucleotide or oligonucleotides, the R2 and R5 stem loops of DiR may be from extra-coding CEBPA (ecCEBPA).

In still another embodiment of any of the above oligonucleotide or oligonucleotides, the targeting portion may target a methylated region of the genomic DNA.

In yet another embodiment of any of the above oligonucleotide or oligonucleotides, the targeting portion may target the genomic DNA region within or near a promoter region or within or near a demethylation core region (for example, a region encompassing a proximal promoter-exon 1-beginning of intron 1 region) of the gene, preferably wherein the targeting portion may target a region at or near the 5′ end of the first exon (for example, a proximal promoter region) or a region at or near the 3′ end of the first exon (for example, a beginning portion of intron 1) of the gene or a middle region (e.g. a region positioned between a proximal promoter on one side and the beginning of intron 1 on the other side) of the first exon of the gene. In certain embodiments, the middle region may comprise any portion or region within exon 1. In certain embodiments, the targeting portion may target a region at or near a proximal promoter region associated with the first exon and/or a region at or near the beginning of the first intron and/or a middle region of the first exon of the gene. Preferably, in certain embodiments, at least two oligonucleotides may be used, one having a targeting portion targeting a region at or near the 5′ end of the first exon (for example, a proximal promoter region), and one having a targeting portion targeting a region at or near the 3′ end of the first exon (for example, a beginning portion of intron 1) of the gene, so as to simultaneously target both ends of the demethylation core region. In certain embodiments, an oligonucleotide may be used having a targeting portion targeting a middle region (e.g. a region positioned between a proximal promoter on one side and the beginning of intron 1 on the other side) of the first exon of the gene. In certain embodiments, at least three oligonucleotides may be used, one having a targeting portion targeting a region at or near the 5′ end of the first exon (for example, a proximal promoter region), one having a targeting portion targeting a region at or near the 3′ end of the first exon (for example, a beginning portion of intron 1) of the gene, and one having a targeting portion targeting a middle region (e.g. a region positioned between a proximal promoter on one side and the beginning of intron 1 on the other side) of the first exon of the gene, so as to simultaneously target both ends and a middle region of the demethylation core region. It is contemplated that where combinations of oligonucleotides are used, the different oligonucleotides may be for administration simultaneously, sequentially, or in combination. Typically, the oligonucleotides may be for administration such that they act simultaneously; however, it is also contemplated that in certain embodiments different oligonucleotides or oligonucleotide combinations may be used at different time points or at different stages, for regulating gene activation.

In still another embodiment of any of the above oligonucleotide or oligonucleotides, the oligonucleotide may comprise the sequence:

(Rₐ)GUUUR_{b}AGAGCUA(R_{c})UAGCAAGUUR_{d}AAAUAAGGCUAGUCCGUUAUCAACUUAGUGGCACCGAGUCGGUGC(Rₑ)A GUGGCACCGAGUCGGUGC(R_{f}) (Formula I)
wherein
Rₐ comprises the targeting portion, and comprises about 20 to about 21 nucleotides in length;
R_{b} is A, G, or C, and R_{d} is the complementary base pair of R_{b};
R_{c} comprises the R2 stem loop of DiR, comprising sequence CCCGGGACGCGGGUCCGGGACAG (SEQ ID NO: 7);
Rₑ comprises the R5 step loop of DiR, comprising sequence CUGAGGCCUUGGCGAGGCUUCU (SEQ ID NO: 8); and
R_{f} is optionally present, and comprises a poly U transcription termination sequence.

In still another embodiment of any of the above oligonucleotide or oligonucleotides, the oligonucleotide may comprise the sequence:

(Rₐ)GUUUGAGAGCUACCCGGGACGCGGGUCCGGGACAGUAGCAAGUUCAAAUAAGGCU AGUCCGUUAUCAACUUCUGAGGCCUUGGCGAGGCUUCUAAGTGGCACCGAGUCGGUG CUUUUUU; (Formula II)
wherein Rₐ comprises the targeting portion, and comprises about 20 to about 21 nucleotides in length.

In still another embodiment of any of the above oligonucleotide or oligonucleotides, the gene may be P16, and Rₐ may comprise:

(SEQ ID NO: 9)
GCUCCCCCGCCUGCCAGCAA;
(SEQ ID NO: 10)
GCUAACUGCCAAAUUGAAUCG;
(SEQ ID NO: 11)
GACCCUCUACCCACCUGGAU;
or
(SEQ ID NO: 12)
GCCCCCAGGGCGUCGCCAGG.

In another embodiment, there is provided herein a plasmid or vector encoding any of the oligonucleotide or oligonucleotides described herein.

In another embodiment, there is provided herein a composition comprising any of the oligonucleotide or oligonucleotides described herein and a dead Cas9 (dCas9).

In another embodiment, there is provided herein a composition comprising any one or more of:
an oligonucleotide as described herein;
a plasmid or vector as described herein;
a pharmaceutically acceptable carrier, excipient, diluent, or buffer;
a dead Cas9 (dCas9); or
an oligonucleotide, plasmid, or vector encoding a dead Cas9 (dCas9).

In another embodiment of the above composition, the dCas9 may comprise D10A and H840A mutations.

In still another embodiment, there is provided herein a composition comprising any of the oligonucleotide or oligonucleotides described herein wherein the targeting portion targets a 5′ region of the first exon of a gene; and any of the oligonucleotide or oligonucleotides described herein wherein the targeting portion targets a 3′ region of the first exon of the gene.

In another embodiment, there is provided herein a composition comprising:
any of the oligonucleotide or oligonucleotides as described herein, wherein the targeting portion targets a region at or near the 5′ end of the first exon (for example, a proximal promoter region) of a gene; and
any of the oligonucleotide or oligonucleotides as described herein wherein the targeting portion targets a region at or near the 3′ end of the first exon (for example a beginning region of intron 1) of the gene; and
optionally, further comprising any of the oligonucleotide or oligonucleotides described herein, wherein the targeting portion targets a middle region of the first exon of the gene;
preferably, wherein the composition comprises an oligonucleotide as described herein wherein the targeting portion targets a region at or near a proximal promoter region associated with the first exon; and an oligonucleotide as described herein wherein the targeting portion targets a region at or near the beginning of the first intron; and optionally further comprises an oligonucleotide as described herein wherein the targeting portion targets a middle region of the first exon of the gene.

In yet another embodiment there is provided herein a combination of any of the oligonucleotide or oligonucleotides described herein wherein the targeting portion targets a region at or near a 5′ end of the first exon of a gene; and any of the oligonucleotide or oligonucleotides described herein wherein the targeting portion targets a region at or near a 3′ end of the first exon of the gene.

In another embodiment, there is provided herein a method for targeted demethylation and/or activation of a gene, said method comprising:
introducing a dead Cas9 (dCas9) and one or more oligonucleotides into a cell, the one or more oligonucleotides each comprising:
   a targeting portion having sequence complementarity and binding affinity with a region of genomic DNA within the gene, near the gene, or both; and
   a single guide RNA (sgRNA) scaffold portion, wherein a tetra-loop portion of the sgRNA is modified and comprises an R2 stem loop of DNMT1-interacting RNA (DiR), and wherein a stem loop 2 portion of the sgRNA is modified and comprises an R5 step loop of DiR thereby demethylating and/or activating the gene by inhibiting DNA methyltransferase 1 (DNMT1) activity on the gene.

In another embodiment of the above method, the targeting portion of at least one of the one or more oligonucleotides may have sequence complementarity and binding affinity with a non-template strand of the genomic DNA within the gene, near the gene, or both.

In still another embodiment of any of the above method or methods, the step of introducing comprises transfecting, delivering, or expressing the one or more oligonucleotides and the dCas9 in the cell.

In yet another embodiment of any of the above method or methods, the one or more oligonucleotides comprise any one or more of the oligonucleotide or oligonucleotides as described herein.

In still another embodiment of any of the above method or methods, at least two oligonucleotides may be introduced into the cell, wherein the targeting portion of a first oligonucleotide targets a 5′ region of the first exon of the gene; and wherein the targeting portion of a second oligonucleotide targets a 3′ region of the first exon of the gene.

In still another embodiment of any of the above method or methods, at least two oligonucleotides may be introduced into the cell, wherein the targeting portion of a first oligonucleotide targets a region at or near a 5′ end of the first exon of the gene; and wherein the targeting portion of a second oligonucleotide targets a region at or near the 3′ end of the first exon of the gene; preferably wherein the targeting portion of the first oligonucleotide targets a region at or near a proximal promoter region associated with the first exon and the targeting portion of the second oligonucleotide targets a region at or near the beginning of the first intron; optionally wherein a third oligonucleotide may be introduced into the cell, wherein the targeting portion of the third oligonucleotide targets a middle region of the first exon.

In another embodiment of any of the above method or methods, the cell may be exposed to the dCas9 and the one or more oligonucleotides for a period of at least about 3 days, at least about 4 days, at least about 5 days, at least about 6 days, at least about 7 days, or at least about 8 days, or about 3 days to about a week.

In another embodiment, there is provided herein a use of any of the oligonucleotide or oligonucleotides, the plasmid or plasmids or vector or vectors, the composition or compositions, or the combination or combinations as described herein, for targeted demethylation and/or activation of a gene.

In another embodiment, there is provided herein a method for treating a disease or disorder associated with decreased expression of at least one gene due to aberrant DNA methylation in a subject in need thereof, said method comprising:
treating the subject with a dead Cas9 (dCas9) and one or more oligonucleotides, the one or more oligonucleotides each comprising:
   a targeting portion having sequence complementarity and binding affinity with a region of genomic DNA within the gene, near the gene, or both; and
   a single guide RNA (sgRNA) scaffold portion, wherein a tetra-loop portion of the sgRNA is modified and comprises an R2 stem loop of DNMT1-interacting RNA (DiR), and wherein a stem loop 2 portion of the sgRNA is modified and comprises an R5 step loop of DiR;
      thereby demethylating and/or activating the gene by inhibiting DNA methyltransferase 1 (DNMT1) activity on the gene, and treating the disease or disorder.

In another embodiment of the above method, the targeting portion of at least one of the one or more oligonucleotides may have sequence complementarity and binding affinity with a non-template strand of the genomic DNA within the gene, near the gene, or both.

In still another embodiment of any of the above method or methods, the step of treating may comprise transfecting, delivering, or expressing the one or more oligonucleotides and the dCas9 in at least one cell of the subject.

In still another embodiment of any of the above method or methods, the one or more oligonucleotides may comprise one or more oligonucleotides as described herein.

In still another embodiment of any of the above method or methods, at least two oligonucleotides may be used, wherein the targeting portion of a first oligonucleotide targets a 5′ region of the first exon of the gene; and wherein the targeting portion of a second oligonucleotide targets a 3′ region of the first exon of the gene.

In still another embodiment of any of the above method or methods, at least two oligonucleotides may be used, wherein the targeting portion of a first oligonucleotide targets a region at or near a 5′ end of the first exon of the gene; and wherein the targeting portion of a second oligonucleotide targets a region at or near a 3′ end of the first exon of the gene; preferably wherein the targeting portion of the first oligonucleotide targets a region at or near a proximal promoter region associated with the first exon and the targeting portion of the second oligonucleotide targets a region at or near the beginning of the first intron; optionally wherein a third oligonucleotide is used, wherein the targeting portion of the third oligonucleotide targets a middle region of the first exon.

In yet another embodiment of any of the above method or methods, the subject may be exposed to the dCas9 and the one or more oligonucleotides for a period of at least about 3 days, at least about 4 days, at least about 5 days, at least about 6 days, at least about 7 days, or at least about 8 days, or about 3 days to about a week.

In another embodiment there is provided herein a use any of the oligonucleotide or oligonucleotides, the plasmid or plasmids or vector or vectors, the composition or compositions, or the combination or combinations as described herein, for treating a disease or disorder associated with decreased expression of at least one gene due to aberrant DNA methylation in a subject in need thereof.

In another embodiment of any of the above methods or uses, the targeting portion of at least one of the one or more oligonucleotides may target a site within or near a promoter region of the gene or within or near a demethylation core region of the gene, preferably wherein the targeting portion targets a region at or near a 5′ end of the first exon or a region at or near a 3′ end of the first exon of the gene.

In another embodiment of any of the above methods or uses, at least two oligonucleotides may be used, wherein the targeting portion of a first oligonucleotide targets a region at or near a 5′ end of the first exon of the gene; and wherein the targeting portion of a second oligonucleotide targets a region at or near a 3′ end of the first exon of the gene.

In another embodiment of any of the above methods or uses, the promoter region may be a CpG-rich region having at least some methylation.

In still another embodiment of any of the above methods or uses, the disease or disorder may comprise cancer.

In yet another embodiment of any of the above methods or uses, the gene may be a tumor suppressor gene.

In another embodiment of any of the above methods or uses, the targeting portion of at least one of the one or more oligonucleotides may target a site within or near a promoter region of the gene or within or near a demethylation core region of the gene, in particular wherein the targeting portion may target a region at or near a 5′ end of the first exon or a region at or near a 3′ end of the first exon of the gene, wherein the gene is a tumor suppressor gene.

In another embodiment of any of the above methods or uses, the promoter region may be a CpG-rich region having at least some methylation.

In still another embodiment of any of the above methods or uses, the targeting portion of at least one of the one or more oligonucleotides may target the D1 or D3 region of the P16 gene.

In another embodiment of any of the above methods or uses, the one or more oligonucleotides may comprise at least one oligonucleotide with a targeting portion targeting the D1 region, and at least one oligonucleotide with a targeting portion targeting the D3 region, and optionally further comprising at least one oligonucleotide with a targeting portion targeting the D2 region.

In another embodiment of any of the above methods or uses, the one or more oligonucleotides may comprise one or more of:

G19sgR2R5 (SEQ ID NO: 1):
GCUCCCCCGCCUGCCAGCAAGUUUGAGAGCUACCCGGGACGCGGGUCCG
GGACAGUAGCAAGUUCAAAUAAGGCUAGUCCGUUAUCAACUUCUGAGGC
CUUGGCGAGGCUUCUAAGUGGCACCGAGUCGGUGCUUUUUU;
G36sgR2R5 (SEQ ID NO: 2):
GCUAACUGCCAAAUUGAAUCGGUUUGAGAGCUACCCGGGACGCGGGUCC
GGGACAGUAGCAAGUUCAAAUAAGGCUAGUCCGUUAUCAACUUCUGAGG
CCUUGGCGAGGCUUCUAAGUGGCACCGAGUCGGUGCUUUUUU;
G110sgR2R5 (SEQ ID NO: 3):
GACCCUCUACCCACCUGGAUGUUUGAGAGCUACCCGGGACGCGGGUCCG
GGACAGUAGCAAGUUCAAAUAAGGCUAGUCCGUUAUCAACUUCUGAGGC
CUUGGCGAGGCUUCUAAGUGGCACCGAGUCGGUGCUUUUUU;
G111sgR2R5 (SEQ ID NO: 4):
GCCCCCAGGGCGUCGCCAGGGUUUGAGAGCUACCCGGGACGCGGGUCCG
GGACAGUAGCAAGUUCAAAUAAGGCUAGUCCGUUAUCAACUUCUGAGGC
CUUGGCGAGGCUUCUAAGUGGCACCGAGUCGGUGCUUUUUU;
G108sgR2R5 (SEQ ID NO: 5):
GUGGCCAGCCAGUCAGCCGAGUUUGAGAGCUACCCGGGACGCGGGUCCG
GGACAGUAGCAAGUUCAAAUAAGGCUAGUCCGUUAUCAACUUCUGAGGC
CUUGGCGAGGCUUCUAAGUGGCACCGAGUCGGUGCUUUUUU;
or
G122sgR2R5 (SEQ ID NO: 6):
GCCGCAGCCGCCGAGCGCACGGUUUGAGAGCUACCCGGGACGCGGGUCC
GGGACAGUAGCAAGUUCAAAUAAGGCUAGUCCGUUAUCAACUUCUGAGG
CCUUGGCGAGGCUUCUAAGUGGCACCGAGUCGGUGCUUUUUU;

or any combinations thereof.

In another embodiment, there is provided herein a method for identifying one or more target sites for demethylation to activate expression of agene in a cell, said method comprising:
treating the cell with a non-specific demethylation agent;
identifying one or more regions around the transcription start site of the gene which are most demethylated by treatment with the non-specific demethylating agent; and
using the identified one or more regions as target sites for demethylation to activate gene expression.

In another embodiment of the above method, the non-specific demethylation agent may comprise Decitabine (2′-deoxy-5-azacytidine).

In yet another embodiment of any of the above method or methods, the treatment with the non-specific demethylation agent may be for about 3 days.

In another embodiment of any of the above method or methods, the step of identifying the one or more regions around the transcription start site of the gene which are most demethylated by treatment with the non-specific demethylating agent may comprise performing Bisulfite Sanger-sequencing or whole genomic Bisulfite sequencing and, optionally, comparing results with a control untreated cell.

In another embodiment of any of the above method or methods, selection of the one or more regions around the transcription start site may favour selection of regions at or near the promoter, at or near the first exon of the gene, at or near a first intron of the gene, at or near a region at or near a 5′ end of the first exon of the gene, at or near a region at or near a 3′ end of the first exon of the gene, at or near a CpG island, at or near another important regulatory region, or any combinations thereof.

In still another embodiment of any of the above method or methods, selection of the one or more regions around the transcription start site may favour selection of at least one region at or near a 5′ region of the first exon of the gene, and at least one region at or near a 3′ region of the first exon of the gene.

In another embodiment of any of the above method or methods, the method may further comprise performing targeted demethylation and gene activation using any of the method or methods described herein, wherein the targeting portions of the one or more oligonucleotides have sequence complementarity with the identified target sites for demethylation.

In another embodiment of any of the above method or methods, the one or more regions may be regions of the non-template strand.

### BRIEF DESCRIPTION OF DRAWINGS

These and other features will become further understood with regard to the following description and accompanying drawings, wherein:

FIG. 1 shows that CRISPR-R2R5 system induced moderate gene activation and demethylation by targeting promoter CpG island. In FIG. 1(a), the structure of sgRNA (no DiR) and sgR2R5 (with DiR), the targeting site G2, and the transfection methods, are shown. In FIG. 1(b) the p16 mRNA expression in each sample after 72 hours treatment is shown. In FIG. 1(c), the MSP data showing the gene demethylation is shown. (Abbreviations—sgOri: Original sgRNA without DiR; sgR2R5: sgRNA fused with R2,R5 loops; G2: guide RNA; MSP: Methylation Specific PCR);

FIG. 2 shows sequence and structural organization of typical single guide RNA (sgRNA);

FIG. 3 shows results of CRISPR-DiR targeting P16 Region D1 and D3 simultaneously, with four guides targeting both strands in each region. FIG. 3(a) shows the targeting strategy; FIG. 3(b) shows the P16 mRNA expression profile; FIG. 3(c) shows the P16 protein restoration profile; FIG. 3(d) shows the methylation in Region D1 and D3 measured by COBRA; and FIG. 3(e) shows the cell cycle analysis of the Day 53 treated samples;

FIG. 4 shows results of CRISPR-DiR targeting P16 Region D1 and D3 simultaneously, with only one DNA strand targeted in each sample. FIG. 4(a) shows the targeting strategy, FIG. 4(b) shows the P16 expression profile, and FIG. 4 (c) shows the methylation profile in Region D1 and D3 measured by COBRA. Targeting means the guide RNA sequence (i.e. targeting portion) is complimentary to the targeted strand. The mRNA sequence (sense strand) is the same as the non-template strand. Thus, in the COBRA data, S (targeting sense strand) refers to targeting non-template strand (NT), AS (targeting antisense) refers to targeting template (T) strand;

FIG. 5 shows the methylation and gene expression profiles for SNU-398 wild type cells treated with 2.5 uM DAC for three and five days. FIG. 5(a) shows the five regions checked for methylation in P16 locus; FIG. 5(b) shows the P16 gene expression in the cell samples; and FIG. 5(c) shows bisulfite sequencing data for wild type cells and DAC treated cells in Region A, C, D and E. Each black or white dot represents a CG site, the black dot indicates methylated C, while white dot represents unmethylated C;

FIG. 6 shows results of CRISPR-DiR targeting P16 Region E with four mixed guide RNAs (G113, G114, G115, G116). In FIG. 6(a), the targeting strategy is shown; in FIG. 6(b), the P16 expression profile traced for three months is shown; in FIG. 6(c) the methylation of CRISPR-DiR treated samples measured by COBRA in Day0, day, Day28 and Day 41 is shown. The red arrows indicate the undigested DNA, which is the demethylated DNA that can't be cut. In FIG. 6(d), the methylation in Region D1 after targeting Region E for 41 days is shown; in FIG. 6(e) the methylation in Region D2 after targeting Region E for 41 days is shown; and in FIG. 6(f) the methylation in Region D3 after targeting Region E for 41 days is shown;

FIG. 7 shows results of CRISPR-DiR targeting Region E with the same guide RNAs but no dCas9. “Not loaded” means there are not enough samples to load; however, the unload samples are uncut control, so the uncut band information can still be obtained from other uncut samples, and the length of all the uncut DNA should be the same;

FIG. 8 shows CRISPR-DiR targeting P16 Region E, or Region A or Region E+A with four mixed guide RNAs for each region. FIG. 8(a) shows the targeting strategy; FIG. 8(b) shows the P16 expression profile; FIG. 8(c) shows the methylation in Region E of CRISPR-DiR treated samples measured by COBRA, Region E was targeted for 72 days while Region A was targeted for 19 days; and FIG. 8(d) shows the methylation in Region A after targeting Region E, Region E was targeted for 72 days while Region A was targeted for 19 days;

FIG. 9 shows CRISPR-DiR targeting P16 Region E, or Region D1 or Region E+D1 with four mixed guide RNAs for each region. In FIG. 9(a), the targeting strategy is shown; In FIG. 9(b) the P16 expression profile is shown; in FIG. 9(c) the methylation in Region E and Region D1 of CRISPR-DiR treated samples measured by COBRA is shown, Region E was targeted for 92 days while Region D1 was targeted for 18 days;

FIG. 10 shows CRISPR-DiR targeting of P16 Region E, D1, D2, and D3 Region or Region D1. Each region was targeted with four mixed guide RNAs. In FIG. 10(a) the targeting strategy is shown; in FIG. 10(b) the P16 expression profile is shown; in FIG. 10(c) the methylation in Region D1 measured by COBRA is shown; in FIG. 10(d) the methylation in Region D3 measured by COBRA is shown; In FIG. 10(e) the methylation in Region E measured by COBRA is shown; in FIG. 10(f) the methylation in Region C measured by COBRA is shown. Region E was targeted for 116 days, Region D1 was targeted for 33 days, Region D2 was targeted for 28 days, Region D3 was targeted for 13 days. The red frames highlight that Region C and E was demethylated even not directly targeted;

FIG. 11 shows the Bisulfite PCR sequencing result for the dynamic demethylation progress of CRISPR-DiR treated samples, and accompanies the data shown in FIG. 3;

FIG. 12 shows the methylation profile in Region C, D1, D2, D3 and E during the whole 53 days CRISPR-DiR treatment, measured by COBRA. CRISPR-DiR targeting p16 Region D1 and D3 simultaneously, with four guides targeting both strands in each region;

FIG. 13 shows results of CRISPR-DiR targeting p16 Region D1 and D3 simultaneously, with only one DNA strand targeted in each sample. FIG. 13(a) shows the targeting strategy; FIG. 13(b) shows the p16 expression profile; and FIG. 13(c) shows the methylation profile in Region D1 and D3 measured by COBRA. Targeting means the guide RNA sequence is complimentary to the targeted strand. The mRNA sequence (sense strand) is the same as the non-template strand. Thus, in the COBRA data, S (sense strand) refers to targeting non-template strand (NT), AS (antisense) refers to targeting template (T) strand;

FIG. 14 shows design of an embodiment of a CRISPR-DiR system. Short DNMT1-interacting RNA loops from ecCEBPA may be fused to the original sgRNA scaffold, tetra loop and stem loop 2 as shown;

FIG. 15 shows results of CRISPR-DiR targeting p16 Region D1 and D3 non-template strand (NT) simultaneously in U2OS cell line. FIG. 15(a) shows the targeting strategy, FIG. 15(b) shows the p16 expression profile, and FIG. 15(c) shows the methylation profile in Region D1 and D3 measured by COBRA;

FIG. 16 shows results of CRISPR-DiR targeting SALL4 non-template strand for demethylation and gene activation with Guide 1.6 sgDiR (sg1.6, GCTGCGGCTGCTGCTCGCCC (SEQ ID NO: 13)). FIG. 16(a) shows the targeting strategy, FIG. 16(b) shows the SALL4 mRNA expression profile, FIG. 16(c) shows the SALL4 protein restoration, and FIG. 16(d) shows the demethylation in the targeted regions of control cells and CRISPR-DiR treated cells;

FIG. 17 shows CEBPA mRNA expression and p14 mRNA expression in U2OS cells with CRISPR-DiR targeted for 51 days;

FIG. 18 shows results from the dcas 9 inducible CRISPR-DiR system in SNU-398 cells. FIG. 18(a) shows the targeting strategy, FIG. 18(b) shows the p16 expression profile, and FIG. 18(c) shows the methylation profile in Region D1 measured by COBRA;

FIG. 19 shows histone markers ChIP-qPCR results of CRISPR-DiR treated fifty-three cells. FIG. 19(a) shows the locations of ChIP-qPCR checked histone markers, P16 is the CRISPR-DiR targeted gene, while P14, P15, downstream 10 Kb are the nearby non-targeted locus; FIG. 19(b) shows the enrichment of active histone marker H3K4me3; FIG. 19(c) shows the enrichment of active histone marker H3K27ac; and FIG. 19(d) shows the enrichment of silencing histone marker H3K9me3;

FIG. 20 shows the development of an embodiment of the CRISPR-DiR system. FIG. 20(a) depicts the rationale of this embodiment of the CRISPR-DiR design. In the Modified sgDiR (MsgDiR), short DNMT1-interacting RNA (DiR) loops R2 and R5 from ecCEBPA were fused to the original sgRNA scaffold, tetra-loop and/or stem-loop 2 regions. FIG. 20(b) shows diagrams of the original sgRNA control and eight different versions of MsgDiR design. All the sgRNA and MsgDiR constructs were utilized guide G2 targeting the p16 gene proximal promoter. FIG. 20(c) shows a schematic representation of gene p16 and the targeting site (G2) of sgRNA control and MsgDiRs. FIG. 20(d) shows Methylation Sensitive PCR (MSP) data demonstrating p16 demethylation in SNU-398 cell lines 72 hours post-transfection. Mock: transfection reagents with H₂O; sgRNA: co-transfection of dCas9+sgRNA (no DiR); Msg1-8: co-transfection of dCas9+MsgDiRs (with DiR) according to the design shown in FIG. 20(c); NTC: none template control. FIG. 20(e) is a schematic representation of a preferred CRISPR-DiR system after screening: dCas9+MsgDiR6, in which R2 is fused to sgRNA tetra-loop 2 while R5 is fused to sgRNA stem-loop 2;

FIG. 21 shows p16 activation correlates with demethylation in exon 1 rather than promoter CpG island. FIG. 21(a) depicts Whole Genomic Bisulfite Sequencing (WGBS) results indicating the methylation profiles in the PrExI region (p16 Promoter (Region D1)-Exon 1 (Region D2)-Intron 1 (Region D3) of wild type SNU-398 (WT) and SNU-398 treated with 2.5 uM Decitabine for 72h (DAC). The height of the blue bar represents the methylation level of each CpG residue. FIG. 21(b) depicts Real Time-Quantitative PCR (RT-qPCR) of p16 gene expression in wild type and Decitabine treated SNU-398 cells, WT: wild type; DAC: Decitabine. FIG. 21(c) is a schematic representation of the location of Region D1, Region D2 and Region D3 in the p16 locus, as well as the CRISPR-DiR targeting sites in these three regions. To target Region D1, guides G36 and G19 were used in CRISPR-DiR; to target Region D2, guides G108 and G123; to target Region D3, guides G110 and G111. FIG. 21(d) shows real Time-Quantitative PCR (RT-qPCR) results of p16 RNA in SNU-398 cell lines stably transduced with CRISPR-DiR lentivirus. Mean f SD, n=3, *P<0.05; **P<0.01; ***P<0.001;

FIG. 22 shows CRISPR-DiR targeting p16 Region D1 and Region D3 simultaneously induced a dynamic process of demethylation and gene reactivation. FIG. 22(a) is a schematic representation of the location of Region D1, Region D2, and Region D3 in p16, CRISPR-DiR targeting strategy: targeting p16 Region D1 (G36, G19) and Region D3 (G110, G111) simultaneously. FIG. 22(b) shows Bisulfite Sequencing PCR (BSP) results indicating the gradual demethylation profile in p16 Region D1, D2, and D3 from Day 0 to Day 53 following CRISPR-DiR treatment in SNU-398 cells. FIG. 22(c) shows Real Time-Quantitative PCR (RT-qPCR) results showing p16 mRNA expression after CRISPR-DiR treatment in SNU-398 cells. FIG. 22(d) shows a Western Blot assessing p16 protein after CRISPR-DiR treatment. Beta actin (ACTB) was used as loading control. FIG. 22(e) shows RT-qPCR results showing p16 gradual mRNA after the same CRISPR-DiR treatment in the human osteosarcoma U2OS cell line. FIG. 22(f) shows Combined Bisulfite Restriction Analysis (COBRA) representing the gradual demethylation profile in p16 Region D1, D2, D3 (PrExI) from Day 0 to Day 53 with the same CRISPR-DiR treatment in U2OS cells. U=uncut, C=cut DNA. The band after cutting (lanes “C”) with migration equal to uncut represents demethylated DNA, and are indicated by red arrows. Mean f SD, n=3, *P<0.05; **P<0.01; ***P<0.001;

FIG. 23 shows CRISPR-DiR effects are maintained for more than a month and PrExI demethylation leads to dynamic change in histone modifications. FIG. 23(a) depicts Real Time-Quantitative PCR (RT-qPCR) results showing p16 mRNA for more than a month in inducible CRISPR-DiR SNU-398 cells. In the inducible system, the same targeting strategy shown in FIG. 22A (Region D1+Region D3) was used, and dCas9 expression was induced for 0 day, 3 days, 8 days, or 32 days following treatment with Deoxycytidine (Dox). All treatments were cultured and assayed at Day 0, Day 3, Day 8 or Day 32. FIG. 23(b) shows Combined Bisulfite Restriction Analysis (or COBRA) representing the demethylation profile of p16 in inducible CRISPR-DiR SNU-398 cells. The demethylation status was maintained for more than a month with as short as three days induction. The band after cutting (lanes C) with equal migration as uncut (lanes U) represents demethylated DNA, indicated by red arrows. FIG. 23(c) is a schematic representation of the location of ChIP-qPCR primers (See Table 7). Neg 1 and Neg 2: negative control primer 1 and 2 located 50 kb upstream and 10 kb downstream of p16, respectively. CpG island is indicated in green. FIG. 23(d) depicts ChIP-qPCR results showing the gradual increase in H3K4Me3 and H3K27Ac and decrease in H3K9Me3 enrichment in the p16 PrExI region in SNU 398 cells stably transduced with CRISPR-DiR targeting D1+D3 as in FIG. 22A. FIG. 23(e) is a dynamic comparison of change in p16 mRNA, methylation, and histone modifications in SNU 398 cells stably transduced with CRISPR-DiR targeting Region D1+D3. Mean f SD, n=3, *P<0.05; **P<0.01; ***P<0.001;

FIG. 24 shows CRISPR-DiR induced specific demethylation of p16 PrExI remodels chromatin structure through CTCF to activate gene expression. FIG. 24(a) is a schematic representation of DNA methylation, histone marks (H3K4Me3, H3K27Ac, and H3K4Me1), and CTCF ChIP-Seq profiles in p16 Region D1, D2, and D3. WGBS methylation data were collected from SNU-398 cells (both wild type and Decitabine treated) performed in our study; histone mark enrichments determined by ChIP-seq cross 7 cell lines (GM12878, H1-hESC, HSMM, HUVEC, K562, NHEK, NHLF) obtained from ENCODE; CTCF binding was analyzed in our study using ChIP-Seq data from cell lines analyzed by TFregulomeR (FB8470, GM12891, GM19240, prostate epithelial cells, and H1-derived mesenchymal stem cells). FIG. 24(b) shows CTCF binding motif predicted in the p16 Exon 1 region. FIG. 24(c) depicts ChIP-qPCR results showing enrichment of CTCF in p16 PrExI region after CRISPR-DiR induced demethylation. Primers are same as histone ChIP-qPCR (FIG. 23C) Mean f SD, n=3, *P<0.05; **P<0.01; ***P<0.001. FIG. 24(d) shows a hypothetical model of CRISPR-DiR induced demethylation of the PrExI region results in recruitment of distal regulatory elements through CTCF enrichment, showing the 4C assay viewpoint 1 (generated by restriction enzyme Csp6I), covering the 800 bp demethylated region (PrExI). FIG. 24(e) shows circularized chromosome conformation capture (4C)-Seq analysis of CRISPR-DiR treated Day 13 samples (GN2 non-targeting control and D1+D3 targeted). Shown are interactions captured by 4C between p16 viewpoint 1 and potential distal regulatory elements. The change in interaction was determined by normalizing the interactions of the targeted sample (D1+D3) to GN2 control; strong interaction changes are represented by the curves at the bottom from color blue to red, and the strongest interactions (potential distal enhancer elements) are highlighted and labelled as E1 to E6. FIGS. 24(f) and 24(g) show the hypothetical model and 4C-Seq analysis of FIGS. 24(d) and 24(e), using viewpoint 2 (generated by restriction enzyme DpnII), covering the 600 bp p16 promoter region and p16 exon 1;

FIG. 25 is a schematic of CRISPR-DiR induced targeted demethylation in the Demethylation Firing Center (PrExI) initiating local and distal chromatin rewiring for gene activation. Gene silencing is coupled with aberrant DNA methylation in the region surrounding the transcription start site (TSS) as well as heterochromatin structure (upper left). Simultaneous targeting of the upstream promoter and beginning of intron 1 regions via CRISPR-DiR induces locus specific demethylation of the Demethylation Firing Center, which initiates an epigenetic wave of local chromatin remodeling and distal long-range interactions, culminating in gene-locus specific activation (on the right);

FIG. 26 shows Transient transfection of MsgDiR6+dCas9 alone induces P16 demethylation and moderate gene activation. FIG. 26(a) is a schematic representation of the p16 gene locus and the target location. Both sgRNA (no DiR) and MsgDiR6 (with R2 and R5) target the p16 promoter CpG island with guide G2. FIG. 26(b) depicts Methylation Sensitive PCR (MSP) data showing the p16 demethylation in SNU-398 cell lines 72 hours post-transfection. Mock: transfection reagents with H₂O; sgRNA: either transfect only a sgRNA (no DiR) or co-transfection of dCas9+sgRNA (no DiR); MsgDiR6: either transfect only MsgDiR6 (with DiR) or co-transfection of dCas9+MsgDiR6 (with DiR) as shown in FIG. 26A. FIG. 26(c) depicts Real Time-Quantitative PCR (RT-qPCR) result showing p16 gene expression in SNU-398 cells 72 hours post transient transfection. The sgRNA and MsgDiR6 were transfected into the cells both with and without dCas9. Mean f SD, n=3, *P<0.05; **P<0.01; ***P<0.001;

FIG. 27 shows the Minimum Free Energy (MFE) structure and Centroid secondary structure analysis of sgRNA, sgSAM, and MsgDiRs. The structures reveal that MsgDiR6 is the only design with same MFE structure and Centroid secondary structure as the sgSAM structure (with MS2 aptamers fused to the sgRNA scaffold). FIG. 27(a) shows Minimum Free Energy (MFE) structure analysis of sgRNA(T), sgRNA(G), sgSAM, and MsgDiRI-8. The analysis is performed by RNAfold (79). The structure is colored by base-pairing probabilities. For unpaired regions, the color denotes the probability of being unpaired. FIG. 27(b) shows Centroid secondary structure analysis of sgSAM and MsgDiR3-7. MsgDiR3-7 all have similar MFE structures as sgSAM, but only MsgDiR6 has both stable MFE and a Centroid secondary structure similar to sgSAM. The analysis was performed by RNAfold. The structure is colored by base-pairing probabilities. For unpaired regions the color denotes the probability of being unpaired;

FIG. 28 shows targeting specific demethylation induced by CRISPR-DiR. FIG. 28(a) is a schematic representation of the p16 gene locus and the location of Region C, Region D1, Region D2, Region D3, and Region E. CRISPR-DiR targeting a single region or combined regions were all stably transduced into SNU-398 cells with the guides via lentivirus. The sgDiR guides are listed in Table 4 (and described in the detailed description below) and the location of each region are listed in Table 5 (and described in the detailed description below). FIG. 28(b) shows a Combined Bisulfite Restriction Analysis (COBRA) analysis of the demethylation profile in p16 Region D1. Region D1 methylation of SNU-398 cells transduced with CRISPR-DiR non-targeting (GN2) control, and targeting Region D1, Region D2, Region D3, and Region D1+Region D3 were all analyzed after 13 days treatment. *** U: uncut sample, C: cut by BstUI. The band after cutting with migration equal to that of the uncut band represents demethylated DNA. FIG. 28(c) shows Combined Bisulfite Restriction Analysis (or COBRA) analysis of the demethylation profile in p16 Region D3, performed as for FIG. 28B. FIG. 28(d) shows Combined Bisulfite Restriction Analysis (or COBRA) representing the demethylation profile in p16 Region C, D1, D2, D3, and E, with CRISPR-DiR targeting Region D1+Region D3 for 53 days. U: uncut, C: cut. Primers and restriction enzymes can be found in Table 6. The demethylation initiated in Region D1 and Region D3 only spread over time to the middle Region D2, but not flanking Regions C or E. FIG. 28(e) shows Real Time-Quantitative PCR (RT-qPCR) result showing p16 gene expression in SNU-398 cells with CRISPR-DiR non-targeting control, targeting Region D1+D3, or targeting Region C+E. FIG. 28(f) shows Real Time-Quantitative PCR (RT-qPCR) result showing the change of gene p14 and gene CEBPA RNA during the 53 day period of CRISPR-DiR targeting p16 Region D1+Region D3 in U2OS cells. p14 is hypermethylated and silenced (undetectable) in U2OS, while CEBPA is not hypermethylated but expressed in U2OS. No significant expression change of these two genes was observed during the CRISPR-DiR targeting p16 process. Mean±SD, n=3,*P<0.05; **P<0.01; ***P<0.001;

FIG. 29 shows distal interactions detected by 4C analysis with viewpoint 1 (Csp6I) and viewpoint 2 (DpnII). FIG. 29 depicts circularized chromosome conformation capture (4C)-Seq analysis of CRISPR-DiR treated Day 13 samples (GN2 non-targeting control and targeted Region D1+Region D3) in SNU-398 cells. The top panel shows interactions captured for viewpoint 1 (Csp6I) while the bottom shows interactions for viewpoint 2 (DpnII). The interaction changes after CRISPR-DiR “Region D1+Region D3” targeted demethylation were normalized to the same time point (Day 13) non-targeting control (GN2) sample, and the strong interaction changes demonstrated by the interaction arcs, with color from blue to red, representing the interaction fold change from two fold to the highest fold change. The potential distal enhancer elements with the strongest interaction were highlighted for both viewpoints on the top, labeled from p16 upstream to downstream (negative orientation) as E1, E2, E3, E4, E5, and E6;

FIG. 30 depicts Bisulfite Sequencing PCR results showing the methylation profile in p15 promoter-exon 1-intron 1 region in wild type Kasumi-1 and KG-1 cells, the less methylated regions are highlighted as Region D1 and Region D3 following the same pattern in p16. Black dots represent methylated CG sites, while white dots represent unmethylated CG sites.; and

FIG. 31 shows sequences of MsgDiRI-8 constructs, as well as regular and modified sgRNA, and sgSAM for comparison.

### DETAILED DESCRIPTION

Described herein are methods and compositions for gene specific demethylation and/or activation. It will be appreciated that embodiments and examples are provided for illustrative purposes intended for those skilled in the art, and are not meant to be limiting in any way.

Methylation of CpG-rich promoters in several tumor suppressor genes (TSG) is associated with long-term gene silencing in malignant cells, thus a therapeutic approach to revert this mechanism may provide a strategy to restore expression of aberrantly methylated genes. Low toxicity and gene-specific demethylating agents have been lacking.

Provided herein is a modified CRISPR-based platform to achieve gene-specific demethylation and activation. For this platform, the two protruding loops of the single guide RNA (sgRNA) scaffold may be replaced with the two stem-loop-like sequences of the DNA methyltransferase 1 (DNMT1) interacting RNA (DiRs) (Di Ruscio et al., 2013). The DiR-modified sgRNA (sgDiR) may block DNMT1 enzymatic activity in a gene-specific manner. Using sgDiRs targeting the tumor suppressor gene P16 as described herein has not only successfully demethylated P16 while restoring both mRNA and protein expression, but induced P16-dependent cell cycle arrest. Similar results were obtained using sgDiRs targeting the SALL4 gene locus, supporting that this strategy may be used as a general approach for multiple genes. In certain embodiments, CRISPR-DiR systems as described herein may be used in tracing the dynamics of epigenetic regulation, and/or may offer a tool to modulate gene-specific DNA methylation by RNA. In certain embodiments, it is contemplated that CRISPR-DiR systems as described herein may provide RNA-based gene-specific demethylating tools for a variety of applications such as, for example, cancer treatment and/or treatment of genetic diseases triggered by aberrant DNA methylation.

In certain embodiments, methods described herein may provide a more natural and targeted demethylation effect as compared with traditional non-specific demethylating agents, and results provided herein observed demethylation and activation over extended periods of time. Remarkably, as described herein it is found that targeting the non-template strand (sense strand) of the genomic DNA with the oligonucleotide(s) provided notably better gene demethylation/activation as compared with targeting the template strand of the genomic DNA. Furthermore, related and/or particularly effective demethylation targeting regions for gene re-activation have been carefully explored in studies from 2 Kb upstream of the gene transcription start site (TSS) to the first intron, and the results clearly indicated that instead of targeting the hypermethylated promoter, the simultaneous targeted demethylation of the 5′ and 3′ of the first exon significantly enhanced the gene activation compared with targeting any single region in the gene promoter or first exon, or targeting any other two regions simultaneously in the studies performed. Targeting of the first exon worked especially well for P16 gene activation, and may also work well for SALL4 activation, for example.

Embodiments ofoligonucleotide constructs described herein may allow for efficient transcription and stable RNA structure. Approaches as described herein may provide for an RNA-based strategy to demethylate a gene locus of interest, and/or may provide for a natural and flexible strategy amendable to modification and/or delivery. It is contemplated that in certain embodiments, approaches as described herein may be for delivering specific TF, or other factors, to a target location, for example.

In certain experiments, it was observed that gene demethylation and activation using embodiments as described herein was initiated and became stable after about a week. Continued tracing of the treated cells showed that demethylation and activation effects may be found to gradually increase and be maintained over at least one month in both consecutive stable line or dCas9-inducible cell lines (inducing dCas9 expression for three days or 8 days, for example). In certain embodiments, it is contemplated that approaches as described herein may be used to explore dynamic regulation mechanism(s) of gene expression, and/or may be used to develop therapeutic strategies for a variety of diseases.

Recent work (Di Ruscio et al., 2013) demonstrated that a class of RNAs, the DNMT1-interacting RNAs (DiRs), binds to the maintenance DNA methyltransferase 1, DNMT1, with higher affinity than DNA and may play an important role in regulating DNA methylation profile genome-wide. Using as a model the methylation sensitive gene CEBPA, a nuclear non-polyadenylated RNA originating from this locus was identified, termed extra-coding CEBPA (ecCEBPA), interacting with DNMT1 with stronger affinity than the DNA corresponding sequence and regulating CEBPA locus DNA methylation. The DNMT1-RNA interaction may rely on ecCEBPA and, more in general, on RNA secondary stem-loop-like structures, thereby inhibiting DNMT1 enzymatic activity and preventing DNA methylation. Moreover, preliminary data suggested that introduction of RNAs able to (1) target the CEBPA locus by forming a RNA-DNA triple helix structure; and (2) interact with DNMT1, led to activation of CEBPA mRNA and gene locus demethylation.

Single guide RNA (sgRNA)-Cas9/dead Cas9 (dCas9) CRISPR systems are being developed for gene-specific targeting. By introducing two point mutations in the catalytic residues (D1OA and H840A) of Cas9 gene, the resultant dCas9 loses the nuclease activity but may serve as a good platform to carry other transcription regulation proteins to the targets, for example. Some studies have attempted fusing transcription activation/repressive domains to dCas9 or sgRNA (Konermann et al., 2015, Gilbert et al., 2014, Gilbert et al., 2013). Crystallographic studies have been performed to explore the atomic structure of sgRNA-dCas9 (Nishimasu et al., 2014). Based on the crystal structure, the plasticity of sgRNA scaffold has been investigated, the structure analyzed, and it was identified that the sgRNA tetraloop and stemloop 2 protrude outside of the dCas9-sgRNA complex, with 4 base pairs of each stem loop free of interactions with dCas9 amino acid side chains. Data indicating that substitutions and deletions in the tetraloop and stem loop 2 sequence do not affect Cas9 catalytic function further showed that these two regions may tolerate the addition of RNA aptamers (sgRNA(MS2)), adding function by recruiting other functional domains via RNA aptamer instead of/along with fusion into dCas9 (referred to as Synergistic Activation Mediators (SAM)) (Konermann et al., 2015). However, effective systems for gene-specific demethylation and activation, particularly those providing a more natural-type effect, have remained highly sought after in the field. Other CRISPR systems appear focused on fusing functional proteins into dCas9 (e.g. dCas9-VP64; dCas9-Tet1), which may result in larger systems, systems for which delivery is difficult, systems which do not mimic natural processes, and/or systems which may have toxicity, for example.

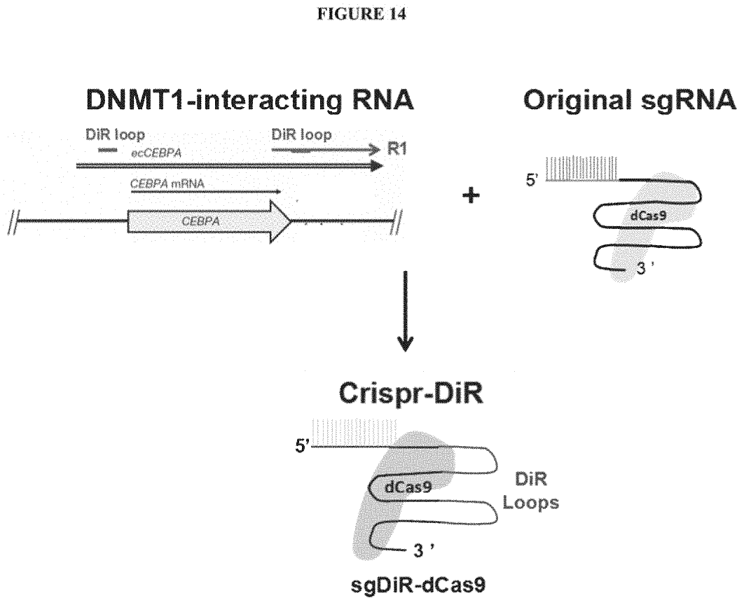

As described herein, by fusing the short DiR loops (R2 and R5 from ecCEBPA) to sgRNA tetra loop and stemloop2, a modified CRISPR demethylation approach has now been developed, referred to herein as CRISPR-DiR (see FIG. 14, showing an example of combination of DNMT1-interacting RNA (DiR) with sgRNA scaffold to arrive at modified oligonucleotide constructs which may be loaded into dCas9).

Provided herein are methods and agents for gene specific demethylation and/or activation. Oligonucleotide constructs are provided, which may be used, together with deactivated (dead) Cas9 (dCas9), for providing gene specific demethylation and/or activation of gene(s) of interest in a cell or subject in need thereof.

In an embodiment, there is provided herein an oligonucleotide comprising:
a targeting portion having sequence complementarity and binding affinity with a region of genomic DNA within a gene, near a gene, or both; and
a single guide RNA (sgRNA) scaffold portion, wherein a tetra-loop portion of the sgRNA is modified and comprises an R2 stem loop of DNMT1-interacting RNA (DiR), and wherein a stem loop 2 portion of the sgRNA is modified and comprises an R5 step loop of DiR.

As will be understood, the targeting portion may comprise any suitable sequence having at least partial sequence complementarity and binding affinity with a region of genomic DNA within a gene, near a gene, or both (or at another site at which demethylation may be desired). Typically, the targeting portion may be designed to be fully or substantially complementary with the intended target region of the genomic DNA so as to provide good target recognition and binding, while reducing instances of off-target binding. In certain embodiments, the targeting portion may comprise a sequence having full complementarity with the intended target region of the genomic DNA, or a sequence having at least about 90%, at least about 91%, at least about 92%, at least about 93%, at least about 94%, at least about 95%, at least about 96%, at least about 97%, at least about 98%, or at least about 99% identity therewith.

In certain embodiments, the targeting portion may be designed or selected using approaches and/or rules developed for other CRISPR strategies. For example, programs and websites are available for design and analysis of a CRISPR guide RNA, using design rules developed in the field. Typically, programs developed for regular CRISPR guide design will provide a list of guide RNAs for a desired target region, which are typically about 20 nt in length and 100% complementary to the targeted DNA region, and provide a predicted on-target score and off-target score. In certain embodiments, the targeted portion may be chosen in such manner, aiming for a high on-target score and a low off-target score. In embodiments where the designed guide RNA starts with a “G”, then it is contemplated that in certain embodiments described herein the targeting portion may comprise or consist of the 20 nt guide RNA sequence beginning with “G”. In embodiments where the designed guide RNA does not start with a “G”, then it is contemplated that in certain embodiments described herein the targeting portion may comprise or consist of the 20 nt guide RNA sequence having an extra “G” optionally added to the beginning of the guide RNA (i.e. the 5′ end) to provide a 21 nt sequence, particularly where it is desirable that a “G” be positioned at the beginning to serve as the transcription start of sgRNA driven by a U6 promoter, for example.

In certain embodiments the region of genomic DNA targeted by the targeting portion may be any suitable region within a gene, near a gene, or both. In certain embodiments, the region of genomic DNA may comprise a region of the genomic DNA which is methylated, or which is near a methylated region. In certain embodiments, the region of genomic DNA may comprise a region of the genomic DNA which is aberrantly methylated in connection with a disease, disorder, or condition, or which is near such a region. In certain embodiments, the region of genomic DNA may comprise a region of the genomic DNA which is aberrantly methylated in connection with a cancer, or which is near such a region. In certain embodiments, the region of genomic DNA targeted by the targeting portion may comprise a genomic DNA region within or near a promoter region of a gene of interest or within or near a demethylation core region or a gene of interest. In certain embodiments, the region of genomic DNA targeted by the targeting portion may comprise a region at or near the 5′ end of the first exon of the gene. In certain embodiments, the region of genomic DNA targeted by the targeting portion may comprise a region at or near the 3′ end of the first exon of the gene.

In certain embodiments, at least two oligonucleotides may be used, wherein the targeting portion of a first oligonucleotide targets a 5′ region of the first exon of the gene; and wherein the targeting portion of a second oligonucleotide targets a 3′ region of the first exon of the gene.

In certain embodiments, a demethylation core region may comprise a genomic region of a gene spanning along the proximal promoter region, exon 1, and at least the beginning portion of intron 1 (which may, in certain embodiments, comprise about 500 nt into intron 1) of the gene.

In certain embodiments, a region at or near the 5′ end of the first exon may comprise a region anywhere within +/−about 500 nt from the beginning of the exon, or any sub-region therein. In certain embodiments, a region at or near the 3′ end of the first exon may comprise a region anywhere within +/−about 500 nt from the end of the exon, or any sub-region therein. A region at or near a 5′ end of the first exon encompasses a proximal promoter region associated with the first exon. A region at or near a 3′ end of the first exon encompasses the beginning of the first intron. In certain embodiments, targeting both a region at or near the 5′ end of the first exon of the gene and a region at or near the 3′ end of the first exon of the gene may be performed. As will be understood, in certain embodiments a region at or near the 5′ end of the first exon of the gene may comprise an upstream or proximal promoter region, and a region at or near the 3′ end of the first exon of the gene may comprise a region at or near the beginning of intron 1, for example.

In certain embodiments, at least two oligonucleotides may be used, wherein the targeting portion of a first oligonucleotide targets a region at or near a 5′ end of the first exon of the gene; and wherein the targeting portion of a second oligonucleotide targets a region at or near the 3′ end of the first exon of the gene; preferably wherein the targeting portion of the first oligonucleotide targets a region at or near a proximal promoter region associated with the first exon and the targeting portion of the second oligonucleotide targets a region at or near the beginning of the first intron; and optionally wherein a third oligonucleotide may be used, wherein the targeting portion of the third oligonucleotide targets a middle region of the first exon.

Preferably, in certain embodiments, at least two oligonucleotides may be used, one having a targeting portion targeting a region at or near the 5′ end of the first exon (for example, a proximal promoter region), and one having a targeting portion targeting a region at or near the 3′ end of the first exon (for example, a beginning portion of intron 1) of the gene, so as to simultaneously target both ends of the demethylation core region.

In certain embodiments, an oligonucleotide may be used having a targeting portion targeting a middle region (e.g. a region positioned between a proximal promoter on one side and the beginning of intron 1 on the other side) of the first exon of the gene.

In certain embodiments, at least three oligonucleotides may be used, one having a targeting portion targeting a region at or near the 5′ end of the first exon (for example, a proximal promoter region), one having a targeting portion targeting a region at or near the 3′ end of the first exon (for example, a beginning portion of intron 1) of the gene, and one having a targeting portion targeting a middle region (e.g. a region positioned between a proximal promoter on one side and the beginning of intron 1 on the other side) of the first exon of the gene, so as to simultaneously target both ends and a middle region of the demethylation core region.

In certain embodiments, it is contemplated that where combinations of oligonucleotides are used, the different oligonucleotides may be for administration simultaneously, sequentially, or in combination. Typically, the oligonucleotides may be for administration such that they act simultaneously or in concert; however, it is also contemplated that in certain embodiments different oligonucleotides or oligonucleotide combinations may be used at different time points or at different stages, for regulating gene activation.

As will be understood, references above to the 5′ end and the 3′ end directionality of the first exon are with respect to orientation and directionality of the gene to be targeted, such that 5′ and 3′ orientations are indicated relative to directionality of the non-template DNA strand (which, by convention, corresponds with direction of the gene).

In studies described herein, it has been found that rather than focusing on targeting the promoter, CRISPR-DiR may induce remarkable gene activation by simultaneously targeting region D1 and D3 as described herein. In other words, it is identified herein that by targeting both the at or near the 5′ region of the first exon and at or near the 3′ region of the first exon of a target gene simultaneously using CRISPR-DiR with different targeting regions, remarkable gene activation was observed in studies described herein. Indeed, a highly efficient demethylating and targeting strategy identified herein for gene activation is not only targeting the upstream/proximal promoter upstream of TSS (which is the most well studied region and most popular target region), but targeting “proximal promoter+beginning of intron 1”. This targeting strategy is shown in both p16 and p15 tumor suppressor genes in the Examples below. Further, data shows that targeting both promoter and intron 1 regions was highly effective, and that the middle exon 1 region is also relevant. As described in Example 3 below, the promoter-exon1-intron1 (PrExI) region is identified as “demethylation firing center (DFC)” having a regulatory role. In certain embodiments, targeting promoter region (e.g. region D1), exon 1 (e.g. region D2), or intron 1 (e.g. region D3) may be performed alone. In results obtained and described hereinbelow, targeting exon 1 (e.g. region D2) actually initiated the highest gene activation when only one of these three regions was targeted. When targeting promoter and intron 1 (e.g. D1 and D3) together, or targeting promoter, exon 1, and intron 1 (e.g. D1, D2, and D3) together, markedly better activation results were obtained, and results were similar between promoter and intron 1, and promoter, exon 1, and intron 1 strategies. Accordingly, in certain embodiments, targeting may be performed at or near both a proximal promoter region of a gene of interest and a beginning of intron 1 region of the gene of interest, and optionally additionally at or near a middle region of exon 1 of the gene of interest (a middle region may comprise a region positioned between a proximal promoter on one side and the beginning of intron 1 on the other side, such that the middle region may, in certain embodiments, comprise generally any region or portion of the first exon of the gene). Results provided hereinbelow indicate that even if the middle of exon 1 is not targeted, demethylation may spread to the middle region of exon 1.

In certain embodiments, the middle region of exon 1 of the gene of interest may be or comprise a region of exon 1 which may be experimentally determined (for example, by whole genomic bisulfite sequencing data of wild-type and decitabine treated SNU-398 samples) as being the most, or a highly, demethylated region as a result of treatment with a non-specific demethylating agent, for example. In another embodiment, and by way of example, Example 3 below indicates that in connection with p16, the middle region of exon 1 of the gene may be or include an important regulatory region which contains CTCF binding site for distal enhancer interaction. In certain embodiments, the middle region of exon 1 of the gene may be or comprise an important methylation associated regulatory region for other targets genome-wide, for example.

In certain embodiments, it is contemplated that these results from targeting at or near the 5′ region and at or near the 3′ region of the first exon of the target gene simultaneously (see results from targeting D1 and D3 regions in the Examples below) may be applied to targeting of other important regulatory region(s) of a given gene, such as regulatory region(s) where one, some, or most regulatory factors bind. In certain embodiments, it is contemplated that targeting both sides around an important regulatory region where important transcription factors or even distal enhancers bind may be desirable. In certain embodiments, rather than, or in addition to, targeting both at or near the 5′ region and at or near the 3′ region of the first exon of the target gene, it may be desirable to target both at or near the 5′ region and at or near the 3′ region of another important regulatory region of the target gene. In certain embodiments, it is contemplated that the important regulatory region may comprise one or more regions at or near the promoter of the gene, at or near the first exon of the gene, at or near a first intron of the gene, at or near a CpG island, at or near another important regulatory region, or any combinations thereof. In certain embodiments, it may be desirable to use CRISPR-DiR systems as described herein for targeting both sides flanking one or more important regulatory regions (such as those where one, some, or most regulatory factors bind) of a target gene. In certain embodiments, the important regulatory region may comprise a region determined to be the most important regulatory region for a given gene, for example.

In certain embodiments, the targeting portion may have complementarity and binding affinity with a non-template strand (i.e. sense strand) of the genomic DNA within the gene, near the gene, or both. Accordingly, in certain embodiments, the targeted portion may be designed to target the non-template (NT) strand of the genomic DNA. As described in the Examples below, targeting the non-template strand may provide more effective demethylation and/or gene activation in the studies described.

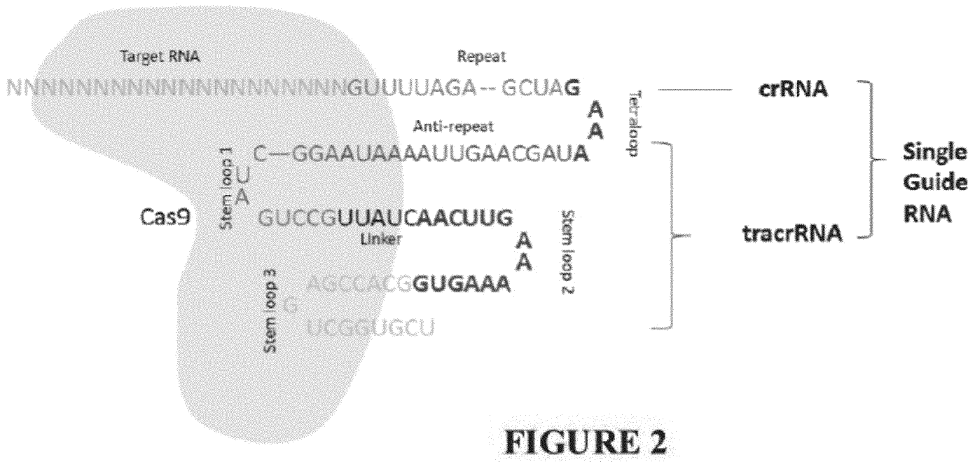

In certain embodiments, the single guide RNA (sgRNA) scaffold portion may comprise any suitable sequence compatible with dCas9, and in which a tetra-loop portion of the sgRNA is modified and comprises an R2 stem loop of DNMT1-interacting RNA (DiR), and in which a stem loop 2 portion of the sgRNA is modified and comprises an R5 step loop of DiR. Structure of typical unmodified single guide RNA (sgRNA), showing tetra-loop and stem loop 2 regions, are shown in FIG. 2 by way of illustrative example.

In the sgRNA scaffold portion of the present oligonucleotides, a tetra-loop portion of the sgRNA may be modified and comprise an R2 stem loop of DNMT1-interacting RNA (DiR), and a stem loop 2 portion of the sgRNA may be modified and comprise an R5 step loop of DiR. In certain embodiments, the R2 and R5 stem loops of DiR may be from extra-coding CEBPA (ecCEBPA). In certain embodiments, the tetra-loop portion of the sgRNA may be modified to comprise an R2 stem loop of DiR comprising sequence CCCGGGACGCGGGUCCGGGACAG (SEQ ID NO: 7), or a sequence having at least about 90/a, at least about 91%, at least about 92%, at least about 93%, at least about 94%, at least about 95%, at least about 96%, at least about 97%, at least about 98%, or at least about 99% identity therewith. In certain embodiments, the stem loop 2 portion of the sgRNA may be modified to comprise an R5 step loop of DiR comprising sequence CUGAGGCCUUGGCGAGGCUUCU (SEQ ID NO: 8), or a sequence having at least about 90%, at least about 91%, at least about 92%, at least about 93%, at least about 94%, at least about 95%, at least about 96%, at least about 97%, at least about 98%, or at least about 99% identity therewith. In certain embodiments, the sgRNA scaffold portion may be positioned 3′ to the targeting portion of the oligonucleotide.

As will be understood, in certain embodiments, sequence of the sgRNA scaffold portion may be modified from that of typical sgRNA at one or more other positions in addition to the tetra-loop and stem loop 2 portions. By way of example, in the embodiment immediately below, the nucleotide at position R_{b} may be changed from the typical U to an A, G, or C, and R_{d} may be changed from the typical A to be the complementary base pair of R_{b}. It is contemplated that such modification may provide for more effective sgDiR transcription driven by U6 promoter for example, and/or may make the RNA structure more stable as described below.

In certain embodiments, the oligonucleotide may comprise the sequence:

(Rₐ)GUUUGR_{b}AGAGCUA(R_{c})UAGCAAGUUR_{d}AAAUAAGGCUAGUCCGUUAUCAACUU(Rₑ)A GUGGCACCGAGUCGGUGC(R_{f}) (Formula I)

wherein:
Rₐ comprises the targeting portion, and comprises about 20 to about 21 nucleotides in length;
and the targeting portion is followed by the sgRNA scaffold portion (shown in underline), wherein
   R_{b} is A, G, or C, and R_{d} is the complementary base pair of R_{b};
   R_{c} comprises the R2 stem loop of DiR, comprising sequence CCCGGGACGCGGGUCCGGGACAG (SEQ ID NO: 7);
   Rₑ comprises the R5 step loop of DiR, comprising sequence CUGAGGCCUUGGCGAGGCUUCU (SEQ ID NO: 8); and
   R_{f} is optionally present, and comprises a poly U transcription termination sequence;
or a sequence having at least about 90%, at least about 91%, at least about 92%, at least about 93%, at least about 94%, at least about 95%, at least about 96%, at least about 97%, at least about 98%, or at least about 99% identity therewith.

In certain embodiments, the oligonucleotide may comprise the sequence:

(Rₐ)GUUUGAGAGCUACCCGGGACGCGGGUCCGGGACAGUAGCAAGUUCAAAUAAGGCU AGUCCGUUAUCAACUUCUGAGGCCUUGGCGAGGCUUCUAAGTGGCACCGAGUCGGUG CUUUUUU; (Formula II)
wherein Rₐ comprises the targeting portion, and comprises about 20 to about 21 nucleotides in length;
or a sequence having at least about 90%, at least about 91%, at least about 92%, at least about 93%, at least about 94%, at least about 95%, at least about 96%, at least about 97%, at least about 98%, or at least about 99% identity therewith.

In certain embodiments, oligonucleotide constructs modified with R2 stem loop modification at the tetra-loop portion and R5 stem loop modification at the stem loop 2 portion may provide for good maintenance of unmodified sgRNA secondary structure, and this secondary structure may be further stabilized by modifying the typical “U” at position R_{b} to “G”, and the typical “A” at position R_{d} to “C” (for complementarity with R_{b}) (which may also assist with transcription efficiency, in certain embodiments). It is contemplated that when designing modified oligonucleotides, maintenance of original sgRNA structure may be desirable in certain embodiments to avoid disruption of binding ability of the oligonucleotide with Cas9/dCas9 to form a complex for targeting a specific DNA region.

In certain embodiments, the targeting portion may designed to target P16 gene, and Rₐ (i.e. the targeting portion) may comprise:

(SEQ ID NO: 9)
GCUCCCCCGCCUGCCAGCAA;
(SEQ ID NO: 10)
GCUAACUGCCAAAUUGAAUCG;
(SEQ ID NO: 11)
GACCCUCUACCCACCUGGAU;
or
(SEQ ID NO: 12)
GCCCCCAGGGCGUCGCCAGG.

As will be understood, plasmids, expression vectors, cassettes, and other sequences (both double and single-stranded, DNA or RNA) comprising, encoding, and/or capable of expressing any of the oligonucleotides as described herein are also contemplated and provided herein, as well as oligonucleotides which are complementary with or capable of binding with any of the oligonucleotides as described herein.

In certain embodiments, plasmids, expression vectors, cassettes, and other sequences comprising or encoding or expressing any of the oligonucleotides as described herein, dCas9 as described herein, or both, are contemplated and provided herein. In certain embodiments, one or more plasmids containing or capable of expressing the sgDiR oligonucleotide and dCas9, may be provided, and may be delivered into cells by lentivirus (for example), where the DNA sequences may be inserted into cell genome and then transcribed to sgRNA or finally translated to dCas9.

In certain embodiments, a delivery vehicle such as lentivirus may be used to deliver DNA constructs into cells, then the DNA may be transcribed into RNA (i.e. oligonucleotides as described herein such as sgR2R5). In certain embodiments, sgR2R5 RNA may be introduced or delivered into cells. Where oligonucleotides as described herein are introduced or delivered into cells, they may be provided to cells separately or in combination with dCas9 in certain embodiments.

The skilled person will be aware of a wide variety of transfection or delivery approaches, reagents, and vehicles suitable for delivering or otherwise introducing oligonucleotides as described herein into cells, and/or for delivering or otherwise introducing dCas9 into cells. In certain embodiments, the oligonucleotides, dCas9, or both, may be expressed within the cells. In certain embodiments, the oligonucleotides, dCas9, or both, may be transfected, introduced, or delivered into cells.

Expression vectors (either viral, plasmid, or other) may be transfected, electroporated, or otherwise introduced into cells, which may then express the oligonucleotides, dCas9, or both. Alternatively, oligonucleotides (such as RNA oligonucleotide constructs) may be introduced into cells, for example via electroporation or transfection (i.e. using a transfection reagent such as Lipofectamine™, Oligofectamine™, or any other suitable delivery agent known in the art), or via targeted nucleic acid vehicles known in the art.

Approaches, reagents, and vehicles suitable for the delivery or introduction of relatively short oligonucleotides into cells are well known. By way of example, a wide variety of strategies have been developed for delivery of gene silencing RNAs (i.e. siRNAs) into cells, and it is contemplated that such approaches may also be used for delivering oligonucleotides as described herein. As well, a wide variety of chemical modifications have been developed for stabilizing RNA sequences, such as gene silencing RNAs (i.e. siRNAs), and it is contemplated that such approaches may also be used for stabilizing oligonucleotides as described herein. By way of example, it is contemplated that any of the oligonucleotides described herein may be modified to include one or more unnatural nucleotides, such as 2′-O-methyl, 2′-Fluoro, or other such modified nucleotides (see, for example, Gaynor et al., RNA interference: a chemist's perspective. Chem. Soc. Rev. (2010) 39: 4196-4184). Many delivery vehicles and/or agents are well-known in the art, several of which are commercially available. Delivery strategies for oligonucleotides are described in, for example, Yuan et al., Expert Opin. Drug Deliv. (2011) 8:521-536; Juliano et al., Acc. Chem. Res. (2012) 45: 1067-1076; and Rettig et al., Mol. Ther. (2012) 20:483-512. Examples of transfection methods are described in, for example, Ausubel et al., (1994) Current Protocols in Molecular Biology, John Wiley & Sons, New York. Expression vector examples are described in, for example, Cloning Vectors: A Laboratory Manual (Pouwels et al., 1985, Supp. 1987).

As referenced herein, percent (%) identity or % sequence identity with respect to a particular sequence, or a specified portion thereof, may be understood as the percentage of nucleotides in the candidate sequence identical with the nucleotides in the subject sequence (or specified portion thereof), after aligning the sequences and introducing gaps, if necessary, to achieve maximum percent sequence identity, as generated by the program WU-BLAST-2.0 with search parameters set to default values (Altschul et al., J. Mol. Biol. (1990) 215:403-410; website at blast.wustl.edu/blast/README.html). By way of example, a % identity may be determined by the number of matching identical nucleotides divided by the sequence length for which the percent identity is being reported. Oligonucleotide alignment algorithms such as, for example, BLAST (GenBank; using default parameters) may be used to calculate sequence identity %.

In another embodiment, there is provided herein a plasmid or vector encoding any of the oligonucleotide or oligonucleotides as described herein.

In another embodiment, there is provided herein a composition comprising any of the oligonucleotide or oligonucleotides as described herein, and a dead Cas9 (dCas9).

There are several sequence versions for Cas9 and dead Cas9. For the examples below, several versions of Cas9 plasmid were first screened and the one with strongest cleavage efficiency was identified. Then point mutations were introduced in the two catalytic residues (D1OA and H840A) of the gene encoding Cas9 to make an effective dead Cas9. mCherry sequence was also added after dCas9 as a selection marker. The sequence of the dCas9-mCherry used is:

(SEQ ID NO: 14)
ATGGACTATAAGGACCACGACGGAGACTACAAGGATCATGATATTGATT
ACAAAGACGATGACGATAAGATGGCCCCAAAGAAGAAGCGGAAGGTCGG
TATCCACGGAGTCCCAGCAGCCGACAAGAAGTACAGCATCGGCCTGGCC
ATCGGCACCAACTCTGTGGGCTGGGCCGTGATCACCGACGAGTACAAGG
TGCCCAGCAAGAAATTCAAGGTGCTGGGCAACACCGACCGGCACAGCAT
CAAGAAGAACCTGATCGGAGCCCTGCTGTTCGACAGCGGCGAAACAGCC
GAGGCCACCCGGCTGAAGAGAACCGCCAGAAGAAGATACACCAGACGGA
AGAACCGGATCTGCTATCTGCAAGAGATCTTCAGCAACGAGATGGCCAA
GGTGGACGACAGCTTCTTCCACAGACTGGAAGAGTCCTTCCTGGTGGAA
GAGGATAAGAAGCACGAGCGGCACCCCATCTTCGGCAACATCGTGGACG
AGGTGGCCTACCACGAGAAGTACCCCACCATCTACCACCTGAGAAAGAA
ACTGGTGGACAGCACCGACAAGGCCGACCTGCGGCTGATCTATCTGGCC
CTGGCCCACATGATCAAGTTCCGGGGCCACTTCCTGATCGAGGGCGACC
TGAACCCCGACAACAGCGACGTGGACAAGCTGTTCATCCAGCTGGTGCA
GACCTACAACCAGCTGTTCGAGGAAAACCCCATCAACGCCAGCGGCGTG
GACGCCAAGGCCATCCTGTCTGCCAGACTGAGCAAGAGCAGACGGCTGG
AAAATCTGATCGCCCAGCTGCCCGGCGAGAAGAAGAATGGCCTGTTCGG
AAACCTGATTGCCCTGAGCCTGGGCCTGACCCCCAACTTCAAGAGCAAC
TTCGACCTGGCCGAGGATGCCAAACTGCAGCTGAGCAAGGACACCTACG
ACGACGACCTGGACAACCTGCTGGCCCAGATCGGCGACCAGTACGCCGA
CCTGTTTCTGGCCGCCAAGAACCTGTCCGACGCCATCCTGCTGAGCGAC
ATCCTGAGAGTGAACACCGAGATCACCAAGGCCCCCCTGAGCGCCTCTA
TGATCAAGAGATACGACGAGCACCACCAGGACCTGACCCTGCTGAAAGC
TCTCGTGCGGCAGCAGCTGCCTGAGAAGTACAAAGAGATTTTCTTCGAC
CAGAGCAAGAACGGCTACGCCGGCTACATTGACGGCGGAGCCAGCCAGG
AAGAGTTCTACAAGTTCATCAAGCCCATCCTGGAAAAGATGGACGGCAC
CGAGGAACTGCTCGTGAAGCTGAACAGAGAGGACCTGCTGCGGAAGCAG
CGGACCTTCGACAACGGCAGCATCCCCCACCAGATCCACCTGGGAGAGC
TGCACGCCATTCTGCGGCGGCAGGAAGATTTTTACCCATTCCTGAAGGA
CAACCGGGAAAAGATCGAGAAGATCCTGACCTTCCGCATCCCCTACTAC
GTGGGCCCTCTGGCCAGGGGAAACAGCAGATTCGCCTGGATGACCAGAA
AGAGCGAGGAAACCATCACCCCCTGGAACTTCGAGGAAGTGGTGGACAA
GGGCGCTTCCGCCCAGAGCTTCATCGAGCGGATGACCAACTTCGATAAG
AACCTGCCCAACGAGAAGGTGCTGCCCAAGCACAGCCTGCTGTACGAGT
ACTTCACCGTGTATAACGAGCTGACCAAAGTGAAATACGTGACCGAGGG
AATGAGAAAGCCCGCCTTCCTGAGCGGCGAGCAGAAAAAGGCCATCGTG
GACCTGCTGTTCAAGACCAACCGGAAAGTGACCGTGAAGCAGCTGAAAG
AGGACTACTTCAAGAAAATCGAGTGCTTCGACTCCGTGGAAATCTCCGG
CGTGGAAGATCGGTTCAACGCCTCCCTGGGCACATACCACGATCTGCTG
AAAATTATCAAGGACAAGGACTTCCTGGACAATGAGGAAAACGAGGACA
TTCTGGAAGATATCGTGCTGACCCTGACACTGTTTGAGGACAGAGAGAT
GATCGAGGAACGGCTGAAAACCTATGCCCACCTGTTCGACGACAAAGTG
ATGAAGCAGCTGAAGCGGCGGAGATACACCGGCTGGGGCAGGCTGAGCC
GGAAGCTGATCAACGGCATCCGGGACAAGCAGTCCGGCAAGACAATCCT
GGATTTCCTGAAGTCCGACGGCTTCGCCAACAGAAACTTCATGCAGCTG
ATCCACGACGACAGCCTGACCTTTAAAGAGGACATCCAGAAAGCCCAGG
TGTCCGGCCAGGGCGATAGCCTGCACGAGCACATTGCCAATCTGGCCGG
CAGCCCCGCCATTAAGAAGGGCATCCTGCAGACAGTGAAGGTGGTGGAC
GAGCTCGTGAAAGTGATGGGCCGGCACAAGCCCGAGAACATCGTGATCG
AAATGGCCAGAGAGAACCAGACCACCCAGAAGGGACAGAAGAACAGCCG
CGAGAGAATGAAGCGGATCGAAGAGGGCATCAAAGAGCTGGGCAGCCAG
ATCCTGAAAGAACACCCCGTGGAAAACACCCAGCTGCAGAACGAGAAGC
TGTACCTGTACTACCTGCAGAATGGGCGGGATATGTACGTGGACCAGGA
ACTGGACATCAACCGGCTGTCCGACTACGATGTGGACGCCATCGTGCCT
CAGAGCTTTCTGAAGGACGACTCCATCGACAACAAGGTGCTGACCAGAA
GCGACAAGAACCGGGGCAAGAGCGACAACGTGCCCTCCGAAGAGGTCGT
GAAGAAGATGAAGAACTACTGGCGGCAGCTGCTGAACGCCAAGCTGATT
ACCCAGAGAAAGTTCGACAATCTGACCAAGGCCGAGAGAGGCGGCCTGA
GCGAACTGGATAAGGCCGGCTTCATCAAGAGACAGCTGGTGGAAACCCG
GCAGATCACAAAGCACGTGGCACAGATCCTGGACTCCCGGATGAACACT
AAGTACGACGAGAATGACAAGCTGATCCGGGAAGTGAAAGTGATCACCC
TGAAGTCCAAGCTGGTGTCCGATTTCCGGAAGGATTTCCAGTTTTACAA
AGTGCGCGAGATCAACAACTACCACCACGCCCACGACGCCTACCTGAAC
GCCGTCGTGGGAACCGCCCTGATCAAAAAGTACCCTAAGCTGGAAAGCG
AGTTCGTGTACGGCGACTACAAGGTGTACGACGTGCGGAAGATGATCGC
CAAGAGCGAGCAGGAAATCGGCAAGGCTACCGCCAAGTACTTCTTCTAC
AGCAACATCATGAACTTTTTCAAGACCGAGATTACCCTGGCCAACGGCG
AGATCCGGAAGCGGCCTCTGATCGAGACAAACGGCGAAACCGGGGAGAT
CGTGTGGGATAAGGGCCGGGATTTTGCCACCGTGCGGAAAGTGCTGAGC
ATGCCCCAAGTGAATATCGTGAAAAAGACCGAGGTGCAGACAGGCGGCT
TCAGCAAAGAGTCTATCCTGCCCAAGAGGAACAGCGATAAGCTGATCGC
CAGAAAGAAGGACTGGGACCCTAAGAAGTACGGCGGCTTCGACAGCCCC
ACCGTGGCCTATTCTGTGCTGGTGGTGGCCAAAGTGGAAAAGGGCAAGT
CCAAGAAACTGAAGAGTGTGAAAGAGCTGCTGGGGATCACCATCATGGA
AAGAAGCAGCTTCGAGAAGAATCCCATCGACTTTCTGGAAGCCAAGGGC
TACAAAGAAGTGAAAAAGGACCTGATCATCAAGCTGCCTAAGTACTCCC
TGTTCGAGCTGGAAAACGGCCGGAAGAGAATGCTGGCCTCTGCCGGCGA
ACTGCAGAAGGGAAACGAACTGGCCCTGCCCTCCAAATATGTGAACTTC
CTGTACCTGGCCAGCCACTATGAGAAGCTGAAGGGCTCCCCCGAGGATA
ATGAGCAGAAACAGCTGTTTGTGGAACAGCACAAGCACTACCTGGACGA
GATCATCGAGCAGATCAGCGAGTTCTCCAAGAGAGTGATCCTGGCCGAC
GCTAATCTGGACAAAGTGCTGTCCGCCTACAACAAGCACCGGGATAAGC
CCATCAGAGAGCAGGCCGAGAATATCATCCACCTGTTTACCCTGACCAA
TCTGGGAGCCCCTGCCGCCTTCAAGTACTTTGACACCACCATCGACCGG
AAGAGGTACACCAGCACCAAAGAGGTGCTGGACGCCACCCTGATCCACC
AGAGCATCACCGGCCTGTACGAGACACGGATCGACCTGTCTCAGCTGGG
AGGCGACAAAAGGCCGGCGGCCACGAAAAAGGCCGGCCAGGCAAAAAAG
AAAAAGGGCGGTGAGGGCAGAGGAAGTCTGCTAACATGCGGTGACGTCG
AGGAGAATCCTGGCCCAATGGTGAGCAAGGGCGAGGAGGATAACATGGC
CATCATCAAGGAGTTCATGCGCTTCAAGGTGCACATGGAGGGCTCCGTG
AACGGCCACGAGTTCGAGATCGAGGGCGAGGGCGAGGGCCGCCCCTACG
AGGGCACCCAGACCGCCAAGCTGAAGGTGACCAAGGGTGGCCCCCTGCC
CTTCGCCTGGGACATCCTGTCCCCTCAGTTCATGTACGGCTCCAAGGCC
TACGTGAAGCACCCCGCCGACATCCCCGACTACTTGAAGCTGTCCTTCC
CCGAGGGCTTCAAGTGGGAGCGCGTGATGAACTTCGAGGACGGCGGCGT
GGTGACCGTGACCCAGGACTCCTCCCTGCAGGACGGCGAGTTCATCTAC
AAGGTGAAGCTGCGCGGCACCAACTTCCCCTCCGACGGCCCCGTAATGC
AGAAGAAGACCATGGGCTGGGAGGCCTCCTCCGAGCGGATGTACCCCGA
GGACGGCGCCCTGAAGGGCGAGATCAAGCAGAGGCTGAAGCTGAAGGAC
GGCGGCCACTACGACGCTGAGGTCAAGACCACCTACAAGGCCAAGAAGC
CCGTGCAGCTGCCCGGCGCCTACAACGTCAACATCAAGTTGGACATCAC
CTCCCACAACGAGGACTACACCATCGTGGAACAGTACGAACGCGCCGAG
GGCCGCCACTCCACCGGCGGCATGGACGAGCTGTACAAGTAG

In another embodiment, there is provided herein a composition comprising one or more vectors expressing any of the oligonucleotide or oligonucleotides as described herein, a dead Cas9 (dCas9), or both.

In another embodiment, there is provided herein a composition comprising any one or more of the oligonucleotide or oligonucleotides as described herein, and a dead Cas9 (dCas9) or mRNA encoding a dCas9; or one or more plasmids or vectors encoding any one or more of the oligonucleotide or oligonucleotides as described herein, and a dead Cas9 (dCas9) or mRNA encoding a dCas9.

As will be known to one of skill in the art, nucleotide sequences for expressing a particular sequence (nucleic acid, protein, or both) may encode or include features as described in “Genes VII”, Lewin, B. Oxford University Press (2000) or “Molecular Cloning: A Laboratory Manual”, Sambrook et al., Cold Spring Harbour Laboratory, 3^{rd} Edition (2001). A nucleotide sequence encoding a particular oligonucleotide sequence and/or protein may be incorporated in a suitable vector, such as a commercially available vector. Vectors may be individually constructed or modified using standard molecular biology techniques, as outlined, for example, in Sambrook et al., Cold Spring Harbour Laboratory, 3^{rd} Edition (2001). The person of skill in the art will recognize that a vector may include nucleotide sequences encoding desired elements that may be operably linked to a nucleotide sequence encoding an oligonucleotide or amino acid sequence of interest. Such nucleotide sequences encoding desired elements may include transcriptional promoters, transcriptional enhancers, transcriptional terminators, translational initiators, translational terminators, ribosome binding sites, 5′-untranslated region, 3′-untranslated region, cap structure, poly A tail, and/or an origin of replication. Selection of a suitable vector may depend upon several factors, including, without limitation, the size of the nucleic acid to be incorporated into the vector, the type of transcriptional and translational control elements desired, the level of expression desired, copy number desired, whether chromosomal integration is desired, the type of selection process that is desired, or the host cell or host range that is intended to be transformed.

As will be understood, a vector may comprise any suitable nucleic acid construct configured for expressing an oligonucleotide or protein of interest in a cell. In certain embodiments, vectors may include a suitable plasmid, vector, or expression cassette, for example.

Several oligonucleotide sequences are provided herein. It will be understood that in addition to the sequences provided herein, oligonucleotides and nucleic acids comprising sequences complementary or partially complementary to the sequences provided herein are also contemplated. It will also be understood that double-stranded forms of single-stranded sequences are contemplated, and vice versa. DNA versions of RNA sequences provided herein are contemplated, and vice versa. For example, where a given single-stranded RNA sequence is provided herein, the skilled person will recognize that various other related oligonucleotides or nucleic acids are also provided such as a double-stranded DNA plasmid, vector, or expression cassette encoding or capable of expressing the single-stranded RNA sequence. Further, sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with any of the sequences provided herein are also contemplated.

In another embodiment, there is provided herein a composition comprising any one or more of:
an oligonucleotide as described herein;
a plasmid or vector as described herein;
a pharmaceutically acceptable carrier, excipient, diluent, or buffer;
a dead Cas9 (dCas9); or
an oligonucleotide, plasmid, or vector encoding a dead Cas9 (dCas9).

In certain embodiments, the dCas9 may comprise D1OA and H840A mutations. In certain embodiments, dCas9 may comprise any suitable catalytically inactive Cas9, which may be accomplished by introducing one or more point mutations or other changes, such that the Cas9 is unable to cleave dsDNA but retains the ability to target DNA.

In another embodiment, there is provided herein a method for targeted demethylation and/or activation of a gene, said method comprising:
introducing a dead Cas9 (dCas9) and one or more oligonucleotides into a cell, the one or more oligonucleotides each comprising:
   a targeting portion having sequence complementarity and binding affinity with a region of genomic DNA within the gene, near the gene, or both; and
   a single guide RNA (sgRNA) scaffold portion, wherein a tetra-loop portion of the sgRNA is modified and comprises an R2 stem loop of DNMT1-interacting RNA (DiR), and wherein a stem loop 2 portion of the sgRNA is modified and comprises an R5 step loop of DiR;
thereby demethylating and/or activating the gene by inhibiting DNA methyltransferase 1 (DNMT1) activity on the gene.

As will be understood, in certain embodiments, demethylation may comprise a reduction in methylation level of the gene, either globally across the gene or at one or more region(s) at or near the site(s) targeted by the targeting portion of the one or more oligonucleotides. In certain embodiments, activation of a gene may comprise an increase in expression level of the gene, either in terms of transcription, translation, or both.

In certain embodiments, it is contemplated that CRISPR-DiR as described herein may be used to demethylate generally any targeted region of interest, whether part of a coding gene or not. Experiments have been performed to look at regions near p16 (2.5 Kb upstream of p16 transcription start site (TSS), all the way to 1.2 KB downstream of p16 TSS). It was found that each region may be demethylated once targeted by CRISPR-DiR. For example, Region A (2.5 Kb upstream of p16 TSS) may be targeted, or Region E (1.2 Kb downstream of p16 TSS) may be targeted, and demethylated. Accordingly, it is contemplated that in certain embodiments a region targeted for demethylation may, or may not, be selected to provide for gene activation, and that in certain embodiments it may be of interest to target and demethylate a region of genomic DNA unrelated to a gene or gene expression for investigational purposes and/or to provide a different effect, for example.

In certain embodiments, the step of introducing may comprise providing the cell with a dead Cas9 and the one or more oligonucleotides. In certain embodiments, the cell may be treated with the dCas9 and the one or more oligonucleotides via, for example, transfection or via cellular delivery with a delivery vehicle. In certain embodiments, the one or more oligonucleotides, the dCas9, or both, may be expressed within the cell via transfection or introduction into the cell of an expression vector or plasmid encoding and expressing the one or more oligonucleotides, the dCas9, or both. In certain embodiments, the dCas9 may be expressed in the cell from an introduced vector, may be introduced into the cell as a protein (for example, via delivery into the cell with a delivery vehicle), or expressed in the cell from an introduced mRNA, for example. In certain embodiments, the one or more oligonucleotides may be expressed in the cell via transcription from a vector or plasmid encoding the one or more oligonucleotides, or the one or more oligonucleotides may be introduced into the cell via transfection with a delivery vehicle, for example. In certain embodiments, oligonucleotide and dCas9 may be introduced by transient transfection of plasmids, or by using lentivirus to make stable cell lines, for example. In certain embodiments, precomplexed CRISPR-DiR guide may be prepared as an oligonucleotide-dCas9 RNP complex and delivered to the cell using a delivery approach such as nanopore particles, Extracellular Vesicles (EVs), or Red Blood Cell Extracellular Vesicles (RBCEVs), for example.

As will be understood, in certain embodiments, inhibiting DNA methyltransferase 1 (DNMT1) activity may comprise reducing DNMT1 methylating activity affecting the gene, either globally across the gene or at one or more region(s) at or near the site(s) targeted by the targeting portion of the one or more oligonucleotides. Reducing DNMT1 methylating activity may include reducing or preventing methylation maintenance activity of the DNMT1, such that over time the gene may become demethylated and/or activated.

In certain embodiments, the targeting portion of at least one of the one or more oligonucleotides may have sequence complementarity and binding affinity with a non-template strand of the genomic DNA within the gene, near the gene, or both.

In certain embodiments, the step of introducing may comprise transfecting, delivering, or expressing the one or more oligonucleotides and the dCas9 in the cell. In certain embodiments, the one or more oligonucleotides may comprise one or more of the oligonucleotides described in detail herein.

In certain embodiments, the cell may be exposed to the dCas9 and the one or more oligonucleotides for a period of at least about 3 days, at least about 4 days, at least about 5 days, at least about 6 days, at least about 7 days, or at least about 8 days. In certain embodiments, the cell may be exposed to the dCas9 and the one or more oligonucleotides for a period of about 3 days to about a week, or any duration falling therebetween, for example.

With consecutive stable line, cells were traced for 53 days and gradually increased demethylation and gene expression was observed. The demethylation was initiated around Day 4-6 and significantly demethylated after 13 days. The gene expression was also initiated early in the first week, but clear and stable gene activation occurred at least one week to 13 days, or longer if the chromatin structure was highly closed. With dCas9 inducible system, if CRISPR-DiR treatment was induced for 3 days or 8 days, gradually increased level of gene demethylation as well as expression was observed, also initiated within the first week but becoming clear and stable after one week. In the inducible system, it was observed that the demethylation and gene activation effect may be maintained for at least one month, with only 3 days or 8 days induction.

In certain embodiments, demethylation of the targeted region may be initiated around day 4-6, and may be gradually increased with time, and robust gene activation may be generally detected at about one week or more, and expression level may gradually increase with longer treatment time, while protein restoration may occur with longer treatment. In the inducible system, both gene demethylation and activation may be maintained for at least one month, particularly if treatment was induced for 8 days following by turning the CRISPR-DiR treatment off.

In another embodiment, there is provided herein a use of any of the oligonucleotide or oligonucleotides, the plasmid(s) or vector(s), or the composition(s) as described herein, for targeted demethylation and/or activation of a gene.

In another embodiment, there is provided herein a method for treating a disease or disorder associated with decreased expression of at least one gene due to aberrant DNA methylation in a subject in need thereof, said method comprising:
treating the subject with a dead Cas9 (dCas9) and one or more oligonucleotides, the one or more oligonucleotides each comprising:
   a targeting portion having sequence complementarity and binding affinity with a region of genomic DNA within the gene, near the gene, or both; and
   a single guide RNA (sgRNA) scaffold portion, wherein a tetra-loop portion of the sgRNA is modified and comprises an R2 stem loop of DNMT1-interacting RNA (DiR), and wherein a stem loop 2 portion of the sgRNA is modified and comprises an R5 step loop of DiR;
      thereby demethylating and/or activating the gene by inhibiting DNA methyltransferase 1 (DNMT1) activity on the gene, and treating the disease or disorder.

In certain embodiments, the disease or disorder may comprise a disease or disorder associated with decreased expression of at least one gene due to aberrant DNA methylation in a subject in need thereof. By way of example, in certain embodiments, the disease or disorder may comprise a cancer. In certain embodiments, the cancer may comprise a cancer characterized by hypermethylation or other methylation-related deactivation of one or more tumor suppressor genes such that the one or more tumor suppressor genes are not expressed, or are expressed at low or insufficient levels. In certain embodiments, the disease or disorder may comprise an imprinting disease or genetic disease such as X fragile syndrome. In certain embodiments, the disease or disorder may comprise a cancer which may be MDS, breast cancer, melanoma, prostate cancer, colon cancer, or another disease triggered by aberrant DNA methylation. In certain embodiments, tumor suppressor genes may be targeted for activation, which may include DAPK1, CEBPA, CADHERIN 1, P15, or P16, for example. For P16, this gene is frequently hypermethylated and silenced in almost all kinds of tumors such as melanoma, prostate cancer, liver cancer, and colon cancer, and therefore it in contemplated that P16 may be targeted and/or that melanoma, prostate cancer, liver cancer, and/or colon cancer may be treated in certain examples.

In certain embodiments, the step of treating the subject may comprise administering a dead Cas9 and the one or more oligonucleotides to the subject, or expressing the dead Cas9 and the one or more oligonucleotides in the subject. such that the dCas9 and the one or more oligonucleotides are able to access the genomic DNA of one or more cells of the subject, particularly one or more cells of the subject related to the disease or disorder to be treated. In certain embodiments, the subject may be treated with the dCas9 and the one or more oligonucleotides via, for example, transfection or via cellular delivery with a delivery vehicle. In certain embodiments, the one or more oligonucleotides, the dCas9, or both, may be expressed within one or more cells of the subject via transfection or introduction into the one or more cells of an expression vector or plasmid encoding and expressing the one or more oligonucleotides, the dCas9, or both. In certain embodiments, the dCas9 may be expressed in the one or more cells from an introduced vector, may be introduced into the one or more cells as a protein (for example, via delivery into the cell with a delivery vehicle), or expressed in the one or more cells from an introduced mRNA, for example. In certain embodiments, the one or more oligonucleotides may be expressed in the one or more cells via transcription from a vector or plasmid encoding the one or more oligonucleotides, or the one or more oligonucleotides may be introduced into the one or more cells via transfection with a delivery vehicle, for example. In certain embodiments, the treatment may be administered to the subject systemically, or locally, or both. In certain embodiments, the step of treating may comprise transfecting, delivering, or expressing the one or more oligonucleotides and the dCas9 in at least one cell of the subject In certain embodiments, the targeting portion of at least one of the one or more oligonucleotides may have sequence complementarity and binding affinity with a non-template strand of the genomic DNA within the gene, near the gene, or both.

In certain embodiments, the one or more oligonucleotides may comprise one or more oligonucleotides as described herein.

In another embodiment, the subject may be exposed to the dCas9 and the one or more oligonucleotides for a period of at least about 3 days, at least about 4 days, at least about 5 days, at least about 6 days, at least about 7 days, or at least about 8 days. In certain embodiments, the subject may be exposed to the dCas9 and the one or more oligonucleotides for a period of about 3 days to about a week, or any duration falling therebetween, for example.

With consecutive stable line, cells were traced for 53 days and gradually increased demethylation and gene expression was observed. The demethylation was initiated around Day 4-6 and significantly demethylated after 8-13 days. The gene expression was also initiated early in the first week, but clear and stable gene activation occurred at least one week to 13 days, or longer if the chromatin structure was highly closed. With dCas9 inducible system, if CRISPR-DiR treatment was induced for 3 days or 8 days, gradually increased level of gene demethylation as well as expression was observed, also initiated within the first week but becoming clear and stable after one week. In the inducible system, it was observed that the demethylation and gene activation effect may be maintained for at least one month, with only 3 days or 8 days induction.

In another embodiment, there is provided herein a use of any of the oligonucleotide or oligonucleotides as described herein, the plasmid(s) or vector(s) as described herein, or the composition(s) as described herein, for treating a disease or disorder associated with decreased expression of at least one gene due to aberrant DNA methylation in a subject in need thereof.

In certain embodiments, the targeting portion of at least one of the one or more oligonucleotides may target a site within or near a promoter region of the gene. In certain embodiments, the promoter region may comprise a CpG-rich region having at least some methylation.

In certain embodiments, the disease or disorder may comprise cancer. In another embodiment, the targeting portion of at least one of the one or more oligonucleotides may target a site within or near a promoter region of the gene, wherein the gene may be a tumor suppressor gene. In yet another embodiment, the promoter region may comprise a CpG-rich region having at least some methylation. In still another embodiment, the targeting portion of at least one of the one or the more oligonucleotides may target the D1 or D3 region of the P16 gene. In still another embodiment, the one or more oligonucleotides may comprise at least one oligonucleotide with a targeting portion targeting the D1 region, and at least one oligonucleotide with a targeting portion targeting the D3 region, and may optionally further comprise at least one oligonucleotide with a targeting portion targeting the D2 region.

Region D1 may be understood as the proximal promoter region (200 bp upstream of p16 transcription start site), or may be considered as a 5′ portion of the first exon, GRCh38/hg38, chr9: 21975134-21975333.

Region D2 may be understood as falling within p16 first exon, in the middle of region D1 and D3, GRCh38/hg38, chr9: 21974812-21975008.

Region D3 may be understood as the region at the end of the first exon and beginning of first intron, or may be considered as a 3′portion of the first exon, GRCh38/hg38, chr9: 21974284-21974811. In another embodiment, the one or more one or more oligonucleotides may comprise any one or more of:

G19sgR2R5 (SEQ ID NO: 1):
GCUCCCCCGCCUGCCAGCAAGUUUGAGAGCUACCCGGGACGCGGGUCCG
GGACAGUAGCAAGUUCAAAUAAGGCUAGUCCGUUAUCAACUUCUGAGGC
CUUGGCGAGGCUUCUAAGUGGCACCGAGUCGGUGCUUUUUU;
G36sgR2R5 (SEQ ID NO: 2):
GCUAACUGCCAAAUUGAAUCGGUUUGAGAGCUACCCGGGACGCGGGUCC
GGGACAGUAGCAAGUUCAAAUAAGGCUAGUCCGUUAUCAACUUCUGAGG
CCUUGGCGAGGCUUCUAAGUGGCACCGAGUCGGUGCUUUUUU;
G110sgR2R5 (SEQ ID NO: 3):
GACCCUCUACCCACCUGGAUGUUUGAGAGCUACCCGGGACGCGGGUCCG
GGACAGUAGCAAGUUCAAAUAAGGCUAGUCCGUUAUCAACUUCUGAGGC
CUUGGCGAGGCUUCUAAGUGGCACCGAGUCGGUGCUUUUUU;
G111sgR2R5 (SEQ ID NO: 4):
GCCCCCAGGGCGUCGCCAGGGUUUGAGAGCUACCCGGGACGCGGGUCCG
GGACAGUAGCAAGUUCAAAUAAGGCUAGUCCGUUAUCAACUUCUGAGGC
CUUGGCGAGGCUUCUAAGUGGCACCGAGUCGGUGCUUUUUU;
G108sgR2R5 (SEQ ID NO: 5):
GUGGCCAGCCAGUCAGCCGAGUUUGAGAGCUACCCGGGACGCGGGUCCG
GGACAGUAGCAAGUUCAAAUAAGGCUAGUCCGUUAUCAACUUCUGAGGC
CUUGGCGAGGCUUCUAAGUGGCACCGAGUCGGUGCUUUUUU;
or
G122sgR2R5 (SEQ ID NO: 6):
GCCGCAGCCGCCGAGCGCACGGUUUGAGAGCUACCCGGGACGCGGGUCC
GGGACAGUAGCAAGUUCAAAUAAGGCUAGUCCGUUAUCAACUUCUGAGG
CCUUGGCGAGGCUUCUAAGUGGCACCGAGUCGGUGCUUUUUU;

or any combinations thereof.

In certain studies described herein, the following method was used for selecting a target site for CRISPR-DiR.

For both genes P16 and SALL4, cells were treated with non-specific demethylation agent Decitabine (2′-deoxy-5-azacytidine) for three days, and then Bisulfite Sanger-sequencing or Whole Genomic Bisulfite Sequencing was performed for both the wild-type cells and the Decitabine treated cells, to compare the most demethylated regions around the TSS of the targeting gene of interest. It was hypothesized that these most demethylated regions may be important regulatory regions of which the demethylation is correlated with gene activation. For P16, several highly demethylated regions were further picked and CRISPR-DiR used to target these regions either separately or simultaneously. Results indicated that though targeting a single region in p16 promoter or first exon can induce targeted demethylation and gene activation, simultaneous targeting the 5′ and 3′ of p16 first exon significantly and remarkably enhanced the gene expression (of note, the 5′ and 3′ regions of p16 first exon (region D1 and D3) are also highly demethylated regions screened by Decitabine treatment). For SALL4, targeting one highly demethylated region within first exon was tested. A similar targeting rule (i.e. targeting 5′ and 3′ of first exon) in P15 gene locus is also suspected based on preliminary data using another system.

In certain embodiments, target site selection may involve treating cells with Decitabine (2′-deoxy-5-azacytidine), or another such agent, first and then determining a few highly demethylated regions in an important regulatory region (e.g. Promoter, CpG island, first exon, first intron), and then exploring the targeting of these region(s). Without wishing to be bound by theory, it is hypothesized that a) demethylation of first exon may be important for gene activation, thus targeting both sides of first exon may make the demethylation more efficient and spread to the middle region to enhance the demethylation of the entire first exon; and/or b) targeting both sides around an important regulatory region where important transcription factors or even distal enhancers bind may be desirable in certain embodiments; and/or c) both region D1 and D3 may be important regulatory regions with important transcription factor bindings; and/or d) directly targeting the most important regulatory regions may be desirable; and/or e) demethylation of promoter CpG island may be important for transcription initiation, while demethylation of the first exon-intron junction may be important for splicing, therefore simultaneous targeting of these two regions may further enhance gene activation.

Because of the typically unfavorable prognosis and lack of therapeutic options in hepatocellular carcinoma (HCC), as well as the important role of P16 in regulating cell cycle, certain of the studies described in the Examples below used p16 in human HCC cell line SNU-398 to develop gene-specific demethylation and activation tools as described herein. In the CRISPR-DiR system, the DiR loops may be delivered to p16 locus specifically through designing p16 sgRNA guides, and in certain embodiments may mimic the endogenous DNMT1-RNA interaction to block DNMT1 methyltransferase activity, thereby reactivating p¹⁶ in a more natural process so as to restore gene expression to a more natural level. Thus, provided herein are CRISPR-DiR systems for gene-specific demethylation and/or gene activation.

During transcription, RNA Pol II binds to the antisense strand (AS), uses the antisense strand as the template strand (T) to synthesize an RNA transcript with complementary bases, which are the same as the sequence of the sense strand (S), also known as non-template strand (NT). The sense strand (S) is the DNA strand whose base sequence corresponds to the base sequence of the RNA transcript produced. Therefore, the sense strand (S) or non-template strand (NT) is in the same genomic orientation as the coding gene. When referring to single guide RNA (sgRNA) with a targeting portion targeting a certain DNA strand, it means the targeting portion of the sgRNA is fully or substantially complementary to the targeted strand. For example, for an sgRNA with a targeting portion targeting the sense strand (non-template strand) of P16 gene, the targeting portion of the sgRNA is complementary to the sense strand (non-template strand) of P16 gene, so the targeting portion sequence is substantially similar to or the same as the antisense strand (template strand).

As shown in the Examples below, fusing DNMT1-interacting RNA (DiR) short loops (R2 and R5) into CRISPR single guide RNA (sgRNA) may provide a strategy for demethylating a chosen target region and/or for restoring gene expression by repurposing CEBPA-DiRs to other specific gene loci of interest.

As described herein, endogenous DNMT1-interacting RNA loops from ecCEBPA may be repurposed to other gene locus (eg. p16, SALL4), which may provide an RNA-based approach for demethylation and/or activation and may in certain embodiments result in a) a more natural way to demethylate and activate genes; b) a more flexible way to modify the system; c) an RNA-based therapy for gene specific regulation; or any combinations thereof.

CRISPR-DiR systems as described herein may use RNA as gene-specific demethylating tool. There is much interest in using RNA molecules as a therapeutic tool, and this technology may provide for targeted therapy. It is contemplated that such an approach may offer advantages over existing hypomethylating-based protocols, such as: a) comparatively high gene specificity; b) comparatively lower cytotoxicity; and/or c) potential absence of certain drug-based off-target side-effects. The ability to control in loco gene expression may have particular interest in clinical applications. It is also contemplated that tools as described herein may be used to further understanding of the epigenetic regulation process and identification of key regulators as well as new targets for therapeutic treatments. In certain embodiments, it is contemplated that CRISPR-DiR systems as described herein may provide an RNA-based gene-specific demethylating tool for disease treatment, for example.

In certain embodiments, CRISPR-DiR systems as described herein may provide a CRISPR-based system for specific targeting genome-wide. In certain embodiments, it is contemplated that regulating gene loci in a specific and efficient manner may be provided, which may be less toxic than genome wide demethylation agents (5aza etc.), and/or may be applied to generally any region of interest in the human genome, even in heterochromatin regions in certain embodiments. Unlike 5aza or other CRISPR systems, it is contemplated that CRISPR-DiR systems as described herein may mimic the endogenous demethylation and epigenetic regulation process, and/or may demethylate and activate specific gene(s) in a more natural way in certain embodiments.

In another embodiment, there is provided herein a method for identifying one or more target sites for demethylation to activate expression of a gene in a cell, said method comprising:
treating the cell with a non-specific demethylation agent;
identifying one or more regions around the transcription start site of the gene which are most demethylated by treatment with the non-specific demethylating agent; and
using the identified one or more regions as target sites for demethylation to activate gene expression.

In another embodiment of the above method, the non-specific demethylation agent may comprise Decitabine (2′-deoxy-5-azacytidine), Azacitidine (5-Azacytidine), or another demethylating agent such as a second generation demethylating agent (see Agrawal et al., Nucleosidic DNA demethylating epigenetic drugs—A comprehensive review from discovery to clinic, Pharmacology & Therapeutics, 2018, 188:45-79, https://doi.org/10.1016/j.pharmthera 2018 02 006 herein incorporated by reference in its entirety), or any combinations thereof.

In yet another embodiment of any of the above method or methods, the treatment with the non-specific demethylation agent may be for about 3 days.

In another embodiment of any of the above method or methods, the step of identifying the one or more regions around the transcription start site of the gene which are most demethylated by treatment with the non-specific demethylating agent may comprise performing sequencing-based techniques, such as single locus genomic Bisulfite sequencing, reduced resolution bisulfite sequencing, whole genomic Bisulfite sequencing, AR, or array-based strategies such as the Infinium Methylation EPIC BeadChip, and, optionally, comparing results with a control untreated cell.

In another embodiment of any of the above method or methods, selection of the one or more regions around the transcription start site may favour selection of regions at or near the promoter, at or near the first exon of the gene, at or near a first intron of the gene, at or near a 5′ region of the first exon of the gene, at or near a 3′ region of the first exon of the gene, at or near a CpG island, at or near another important regulatory region, or any combinations thereof.

In still another embodiment of any of the above method or methods, selection of the one or more regions around the transcription start site may favour selection of at least one region at or near a 5′ region of the first exon of the gene, and at least one region at or near a 3′ region of the first exon of the gene.

In another embodiment of any of the above method or methods, the method may further comprise performing targeted demethylation and gene activation using any of the method or methods described herein employing CRISPR-DiR, wherein the targeting portions of the one or more oligonucleotides of the CRISPR-DiR system have sequence complementarity with the identified target sites for demethylation.

In another embodiment of any of the above method or methods, the one or more regions may be regions of the non-template strand.

As described in the following Examples, CRISPR-DiR systems targeting p16 and SALL4 have been transduced into HCC cell line SNU-398 and SNU-387, respectively, via lentivirus, and gene-specific demethylation and activation, as well as functional restoration, were successfully achieved in both genes in cellular level in the studies described below.

### Example 1—Initial CRISPR-DiR Studies with P16

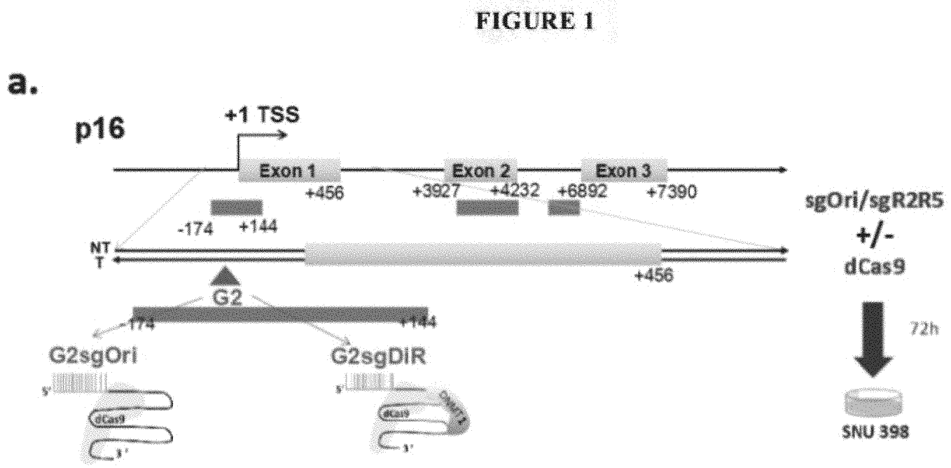

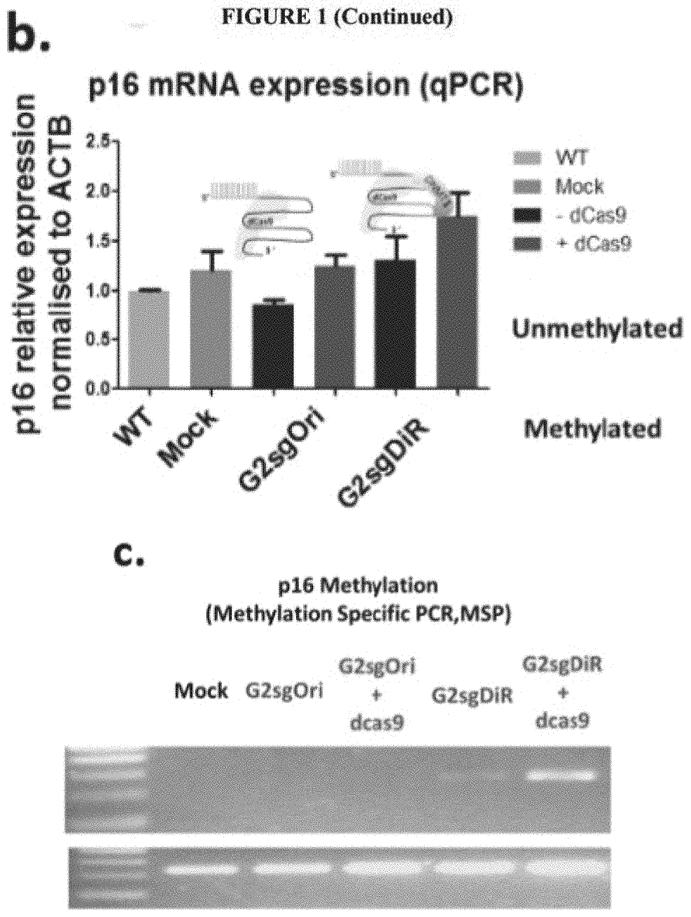

In this example, a modified CRISPR/dCAS9 system for gene activation and demethylation was developed and tested with P16. The DiR localized in CEBPA locus (ecCEBPA) is repurposed to other specific gene target(s) for demethylation and reactivation. The RNA stem-loops (R2 and R5), interacting with DNMT1(1), were fused to the tetra- and stem-loop 2 in a single guide RNA (sgRNA) scaffold to obtain a modified sgRNA (MsgRNA, sgDiR in FIG. 1, also referred to as MsgDiR6 in Example 3 below). The hepatocellular carcinoma (HCC) cell line SNU-398, in which P16 is silenced by promoter methylation, was transiently transfected with dCas9 μlasmid and one MsgRNA (using guide G2: GCACUCAAACACGCCUUUGC (SEQ ID NO: 29), MsgRNA with guide G2 is shown as G2sgDiR in FIG. 1, as targeting portion) targeting the template strand of P16 promoter.

Seventy-two hours after transfection, a two-fold increase of P16 mRNA was observed by qRT-PCR in the cell line treated with the MsgRNA (FIG. 1a). A loss of DNA methylation of the locus was also observed compared to cells transfected with the unmodified sgRNA, by Combined Bisulfite Restriction Analysis (COBRA) (FIG. 1b, c).

FIG. 1 shows that CRISPR-R2R5 system induced moderate gene activation and demethylation by targeting promoter CpG island. In FIG. 1(a), the structure of sgRNA (no DiR) and sgR2R5 (with DiR), the targeting site G2, and the transfection methods, are shown. In FIG. 1(b) the p16 mRNA expression in each sample after 72 hours treatment is shown. In FIG. 1(c), the MSP data showing the gene demethylation is shown. (Abbreviations—sgOri: Original sgRNA without DiR; sgR2R5: sgRNA fused with R2,R5 loops; G2: guide RNA; MSP: Methylation Specific PCR).

The oligonucleotide construct (MsgRNA) in this study had the following structure:

(SEQ ID NO: 15)
GCACUCAAACACGCCUUUGCGUUUUAGAGCUACCCGGGACGCGGGUCCG
GGACAGUAGCAAGUUAAAAUAAGGCUAGUCCGUUAUCAACUUCUGAGGC
CUUGGCGAGGCUUCUAAGUGGCACCGAGUCGGUGCUUUUUU

wherein plain underlined text indicates the targeting portion, plain text indicates the single guide RNA (sgRNA) scaffold portion, bold text indicates an R2 stem loop of DNMT1-interacting RNA (DiR) which has been incorporated at the tetra-loop portion of the sgRNA, and bold underlined text indicates an R5 stem loop of DNMT1-interacting RNA (DiR) which has been incorporated at the stem loop 2 portion of the sgRNA.

This design allowed incorporation of DNMT1-interacting RNA loops into sgRNA structure, while keeping the secondary structure of the MsgRNA similar to the original unmodified sgRNA (RNA secondary structure was predicted via RNAfold—http://rna.tbi.umivie.ac.at/cgi-binRNAWebSuite/RNAfold.cgi). Structure of typical single guide RNA (sgRNA), showing regions such as target, tetra-loop, and stem loop 2, are shown in FIG. 2.

This study transiently transfected dCas9 μlasmid together with one MsgRNA plasmid (G2sgDiR) to SNU398 cells, culturing three days without selection for the positively transfected cells. Guide G2 targets one site in the template strand (also known as antisense strand) of P16 promoter; and was initially chosen for two reasons: 1) the sequence is one of the three guides used in a P16 gene study (3), and 2) G2 targets the P16 promoter region, the methylation and demethylation of which has been considered as an important factor for gene regulation. In addition, among the three guides reported for P16 (3), guide G2 showed the lowest off-target effects as predicted by the online tool available at https://portals.broadinstitute.org/gpp/public/analysis-tools/sgrna-design, and may have the highest targeting efficiency since it worked in a SAM system (3).

These initial results achieved increase in P16 mRNA levels and decrease in DNA methylation of the locus as measured. While encouraging, the P16 demethylation and activation in this study may have been relatively modest. It was contemplated that technical limitations in this initial study when assessing P16 mRNA expression may have somewhat elevated P16 expression measurement. The qPCR primer set used to assess P16 reactivation were located within exon1, without spanning the exon junctions (Forward: CCCCTTGCCTGGAAAGATAC (SEQ ID NO: 16), Reverse: AGCCCCTCCTCTITCTTCCT (SEQ ID NO: 17)). Therefore, the measured two-fold increase of P16 mRNA measured may, at least in part, have included an increase of P16 exon1, but not necessarily of the entire P16 mRNA or alternative splicing variants, for example. Further, P16 protein activation was not detected in this study.

Accordingly, building from the results of this study, further investigation, development, and enhancement studies were performed in an effort to further improve this technology. Example 2 below details the results of these further developments.

### Example 2—CRISPR-DiR for Gene-Specific Demethylation and Activation

In this Example, sequence design used for the oligonucleotide constructs was enhanced over what was developed in Example 1 above, including further development of target region(s), guides, targeting strand, and sgRNA scaffold modifications to provide stability and/or transcriptional efficiency. In addition, the transient (72 hour) system of Example 1 was replaced with a stable system in which cells were selected and traced for up to 53 days. As discussed herein, improvements in stability and efficiency of the CRISPR-DiR system were observed, as well as significant enhancement of P16 demethylation and restoration (in terms of both mRNA expression and protein function).

The stable system used in this Example is informative for DNA methylation and dynamic epigenetic regulation, as DNA methylation changes occur and become evident when the cells cycle and the majority of the cells acquire a similar phenotype. As well, the stable system mimics and enables tracing of the natural epigenetic regulation process of DNA methylation, histone modifications, chromatin structures, etc. In the system of Example 1, changes were observed by MSP, but it was not determined how methylation pattern may remain, or change, after a week for example, nor the dynamic regulation process.

As well, in this Example, the region of the gene targeted by the targeting portion of the oligonucleotide construct was also investigated. Notably, as described below, it was found that CRISPR-DiR systems as described herein may provide for demethylation-based P16 gene activation through targeting and demethylation of not only the promoter region, but also the beginning of the first intron. P16 promoter is the major region which has been widely considered as the important region for gene regulation and being associated with aberrant methylation, and previous CRISPR-activation domain (VP64,VP16 etc.) systems typically suggest targeting in the proximal promoter regions. However, the presently described CRISPR-DiR systems may mimic natural gene regulation process, and also indicate here the importance of the first intron region in terms of DNA demethylation and gene restoration. The regulation function of the first intron region is still being explored, and it is contemplated that these results may guide or assist with design of CRISPR-DiR guides targeting other genes genome-wide.

As also described in this example, the CRISPR-DiR systems in this study showed a striking strand preference in which designing the targeting portion of the guide oligonucleotide construct to target the non-template strand of the genomic DNA provided specific gene demethylation and activation results notably better than those obtained when targeting the template strand of the genomic DNA in comparison studies.

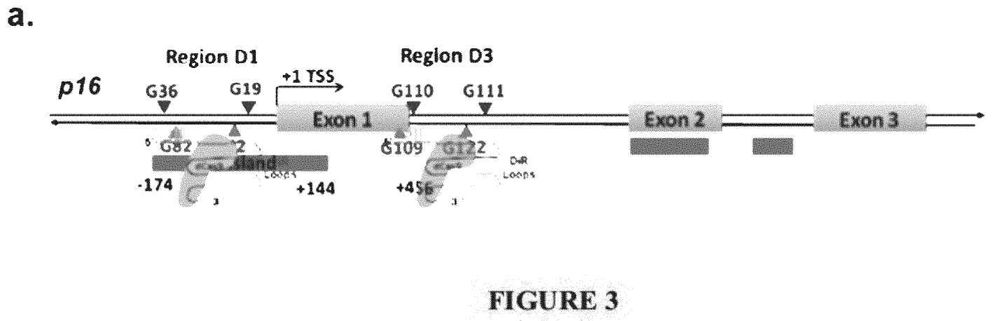

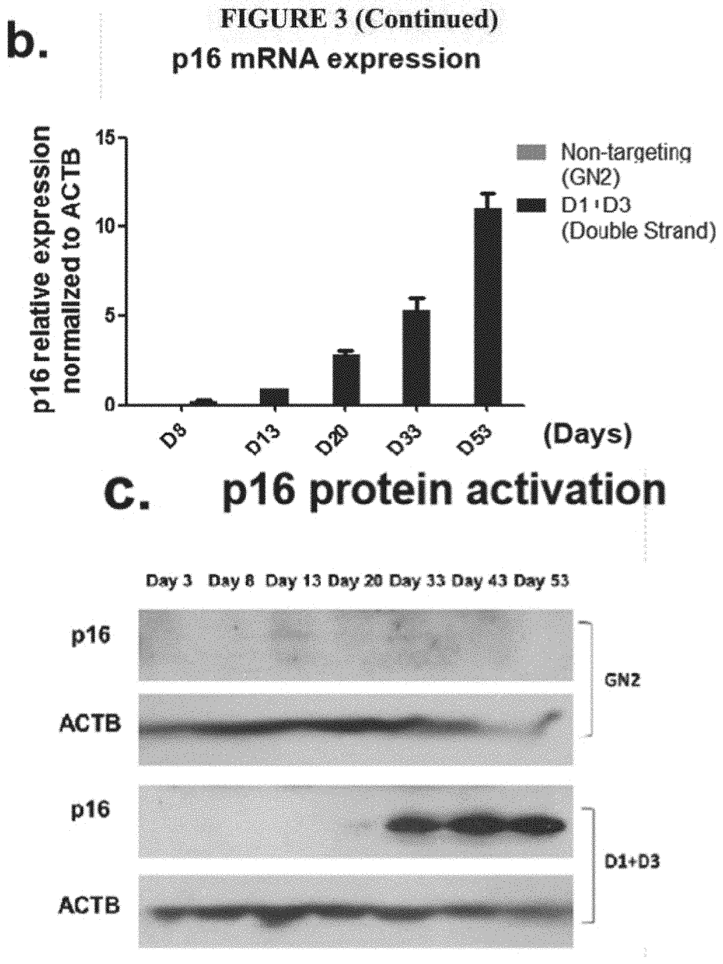

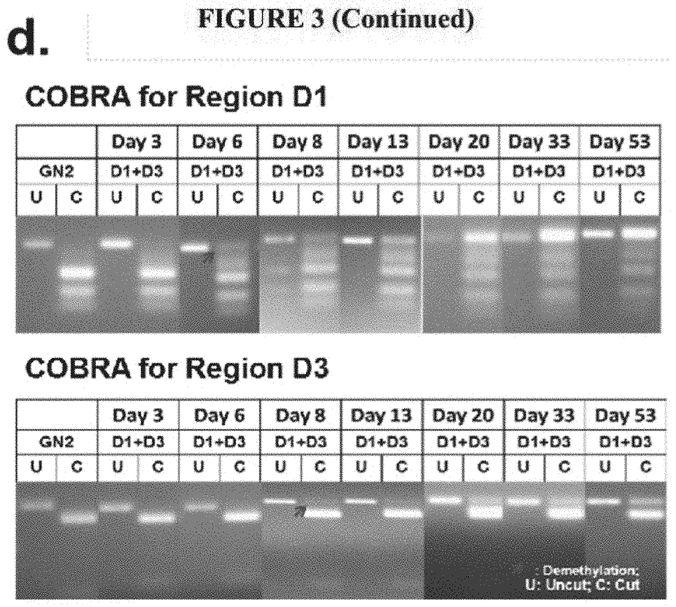

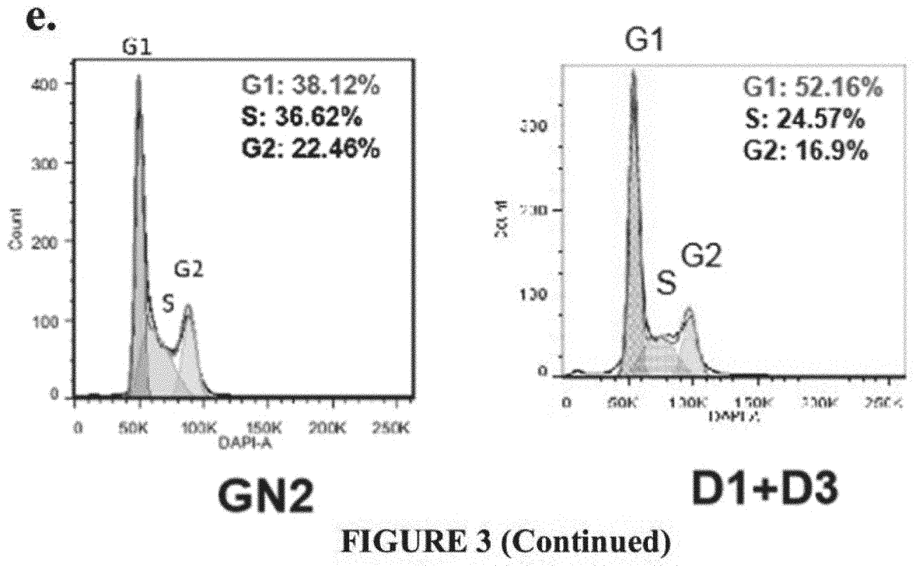

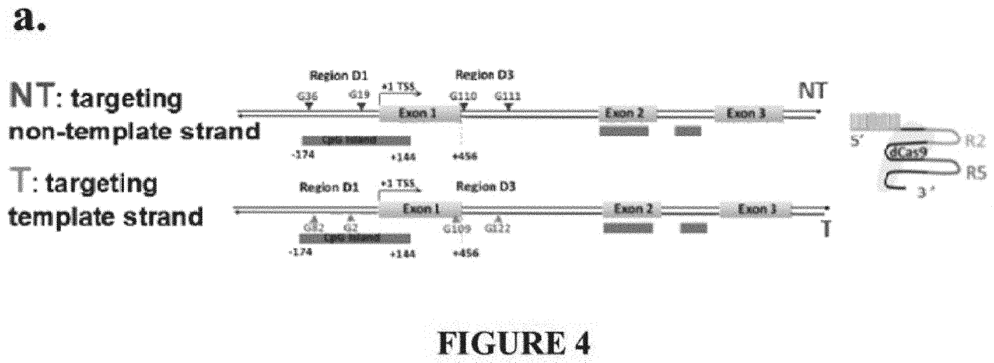

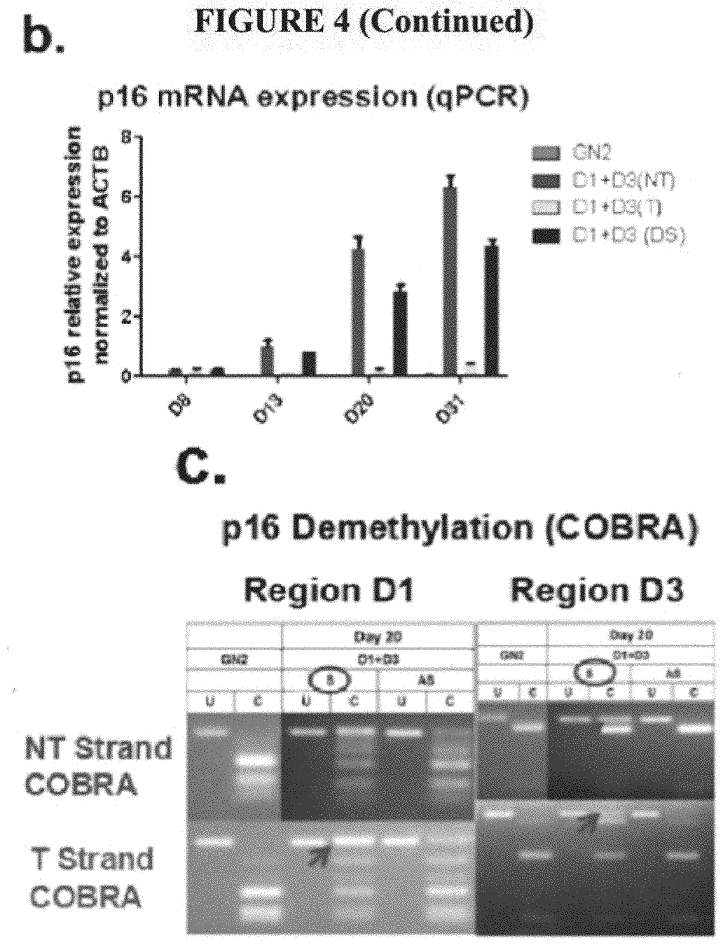

In the studies described herein, the qPCR primer set for measuring P16 mRNA levels was re-designed to span the exon junctions (Forward: CAACGCACCGAATAGTTACG (SEQ ID NO: 18), Reverse: AGCACCACCAGCGTGTC (SEQ ID NO: 19)), providing a better assessment of P16 mRNA levels. As shown in FIG. 3 and FIG. 4, and described in further detail below, the CRISPR-DiR system in this example, when targeting the non-template strand (also known as sense strand) of P16 regions D1 (promoter) and D3 (the beginning of intron 1), provided demethylated P16 targeted regions, and restored P16 mRNA as well as protein expression.

### Oligonucleotide Design:

Typical single guide RNA (sgRNA) sequence is as follows:

(G)nnnnnnnnnnnnnnnnnnnGUUUUAGAGCUAGAAAUAGCAAGUUAAAAUAAGGCUAGUCCGUU AUCAACUUGAAAAAGUGGCACCGAGUCGGUGCUUUUUU (Formula III)

and may include 4-6 Us (6 are shown) at the end for termination sequence for U6 promoter, (see also, FIG. 2) wherein the first 20 bases (plain underlined text) represent the targeting portion (also referred to as guide RNA) designed to be complementary to the targeted DNA strand (i.e. each “n” is selected such that the targeting portion is substantially or fully complementary to the target sequence); the next 76 bases represent the sgRNA scaffold portion, which is conserved in typical sgRNA with different guide RNA and is used for recruiting and forming complex with Cas9/dCas9 proteins, where the bold and bold underline text indicates nucleotides which were changed or replaced with R2 and R5 DiR loop sequence in CRISPR-DiR systems described herein; and the last 4 to 6 Uracils (UUUUUU) are termination signal for sgRNA transcription.

According to the crystal structure of sgRNA-Cas9/dCas9 complex (3, 4), the tetra-loop (GAAA, in bold, changed to R2 stem loop in CRISPR-DiR system) and stem-loop 2 (GAAAA, in bold underline, changed to R5 stem loop in CRISPR-DiR system) in the sgRNA scaffold protrude outside of the Cas9/dCas9-sgRNA ribonucleoprotein complex, with the distal 4 base pairs (bp) of each stem completely free of interactions with Cas9/dCas9 amino acid side chains, and were shown to be amenable to replacement with other RNA stem loops (eg. RNA aptamers MS2, PP7, boxB) (3, 5). The last 4 to 6 uracils (TTTTTT in black) are the termination signal for sgRNA transcription.

The construct used in Example 1 above had the structure:

(G)nnnnnnnnnnnnnnnnnnnGUUUUAGAGCUACCCGGGACGCGGGUCCGGGACAGUAGCAAGUU AAAAUAAGGCUAGUCCGUUAUCAACUUCUGAGGCCUUGGCGAGGCUUCUAAGUGGCACC GAGUCGGUGCUUUUUU (Formula IV)

wherein plain underlined text indicates the targeting portion (i.e. each “n” is selected such that the targeting portion is substantially or fully complementary to the target sequence), plain text indicates the single guide RNA (sgRNA) scaffold portion, bold text indicates an R2 stem loop of DNMT1-interacting RNA (DiR) which has been incorporated at the tetra-loop portion of the sgRNA, and bold underlined text indicates an R5 stem loop of DNMT1-interacting RNA (DiR) which has been incorporated at the stem loop 2 portion of the sgRNA.

In this study, it was recognized that the most popular promoter typically to transcribe single guide RNA is the U6 promoter, which is an optimal RNA Polymerase III (RNAPIII) promoters to produce short RNAs (shRNAs, sgRNAs, tRNAs, rRNA 5S, etc.). It allows expression of non-coding RNA molecules with precise 5′ and 3′ sequences (5′ starts with G and 3′ terminates with a series of T's (5-6) in a row). However, there is a putative POL-III terminator (4 consecutive T's) in the beginning of the typical sgRNA scaffold, and in the sgRNA scaffold used in Example 1, that may cause some premature termination, thus potentially reducing the efficiency. Few studies have tried to modify the sgRNA scaffold to increase their stability. One option may be to remove this putative POL-III terminator (4 consecutive U's) by replacing the fourth UT to A (6), or C (7), or G (7). U/T to C or UT to G replacement may work more efficiently than U/T to A replacement (7) in other systems. Thus, in this study, the fourth U/T (in bold, italic, underline below) was substituted with G, to make the structure more stable by enabling efficient transcription, while keeping substantially the same secondary structure and decreasing the minimum free energy (MFE). Accordingly, the corresponding A was substituted with C (in bold, italic below) to preserve base-pairing with the “G”.

This afforded the second generation oligonucleotide construct design used in these studies, as follows:

(G)nnnnnnnnnnnnnnnnnnnGUUUGAGAGCUACCCGGGACGCGGGUCCGGGACAGUAGCAAGUU CAAAUAAGGCUAGUCCGUUAUCAACUUCUGAGGCCUUGGCGAGGCUUCUAAGUGGCACCG AGUCGGUGCUUUUUU (Formula V)

wherein each “n” is selected such that the targeting portion is substantially or fully complementary to the target sequence of interest.

In this study, several guide RNAs (i.e. targeting portions) were designed to target different regions of P16 gene locus. Instead of using guide G2 to target the template strand of P16 promoter as in Example 1, the targeting portions were carefully designed in an effort to develop effective guides targeting both DNA strands. As described below, two guides (G19 and G36) to target the non-template strand of P16 promoter (Region D1), and two other guides (G110 and G111) to target the non-template strand of P16 Intron 1 (Region D3) were arrived at. Sequences are provided below. Methods for determining good targeting regions and targeting strand are provided in detail below.

G19sgR2R5 (SEQ ID NO: 1):
GCUCCCCCGCCUGCCAGCAAGUUUGAGAGCUACCCGGGACGCGGGUCCG
GGACAGUAGCAAGUUCAAAUAAGGCUAGUCCGUUAUCAACUUCUGAGGC
CUUGGCGAGGCUUCUAAGUGGCACCGAGUCGGUGCUUUUUU;
G36sgR2R5 (SEQ ID NO: 2):
GCUAACUGCCAAAUUGAAUCGGUUUGAGAGCUACCCGGGACGCGGGUCC
GGGACAGUAGCAAGUUCAAAUAAGGCUAGUCCGUUAUCAACUUCUGAGG
CCUUGGCGAGGCUUCUAAGUGGCACCGAGUCGGUGCUUUUUU;
G110sgR2R5 (SEQ ID NO: 3):
GACCCUCUACCCACCUGGAUGUUUGAGAGCUACCCGGGACGCGGGUCCG
GGACAGUAGCAAGUUCAAAUAAGGCUAGUCCGUUAUCAACUUCUGAGGC
CUUGGCGAGGCUUCUAAGUGGCACCGAGUCGGUGCUUUUUU;
G111sgR2R5 (SEQ ID NO: 4):
GCCCCCAGGGCGUCGCCAGGGUUUGAGAGCUACCCGGGACGCGGGUCCG
GGACAGUAGCAAGUUCAAAUAAGGCUAGUCCGUUAUCAACUUCUGAGGC
CUUGGCGAGGCUUCUAAGUGGCACCGAGUCGGUGCUUUUUU;

All other guides (targeting portion in sgDiR) in this example share the same sgDiR scaffold, which is the same as above:

[Guide]GUUUGAGAGCUACCCGGGACGCGGGUCCGGGACAGUAGCAAGUUCAAAUAAGGCU AGUCCGUUAUCAACUUCUGAGGCCUUGGCGAGGCUUCUAAGUGGCACCGAGUCGGUGCUU UUUU (Formula VI)

A list of the other guide sequences is as follows:

| Guide | Targeting Region | Target Strand | Sequence |
|---|---|---|---|
| GN2 | Non-targeting | | GUUAGGAAUAAAAGCUUUGA |
| | | | (SEQ ID NO: 20) |
| G100 | Region A | Template Strand (anti- | GUGAACCGAGAGAGAUCGUG |
| | | sense) | (SEQ ID NO: 21) |
| G101 | | Template Strand (anti- | GCCCCCAUUAAGAACCACUGU |
| | | sense) | (SEQ ID NO: 22) |
| G102 | | Non-template Strand | GGUUGCCAGGAUGGGAGGGA |
| | | (sense) | (SEQ ID NO: 23) |
| G103 | | Non-template Strand | GUUCUUCUCAAAAAAGAAAGU |
| | | (sense) | (SEQ ID NO: 24) |
| G25 | Region C | Template Strand (anti- | GACAGGACAGUAUUUGAAGC |
| | | sense) | (SEQ ID NO: 25) |
| G126 | | Non-template Strand | GGUUUAUUUAAUACGGACGG |
| | | (sense) | (SEQ ID NO: 26) |
| G127 | | Template Strand (anti- | GACAGCCGUUUUACACGCAGG |
| | | sense) | (SEQ ID NO: 27) |
| G128 | | Template Strand (anti- | GCAGGUGAUUUCGAUUCUCGG |
| | | sense) | (SEQ ID NO: 28) |
| G2 | Region D1 | Template Strand (anti- | GCACUCAAACACGCCUUUGC |
| | | sense) | (SEQ ID NO: 29) |
| G82 | | Template Strand (anti- | GUAUCGCGGAGGAAGGAAACG |
| | | sense) | (SEQ ID NO: 30) |
| G107 | Region D2 | Template Strand (anti- | GCAUGGAGCCUUCGGCUGAC |
| | | sense) | (SEQ ID NO: 31) |
| G108 | | Non-template Strand | GUGGCCAGCCAGUCAGCCGA |
| | | (sense) | (SEQ ID NO: 32) |
| G122 | | Non-template Strand | GCCGCAGCCGCCGAGCGCACG |
| | | (sense) | (SEQ ID NO: 33) |
| G123 | | Template Strand (anti- | GAGGGGCUGGCUGGUCACCAG |
| | | sense) | (SEQ ID NO: 34) |
| G109 | Region D3 | Template Strand (anti- | GCACCGAAUAGUUACGGUCGG |
| | | sense) | (SEQ ID NO: 35) |
| G112 | | Template Strand (anti- | GAAAAAGGGGAGGCUUCCUG |
| | | sense) | (SEQ ID NO: 36) |
| G113 | Region E | Template Strand (anti- | GGAUUAUCAGUGGAAAUCUG |
| | | sense) | (SEQ ID NO: 37) |
| G114 | | Template Strand (anti- | GAAAGAAAUGUAAGAUGUGCU |
| | | sense) | (SEQ ID NO: 38) |
| G115 | | Non-template Strand | GAAGAAAGAUAAGCUCCAUCC |
| | | (sense) | (SEQ ID NO: 39) |
| G116 | | Non-template Strand | GUGAAGGGAUUACAAGGCGUG |
| | | (sense) | (SEQ ID NO: 40) |

In addition to further optimizing oligonucleotide design, as well as targeting region(s) and targeted strand(s), this study also changed the 72h transient transfection system of Example 1 to a stable system, allowing for tracing of P16 demethylation and activation for almost two months. Lentiviris was used to introduce dCas9 as well as sgR2R5 with different guides into SNU-398 cells, and the mCherry (for dCas9 positive cells) and GFP (for sgR2R5 positive cells) double positive cells were sorted. As mentioned above, when checking the P16 mRNA expression, a new pair of primers (Forward: CAACGCACCGAATAGTITACG (SEQ ID NO: 41), Reverse: AGCACCACCAGCGTGTC (SEQ ID NO: 42)) were used which expand the exon-exon junction, revealing that it took about 6-8 days to initiate the P16 demethylation, and more than 13 days for the activation of P16 mRNA under the conditions tested (see FIG. 3). Furthermore, with the stable CRISPR-DiR second generation system described herein, the P16 protein restoration was observed as well as its cell cycle arrest function.

The stable system of this study is informative for DNA methylation and the dynamic epigenetic regulation, as DNA methylation changes occur and become evident if the cells cycle and the majority of the cells acquire a similar phenotype. In addition, the stable system mimics and allows for tracing of the natural epigenetic regulation process of DNA methylation, histone modifications, chrmatine structures etc. In the system of Example 1, changes were observed by MSP, but it was not determined how methylation pattern may remain, or change, after a week for example, nor the dynamic regulation process.

### Results:

FIG. 3 shows results of CRISPR-DiR targeting P16 Region D1 and D3 simultaneously, with four guides targeting both strands in each region. FIG. 3(a) shows the targeting strategy, FIG. 3(b) shows the P16 expression profile, FIG. 3(c) shows the P16 protein restoration in Day 53, FIG. 3(d) shows the methylation in Region D1 and D3 measured by COBRA, and FIG. 3(e) shows the cell cycle analysis of the Day 53 treated samples. FIG. 11 shows the Bisulfite PCR sequencing result for the dynamic demethylation progress of CRISPR-DiR treated samples, and accompanies the data shown in FIG. 3.

The targeting strand specificity of the CRISPR-DiR was then further investigated. With the same guide RNAs targeting P16 region D1 and D3, effects of only targeting one DNA strand were compared with targeting both strands.

FIG. 4 shows results of CRISPR-DiR targeting P16 Region D1 and D3 simultaneously, with only one DNA strand targeted in each sample. FIG. 4(a) shows the targeting strategy, FIG. 4(b) shows the P16 expression profile, and FIG. 4 (c) shows the methylation profile in Region D1 and D3 measured by COBRA. Targeting means the guide RNA sequence (i.e. targeting portion) is complimentary to the targeted strand. The mRNA sequence (sense strand) is the same as the non-template strand. Thus, in the COBRA data, S (sense strand) refers to targeting non-template strand (NT), AS (antisense) refers to targeting template ( ) strand.

Next, CRISPR-DiR was applied to another cell line and another gene, in order to show broad applicability.

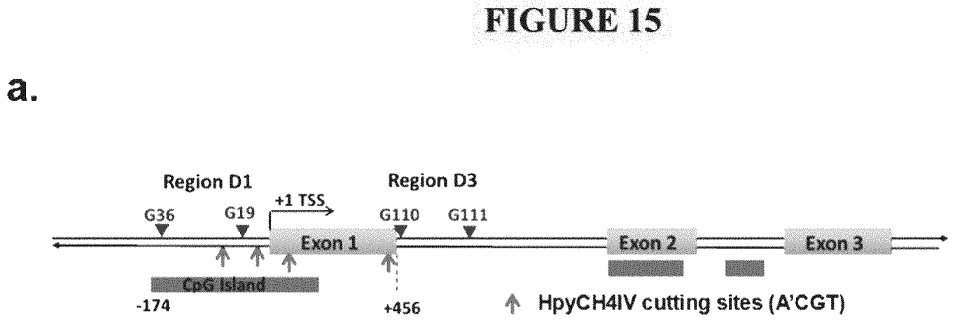

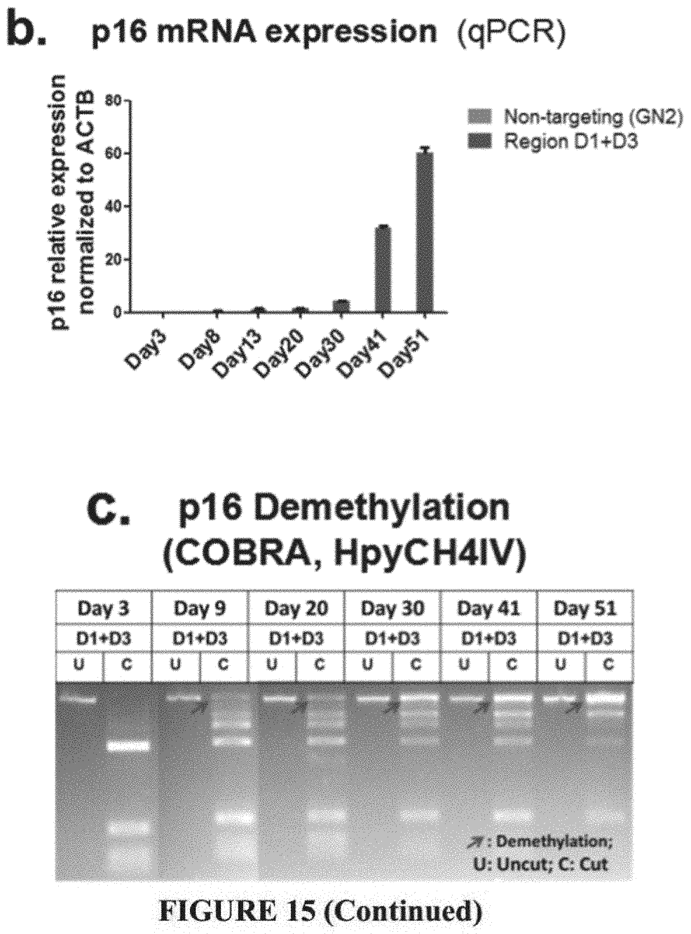

Given the good results for p16 gene in SNU-398 cell line, the CRISPR-DiR approach was tested for application in other cell lines and to other genes. U2OS human osteosarcoma cells are a good model because both p14 and p16 gene is hypermethylated and silenced in this cell line. U2OS-dCas9 stable line was made and the same sgR2R5 lentivirus was transduced to the cells to target the non-template strand of p16 Region D1 and D3. As shown in FIG. 15, the cells were also traced for 53 days and the gene expression and demethylation was analyzed. Similar to the SNU-398 results, the demethylation of p16 Region D1 and D3 occurred around Day8, while the mRNA activation was several days later. In U2OS cells, the p16 mRNA was stably activated during Day 20 to Day 30, which is slower than in SNU-398 cells. Without wishing to be bound by theory, it was contemplated that the p14 gene may be hypermethylated and silenced in U2OS but not SNU-398, therefore, the chromatin structure in p14/p16 locus may be more condense in U2OS cells than in SNU-398, thus it may have taken longer for p16 locus in U2OS cells to be opened and re-expressed. Such a system may be of interest to further explore histone modifications and chromatin accessibility of the p16 locus during the whole demethylation and gene expression process.

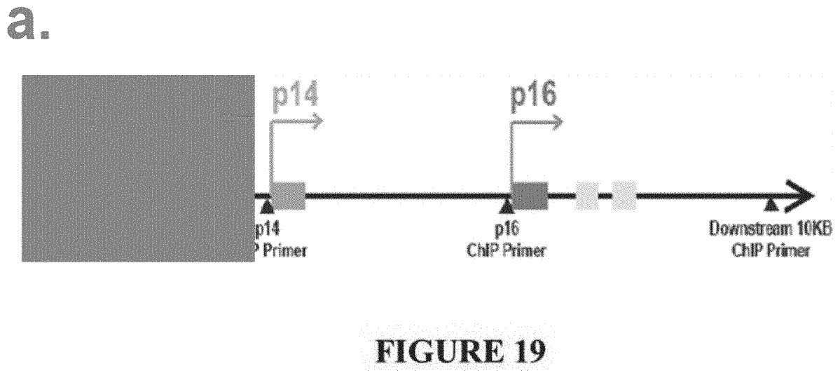

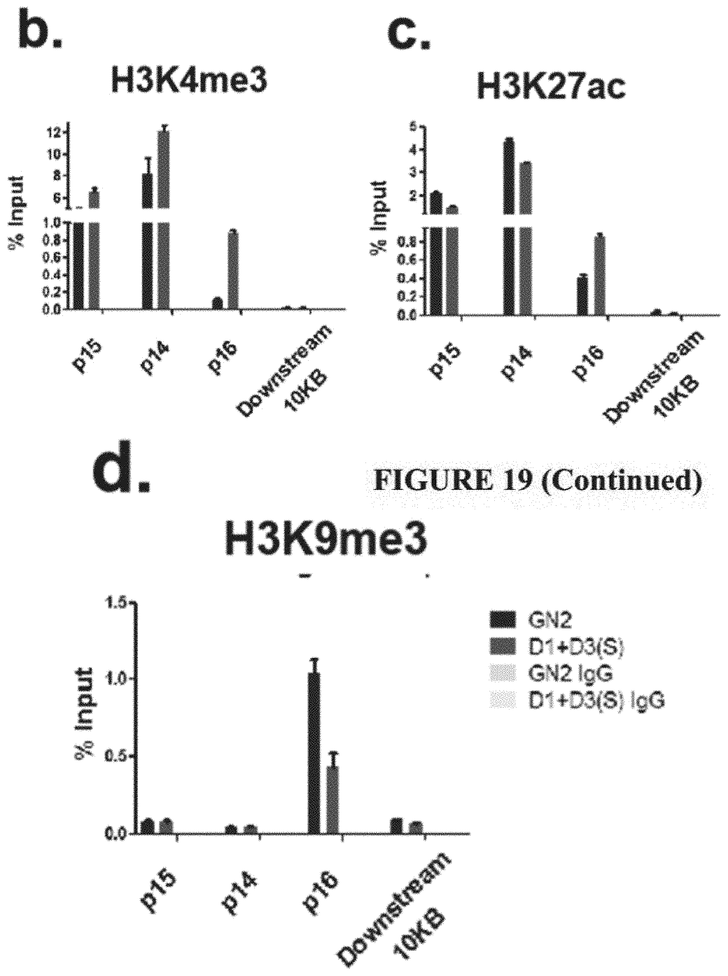

FIG. 19 shows ChIP-qPCR data for histone markers. The histone markers change in CRISPR-DiR treated Day 53 SNU-398 cells were studied by ChIP-qPCR. As shown in FIG. 19, in p16 proximal promoter region, there were significant increase of gene activation markers H3K4me4 and H3K27ac, while decrease of gene silencing marker H3K9me3. These histone changes are specific in p16 locus, as there are no changes in the nearby genes (P14, P15) and downstream negative region (10 Kb downstream of P16). The histone changes are consistent with CRISPR-DiR induced P16 demethylation and activation, and the specificity also indicated that CRISPR-DiR is a gene specific method. FIG. 19 shows histone markers ChIP-qPCR results of CRISPR-DiR treated fifty-three cells. FIG. 19(a) shows the locations of ChIP-qPCR checked histone markers, P16 is the CRISPR-DiR targeted gene, while P14, P15, downstream 10 Kb are the nearby non-targeted locus; FIG. 19(b) shows the enrichment of active histone marker H3K4me3; FIG. 19(c) shows the enrichment of active histone marker H3K27ac; and FIG. 19(d) shows the enrichment of silencing histone marker H3K9me3.

FIG. 15 shows results of CRISPR-DiR targeting p16 Region D1 and D3 non-template strand (NT) simultaneously in U2OS cell line. FIG. 15(a) shows the targeting strategy, FIG. 15(b) shows the p16 expression profile, and FIG. 15(c) shows the methylation profile in Region D1 and D3 measured by COBRA.

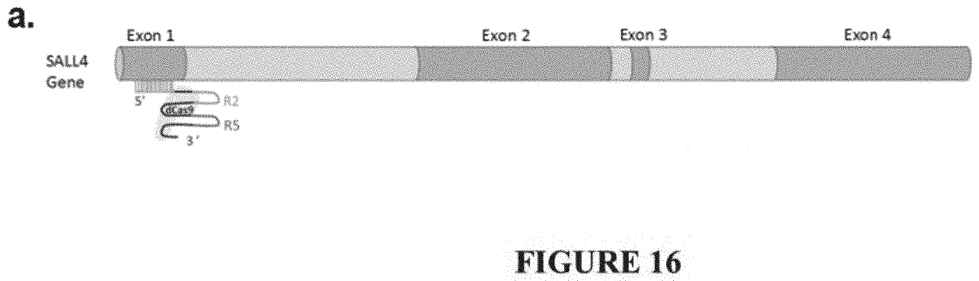

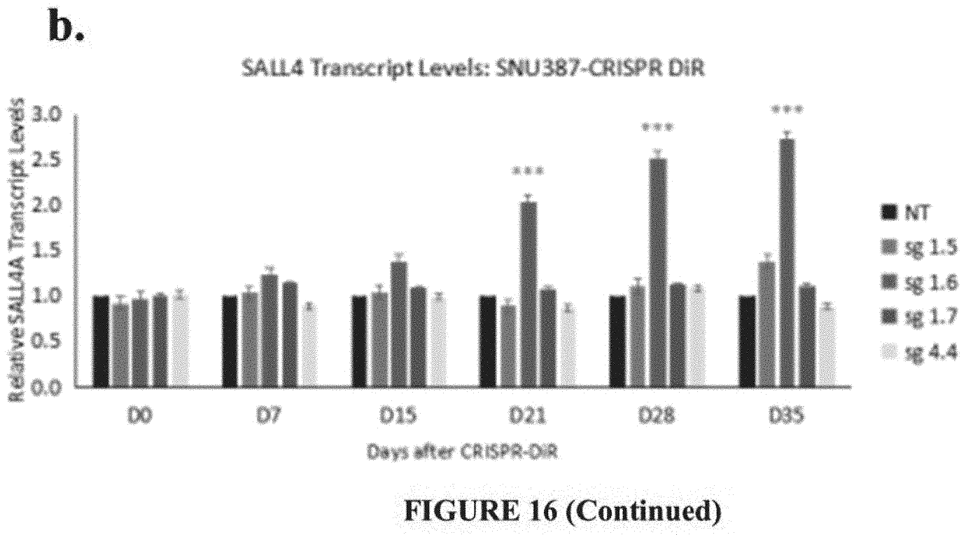

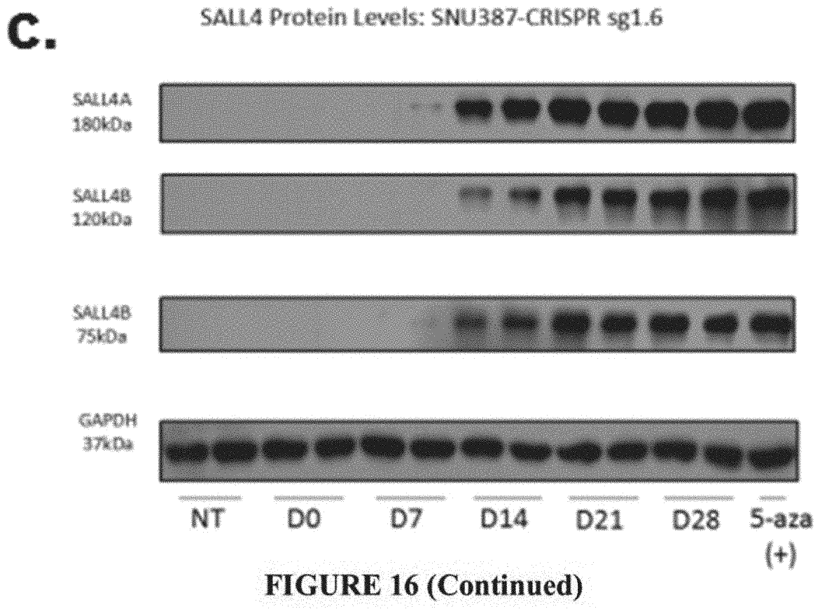

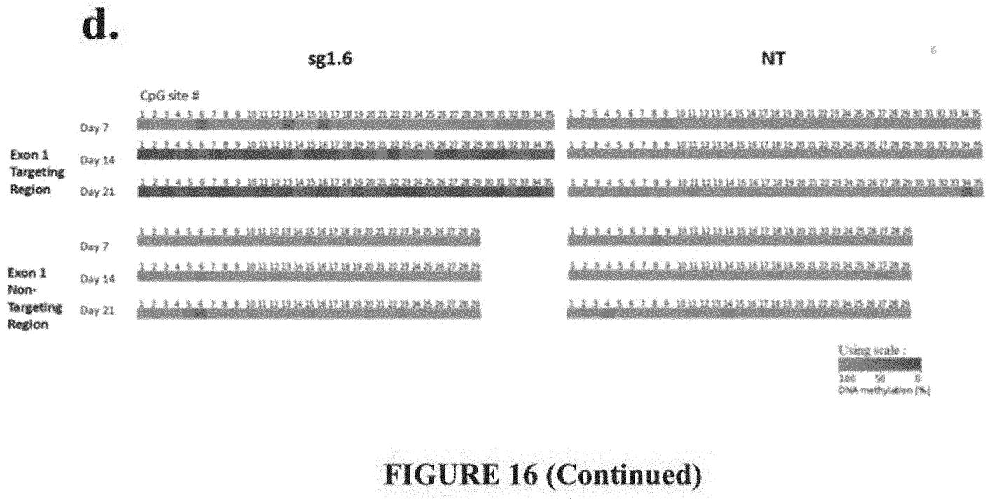

In addition, the CRISPR-DiR approach was applied for SALL4 gene which is hypermethylated and silenced in SNU-387 HCC cell line. Consistently, the gene was successfully demethylated and SALL4 expression as well as function was restored (see FIG. 16). It was also observed that CRISPR-DiR only provided significant effect when targeting the non-template strand (NT) of SALL4.

FIG. 16 shows results of CRISPR-DiR targeting SALL4 non-template strand for demethylation and gene activation with Guide 1.6 sgDiR (sg1.6, GCUGCGGCUGCUGCUCGCCC. SEQ ID NO: 13). FIG. 16(a) shows the targeting strategy, FIG. 16(b) shows the SALL4 mRNA expression profile, FIG. 16(c) shows the SALL4 protein restoration, and FIG. 16(d) shows the demethylation in the targeted regions of control cells and CRISPR-DiR treated cells.

These studies show that the CRISPR-DiR approach was not limited to only p16 locus in SNU-398 cells, but may be applied more broadly to other cells and genes, as shown by this further data using another cell line (U2OS) and another gene (SALL4). Thus, it is contemplated that this approach may be applicable to a wide variety of suitable genes. In certain embodiments, it is contemplated that CRISPR-DiR approaches as described herein may be used to further target other genes, and/or to make a guide RNA pool for multiple targeting, to expand the usage of this tool. In certain embodiments, CRISPR-DiR may be used to further understanding of the epigenetic regulation in other loci.

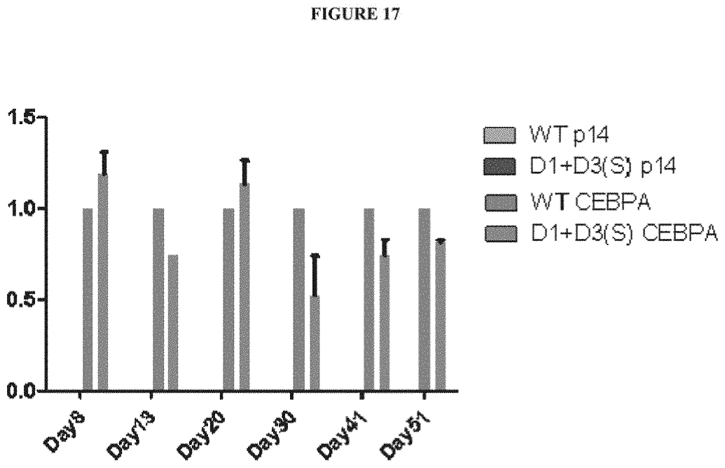

Because of the potential off-target effects of certain CRISPR systems, locus specificity of the CRISPR-DiR approach was further investigated. The p14, p15 genes close to p16 locus were first checked, as well as CEBPA and SALL4 genes which are located far away from p16. In U2OS cell line, both p14 and p16 are hypermethylated and silenced, while CEBPA is expressed. As shown in FIG. 17, when CRISPR-DiR treated U2OS cells were traced from Day 0 to Day 51, no activation of p14 was observed (undetectable), and not significant changes of CEBPA as observed. These results suggest high specificity of CRISPR-DiR approach to the targeted locus. FIG. 17 shows CEBPA mRNA expression and p14 mRNA expression in U2OS cells with CRISPR-DiR targeted for 51 days.

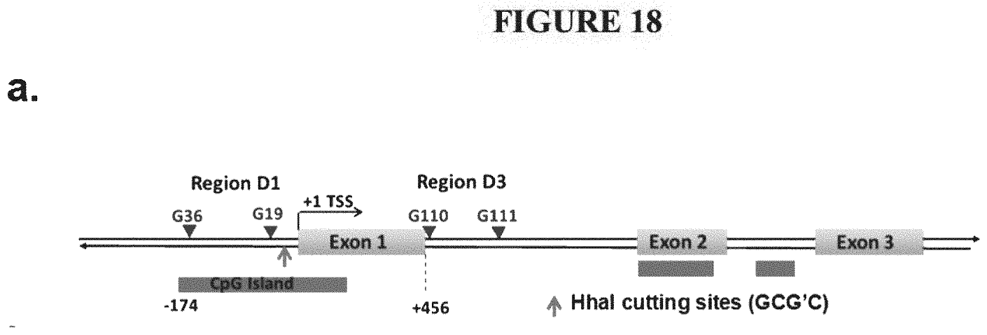

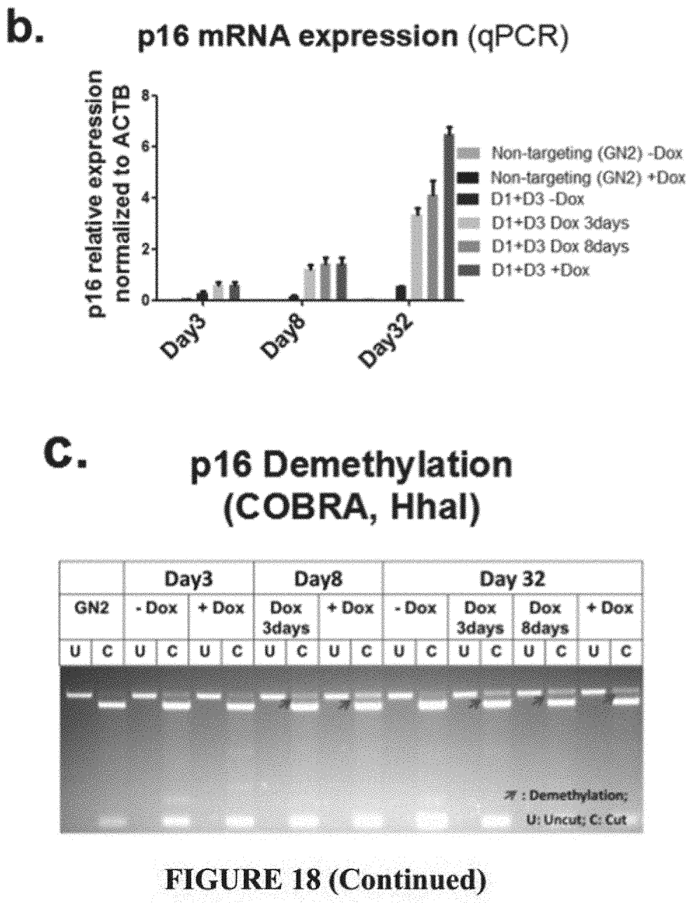

Duration of CRISPR-DiR effect maintenance was also investigated. It was hypothesized that once CRISPR-DiR induced the demethylation of p16, other epigenetic regulation and perhaps RNA regulation may be involved in a dynamic process to activate the gene. Therefore, it was first investigated whether the CRISPR-DiR effects may be maintained or not if the treatment is withdrawn once the demethylation is initiated. Previous results showed that the CRISPR-DiR worked in the presence of not only sgR2R5 but also dCas9. Therefore, a Tet-On dCas9 inducible SNU-398 cell line was amide, in which the dCas9 would only express if Doxycycline was added. The same sgR2R5s were used to target the non-template strand (NT) of Region D1 and D3, and in this way, the CRISPR-DiR treatment may be started or stopped by adding or withdrawing Doxycycline to control the existence of dCas9. Since the demethylation occurred in Day8 in both SNU-398 and U2OS cells, Doxycycline was added for eight days to initiate the demethylation, then addition was stopped to withdraw the CRISPR-DiR treatment but culturing the cells was continued to trace the changes. The cells were harvested in Day0, Day8, Day13, Day20, Day32, and the cells with no Doxycycline, Doxycycline treated for eight days, and always with Doxycycline were compared. FIG. 18b shows the gene expression level for differently treated cells in each time point, and FIG. 18c is the demethylation profile of Region D1 for each sample. It was found that the inducible system worked well since there was no gene activation or demethylation in any time point if the cells had never been induced by Doxycycline, while the demethylation and gene activation were consistent with previous non-inducible systems if Doxycycline was always added to the cells. Interestingly, when comparing the cells with CRISPR-DiR on for eight days (Doxycycline added for eight days) and the cells always with CRISPR-DiR on (Doxycycline always added), a sharp increase of p16 mRNA was observed right after withdrawing the drug, which is higher than that of the cells always with CRISPR-DiR on at the same time point. However, the p16 in the CRISPR-DiR treated eight days cells gradually decreased to the same level as the always CRISPR-DiR on cells. Although there was an increase and decrease change of the p16 expression in the Doxycycline treated eight days cells, the p16 activation and demethylation were maintained for more than two weeks after withdrawing Doxycycline. Without wishing to be bound by theory, these results may support a hypothesis that once the demethylation is initiated in the gene locus, several other regulation mechanism may be involved to maintain the demethylation status or open the chromatin structure, and to turn on the gene. A decrease of sgR2R5 expression was observed after withdrawing Doxycycline, which may, without wishing to be bound by theory, indicate that sgR2R5 RNA may be less stable without dCas9. Overall, these results indicated CRISPR-DiR effects were maintained for about a month in these study conditions.

FIG. 18 shows results from the dcas9 inducible CRISPR-DiR system in SNU-398 cells. FIG. 18(a) shows the targeting strategy, FIG. 18(b) shows the p16 expression profile, and FIG. 18(c) shows the methylation profile in Region D1 measured by COBRA.

A detailed exploration and data supporting the above results has been provided here.

Determining Effective Target Regions of P16:

It was sought to explore 1) the best targeting region(s) where the demethylation would correlate with P16 activation; and 2) longer time points for the effects, using the CRISPR-DiR design (CRISPR-R2R5).

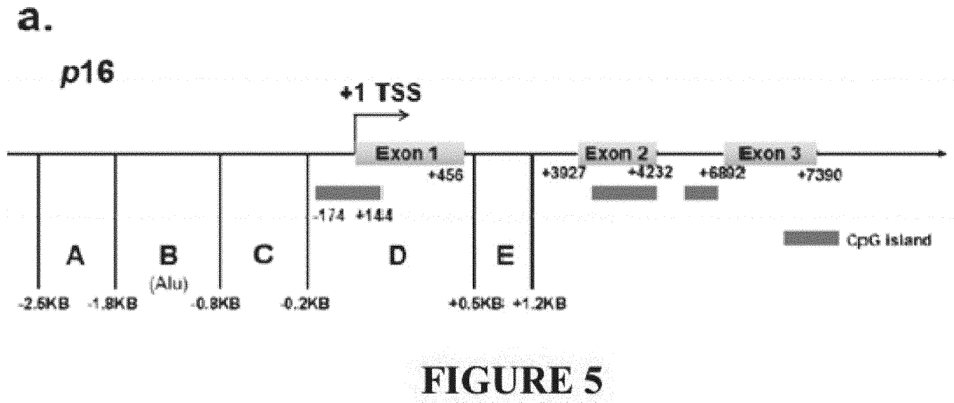

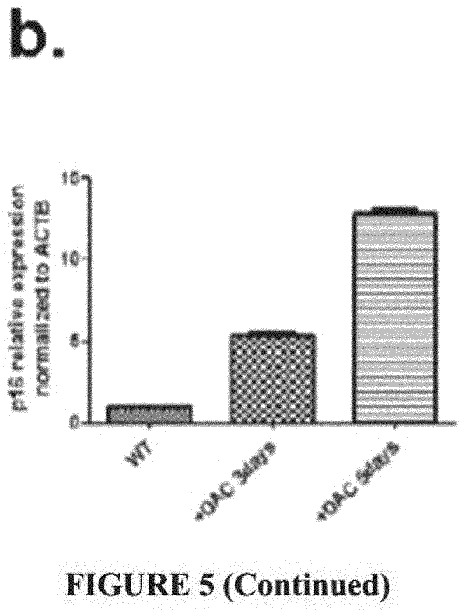

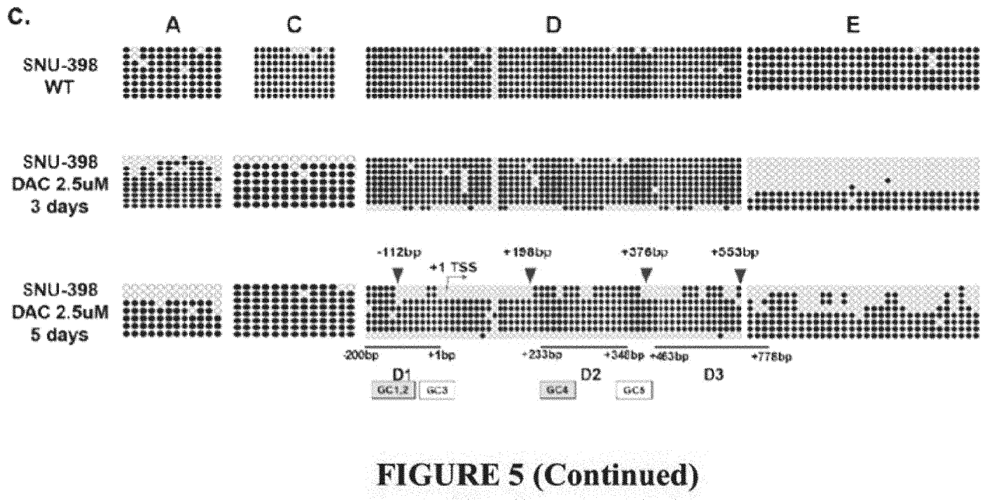

Although theoretically the DNMT1 inhibitors 5aza and decitabine are genome-wide demethylation agents, scientists have reported the existence of functional demethylation regions (8). In order to study the functional demethylation regions of P16 and the correlation between these regions and P16 mRNA expression, wild type SNU-398 cells were treated with Decitabine for 3 days and 5 days, and mRNA expression was then checked for each sample as shown in FIG. 5b. Bisulfite sequencing PCR (BSP) was performed from −2.5 Kb to +1.2 Kb (+1 refers to transcription start site) of P16 gene, to get the methylation change of each CG dinucleotide. The reason that it was decided to study the −2.5 Kb to +1.2 Kb region is because most of the CG dinucleotides are located in this region. This long region was further divided into five sub-regions (Region A, B, C, D and E) as shown in FIG. 5a, and BSP was performed for all the sub-regions except Region B, because there are two Alu repetitive elements which are intrinsically difficult to sequence and map. Genomic DNA was extracted from SNU-398 wild type cells as well as the cells treated with Decitabine for 3 days and 5 days, then the three DNA samples were bisulfite converted and PCR amplified by four pair of primers covering Region A, C, D and E. Then TA cloning was performed to clone the PCR products into vectors and 10 clones of each PCR products were purified and sent for Sanger sequencing. The methylation level of each sub-region was checked and compared the methylation between wild type SNU-398 and Decitabine treated samples. Surprisingly, the BSP results shown in FIG. 5c indicated that the most demethylated region after Decitabine treatment for 3 days was not only the promoter CpG island, but also the first exon region downstream of the CpG island (Region E) This result indicated that P16 activation by Decitabine was not perfectly correlated with promoter CpG island demethylation, instead, Region E may be either the important functional demethylation region or simply an easily demethylated region. It's not consistent with a general belief that the hypermethylation of promoter CpG island is associated with gene silencing. However, interestingly, stronger demethylation in Region D (CpG island) was observed in the Decitabine treated five days sample, and the demethylation was not evenly distributed in Region D. The 5′ and 3′ of Region D were demethylated, while the middle of Region D was still hypermethylated. It was shown that SP1 is one of the positive transcription factors that positively regulate P16 transcription. The binding motif of SP1 is GC box. There are five GC boxes in P16 promoter and exon 1 region, and GC box 1, 2, 4 were reported to play a key role in P16 regulation (9). It was reported that the methylation at CG sites outside of the consensus Sp1-binding site may directly reduce the ability of Sp1/Sp3 to bind its DNA. Therefore, based on the distribution of demethylation and GC boxes in Region D, it was further divided to Region D1, D2 and D3. Region D1 (comprises GC box 1, 2 and 3) and Region D3 were hypermethylated, while Region D2 (comprises GC box 4 and 5) was not demethylated. Meanwhile, Region A got moderate demethylation in both Decitabine treated Day 3 and Day 5 samples.

FIG. 5 shows the methylation and gene expression profiles for SNU-398 wild type cells treated with 2.5 uM DAC for three and five days. FIG. 5(a) shows the five regions checked for methylation in P16 locus; FIG. 5(b) shows the P16 gene expression in the cell samples; and FIG. 5(c) shows bisulfite sequencing data for wild type cells and DAC treated cells in Region A, C, D and E. Each black or white dot represents a CG site, the black dot indicates methylated C, while white dot represents unmethylated C.

Taken together, these BSP results provide important information that P16 activation was not only correlated with promoter CpG island (Region D1), but also Region E, D3 and A. Particularly, Region E may be a good region to target since the demethylation in Region E occurred even earlier and stronger than Region D1, D3, and A. And the previous D1 region which was targeted by G2 guide RNA (see Example 1) may not be the most effective targeting region, especially if only tracing the cells for three days. However, the possibility that the demethylation in Region D1, D3, and A are also important so they are harder to be demethylated and may take waiting for a longer time cannot be ruled out. Moreover, the methylation was traced in the short region around P16 transcription start site, and genome-wide methylation profile may provide additional information about the highly demethylated regions and hard-to-be-demethylated regions even with a genome-wide demethylation agent.

Thus, Whole Genome Bisulfite Sequencing (WGBS) was performed for wild type SNU-398 cells and Decitabine treated three days cells. The demethylation regions in −2.5 Kb to +1.2 Kb of P16 shown by WGBS data was consistent with BSP data. When combining RNA-seq, H3K27me3 CHIP-seq, and WGBS data of this cell line together, a list of genes that are silenced in SNU-398 cells and their demethylation regions around transcription start sites was obtained. This information may be useful for targeting other genes or making a sgRNA pool to target different genes for CRISPR-DiR induced demethylation and activation.

For P16 regions A, D, E, CRISPR-DiR may be designed with guide RNAs targeting these regions both separately and simultaneously, but technically it was decided to target Region E first and trace the CRISPR-DiR treated cells for longer time since BSP results showed that the demethylation of P16 is not as fast as initially thought even with high concentration Decitabine treatment. The demethylation mechanism of CRISPR-DiR is believed to be based on the block of DNMT1, which takes several cycles for demethylation. In addition, clinically, even using 5′aza for MDS may take several months to respond.

CRISPR-DiR targeting Region E for Demethylation:

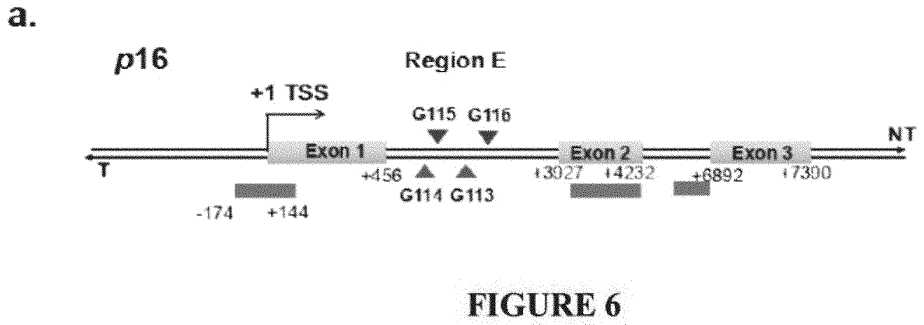

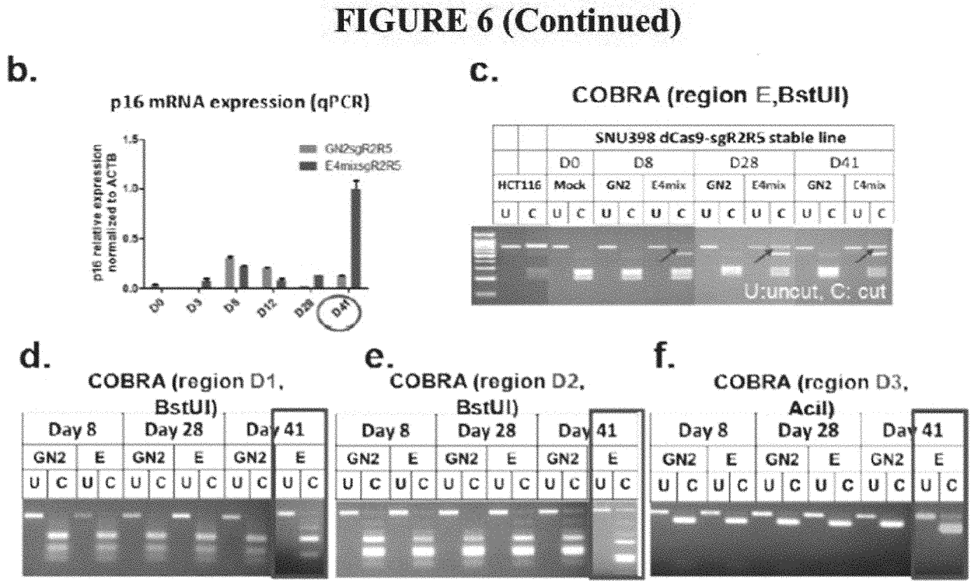

In order to target Region E and trace the treated cells for a longer time, several guide RNAs targeting both DNA strands of Region E were designed, and then an in vitro cleavage assay was performed to test the efficiency of each guide. G113 and G114 were picked to guide CRISPR-DiR to target the template strand (T) in Region E, while G115, G116 were chosen to target the non-template strand (NT) in the same locus (see FIG. 6(a)). All these sgR2R5 oligonucleotide constructs (G113sgR2R5, G114sgR2R5, G115sgR2R5, G116sgR2R5) were prepared as lentivirus, and were transduced into wild type SNU398 and SNU398-dCas9 stable line either separately (one guide in one cell line) or together (G113sgR2R5, G114sgR2R5, G115sgR2R5, G116sgR2R5 lentivirus were mixed equally). The reason the sgR2R5 were transduced into both wild type cells and dCas9 stable cells was because it was desired to explore whether dCas9 may be needed for demethylation and activation, or if only sgR2R5 may still provide demethylation and/or activation.

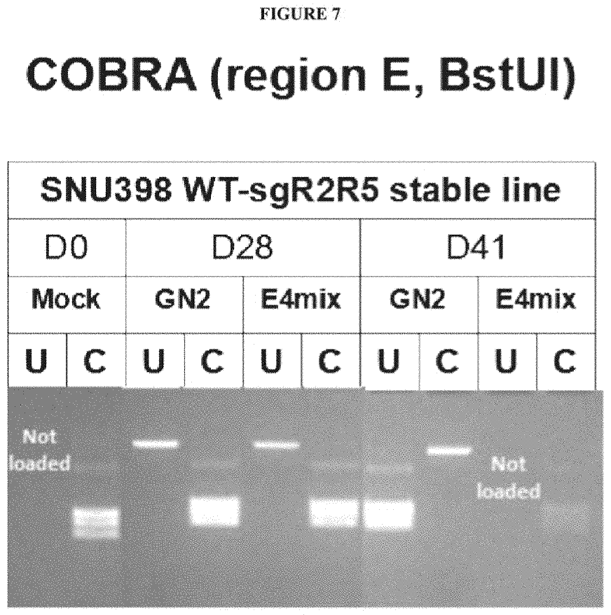

RFP signal was used to sort dCas9 positive cells, while GFP signal was used to check the successful integration of sgR2R5. These CRISPR-DiR targeted cells were cultured for three months after making the SNU398-CRISPR-DiR stable line, and the P16 expression was analyzed as well as demethylation in several time points. The qPCR results (see FIG. 6(b)) and Combined Bisulfite Restriction Analysis (COBRA) results (see FIG. 6(c)) indicated that CRISPR-DiR targeting Region E by the mix of four guide RNAs indeed successfully activated P16 expression and induced the demethylation of Region E. Of note, the mix of four guide RNAs targeting both template and non-template strands worked better than only one guide RNA in P16 mRNA activation, and the CRISPR-DiR functioned far better with dCas9 since there was no detected gene activation or demethylation if only transducing sgR2R5 into wild type SNU-398 without dCas9 (FIG. 7).

FIG. 6 shows results of CRISPR-DiR targeting P16 Region E with four mixed guide RNAs (G113, G114, G115, G116). In FIG. 6(a), the targeting strategy is shown; in FIG. 6(b), the P16 expression profile traced for three months is shown; in FIG. 6(c) the methylation of CRISPR-DiR treated samples measured by COBRA in Day0, day, Day28 and Day 41 is shown. The red arrows indicate the undigested DNA, which is the demethylated DNA that can't be cut. In FIG. 6(d), the methylation in Region D1 after targeting Region E for 41 days is shown; in FIG. 6(e) the methylation in Region D2 after targeting Region E for 41 days is shown; and in FIG. 6(f) the methylation in Region D3 after targeting Region E for 41 days is shown.

FIG. 7 shows results of CRISPR-DiR targeting Region E with the same guide RNAs but no dCas9. “Not loaded” means there are not enough samples to load; however, the unload samples are uncut control, so the uncut band information can still be obtained from other uncut samples, and the length of all the uncut DNA should be the same.

Surprisingly, the results showed that both the demethylation and gene activation took a relatively long time to be initiated and kept stable. CRISPR-DiR induced Region E slightly demethylation from Day 8, and a more obvious demethylation in Day 28 and onwards. However, P16 mRNA expression start from Day 28 and has been increased significantly and stably after Day 41. Considering the hypermethylation of P16 and the heterochromatin structure, it is hypothesized that it took several cell cycles for CRISPR-DiR to target P16 and to initiate the demethylation, then the demethylation in Region E may have further led to the DNA demethylation of nearby regions or initiated other histone modification changes that finally opened the chromatin structure and activated the mRNA transcription in a later time point.

In order to test this hypothesis, first the demethylation of the CpG island regions (D1, D2 and D3) of the CRISPR-DiR targeted Day8, Day28 and Day 41 samples was checked. As shown in FIGS. 6(d), 6(e), and 6(f), when CRISPR-DiR targeted Region E, the demethylation of Region E started from Day8 and increased gradually. When in Day 41, the demethylation of Region E was quite strong, and at this time point, it was also observed slight demethylation of Region D1, and stronger demethylation in Region D2 and D3. This result indicated that the DNA demethylation may spread slowly to nearby regions or perhaps important transcription regulation regions. This thus may be an interesting system to explore dynamic epigenetic regulation (DNA demethylation spreading, functional demethylation regions, histone modification changes and chromatin accessibility changes, etc.) of a specific gene locus (10).

CRISPR-DiR Targeting Region E and A for Demethylation: After targeting Region E for months, it was noticed that even though it indeed induced demethylation and gene activation, exploring other regions was also of interest to get a complementary understanding about the important demethylation and regulation regions, as well as to get toward maximum gene restoration.

Based on the BSP results (see FIG. 5c) other regions showing demethylation after Decitabine treatment include Region A, D1 and D3. Among these three region, it appeared that Region A got demethylated earlier than D1 and D3. In addition, according to the demethylation spreading from Region E to the nearby regions (Region D2, D3 got clear demethylation, while D1 was slightly demethylated), it was asked whether it would be better to target Region E and A together, so that two regions could be targeted which got demethylation via Decitabine, and the demethylation of these two regions might be able to spread to region D because they are in both 5′ and 3′ of Region D.

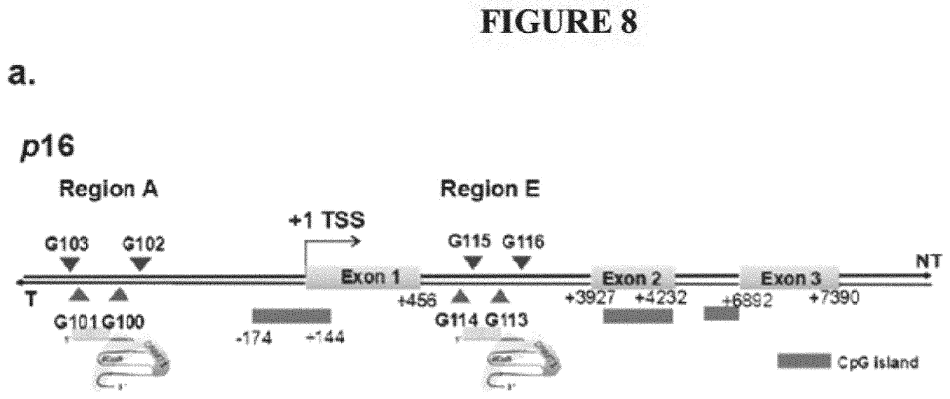

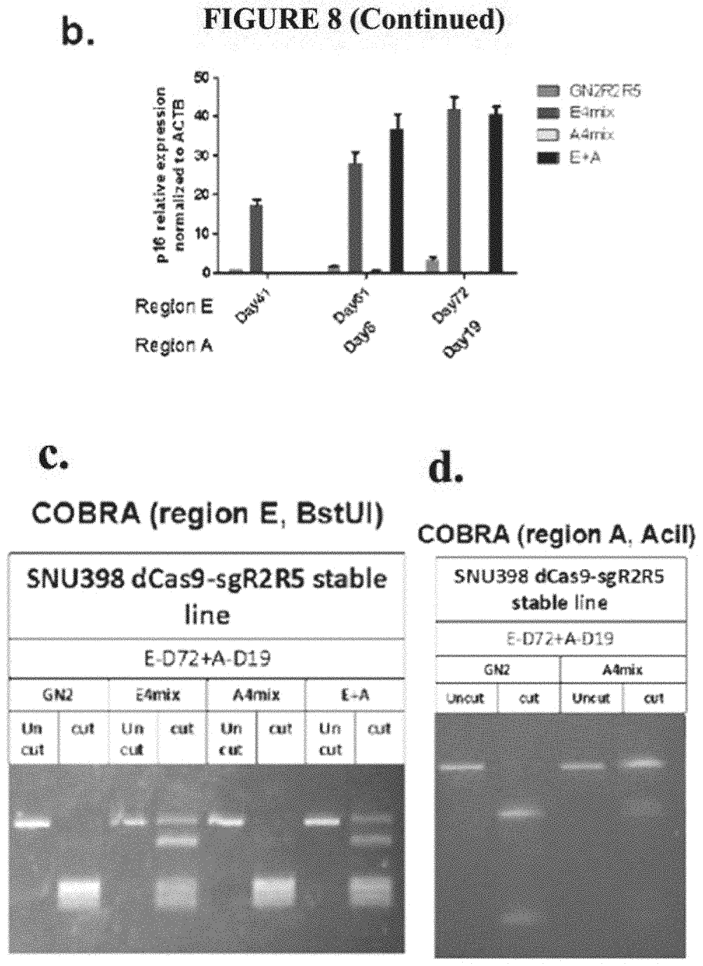

Therefore, several guide RNA targeting both strands of Region A DAN were designed and screened, and guide G100, G101 targeting template strand (T), and G102, G103 targeting non-template strand (NT) were picked (see Table above for sequences). Again, lentivirus were made for these four sgR2R5 (G100sgR2R5, G101 sgR2R5, G102sgR2R5, G103sgR2R5) (see Table above for sequences), and they were transduced together to either SNU-398-dCas9 cell line or the cell line with CRISPR-DiR targeting Region E for 53 days. In this way, cell lines with 1) only CRISPR-DiR targeting Region A, 2) only CRISPR-DiR targeting Region E, and 3) CRISPR-DiR targeting both Region E and A was obtained. Cells were then cultured for another month to compare the effects by different targeting. However, as shown in FIG. 8(b), 8(d), although only targeting Region A indeed resulted in the demethylation of Region A, it failed to activate mRNA expression efficiently, and the demethylation is only in Region A, not in Region E. If Region A and E were targeted together, both regions were demethylated, but the gene activation level of targeting E and A was the same as that of targeting only Region E. This indicated that CRISPR-DiR is able to demethylate any targeted region specifically, but this demethylation does not necessarily lead to gene activation. Therefore, although Region A was demethylated by Decitabine treatment and also can be demethylated by CRISPR-DiR, the demethylation of this region may not correlate with P16 expression, as it may not be a functional demethylation region for P16.

FIG. 8 shows CRISPR-DiR targeting P16 Region E, or Region A or Region E+A with four mixed guide RNAs for each region. FIG. 8(a) shows the targeting strategy; FIG. 8(b) shows the P16 expression profile; FIG. 8(c) shows the methylation in Region E of CRISPR-DiR treated samples measured by COBRA, Region E was targeted for 72 days while Region A was targeted for 19 days; and FIG. 8(d) shows the methylation in Region A after targeting Region E, Region E was targeted for 72 days while Region A was targeted for 19 days.

Though the activation was not further enhanced by targeting region A, the results here provided valuable information: 1) CRISPR-DiR may demethylate and only demethylate the targeted region (though the demethylation may spread in the later time points, perhaps because of other epigenetic process(es)); and 2) the CRISPR-DiR initiated P16 activation may be achieved by targeting the demethylation in key regions region(s) (Region A may provide a negative control).

CRISPR-DiR targeting Region E and D1 for demethylation: Though Region A was not a strong targeting region, Region D1, D2, and D3 were explored because 1) they are the promoter-exon 1 CpG island regions which have been reported to correlate with gene expression; 2) Region D1 and D3 were indeed demethylated after 5 days of Decitabine treatment; 3) the demethylation of Region E spread to Region D1, D2 and D3 in the later time point (Day 41); and 4) there are several GC boxes in these regions that may be important for SP1 binding as well as P16 expression. Thus, these three regions were explored starting from Region D1.

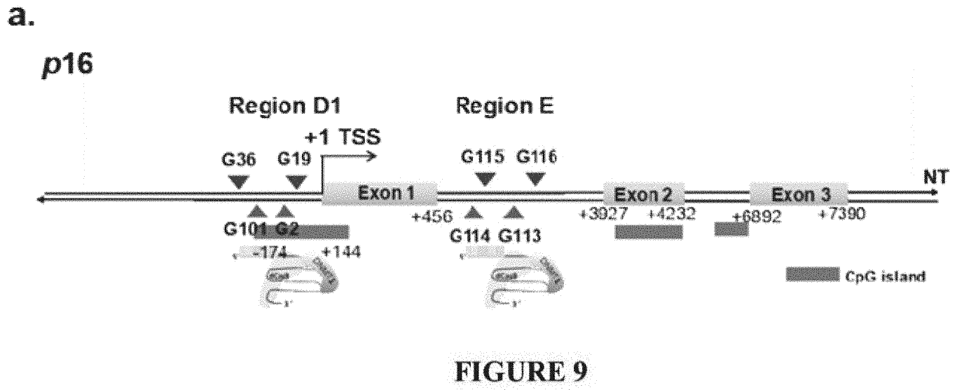

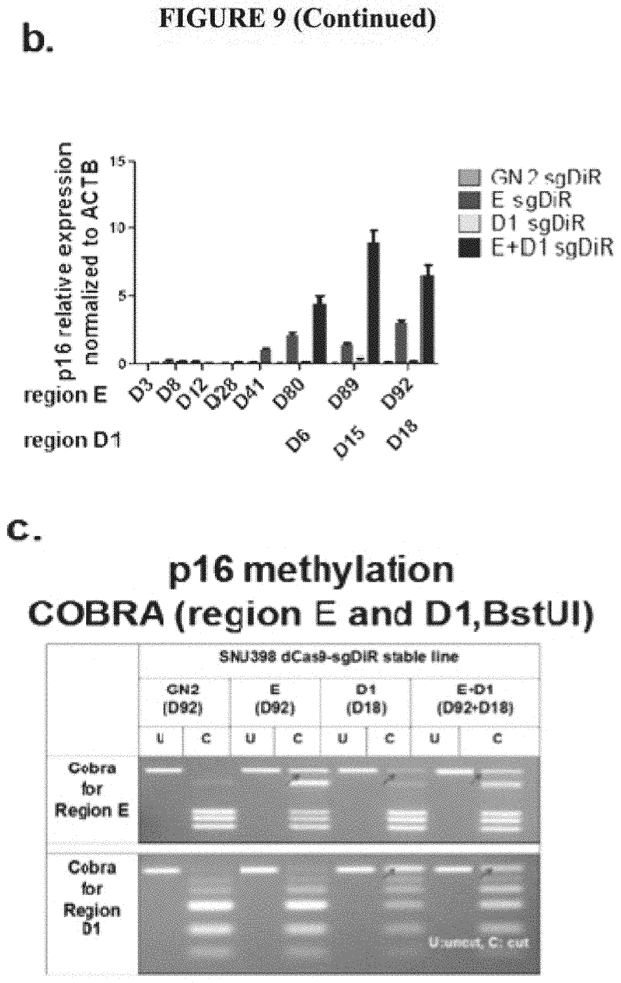

Several guide RNAs targeting both strands of Region D1 DAN were designed and screened, and guides G2, G82 targeting template strand (T), and G19, G36 targeting non-template strand (NT) were selected (see Table above for sequences). Lentivirus were made for these four sgR2R5 (G2sgR2R5, G19sgR2R5, G36sgR2R5, G82sgR2R5) (see Table above for sequences), and these were transduced together to either SNU-398-dCas9 cell line or the cell line with CRISPR-DiR targeting Region E for 83 days. In this way, cell lines with 1) only CRISPR-DiR targeting Region D1, 2) only CRISPR-DiR targeting Region E, and 3) CRISPR-DiR targeting both Region E and D1 were obtained. Cells were then cultured for 18 days to compare the effects by different targeting. Cell samples were harvested in Day 6, Day 15, Day 18 after transducing sgR2R5 lentivirus for Region D1, and the gene expression and demethylation for non-targeting control cells (GN2), Region D1 targeted cells, Region E targeted cells, and both Region E and D1 targeted cells was analyzed. Interestingly, though only targeting Region D1 did not significantly activate P16 expression too much, the combination of targeting E and D1 indeed enhanced the gene expression significantly higher than only targeting Region E (FIG. 9b). As for demethylation, the Region D1 targeted Day 18 samples were taken, and Region E was targeted 92 days at that time point. Combined Bisulfite Restriction Analysis (COBRA) assay was performed to check the demethylation occurred in Region D1 and region E. Targeting both Region E and D1 resulted in demethylation in both regions, while targeting Region E mainly demethylated Region E, only very weak demethylation in Region D1. However, interestingly, targeting only Region D1 led to demethylation not only in Region D1, but also Region E, though the demethylation in Region E was even stronger if this region was targeted by CRISPR-DiR (FIG. 9c). Of note, this demethylation spreading from Region D1 to region E was clearly observed in Day18 when D1 was targeted, so the initiation of the spreading may be even earlier, and checking the earlier time points and also region D2, D3 may provide even further insight of this demethylation spreading.

FIG. 9 shows CRISPR-DiR targeting P16 Region E, or Region D1 or Region E+D1 with four mixed guide RNAs for each region. In FIG. 9(a), the targeting strategy is shown; In FIG. 9(b) the P16 expression profile is shown; in FIG. 9(c) the methylation in Region E and Region D1 of CRISPR-DiR treated samples measured by COBRA is shown, Region E was targeted for 92 days while Region D1 was targeted for 18 days.

This study observed enhanced P16 activation by CRISPR-DiR targeting both Region D1 and E, though only targeting Region D1 didn't work as well. Both Region D1 and E were demethylated when targeting Region D1 for 18 days, showing demethylation spreading process. These results indicated an important role of Region D1, and led to further exploration of Region D2 and D3 to further understand these regions.

CRISPR-DiR Targeting Region E, D1, D2, D3 for Demethylation: Several guide RNAs targeting both strands of Region D2 and D3 DAN were designed and screened, and guide G107, G123 targeting template strand (T) of Region D2, G108, G122 targeting template strand (T) of Region D2, G109, G112 targeting template strand (T) of Region D3, and G110, G1111 targeting non-template strand (NT) of Region D3 were selected. Lentivirus were produced for these eight sgR2R5 (G107sgR2R5, G108sgR2R5, G122sgR2R5, G123sgR2R5, G109sgR2R5, G110 sgR2R5, G111sgR2R5, G112sgR2R5), and they were transduced to several cell lines obtaining cell lines with 1) Only CRISPR-DiR targeting Region D1, 2) Only CRISPR-DiR targeting Region D2, 3) Only CRISPR-DiR targeting Region D3, 4) Only CRISPR-DiR targeting Region E, 5) CRISPR-DiR targeting both Region E and D1, 6) CRISPR-DiR targeting Region D1 and D3, 7) CRISPR-DiR targeting Region D2 and D3, 8) CRISPR-DiR targeting region D1, D2 and D3.

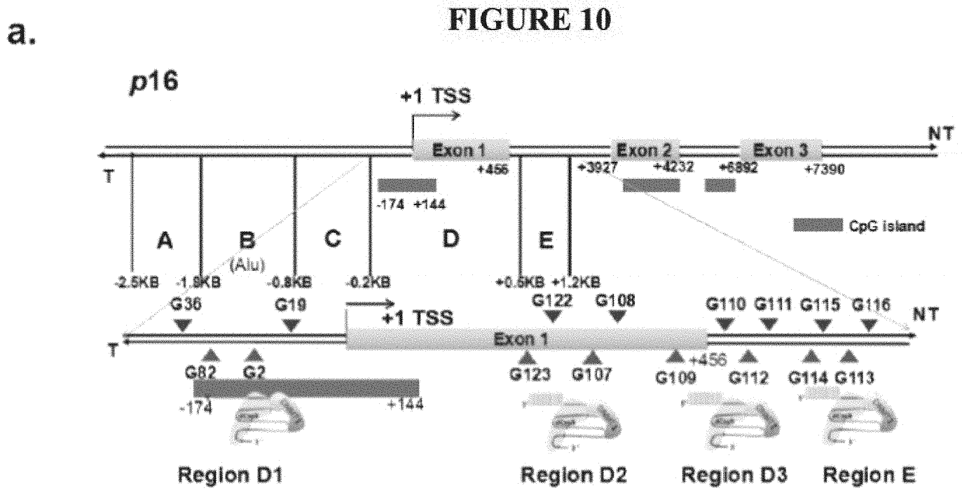

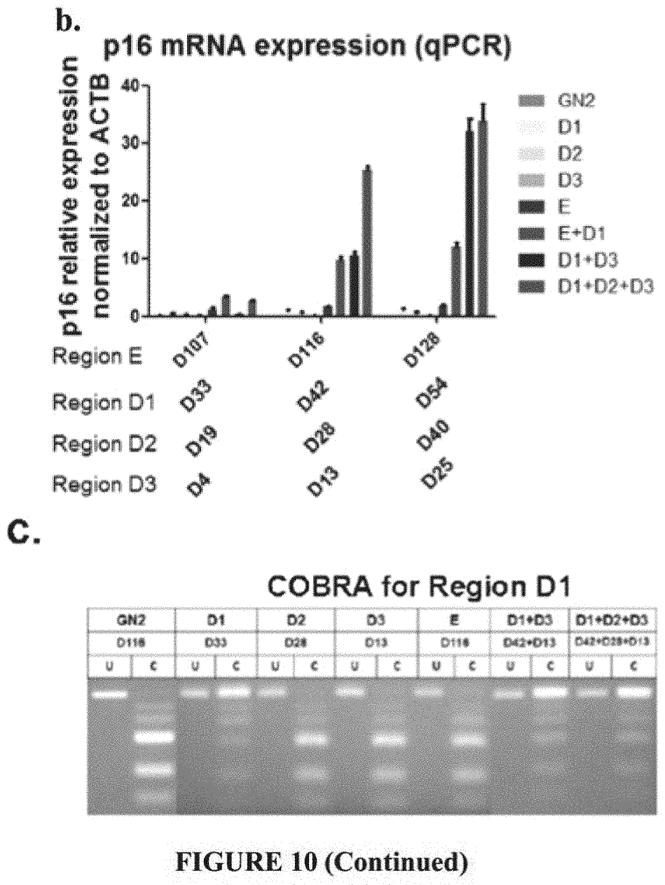

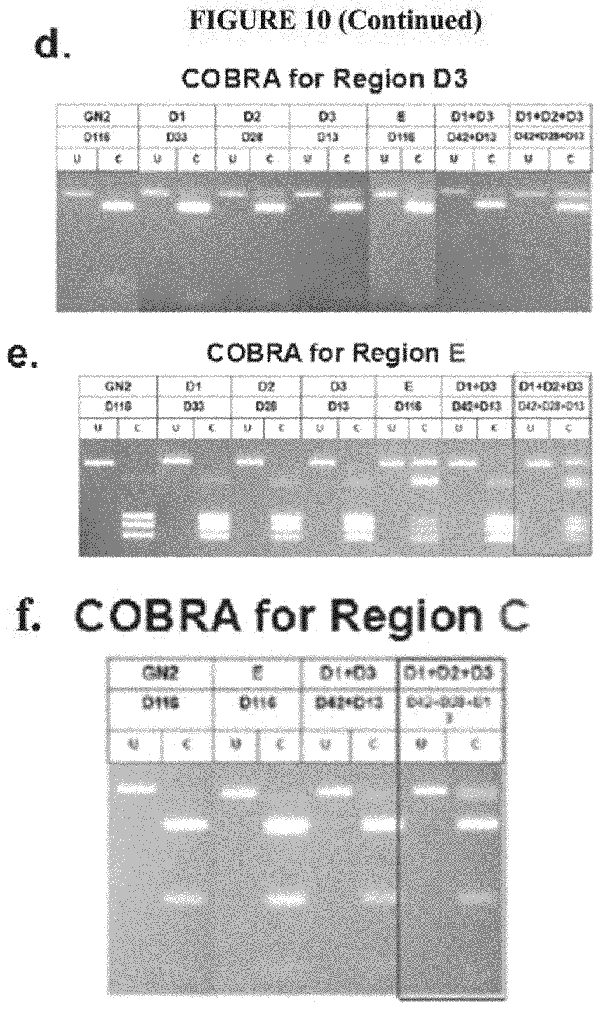

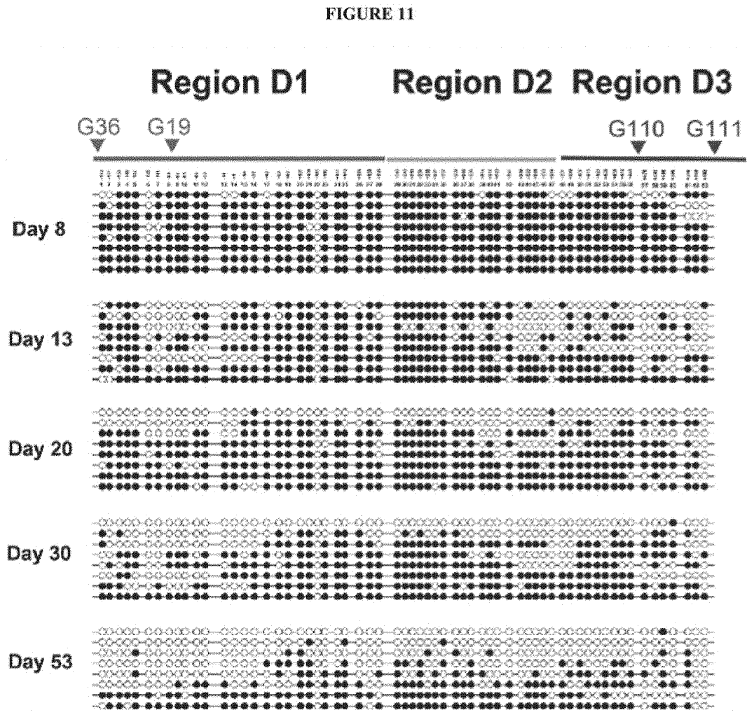

Three time points were selected to check gene expression, even though each region was not CRISPR-DiR treated with the same time. As shown in FIG. 10(b), only targeting Region D1 or D2 or D3 did not appreciably activate P16, while only targeting Region E initiated moderate gene expression. However, targeting both Region D1 and E enhanced the gene expression, while the combination of targeting D1, D3 or D1, D2, D3 together resulted in the highest activation. To note, even though targeting D1, D2, D3 all together had higher gene expression than that of targeting D1 and D3 in early time point, the gene expression level became the same in a later time points. In addition, the second time point samples were used to study the demethylation of Region C, D1, D2, D3 and E. Each region got demethylated when this region was targeted by CRISPR-DiR, and Region C and E were also dymethylated if D1, D2 and D3 were all targeted. Again, results show that 1) CRISPR-DiR may induce locus specific demethylation, 2) the demethylation in Region D may spread to the flanking regions, and 3) demethylation in key regulation regions led to gene activation.

FIG. 10 shows CRISPR-DiR targeting of P16 Region E, D1, D2, and D3 Region or Region D1. Each region was targeted with four mixed guide RNAs. In FIG. 10(a) the targeting strategy is shown; in FIG. 10(b) the P16 expression profile is shown; in FIG. 10(c) the methylation in Region D1 measured by COBRA is shown; in FIG. 10(d) the methylation in Region D3 measured by COBRA is shown; In FIG. 10(e) the methylation in Region E measured by COBRA is shown; in FIG. 10(f) the methylation in Region C measured by COBRA is shown. Region E was targeted for 116 days, Region D1 was targeted for 33 days, Region D2 was targeted for 28 days, Region D3 was targeted for 13 days. The red frames highlight that Region C and E was demethylated even not directly targeted.

The multiple regions targeting results indicated that among all of the regions, Region D1 and D3 may be the key regions where the demethylation correlates with highest gene activation. Therefore, highly effective identified targeting regions were identified for P16 demethylation and activation via CRISPR-DiR. Based on all these studies and results, the CRISPR-DiR system is found to be very interesting as it not only repurposes the endogenous RNA loops to specifically demethylate another gene locus and restore gene expression, but also it may to mimic a more natural demethylation and epigenetic regulation process, which may provide for tracing the entire epigenetic regulation and transcription mechanism starting from the demethylation of silenced genes. Thus, this system was used to explore the dynamic changes of gene regulation. These studies were focused mainly on Region D1 and D3 to make new stable cell lines targeting multiple regions at the same time, and the cells were traced from the very beginning of CRISPR-DiR treatment.

CRISPR-DiR Targeting Region D1 and D3 for the Most Effective P16 Demethylation and Activation Identified:

Since particularly effective CRISPR-DiR design (sgR2R5-dCas9) and particularly effective targeting regions (D1 and D3) were identified, all the sgR2R5 with guides targeting Region D1 and D3 (G2sgR2R5, G19sgR2R5, G36sgR2R5, G82sgR2R5, G109sgR2R5, G110sgR2R5, G111sgR2R5, G112sgR2R5) (see Table above for sequences) were transduced into SNU398-dCas9 stable cell line. The day of transducing sgR2R5 was Day 0, and the cells were cultured for 53 days to study the gene expression and demethylation process.

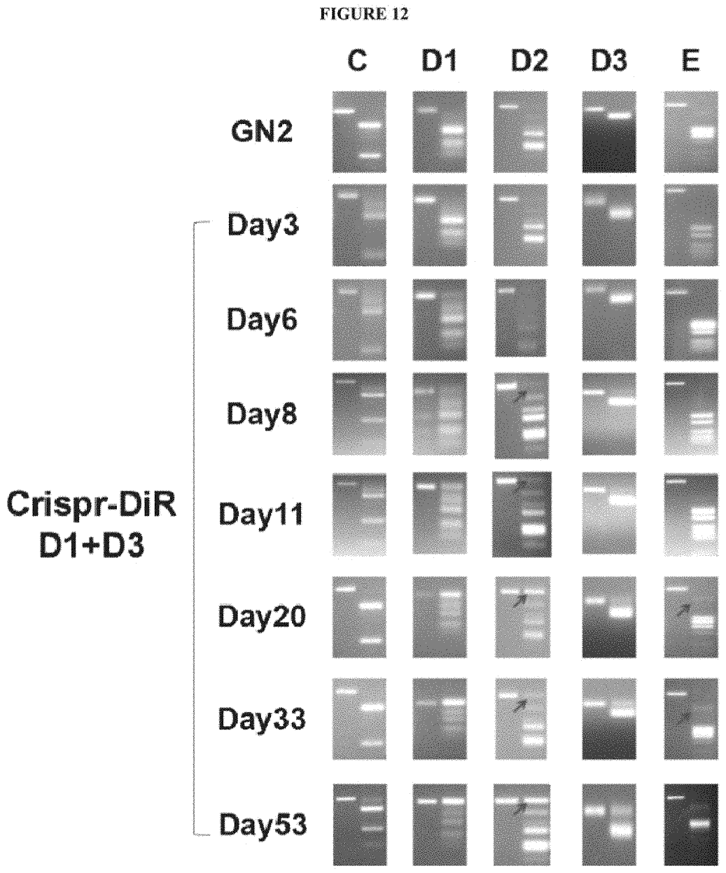

CRISPR-DiR Successfully Induced P16 Demethylation and Restored both Gene Expression and Function: P16 expression and demethylation of Region D1 and D3 targeted cells was checked in Day0, Day3, Day6, Day8, Day13, Day20, Day30, Day43 and Day53. The qPCR results showed that P16 mRNA expression was stably activated in Day13 and increased gradually in the whole process (FIG. 3b). COBRA data shown in FIG. 3d indicated that Region D1 demethylation started in Day6 while Region D3 demethylation started in Day8. The demethylation in the nearby regions which were not targeted (Region C, D2 and E) was also checked. As shown in FIG. 12, the 5′ flanking region C had no demethylation in the whole process, while the 3′ flanking region E got partial demethylation from Day 20. As for the middle Region D2, it was demethylated from Day 8 even though not directly targeted. The successful P16 demethylation and activation has been reproduced when the cells were targeted in Region D1 and D3 simultaneously, and it's consistent that gene demethylation occurred prior to mRNA expression, and the demethylation may spread to nearby regions which is hypothesized to be easier or important to undergo demethylation through the gene activation process. Of note, the moderate spreading of demethylation from Region D to E took a month to occur, and Region C was not demethylated through the 53 days tracing period, which is consistent with the BSP data (FIG. 5c) that Region C was not demethylated even with Decitabine treatment for three or five days. This indicated that P16 activation may be achieved if demethylation in certain regions, instead of the whole promoter, is achieved and CRISPR-DiR induced demethylation is highly locus specific. Genome wide methylation analysis and RNA-seq may be performed to further investigate off-target effect.

FIG. 3 shows CRISPR-DiR targeting P16 Region D1 and D3 simultaneously, with four guides targeting both strands in each region. FIG. 3(a) shows the targeting strategy; FIG. 3(b) shows the P16 expression profile; FIG. 3(c) shows the P16 protein restoration in Day 53; FIG. 3(d) shows the methylation in Region D1 and D3 measured by COBRA; and FIG. 3(e) shows the cell cycle analysis of the Day 53 treated samples. FIG. 11 shows the Bisulfite PCR sequencing result for the dynamic demethylation progress of CRISPR-DiR treated samples, and accompanies the data shown in FIG. 3.

FIG. 12 shows the methylation profile in Region C, D1, D2, D3 and E during the whole 53 days CRISPR-DiR treatment, measured by COBRA. CRISPR-DiR targeting p16 Region D1 and D3 simultaneously, with four guides targeting both strands in each region.

P16 is an important cell cycle regulator which decelerates the cell's progression from G1 phase to S phase. Therefore, since P16 mRNA has been successfully activated in these studies, and a slower growth of the D1, D3 targeted cells was observed, the functional restoration of P16 was further checked. The Day53 cells with the highest gene expression were taken, and the protein restoration as well as the cell cycle was studied. As shown in FIG. 3c, P16 protein was re-expressed in the Day 53 cells with CRISPR-DiR targeting Region D1 and D3, but not in the non-targeting control cells in the same time point. In addition, the increase of G1 phase population and decrease of S and G2 population were observed in the targeted cells compared with non-targeting cells in the same day (FIG. 3e). Therefore, the CRISPR-DiR induced demethylation not only initiated mRNA expression, but also the restoration of the gene function.

### CRISPR-DiR has Strand Specificity:

### CRISPR-DiR Preference for Targeting Gene Non-Template Strand for Demethylation and Activation:

The strand specificity of the CRISPR-DiR approaches described herein was then further investigated. Initially, guide RNAs were designed for Region E, targeting both DNA strands considering the role of DNMT1 in maintaining DNA methylation in the hemi-methylated DNA. When the demethylation and gene activation effects were compared between the mix of four guides and single guide, it was observed that the mixture of four guide RNAs worked better than any single guide, therefore four gRNAs mixture were used in the following experiments to target both strands. It was later realized that the most effective guides were those targeting Region D1 and D3 instead of Region E.

Therefore, the effects of only targeting one DNA strand and targeting both strands was next compared. SNU-398-dCas9 stable cells were newly transduced with four sgR2R5 lentivirus targeting (being complementary to) either the sense strand (non-template strand, NT) in the same genomic orientation as P16 (S) in D1 and D3 regions (G19, G36, G110, G111) or in the antisense (template strand, T) direction (AS) in D1 and D3 regions (G2, G82, G109, G112) (see Table above for sequences). Surprisingly, during the 20 days the treated cells were traced, P16 activation was only observed by CRISPR-DiR (second generation) targeting the S strand (non-template strand, NT). The expression levels of P16 in Sense strand (non-template strand) sgR2R5 targeted cells were even higher than those targeted with both S and AS gR2R5. By contrast, there was only very weak gene activation in the AS (template strand targeted) gR2R5 targeted cells. The extent of DNA demethylation was further analyzed in both cell lines. Region D1 and D3 were highly demethylated for both DNA strands when the S strand (non-template) was targeted, while there was only a weak demethylation in region D1 and no demethylation in Region D3 when only the AS strand (template) was targeted. COBRA was performed for both DNA strands to check methylation in the same regions and the same result was obtained. These results ruled out the possibility that CRISPR-DiR (second generation) only demethylated the AS (template) strand, showing that only CRISPR-DiR targeting the S (non-template) strand in Region D1 and D3 induced gene demethylation in both strands and therefore initiated mRNA activation.

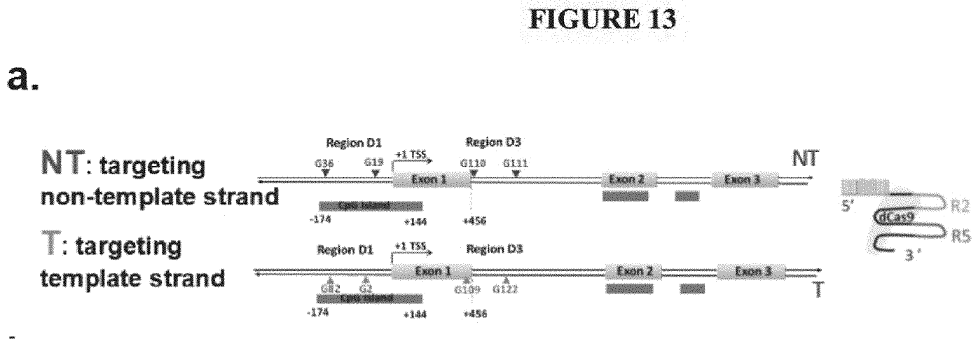

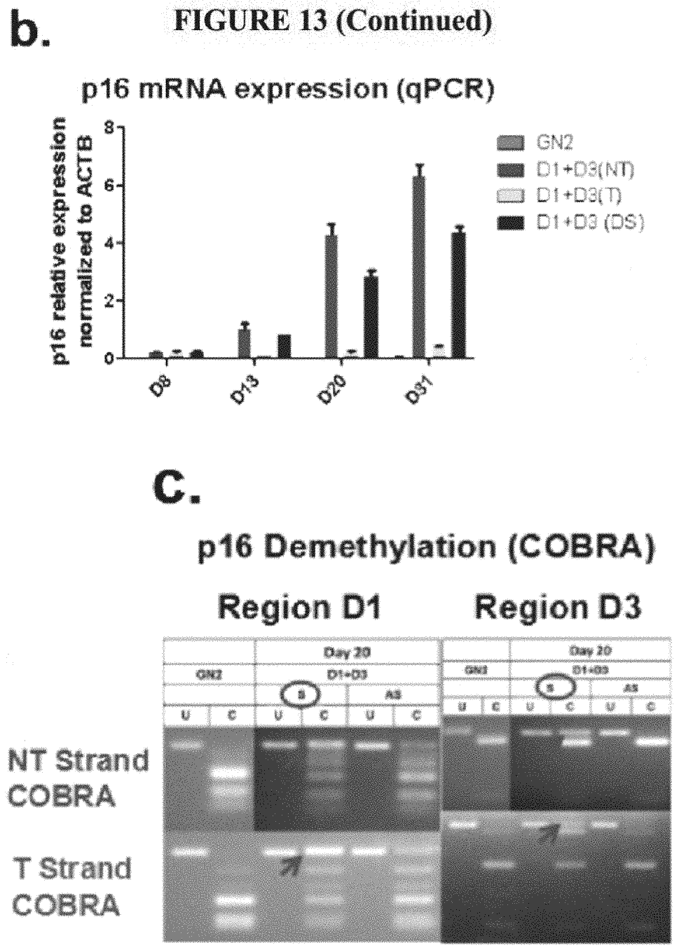

FIG. 13 shows results of CRISPR-DiR targeting p16 Region D1 and D3 simultaneously, with only one DNA strand targeted in each sample. FIG. 13(a) shows the targeting strategy; FIG. 13(b) shows the p16 expression profile; and FIG. 13(c) shows the methylation profile in Region D1 and D3 measured by COBRA. Targeting means the guide RNA sequence is complimentary to the targeted strand. The mRNA sequence (sense strand) is the same as the non-template strand. Thus, in the COBRA data, S (sense strand) refers to targeting non-template strand (NT), AS (antisense) refers to targeting template (T) strand.

In this study several CRISPR-DiR structure designs were explored to identify particularly effective CRISPR-DiR designs (i.e. sgR2R5-dCas9) and particularly effective targeting regions of p16 (i.e. D1 and D3). All the sgR2R5 with guides targeting Region D1 and D3 (G2sgR2R5, G19sgR2R5, G36sgR2R5, G82sgR2R5, G109sgR2R5, G110sgR2R5, G111sgR2R5, G112sgR2R5) (see Table above for sequences) were transduced into SNU398-dCas9 stable cell line (see FIG. 3). The day of transducing sgR2R5 was Day 0, and the cells were cultured for 53 days to study the gene expression and demethylation process. CRISPR-DiR successfully induced p16 demethylation and restored both gene expression and function in these studies. p16 expression and demethylation of Region D1 and D3 targeted cells was checked in Day0, Day3, Day6, Day8, Day13, Day20, Day30, Day43 and Day53. The qPCR results showed that p16 mRNA expression was stably activated in Day13 and increased gradually in the whole process (FIG. 3b). COBRA data shown in FIG. 3d indicated that Region D1 demethylation started in Day6 while Region D3 demethylation started in Day8. The demethylation in the nearby regions which were not targeted (Region C, D2 and E) was also checked. As shown in FIG. 12, the 5′ flanking region C had no demethylation in the whole process, while the 3′ flanking region E got partial demethylation from Day20. As for the middle Region D2, it was demethylated from Day 8 even though not directly targeted. The successful p16 demethylation and activation has been reproduced when the cells were targeted in Region D1 and D3 simultaneously, and it's consistent that gene demethylation occurred prior to mRNA expression, and the demethylation may spread to nearby regions which it is contemplated may be easier or important to undergo demethylation through the gene activation process. Of note, the moderate spreading of demethylation from Region D to E took a month to occur, and Region C was not demethylated through the 53 days tracing period, which is consistent with BSP data that Region C was not demethylated even with Decitabine treatment for three or five days. This indicated that p16 activation may be provided by demethylation in certain regions instead of the whole promoter, and CRISPR-DiR induced demethylation may be highly locus-specific.

In CRISPR-Cas9 systems, the Cas9 protein with nuclease activity is guided to genomic loci by a typically 20 nt single guide RNA (sgRNA) complementary to the genomic target site (11, 12). The Cas9-sgRNA complex unwinds the target double-stranded DNA and induces base paring of the sgRNA with the target DNA, and subsequently enables double-strand breaks (DSB) at the target DNA for gene knock-in or knock-out applications. Accordingly, there is typically no targeting strand selectivity in these applications. In terms of dead Cas9 (dCas9), it's a nuclease-deficient mutant of Cas9, with mutations in the RuvC and HNH nuclease domains, that preserves the ability to form a complex with sgRNA and DNA-binding proficiency guided by sgRNAs (13). In most CRISPR-dCas9 systems used for gene transcription regulation in eukaryotic cells, the dCas9 is fused with several regulatory domains to potentiate either the transcriptional activation or repression. To promote transcription, activation domains, commonly used as effectors to upregulate gene expression in eukaryotic cells (14), such as VP64 (4 copies of VP16), p65, VP160 (10 copies of VP16), VP192 (12 copies of VP16) and tandem repeats of a synthetic GCN4 peptide (SunTag) have been fused to dCas9 protein: i.e. dCas9-VP64, dCas9-p65, dCas9-VP160, dCas9-VP192 and dCas9-SunTag(15, 16) These activation domains are guided to specific gene loci by the sgRNAs and reinforce the expression of the targeted endogenous genes in mammalian cells (17-19). To repress transcription, Kruppel-associated box (KRAB) domain (20) and four copies of mSin3 interaction domain (SID4X) may be fused to dCas9 (dCas9-KRAB and dCas9-SID4X) as transcriptional repression systems. However, none of the above CRISPR-Cas9/dCas9 systems depends on strand-specificity as found for the presently described CRISPR-DiR systems. Indeed, targeting either template or non-template strand typically shows an equal effect on gene regulation in other systems. Remarkably, strand specificity/preference for the presently described CRISPR-DiR systems has been found, and may be used to provide particularly effective demethylation and/or gene activation.

The presently described CRISPR-dCas9 systems achieve specific gene demethylation and activation, based on naturally modified gRNAs, and show a non-template strand selectivity/preference. As indicated in FIG. 4, targeting the non-template strand of P16 region D1 and D3 (the guide RNAs G19, G36, G110, G111 are complementary to the non-template strand) led to higher P16 expression at the same time points as compared to the gRNAs targeting both template and non-template strand for the same regions (guide RNAs G19, G36, G110, G111 are complementary to the non-template strand, while guide RNAs G2, G82, G109, G112 are complimentary to the template strand) (FIG. 4a). Importantly, guide RNAs targeting the template strand (guide RNAs G2, G82, G109, G112 are complimentary to the template strand) did not induce significant demethylation in Region D3 and very weak in Region D1 with no effect on P16 mRNA expression.

While off-target effects may be possible to some extent in certain conditions, in the present studies when p16 was targeted by CRISPR-DiR, the methylation and mRNA expression level of several genes either close to p16 (p14 and p15) or far away from the targeted region (CEBPA, SALL4) were analyzed, and data showed no change in any of these gene locus.

The CRISPR-dCas9 DiR systems described herein are based on sgRNA modifications using natural existing sequences without requiring fusing of proteins to dCas9, and have been found to works notably better when targeting non-template strand instead of template strand in the studies described herein. The non-template strand specificity/preference of CRISPR-DiR may provide a key design rule when seeking to design particularly effective oligonucleotide constructs for demethylation and/or gene activation. The presently developed CRISPR-DiR systems are observed herein to be gene-specific demethylating and activating tools using DNMT1-interacting RNA short loops to block DNMT1 methyltransferase activity at specific loci.

DNA methylation abnormalities play a significant role in cancer diseases. Development of demethylating agents (azacitidine, decitabine) to treat hypermethylation associated diseased has been investigated, but the lack of specificity for the genetic loci and the high toxicity has presented challenges. A specific class of RNAs (DNMT1-interacting RNAs, DiRs) is able to bind to DNMT1 with stem-loop structure and to protect numerous DiR-expressing loci from methylation and silencing. As described herein, a CRISPR-DiR system has now been developed as a gene-specific demethylation and activation tool. In this system, DNMT1-interacting RNA (DiR) stem loops are fused to single CRISPR guide RNA (sgRNA) scaffold. As a result, the DiR loops may be delivered to a specific locus and interact with DNMT1 to block methyltransferase activity. By designing CRISPR-DiR guides specifically targeting the p16 promoter CpG island as well as the first Exon, p16 was successfully demethylated and this tumor suppressor gene mRNA and protein expression was restored as well as the cell cycle arrest function in SNU-398 HCC cell line and U2OS osteosarcoma cells. Interestingly, the CRISPR-DiR induced demethylation took about a week to occur, while the initiation of gene transcription took even longer. Accordingly, it is contemplated that this approach may not only provide a powerful locus-specific tool for demethylation, but may also more closely mimic a more natural demethylation process, which may allow for further tracing of the entire regulation process. In addition, the successful application of CRISPR-DiR to SALL4 gene indicated that the presently described systems may be a general approach for multiple genes.

The histone makers change in CRISPR-DiR treated Day53 cells were studied by ChIP-qPCR. As shown in FIG. 19, in p16 proximal promoter region, there were significant increase of gene activation markers H3K4me4 and H3K27ac, while decrease of gene silencing marker H3K9me3. These histone changes are specific in p16 locus, as there are no changes in the nearby genes (P14, P15) and downstream negative region (10 Kb downstream of P16). The histone changes are consistent with CRISPR-DiR induced P16 demethylation and activation, and the specificity also indicated that CRISPR-DiR is a gene specific method.

As described in detail herein, CRISPR-DiR have now been developed as RNA-based tools for gene-specific demethylation. There is much interest in using RNA molecules as a therapeutic tools (Kole et al., 2012, Reebye et al., 2014). It is contemplated that in certain embodiments, approaches as described herein may provide benefit over the existing hypomethylating-based protocols. For example, it is contemplated that in certain embodiments high gene specificity; lower cytotoxicity (versus certain other drugs); and/or c) absence of certain drug-associated off-target side-effects may be provided. Controlling in loco gene expression may be of particular interest in clinical applications, and it is also contemplated that tools as described herein may be used to further investigate the epigenetic regulation process and/or for identification of key regulators and/or targets for therapeutic treatments. In certain embodiments, CRISPR-DiR systems as described herein may provide RNA-based gene-specific demethylating tools for disease treatment, for example.

### Example 3-Targeted Intragenic Demethylation Initiates Chromatin Rewiring for Gene Activation

Building from the results of Examples 1 and 2 above, this Example further investigates and describes Crispr-DiR and gene activation. Results from Examples 1 and 2 (re-iterated below), and additional results set out in this Example, indicate that locus demethylation via CRISPR-DiR reshapes chromatin structure and specifically reactivates its cognate gene.

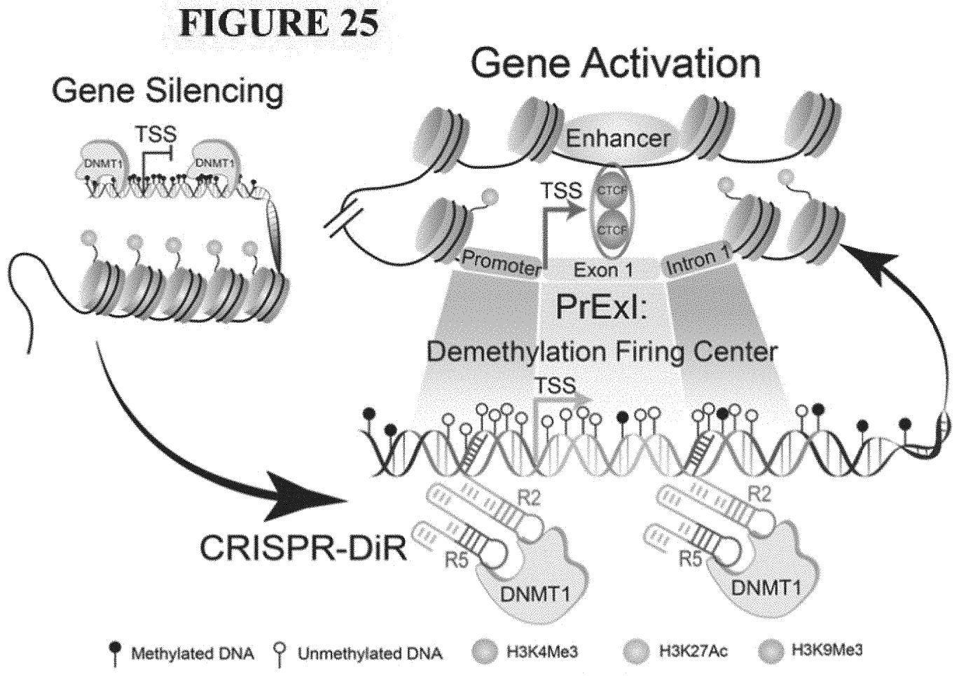

Results in this Example indicate direct evidence that instead of solely the methylated proximal promoter, a specialized “demethylation firing center (DFC)” covering the proximal promoter-exon 1-intron 1 (PrExI) region correlates more with gene reactivation by initiating a wave of both local epigenetic modifications and 500 kb distal chromatin remodeling (See FIG. 25). This finding is demonstrated in a gene locus specific manner via CRISPR-DiR, which reverts the methylation status of the targeted region by RNA-based blocking of methyltransferase activity.

Aberrant DNA methylation in the region surrounding the transcription start site is a hallmark of gene silencing in cancer. In the field, currently approved demethylating agents lack specificity, and exhibit high toxicity. Aberrant DNA methylation, especially methylation in the 5′ promoter region upstream of the transcription start site, has been frequently reported to be associated with tumor suppressor gene silencing in cancers. However, studies involving non-specific hypomethylating agents, such as 5 azacytidine in myelodysplastic syndrome, have not demonstrated good correlations with demethylation of this upstream region and gene reactivation. In addition, it has been unclear what other potential elements work with the promoter region resulting in locus-specific DNA demethylation culminating in gene activation, or whether other local and distal chromatin remodeling events are initiated by demethylation of a short functional intragenic region. One of the reasons for lack of clarity on these issues is because of the previous lack of a locus specific demethylation tool which can efficiently initiate target specific demethylation and allow the downstream endogenous epigenetic regulatory process.

This Example provides new insights into these questions using a locus specific demethylation system, CRISPR-DiR. DNA methyltransferase I (DNMT1), which mediates methylation of tumor suppressors, is regulated by and can be inhibited by certain noncoding RNAs (ncRNAs, which are referred to as DNMT1-interacting RNAs, or DiRs) in a gene selective manner, and the interaction is based on RNA secondary stem-loop structure (Di Ruscio, et al., Nature, 2013). In the CRISPR-DiR system, short DiR stem loops have been inserted into CRISPR single guide RNA (sgRNA) scaffold and therefore can be repurposed to virtually any target site for demethylation. In this Example, using one of the most reported hypermethylated tumor suppressor genes, p16, as a model, the application of CRISPR-DiR to several different regions around transcription start site revealed the critical epigenetic regulation functions are located in both the upstream promoter region and the intragenic exon 1-intron 1 region. This proximal promoter-exon 1-intron 1 region (PrExI) is characterized as a “demethylation firing center (DFC)”, which initiates epigenetic waves to reshape both local histone modifications and distal chromatin interactions and thus restoring gene expression.

Indeed, this Example shows, using the p16 gene as an example, that targeted demethylation of the promoter-exon 1-intron 1 (PrExI) region initiates an epigenetic wave of local chromatin remodeling and distal long-range interactions, culminating in gene-locus specific activation. Through development of CRISPR-DiR, in which ad hoc edited guides block methyltransferase activity in a locus-specific fashion, this Example indicates that demethylation is coupled to epigenetic and topological changes. These results suggest the existence of a specialized “demethylation firing center (DFC)”, which may be switched on by an adaptable and selective RNA-mediated approach for locus-specific transcriptional activation. DNA methylation is a key epigenetic mechanism implicated in transcriptional regulation, normal cellular development, and function (29). The addition of methyl groups that occurs mostly within CpG dinucleotides is catalyzed by three major DNA methyltransferase (DNMT) family members: DNMT1, DNMT3a, and DNMT3b. Numerous studies have established a link between aberrant DNA methylation and gene silencing in diseases (30, 31).

Tumor suppressor gene (eg. p16, p15, MLH1, DAPK1, CEBPA, CDH1, MGMT, BRCA1) silencing is frequently associated with abnormal 5′CpG island (CGI) DNA methylation (32) and it is considered as the hallmark of most if not all cancers (33). Since 70% annotated gene promoters overlap with a CGI (34), the majority of studies in the field have only concentrated on the correlation of CpG island promoter methylation and transcriptional repression, specifically focusing on the region just upstream of the transcription start site (TSS) (30, 35-37), but neglecting some studies showing the regulatory importance of regions downstream of TSS (38, 39). Therefore, the regulatory importance and mechanism of intragenic methylation on gene expression has been unclear. Traditional demethylating agents are of limited utility, experimentally and therapeutically, because they act indiscriminately on the entire genome (40). Thus, an approach that is able to selectively modulate DNA methylation represents a powerful tool for locus specific epigenetic regulation and study thereof, and a potential non-toxic therapeutic option to restore expression of genes aberrantly silenced by DNA methylation in pathological conditions.

Epigenetic control hinges on a fine interplay between DNA methylation, histone modifications, nucleosome positioning, and their respective genetic counterparts: DNA, RNA, and distal regulatory sequences involved in the formation of specific topological domains (41). This structured organization is the driver for both gene activation or repression (42). It has been unclear to what extent locus-specific DNA demethylation contributes to chromatin structural rearrangements culminating in activation of silenced genes. In the field, the lack of methods promoting naïve and localized specific demethylation have been a major constraint to understand the sequential mechanistic aspects enabling locus-specific activation.

Previously, our group (43) identified RNAs inhibiting DNMT1 enzymatic activity and protecting against gene silencing in a locus specific modality, termed DNMT1-interacting RNAs (DiRs). This interaction relies on the presence of RNA stem-loop-like structures, and is lost in their absence. As described herein, by combining the demethylating features of DiRs with the targeting properties of the CRISPR-dead Cas9 (dCas9) system, a CRISPR-DiR platform has been developed, to induce precise locus specific demethylation and activation. The incorporation of DiR-baits into the single-guide RNA (sgRNA) scaffold enables the delivery of an RNA DNMT1-interacting domain to a selected location while recruiting dCas9 (36, 44, 45).

p16 was selected in this Example to further test the CRISPR-DiR platform, because it is one of the first tumor suppressor genes more frequently silenced by promoter methylation in cancer (46). As described, it was observed that gene-specific demethylation not only in the upstream promoter, but also in the exon 1-intron 1 region, initiates a robust stepwise process, followed by the acquisition of active chromatin marks (eg. H3K4Me3 and H3K27Ac), enrichment of methylation sensitive regulators (eg. CTCF), and interaction with distal regulatory elements, ultimately leading to stable gene-locus transcriptional activation. Overall, these studies point to discovery and development of a specialized promoter-exon 1-intron 1 region as a “demethylation firing center” responsive to RNA-mediated control and governing gene-locus transcriptional activation, elucidating the previously unknown importance of intragenic exon 1-intron 1 demethylation for active gene transcription.

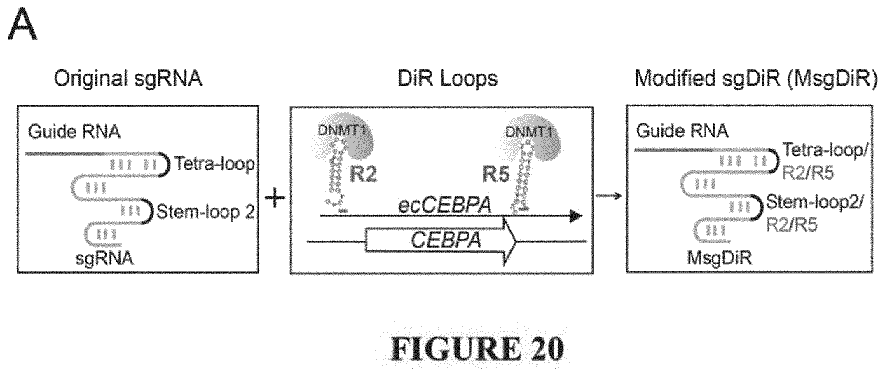

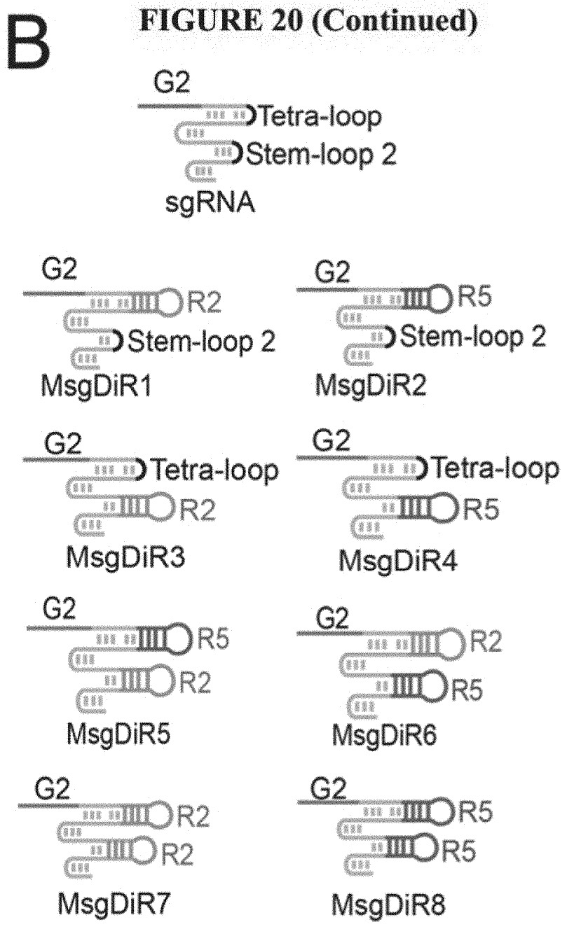

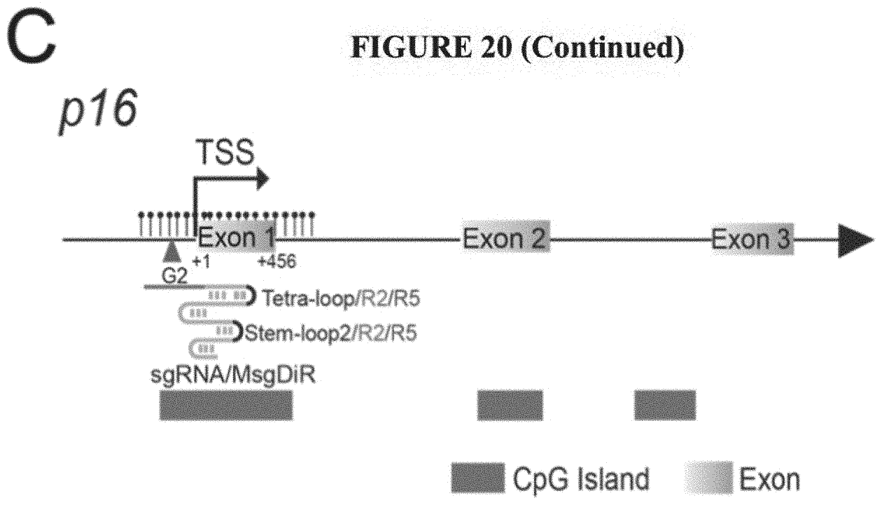

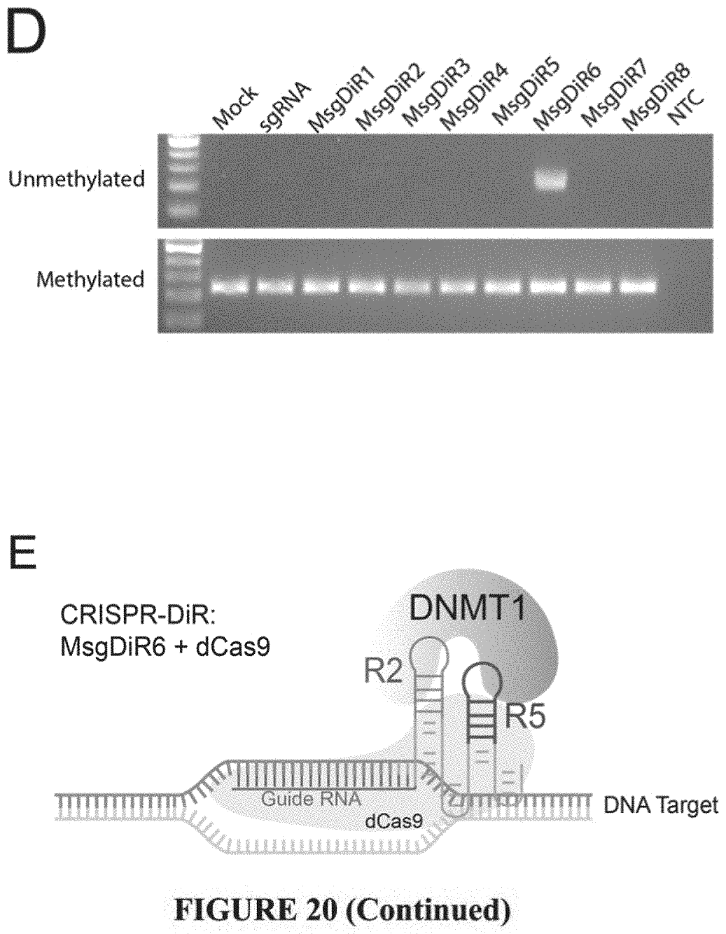

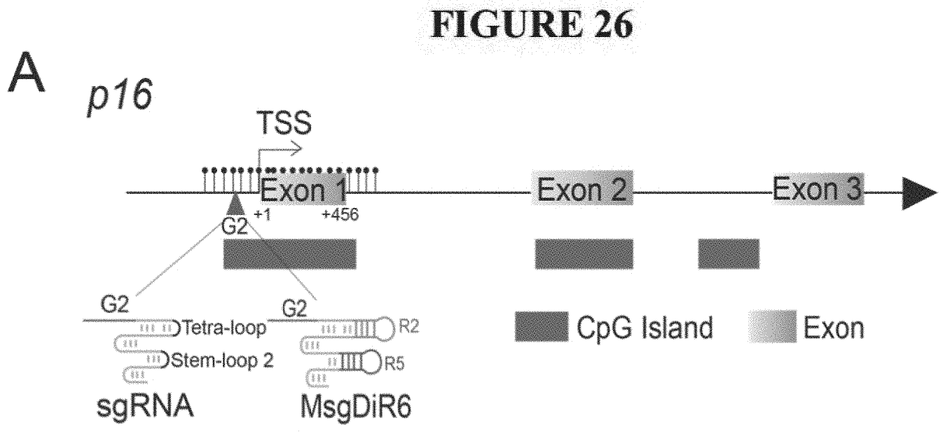

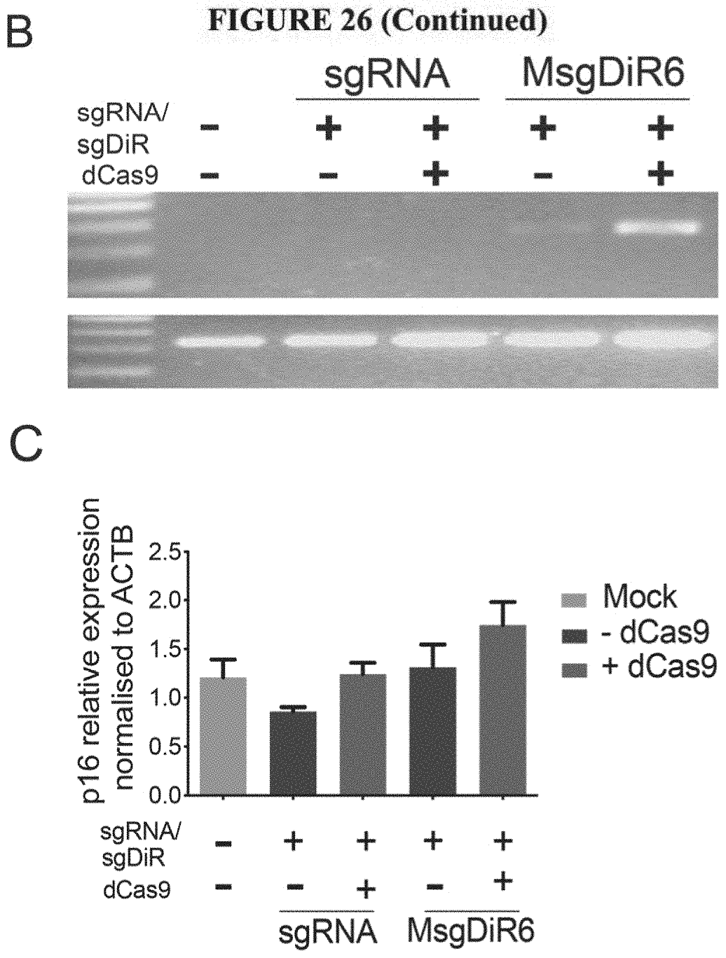

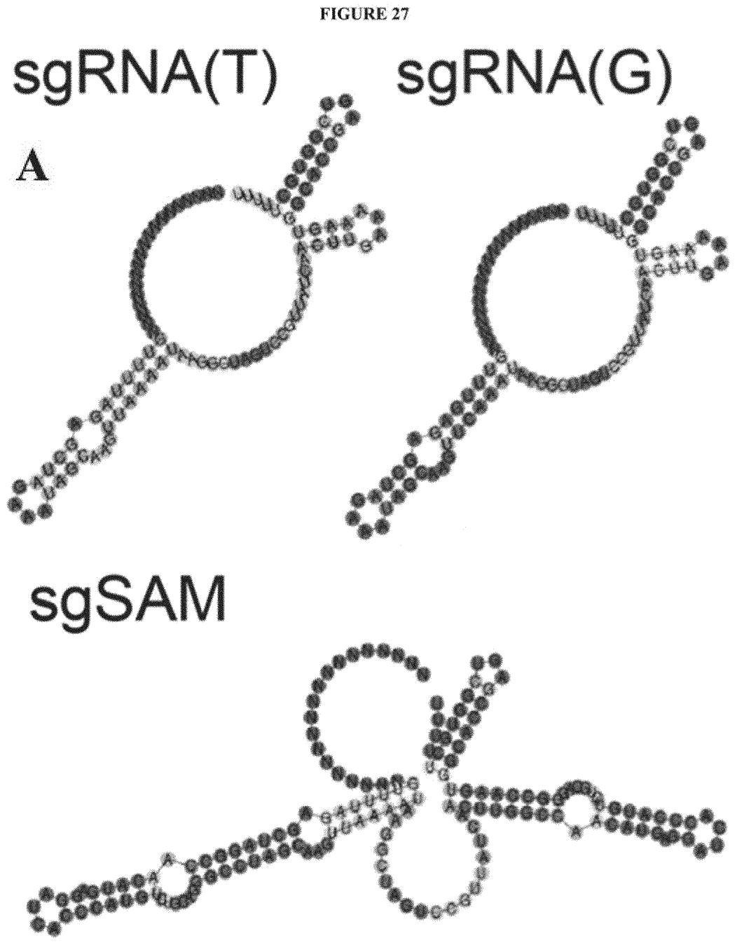

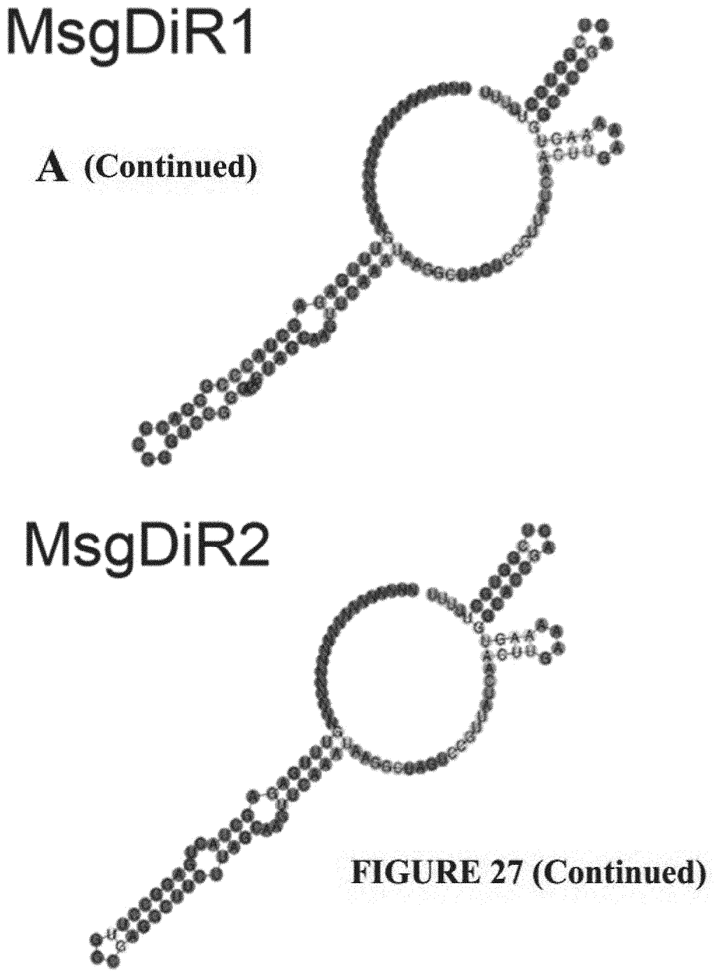

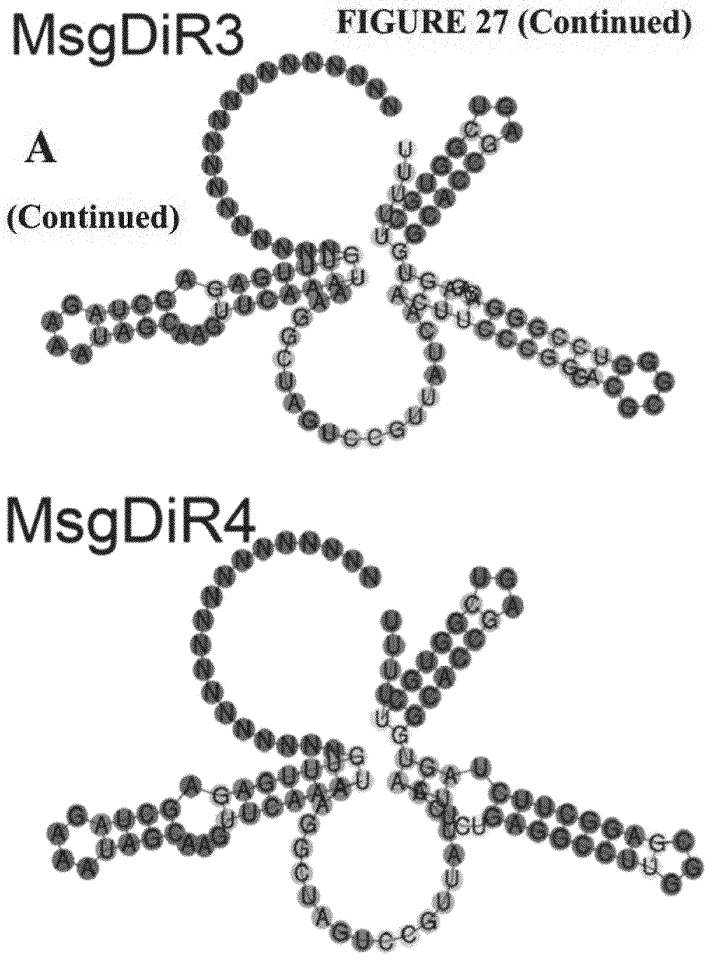

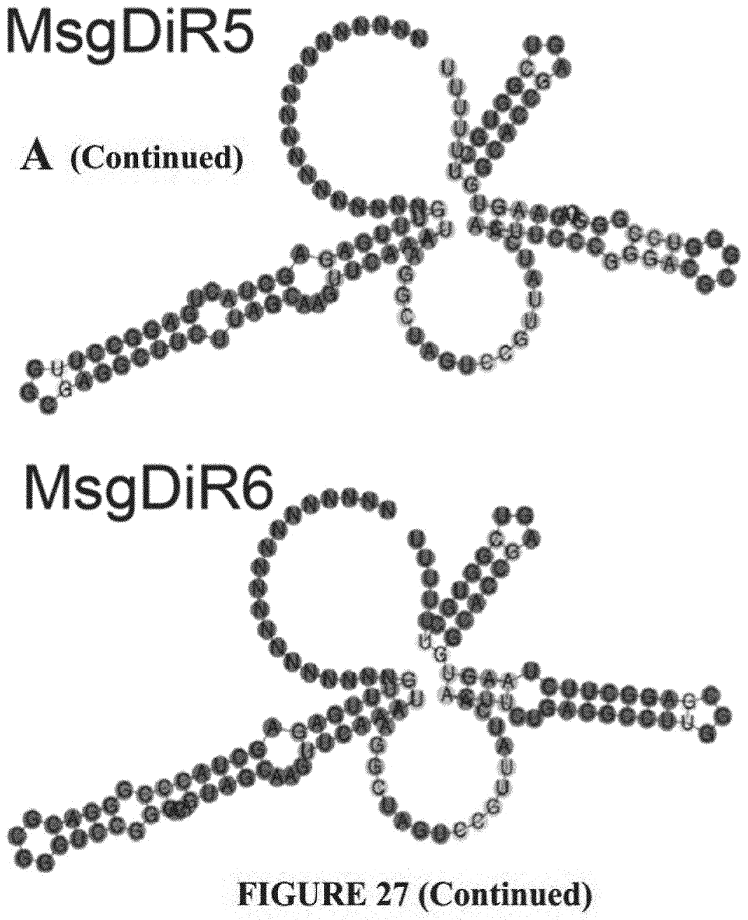

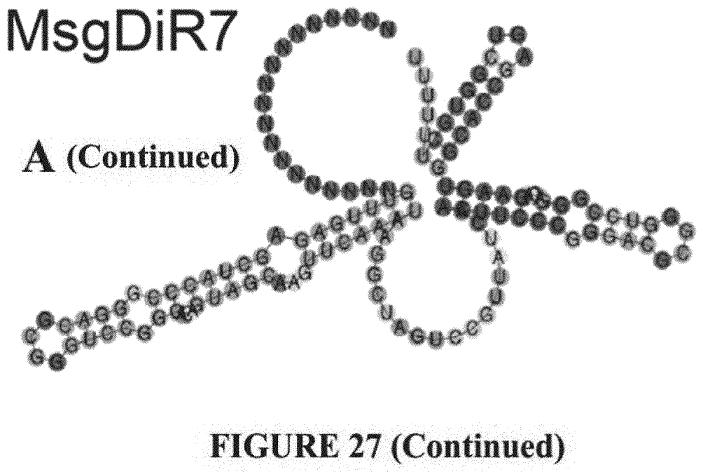

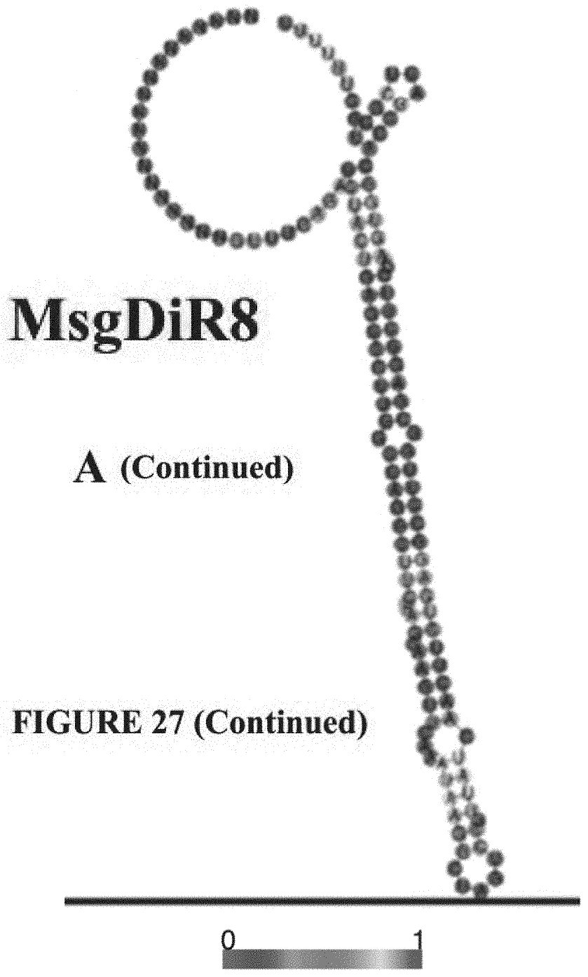

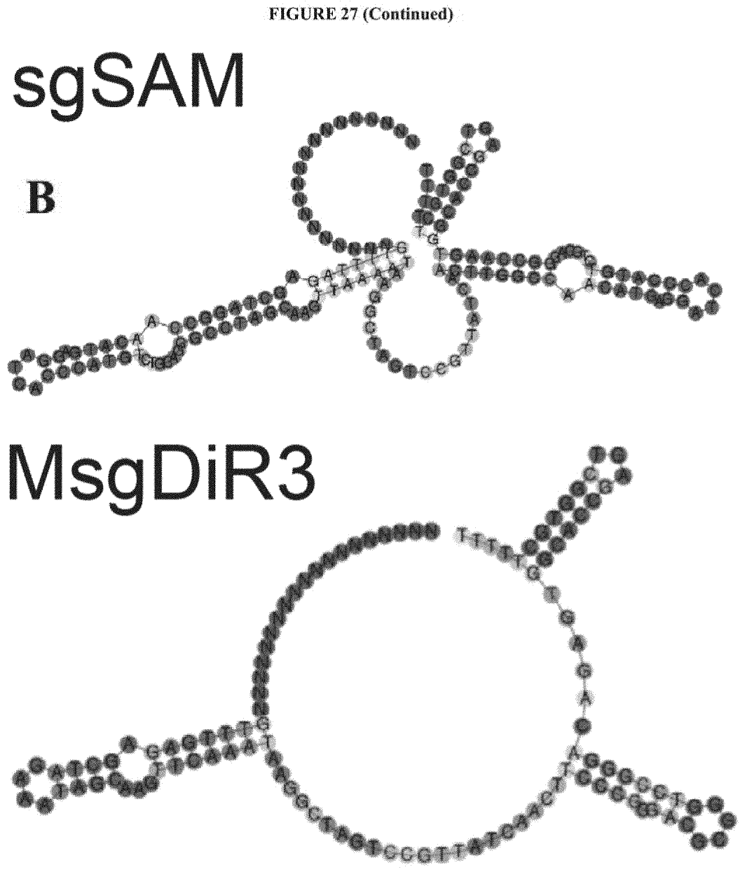

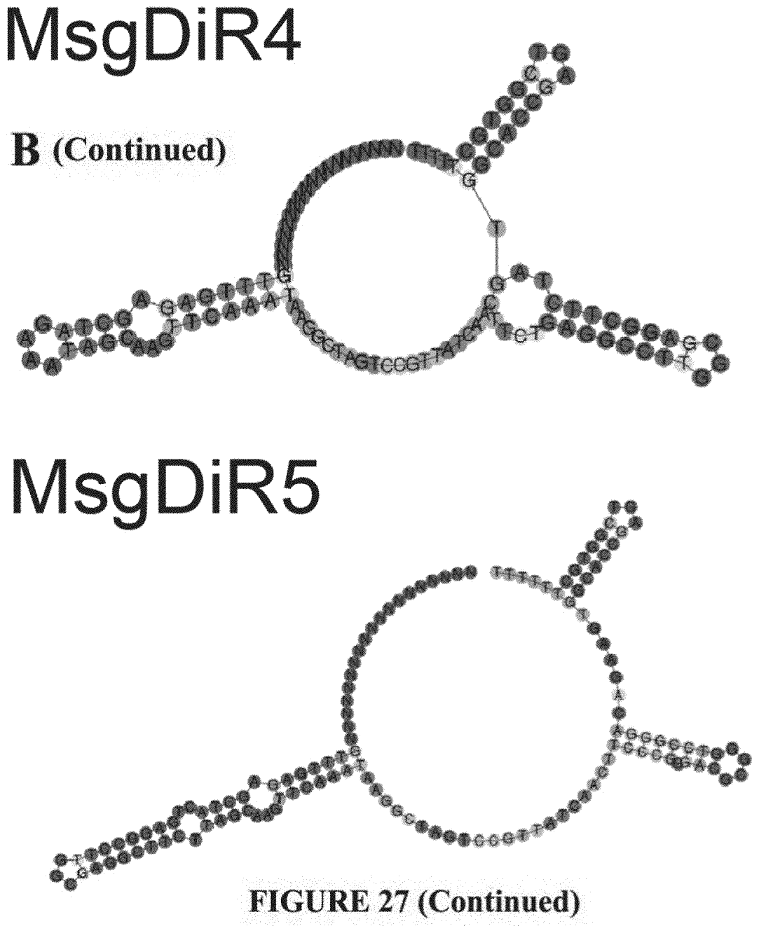

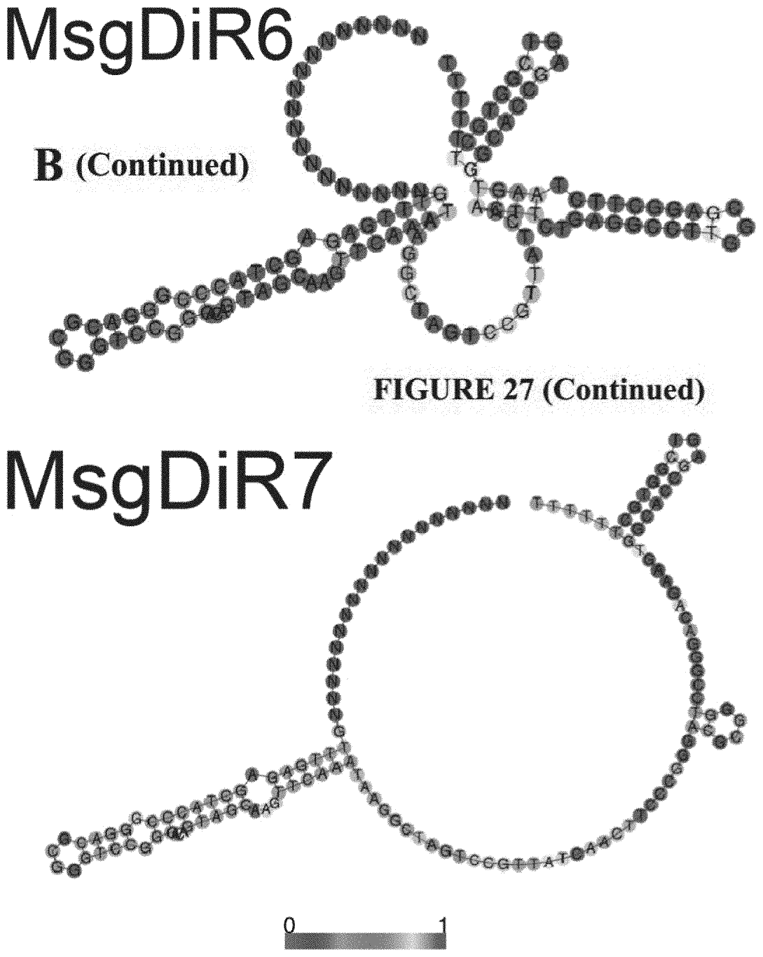

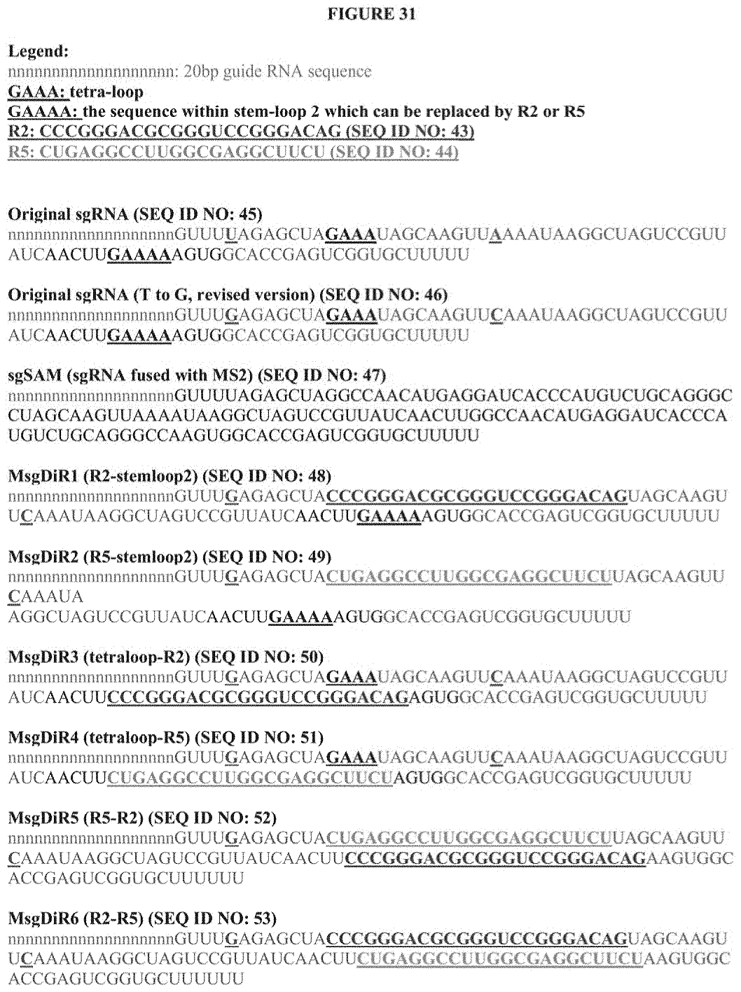

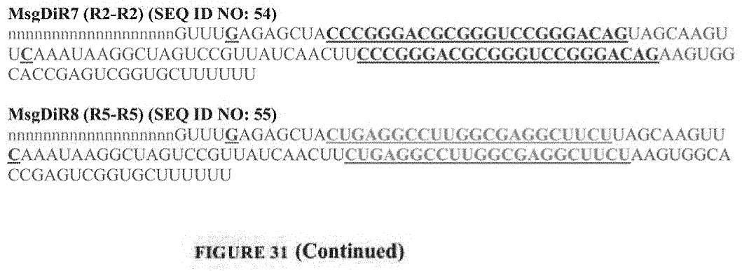

Development of the CRISPR-DiR system: Development of the CRISPR-DiR system has been described in detail in Examples 1 and 2 above. The initial screening results of 8 different designs, indicating CRISPR-DiR systems with R2-R5 designs (such as those used in Examples 1 and 2 above) as being preferred and effective designs, are further described here. As indicated, the tumor suppressor gene p16 (also known as p16^{INK4a}, CDKN2A) is one of the first genes commonly silenced by aberrant DNA methylation in almost all cancer types, including hepatocellular carcinoma (HCC) (32, 47, 48), and therefore it was chosen as a model to study the effect(s) of gene-specific demethylation. Studies on the secondary structure of the Cas9/dCas9-sgRNA-DNA complex, including evolution of the original system such as CRISPR-SAM (36) and CRISPR-Rainbow (44), suggested that the tetra-loop and stem loop 2 of the sgRNA scaffold are replaceable by RNA aptamers such as MS2 and PP7 without compromising the stability of the complex or its functionality. As discussed herein, incorporation of short loop sequences corresponding to R2 and R5 of the ecCEBPA DiR (43) may enable binding and inhibition of DNMT1 in a gene-specific manner (see FIG. 20A). Several R2/R5-tetra/stem loop 2 designs were tested (see FIG. 20B) in an effort to attain a platform in which 1) the sgRNA-dCas9 complex structure is stable; and 2) delivers efficient demethylation and gene activation. Starting with a guide sequence (G2) successfully used in other studies (36), modified sgRNAs (MsgDiR) were designed in which the tetra-loop and stem-loop embodied different combinations of R2 and R5 DiR loops (see FIG. 20B) targeting the p16 proximal promoter (see FIG. 20C). 8 different designs were tested, as shown. Sequences are shown in FIG. 31 and Table 3. dCas9 was co-transfected with either unmodified sgRNA (without DiR loops) or modified MsgDiR into SNU 398, a HCC cell line in which p16 is methylated and silenced. Seventy-two hours after transfection, only the MsgDiR6 model induced p16 demethylation (see FIG. 20D). Further validation of MsgDiR6 with or without dCas9 in cells with either a non-targeting control guide (GN2) or p16 guide (G2; localizing to a region of the p16 promoter) for demethylation (see FIG. 26A, 26B) demonstrated moderate activation of p16 in dCas9 positive cells, while no effects were observed in absence of dCas9 (see FIG. 26C). Intriguingly, MsgDiR6, which incorporates DiR loop R2 into the sgRNA tetra-loop and DiR loop R5 into sgRNA stem loop 2 (see FIG. 20B, 20E, hereafter referred to as sgDiR), was the only design able to form a compatible predicted and functional secondary structure as reported for original sgRNA and sgSAM (see FIG. 27A, 27B) (36, 45), suggesting that preserving the original structure is desirable when editing the protruding loops in the sgRNA design. The predicted secondary structure of dCas9-R2R5 system is closer to original Crispr systems, indicating dCas9-R2R5 may be comparatively more stable and/or efficient in terms of targeting, for example. The CRISPR-DiR platform induced locus-specific demethylation. Results indicate that in the system and conditions tested, some fusions of functional RNA into sgRNA tetra-loop and stem-loop 2 were not strong activators, whereas MsgDiR6 in particular was the best performing construct of the group.

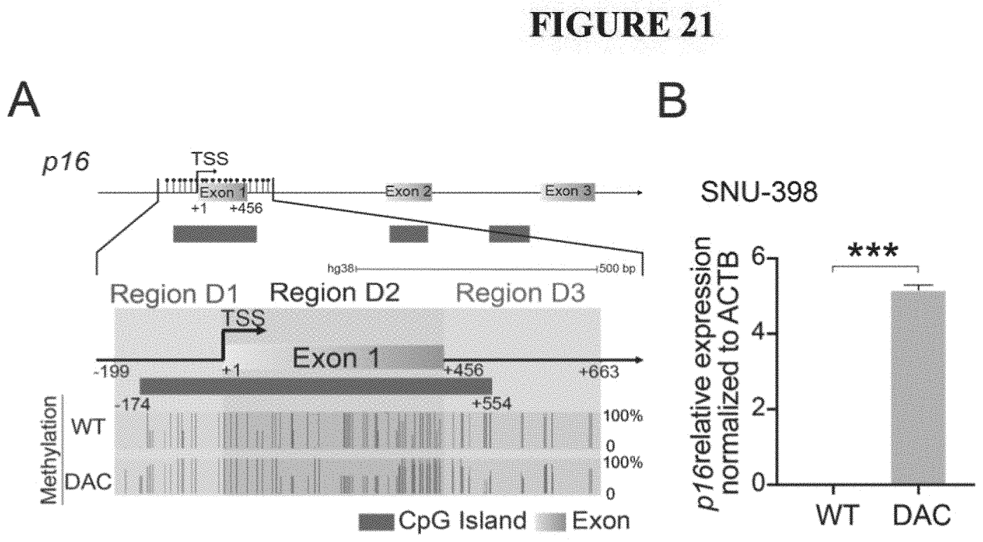

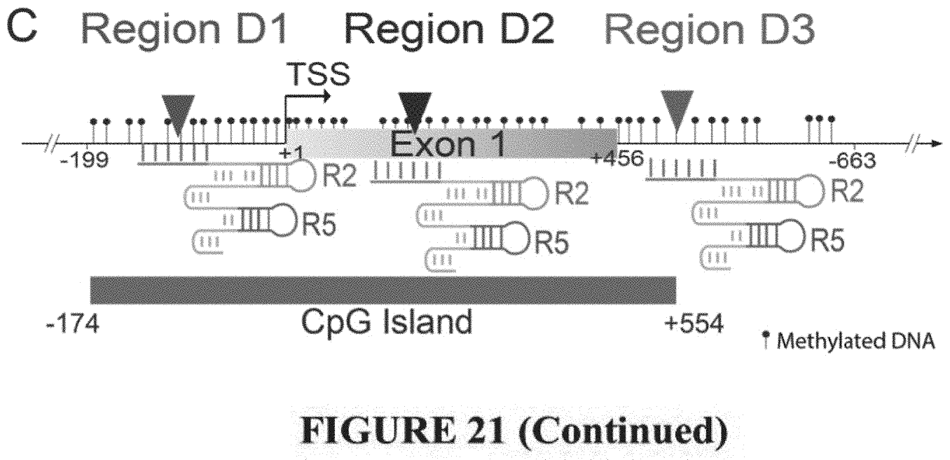

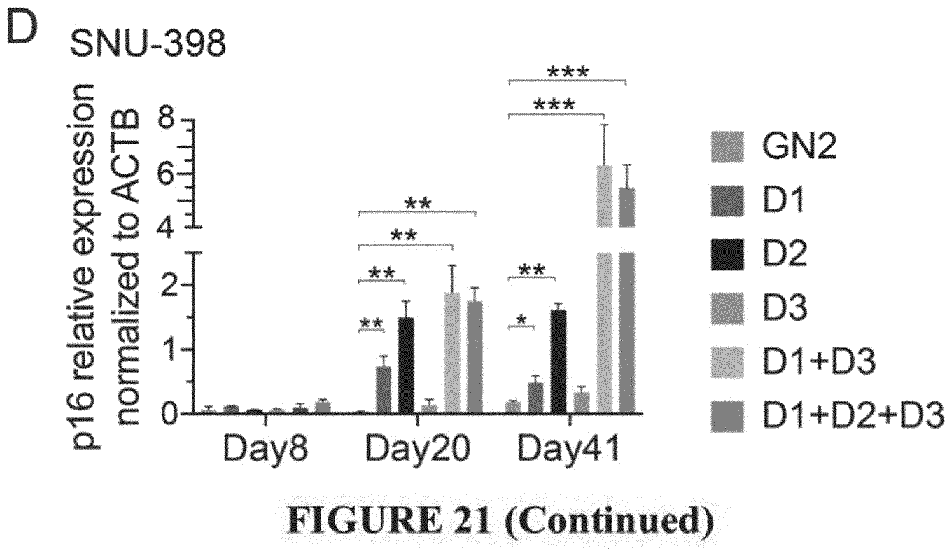

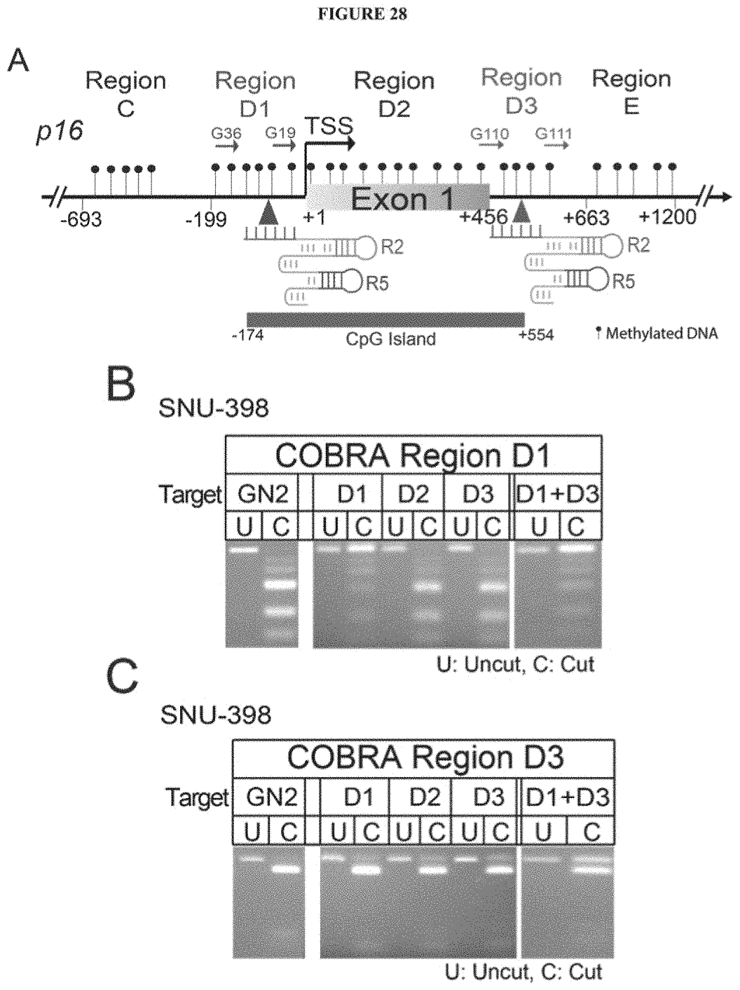

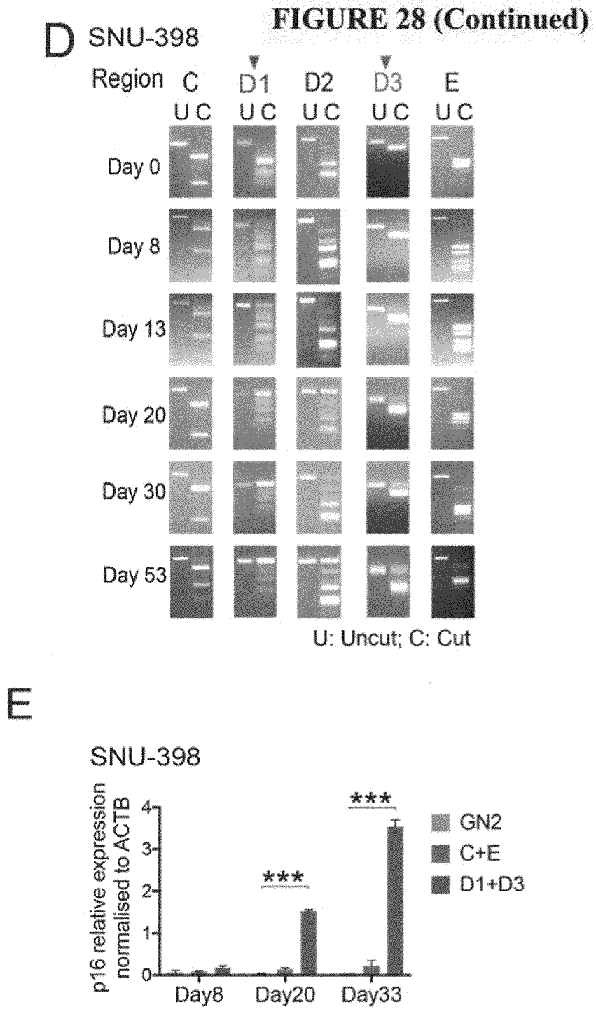

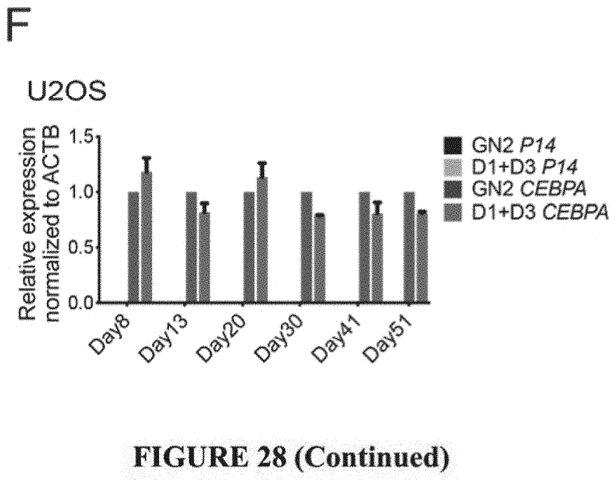

CRISPR-DiR unmasks the p16 transcriptional activator core: Although the initial analysis confirmed locus-specific demethylation, only a moderate activation of the mRNA was observed by the sgDiR (G2) targeting the p16 proximal promoter upstream of transcription start site (TSS) (see FIG. 26C). Understanding was sought whether other than the promoter, the demethylation of additional intragenic regions within the locus were desirable or important for transcriptional activation. To identify demethylation-responsive elements, it was decided to analyze the methylome of SNU-398 cells treated with the hypomethylating agent Decitabine (DAC), by Whole Genomic Bisulfite Sequencing (WGBS). As also described in Example 2 above, demethylation was expected in the well-studied upstream promoter region (Region D1, comprised between −199 and −1 base pairs (bp) from the p16 TSS). However, a higher degree of demethylation within p16 exon 1 (Region D2, from +1 to +456 bp relative to the TSS) and in the first 200 bp of intron 1 (Region D3, including the region from +457 to +663 bp relative to the TSS) was detected, suggesting a potential correlation between intragenic region demethylation and gene activation (also see FIG. 21A, 21B). To examine the contribution of the D2 and D3 regions on gene activation, multiple sgDiRs specific to Region D1, D2, or D3 were designed, targeting either a single region individually or multiple regions in combination (see FIG. 21C). sgDiRs targeting each region individually (see FIG. 21C, 28A) could induce some degree of demethylation (see FIG. 28B, 28C) and RNA production (see FIG. 21D), with CRISPR-DiR targeting Region D2 leading to a greater than twofold increase in p16 RNA (see FIG. 21D).

Optimization of the system was also investigated, particularly with respect to targeting strategy and targeting both the 5′ proximal promoter and 3′ beginning intron 1 region. In the studies described in this Example, it was further investigated whether a) simultaneously targeting Region D1+D2+D3 or b) targeting demethylation in both the 5′ and 3′ ends (Region D1+D3) flanking a potential “seed” region D2, would lead to gene activation greater than any individual region alone. Indeed, the combined action of CRISPR-DiR targeting either Regions D1+D2+D3 or Region D1+D3 induced significantly greater increases in p16 RNA than targeting any single region. Targeting Region D1+D3 achieved gene activation as great as targeting Region D1+D2+D3 all together (see FIG. 21D), thus representing the simplest and most efficient targeting strategy for gene activation.

Application of Crispr-DiR, and a proximal promoter+beginning of intron 1 targeting strategy, to another important and hypermethylated tumour suppressor gene, p15, is also described. As described, in order to explore the most demethylation-gene reactivation correlated region(s), the p16 transcription start site (TSS) surrounding region was divided into Region D1 (TSS upstream proximal promoter), Region D2 (exon 1), and Region D3 (beginning of intron 1). Comparing the gene demethylation and p16 reactivation efficiency via targeting these regions individually or in combination, it was observed that although targeting each of these three regions induced certain level of p16 activation, demethylation in Region D1 (upstream promoter) doesn't correlate most with gene activation (FIG. 21A, 21C, 21D); instead, the combined action of CRISPR-DiR targeting either Regions D1+D2+D3 or Region D1+D3 induced significantly greater increases in p16 RNA than targeting any single region (FIG. 21D). Targeting Region D1+D3 achieved gene activation as great as targeting Region D1+D2+D3 all together (FIG. 21D), thus representing the simplest and most efficient targeting strategy for gene activation.

Collectively, these results demonstrate that the core epigenetic regulatory element of a gene is not necessarily contained within the promoter upstream of the TSS, but is augmented by the downstream exon 1 and adjacent intron 1 regions. The “Region D1+D3” targeting strategy, or targeting “proximal promoter+beginning of intron 1” is demonstrated, rather than most other strategies only focusing on proximal promoter, as an efficient targeting strategy for CRISPR-DiR induced demethylation and gene activation.

Based on the genome-wide specific targeting ability of CRISPR system, CRISPR-DiR demethylation and gene activation system may be used for virtually any target site via designing specific guides complimentary to the target site. Examples above describe the successful application of CRISPR-DiR to another gene locus, SALL4, supporting the wide usage of CRISPR-DiR genome-wide. It was further investigated that 1) whether CRISPR-DiR can be also applied to yet another tumor suppressor gene, and 2) whether the targeting “proximal promoter+beginning of intron 1” strategy is not only efficient for p16 locus, but also for other gene loci. To determine this, another important tumor suppressor gene p15 has been used as the model. p15 is the gene most frequently silenced by aberrant promoter methylation in MDS and AML (approximately 60-70/6, reaching 80% in secondary AML) (30, 32, 84). Strikingly, p15 promoter methylation is associated with poor prognosis and correlates with MDS progression to AML (84).

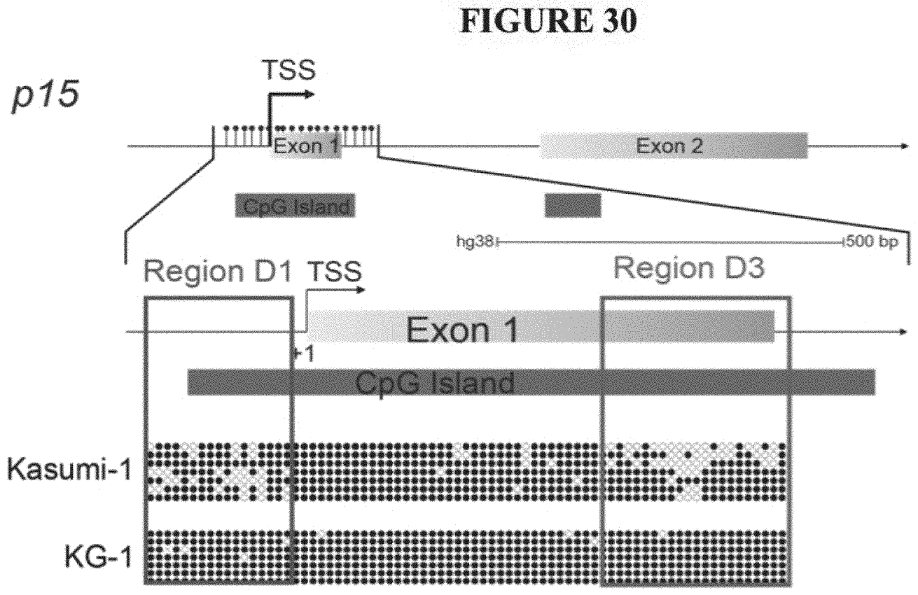

Successful re-expression of p15 in clinical treatment regimens may not only result in control of the leukemic cells but may improve the anti-leukemic function of the immune system. In order to test the most demethylation-gene expression correlated region, bisulfite sequencing PCR (BSP) was performed for both wild type AML cell line Kasumi-1 and KG1 covering the entire proximal promoter-exon 1-beginning of intron 1 region (PrExI) in p15 locus (more than 90 CpG sites) (FIG. 30). p15 was reported to be hypermethylated both in Kasumi-1 and KG1 cell lines, while Kasumi-1 has higher basal level of p15 expression and easier to demethylate than KG1 (81). Therefore, it was hypothesized that p15 is less methylated in Kasumi-1 than KG-1. Consistently, the BSP result indicated that p15 in Kasumi-1 is less methylated than in KG1, and more importantly, the unmethylation region was exactly proximal upstream promoter (Region D1) and beginning of intron 1 (Region D3). This indicated that in another tumor suppressor, p15, the most demethylation-gene expression correlated region also fit the pattern discovered in p16 gene locus, which is “proximal promoter+beginning of intron 1”, or “Region D1+D3” (FIG. 30).

Collectively, these results demonstrate that the core epigenetic regulatory element of a gene is not necessarily contained within the promoter upstream of the TSS, but is augmented by the downstream exon 1 and adjacent intron 1 regions.

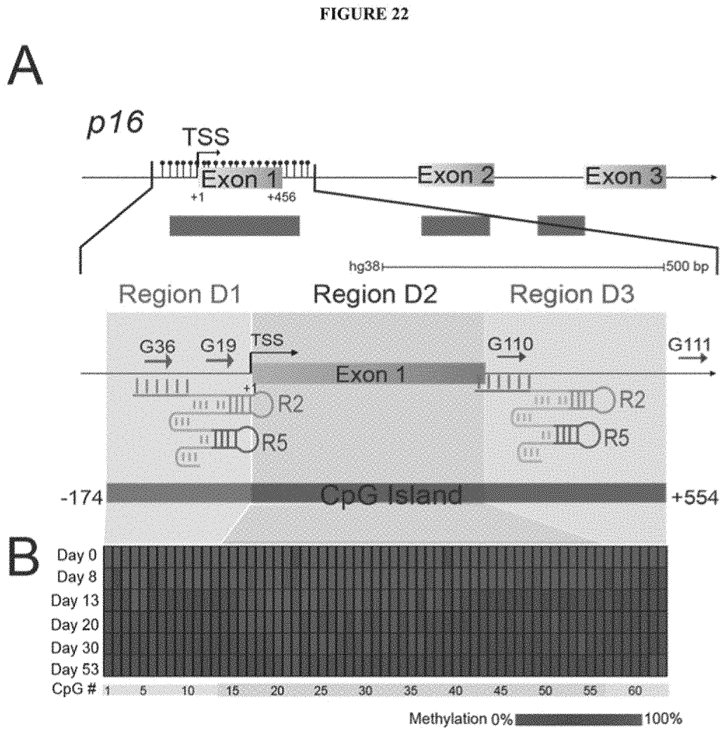

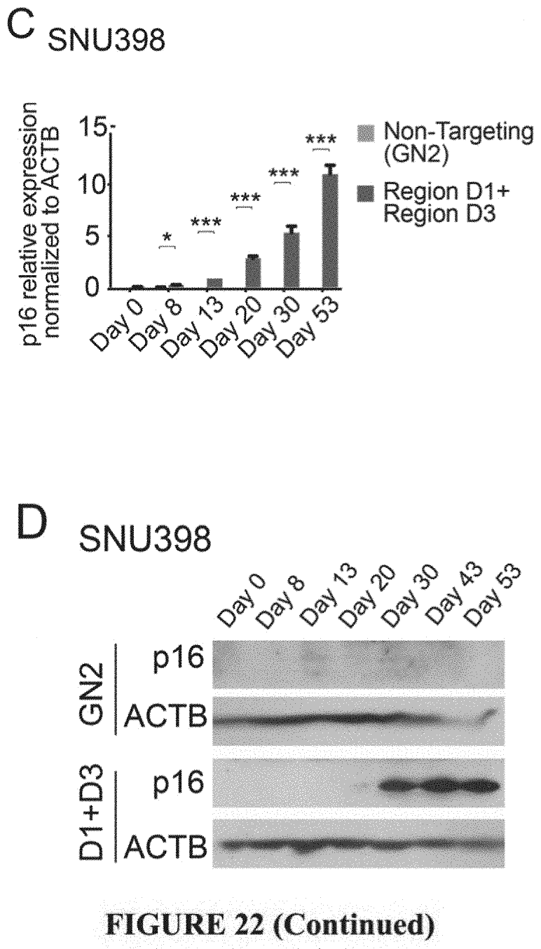

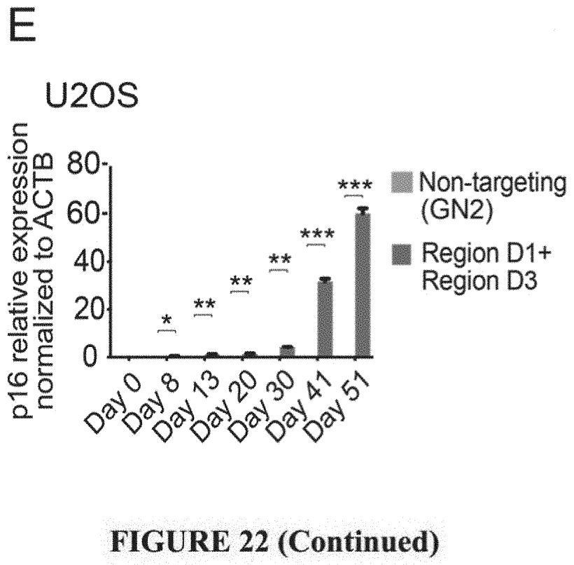

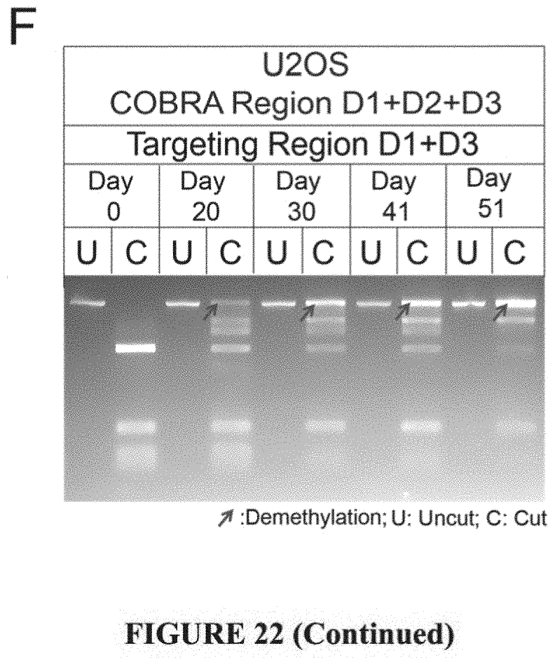

CRISPR-DiR mediated intragenic demethylation for gene activation (demethylation initiated in D1 and D3 regions can spread to the middle Exon 1 region): The observation that the p16 transcription pattern takes over a week to begin to change (see FIG. 21D) in stably expressing CRISPR-DiR cells prompted us to trace the dynamic changes in p16 demethylation over an extended period. Thus, p16 demethylation and the respective gene expression was tracked for 53 days upon delivery of the most efficient targeting strategy, D1+D3, in SNU 398 cells. Bisulfite sequencing PCR (BSP) analyses revealed that demethylation initiated from regions D1 and D3 gradually increased from day 8 onwards, spreading to the intervening D2 region by Day 13 (see FIG. 22A, 22B). Consistently, p16 mRNA expression increased significantly after Day 13 (see FIG. 22C), and p16 protein levels peaked after Day 20 (see FIG. 22D), indicating that CRISPR-DiR initiated demethylation preceded transcriptional activation and protein expression. Strikingly, no demethylation “spreading” to surrounding regions (regions C and E) (see FIG. 28A, 28D, 28E) was observed, suggesting that CRISPR-DiR mediated demethylation might be confined, and spreading exclusively within a regulatory core region (D2) (49). To demonstrate this effect was not confined to a single cell line, CRISPR-DiR was delivered into U2OS, a human osteosarcoma line with silenced p16, (see FIG. 22E, 22F), and a similar trend in demethylation profiles and RNA expression was observed. In addition, no changes in RNA of the adjacent p14 gene (located 20 Kb upstream of p16, which is also methylated with no detectable expression), or CEBPA (located on another chromosome and actively expressed) was detected, thereby supporting the selectivity of the approach (see FIG. 28F).

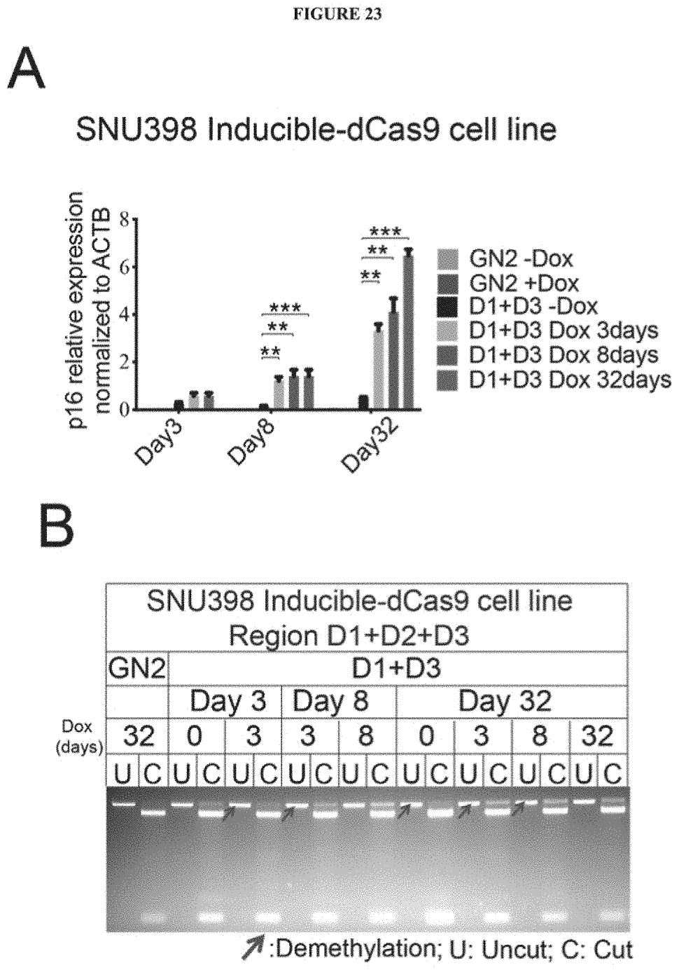

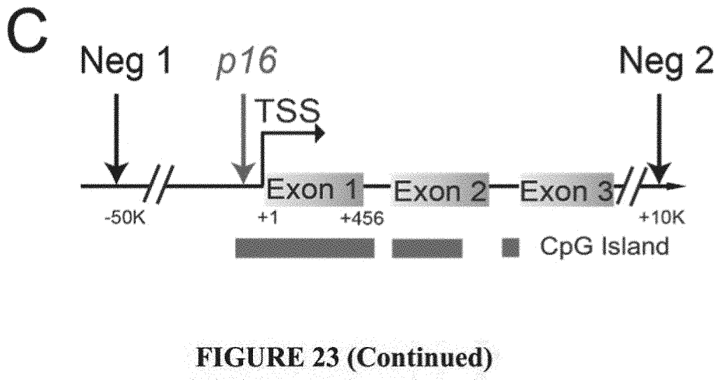

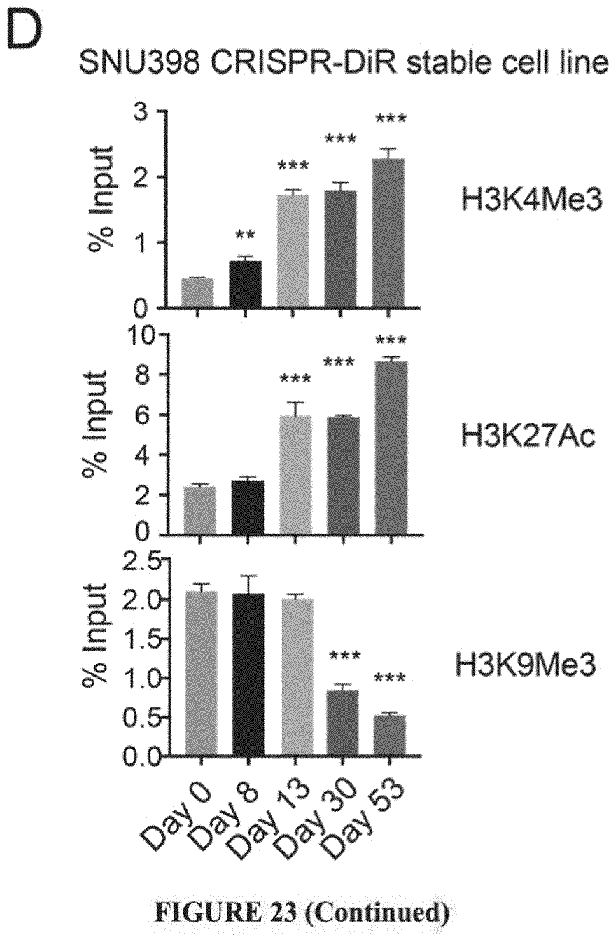

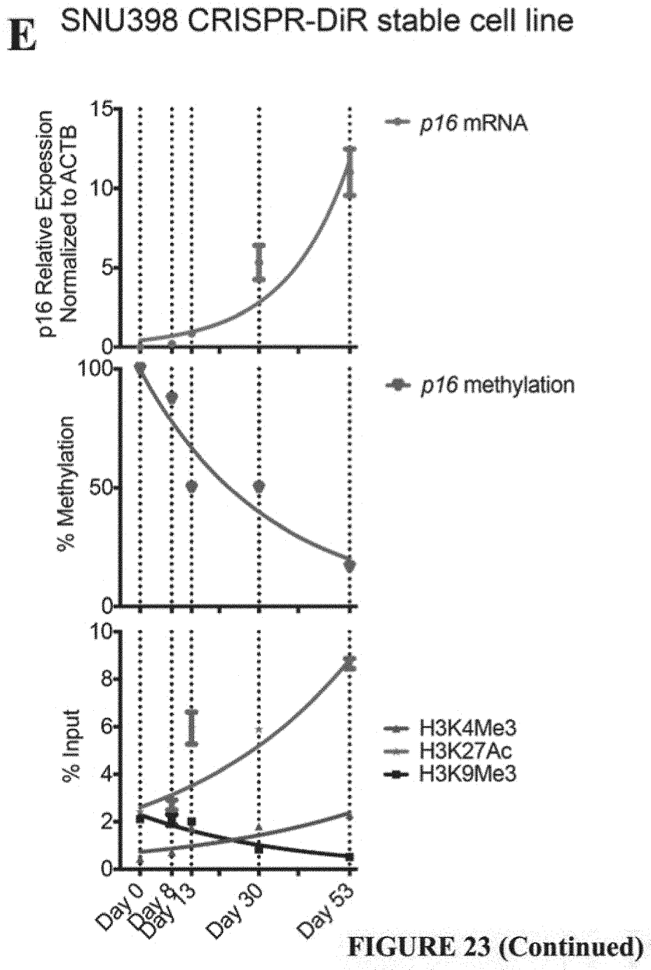

Targeted intragenic demethylation induces chromatin remodeling: To better evaluate whether demethylation by CRISPR-DiR, once initiated, was a lasting effect, a Tet-On dCas9-stably expressing SNU-398 cell line was generated, wherein dCas9 can be conditionally induced and expressed upon doxycycline addition (see also Example 2 above). Within as soon as three days of induction treatment, p16 demethylation and activation was observed and persisted for at least a month (see FIG. 23A, 23B). These findings, along with our previous observations demonstrating steady increase in demethylation and RNA over nearly 2 months (see FIG. 22B, 22C), pointed to the potential involvement of other epigenetic changes arising from the initial demethylation event and gene activation.

It was therefore hypothesized that loss of DNA methylation within the promoter-exon 1-intron 1 (PrExI) demethylation core region would facilitate histone changes and chromatin configuration for gene activation.

To establish a direct correlation between these two events, Chromatin Immunoprecipitation (ChIP) with antibodies to the activation histone marks H3K4Me3 and H3K27Ac, or the repressive mark H3K9Me3, coupled with quantitative PCR (ChIP-qPCR) (see FIG. 23C) was carried out in wild type and CRISPR-DiR treated (D1+D3) SNU-398 cells. An enrichment of H3K4Me3 and H3K27Ac marks between Day 8 to 13 within the p16 PrExI demethylation core region was observed, inversely correlated with a progressive loss of the H3K9Me3 silencing mark (see FIG. 23D, 23E), which corroborates the hypothesis that demethylation may be the first event induced by CRISPR-DiR (Day 8), followed by gain of transcriptional activation marks in parallel to a loss of silencing marks (Day 8-13).

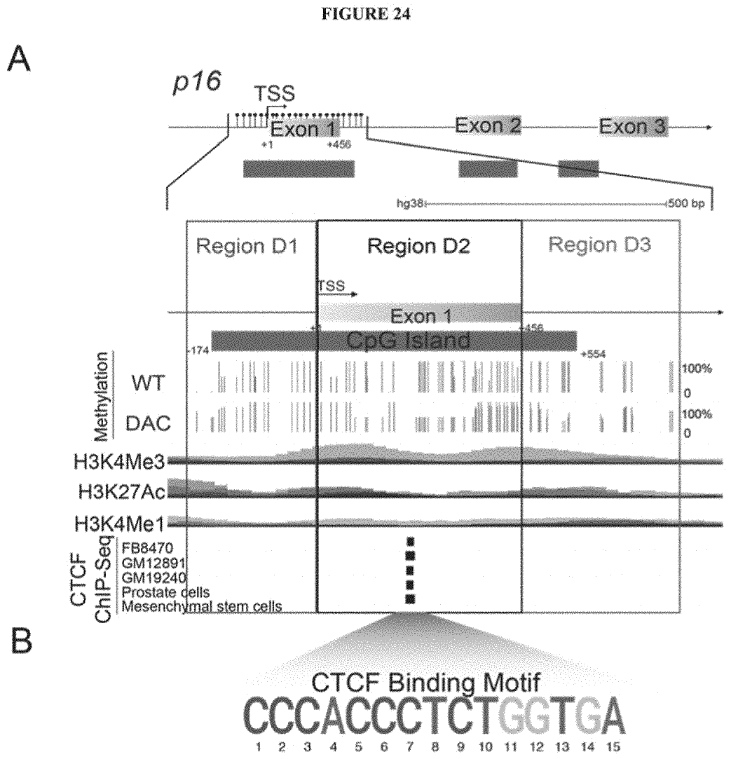

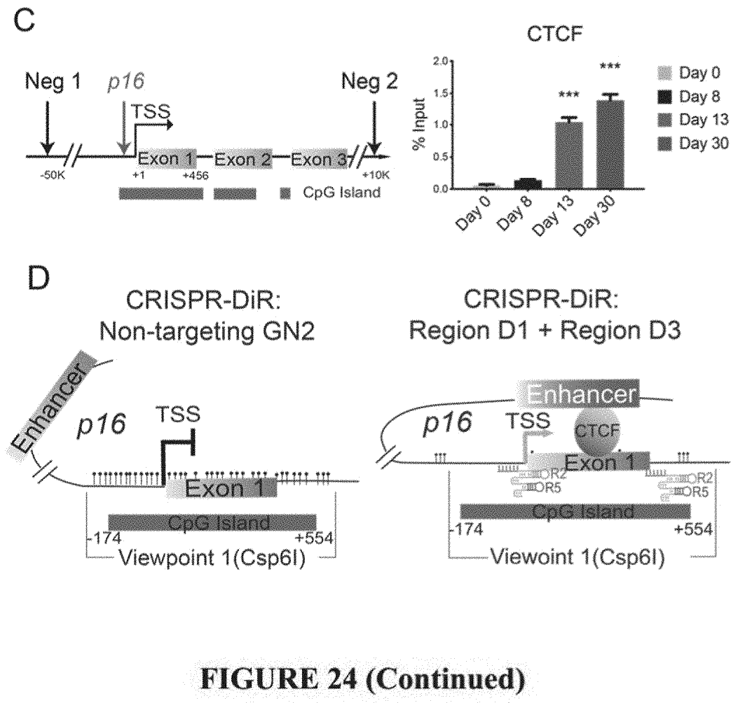

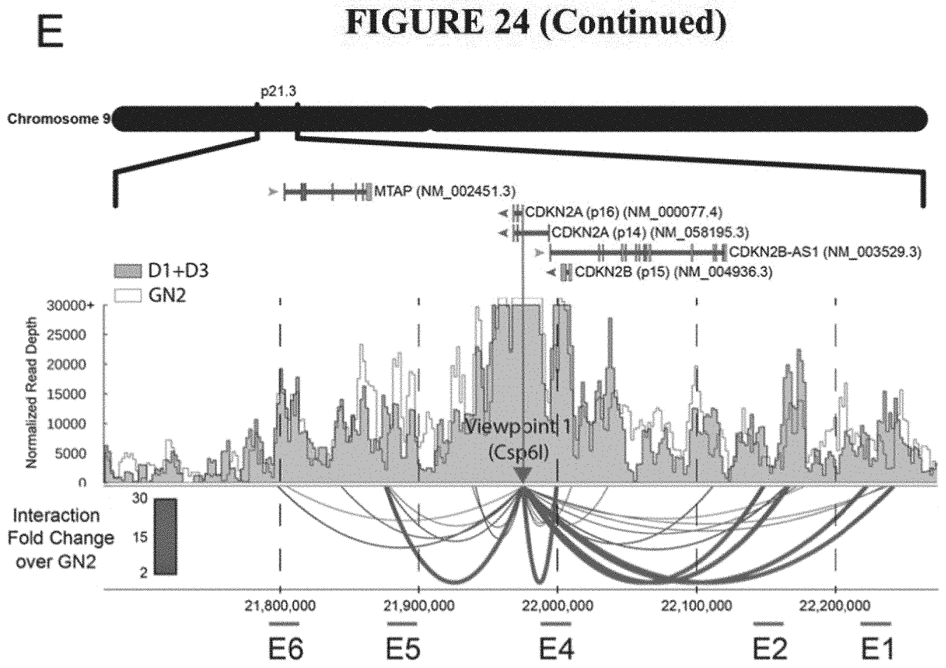

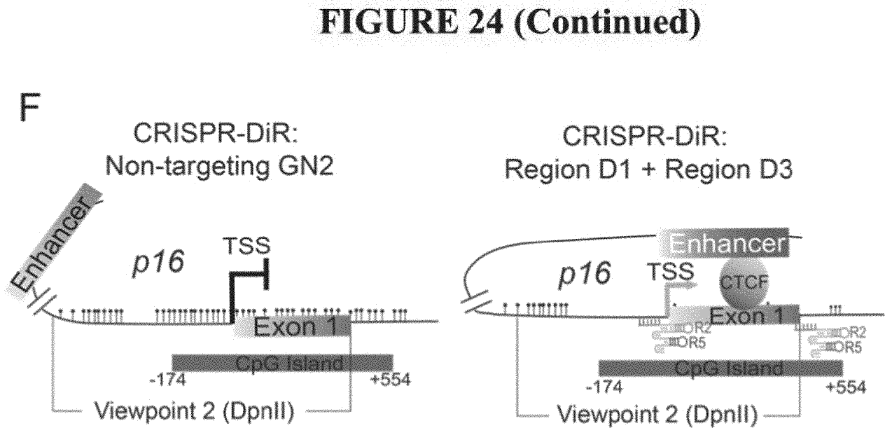

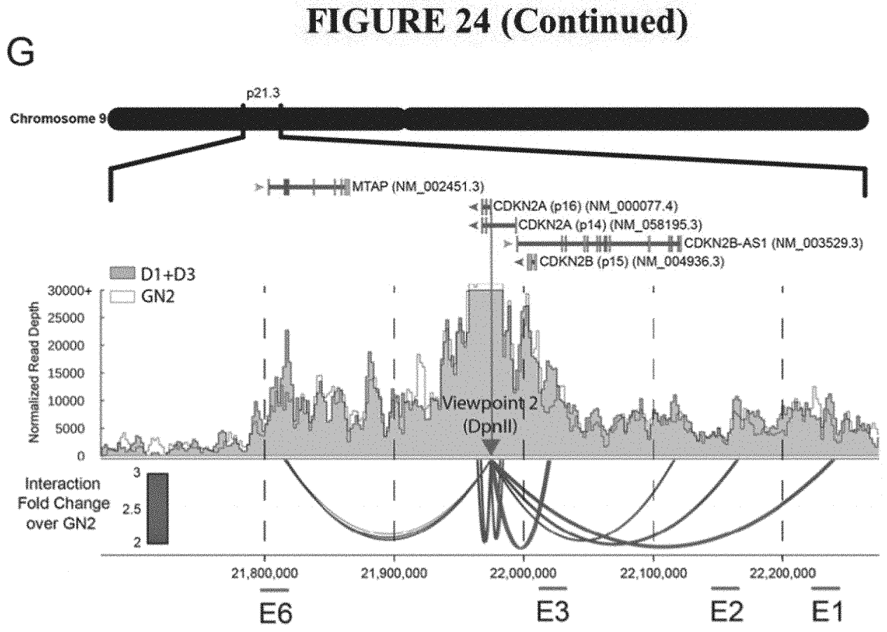

Locally induced demethylation is important to initiate distal long-range interactions: Most transcription factors binding to the p16 promoter are sensitive to DNA methylation (33, 50, 51), since DNA methylation will prevent access to their recognition site. Thus, it was proposed that after CRISPR-DiR induced demethylation within the PrExI region spanning the promoter-exon-intron 1, transcription factors could re-gain access and be able to bind to this region. Using the motif analysis tool TFregulomeR (52), a TF binding site analysis tool linking to a large compendium of ChIP-seq data, CTCF (CCCTC-binding factor) binding peaks in exon 1 was found across five different cell lines (see FIG. 24A), along with an additional predicted binding site (see FIG. 24B). CTCF, a master regulator of chromatin architecture, can function as an insulator, to define chromatin boundaries and mediate loop formation, hence promoting or repressing transcription (53). Furthermore, CTCF is a positive regulator of the p15-p14-p16 locus (51, 54), and can be displaced by DNA methylation (55, 56). This led us to test whether CRISPR-DiR-mediated demethylation could restore CTCF binding. Indeed, it was observed that CTCF was enriched in the 800-bp demethylated core region (see FIG. 24C), 13 days following induction of CRISPR-DiR, the time point at which strong demethylation occurred (see FIG. 22B, 23E). This finding supports a model of restoring CTCF binding upon demethylation, contributing to enhancement of p16 mRNA expression after Day13.

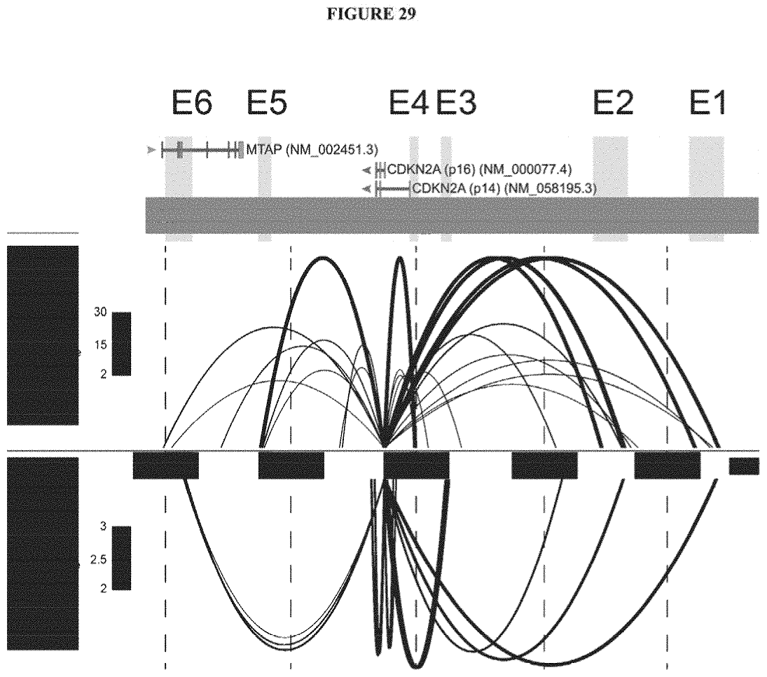

A few studies have reported a p16 enhancer region located ˜150 kilobases (kb) upstream of the p16 TSS (57-59). Yet, long range interactions with the p16 locus and the impact of locus-specific demethylation has been unexplored. It was further proposed that the CRISPR-DiR-induced demethylation and resulting CTCF binding would initiate long-range interactions between distal regulatory elements and the p16 gene locus, and therefore rewire the chromatin structure and promote gene transcription. To assess the impact of loss of DNA methylation on p16 locus topology, Circularized Chromosome Conformation Capture (4C) was performed for CRISPR-DiR non-targeting (GN2) or targeting (D1+D3) Day 13 samples. Two viewpoints (‘baits’) were designed as close to the promoter-exon 1-intron 1 demethylation core region as possible: Viewpoint 1, covering the exact targeted region D1 to D3 (see FIG. 24D); while Viewpoint 2, covering the upstream promoter-exon 1 region (see FIG. 24F). While Viewpoint 1 provides a closer examination of the targeted region, Viewpoint 2 overlaps more of the promoter. This two viewpoints design enables both an internal validation of the long-range interactions, and a careful analysis of the different interplay between distal regulatory elements and the promoter-exon 1 (viewpoint 2) or the exon 1-intron 1 (viewpoint 1) region, respectively (see FIG. 29). Comparing the targeting (D1+D3) sample with the non-targeting (GN2) control, interaction changes between distal elements and the p16 demethylated locus were detected, scattered within 500 kb encompassing the p16 targeted demethylation core region (PrExI) (see FIG. 24E, 24G, 29). The strongest interaction increases initiated by demethylation for both viewpoints were identified (see FIG. 29), which can represent potential distal enhancers for p16 gene transcription. To note, among these strong interactions upon CRISPR-DiR induced demethylation, novel interaction regions located more than 200 kb upstream (E1) were not only detected within the Anril-p15-p14 locus (E3, E4), or more than 100 kb downstream (E5, E6) of the p16 TSS, but interactions with the enhancer region previously described at ˜150 kb upstream of p16 TSS (E2) (57-59) were also observed. These results indicated the reproducibility and reliability of the analysis, since the strong interactions overlap quite well between the two viewpoints (see FIG. 29) and include the known enhancer regions. Furthermore, they point to potential novel p16 enhancer elements and highlight a close interplay between p16 and the neighboring gene loci Anril-p15-p¹⁴.

### DISCUSSION

This Example explores the functionalization of endogenous RNAs into an innovative locus-specific demethylation and activation technology herein referred to as CRISPR-DiR (DNMT1-interacting RNA).

By incorporating short functional DiR sequences into the scaffold of single guide RNAs, a scalable, customizable, and precise system for naïve and localized demethylation and activation has been developed.

Using as a model the p16 locus, a tumor suppressor gene frequently silenced by DNA methylation in cancers, this Example shows that the core epigenetic regulatory element of gene activation is not contained within the extensively studied CpG-rich promoter region upstream of the p16 TSS, but encompasses the proximal promoter-exon 1-intron 1 region (PrExI) (Region D1 to D3, −199 to +663 relative to the TSS). By simultaneously engaging the 5′ (promoter) and 3′ (intron) regions flanking the activator core, the present designs provided consistent and effective demethylation spreading (Region D2, see FIG. 22B) and provide features that are missing in other CRISPR-based platforms targeting exclusively the immediate promoter (35-37, 60). Intriguingly, the CRISPR-DiR induced demethylation wave propagated inward into the middle of exon 1 region, while no demethylation was observed in the surrounding regions (Region C and E, see FIG. 28D, 28E), despite the high CpG content, in contrast to what was previously suggested (61, 62). These findings demonstrate how demethylation of a key regulatory core region is an important condition for gene activation for p16, which were also demonstrated for the SALL4 gene locus (63). Results indicate the demethylation wave initiates a stepwise process followed by acquisition of active histone marks, recruitment of the architectural protein CTCF (which binds to non-methylated DNA), and chromatin reconfiguration of the p16 locus, ultimately steering long-range interactions with distal regulatory elements (see FIGS. 24 and 25). In addition to a previously reported enhancer region located approximately 150 KB upstream of p16 (57-59), it is demonstrated that demethylation of the core region promoted interactions with a number of elements located as far as 500 kb away, indicating that localized and very specific adjustments of DNA methylation can broadly impact chromatin configuration and topological rearrangements (see FIG. 25).

A dual R2 design was described by Lu et al., Reprogrammable CRISPR/dCas9-based recruitment of DNMT1 for site-specific DNA demethylation and gene regulation, Cell Discovery, 2019, 5:22 (80). However, as described hereinabove, a construct with R2/R2 configuration was tested herein and was not effective in the conditions tested herein, and performed poorly in contrast to MsgDiR6, which incorporates DiR loop R2 into the sgRNA tetra-loop and DiR loop R5 into sgRNA stem loop 2. Additionally, the studies described herein show that demethylation in promoter alone doesn't correlate well with strong gene expression (Lu et al. focused on targeting proximal promoter upstream of transcription start site (TSS)), especially when compared with strategies described herein in which significantly enhanced gene activation was observed by targeting gene “proximal promoter+beginning of intron 1”, a strategy not only applied to p16, but also p15, indicating versatility and broad or genome-wide applicability. The gene targets in Lu et al., 2019 contain very limited number and sparsely distributed CpG sites, indicating a more open chromatin structure likely easier to access and regulate. CG density in Lu et al. targeted gene is only 1 CpG site per 100 bp (about 4 CG sites in the targeted region), while the gene p16 that targeted in the present studies has a very condensed CG ratio (63 CG sites in a 800 bp region, so approximately 9 CG sites per 100 bp) and closed chromatin structure. In most real silenced tumor suppressor gene cases (eg. p16, p15), there are super condensed CG sites around TSS and heterochromatin structure, which makes the region hard to access or demethylate. The presently described CRISPR-DiR system was developed and tested as described herein with a real, hard to demethylate and activate, gene example (p16—very condensed CG ratio and closed chromatin structure, similar to most tumor suppressor genes) instead of other easy to manipulate genes, using stable cell line and inducible system configuration instead of transient transfection, supporting the presently described systems as reliable and powerful tools even for condensed CG sites and heterochromatin structure. In experiments described herein, the presently described Crispr-DiR systems also restored protein expression, as well as gene function, in stringent tests assessing both demethylation and gene activation. Long-lasting effect was also observed herein for the presently described CRISPR-DiR inducible system (histone modifications and distal interactions induced by CRISPR-DiR targeted demethylation in the core PrExI region (promoter-exon 1-intron 1)). Both the high efficiency of CRISPR-DiR systems as described herein, as well as the real regulatory core PrExI region targeted herein (promoter-exon 1-intron 1), support a broad or genome-wide targeting strategy which may work efficiently even on gene locus heavily enriched for CpGs and highly methylated (and thus in a heterochromatin state). As described herein, Crispr-DiR also restored protein expression, as well as gene function.

This example describes further development and optimization of RNA based CRISPR-DiR technology, to repurpose functional RNA segments to a specific target site and manipulate methylation profiles, epigenetic marks, and gene expression in a locus specific manner. As well, direct evidence that demethylation solely in the upstream promoter region was weakly correlated with gene activation, while demethylation in the entire promoter-exon 1-intron 1 region (PrExI) significantly enhanced gene transcription, is provided. Example 2 shows CRISPR-DiR targeting the SALL4 gene 5′UTR-Exon 1-Intron 1 region effectively restored gene expression and function. Therefore, the PrExI targeting region and “demethylation firing center (DFC)” mechanism may be a common mechanism genome-wide. Results herein elucidate that the demethylation of an 800 by “demethylation firing center (DFC)” initiated the remodeling wave for the interplay between DNA methylation, histone modifications, and chromatin remodeling. This stepwise process consisted of local demethylation, acquisition of active chromatin marks (eg. H3K4Me3 and H3K27Ac), enrichment of methylation sensitive regulators (eg. CTCF), and also interactions with presumptive distal regulatory elements as far as 500 kb away, ultimately leading to stable gene-locus transcriptional activation. Distal interactions observed by the CRISPR-DiR demethylation included both the previously reported p16 enhancer elements, and new enhancer candidates for p16. Indeed, the newly characterized distal interactions also suggested a self-regulating mechanism within the Anril-p15-p14-p16 region. Results highlight the possibility to repurpose RNA based regulation of DNA methylation to any selected gene locus by fusing functional endogenous RNAs into the CRISPR system, supporting RNA-based gene-specific demethylation therapies for cancer and other diseases, for example.

Targeting Region D1+D3 provided an enhanced targeting strategy for Crispr-DiR based demethylation and activation of p16 gene locus, likely (without wishing to be bound by theory) by eliciting a demethylation wave within the “seed” region (e.g. middle region of exon 1) from both sides, not only inducing the demethylation in the entire core region but also spreading the demethylation to the middle seed region, therefore achieving high activation using the least number of sgDiRs, which may also provide for reduced off-target risk due to less targets.

In conclusion, the present data show how CRISPR-DiR induced demethylation of a small core element retained in approximately 800 bp was able to propagate as far as 500 kb away, demonstrating the existence of an intragenic transcriptional initiator core which controls gene activation while acting as multiplier factor coordinating chromatin interactions. CRISPR-DiR initiated locus specific 800 bp demethylation rewiring 500 kb chromatin structure. The demethylation in not only upstream promoter, but also the intragenic exon1-intron 1 region for both local and distal chromatin remodeling and gene activation, suggests both a novel regulation mechanism and targeting strategy for gene regulation. This may be of particular importance in cancer, in which many important tumor suppressors are silenced and methylated. Traditional general demethylating agents are being employed in the clinic, but their efficacy is hampered by their lack of specificity. Results support CRISPR-DiR gene-specific demethylation and activation platform, working in a locus specific manner, for methylation studies, target candidate screening, and for RNA-based therapies, for example. Results indicate the system as solid, reproducible, and efficient, and which may be applied even to densely hypermethylated tumor suppressor gene locus with heterochromatin structure (e.g. p16).

Results shows the system maintained demethylation and gene activation effect for more than a month once induced for as short as 3 days. The features of this technology may aid in the identification of novel targets for clinical applications, developing alternative demethylation-based screening platforms, and designing therapeutic approaches to cancer or other diseases accompanied by DNA methylation, for example.

### Materials and Methods:

### Cell Culture

The human hepatocellar carcinoma (HCC) cell line SNU-398 was cultured in Roswell Park Memorial Institute 1640 medium (RPMI) (Life Technologies, Carlsbad, Calif.) supplemented with 10% fetal bovine serum (FBS) (Invitrogen) and 2 mM L-Glutamine (Invitrogen). Human HEK293T and human osteosarcoma cell line U2OS were maintained in Dulbecco's Modified Eagle Medium (DMEM) supplemented with 10% fetal bovine serum (FBS). All cell lines were maintained at 37° C. in a humidified atmosphere with 5% CO₂ as recommended by ATCC and were cultured in the absence of antibiotics if not otherwise specified.

### RNA Isolation

Total RNA was either extracted using AllPrep DNA/RNA Mini Kit (Qiagen, Valencia, Calif.) and treated with RNase-free DNase Set (Qiagen) following the manufacturer's instructions, or isolated with TRIzol (Invitrogen). If the RNA isolation was carried out with the TRIzol method, all RNA samples used in this study were treated with recombinant RNase-free DNase I (Roche) (10 U of DNase I per 3 mg of total RNA; 37° C. for one hour; in the presence of RNase inhibitor). After DNase I treatment, RNA samples were extracted with acidic phenol (pH 4.3) to eliminate any remaining traces of DNA.

### Genomic DNA Extraction

Genomic DNA was extracted by either the AllPrep DNA/RNA Mini Kit (Qiagen, Valencia, Calif.) for BSP, MSP, and COBRA assays or by Phenol-chloroform method if extremely high-quality DNA samples were required for whole genomic bisulfite sequencing (WGBS). The Phenol-chloroform DNA extraction was performed as described (64). Briefly, the cell pellet was washed twice with cold PBS. 2 mL of gDNA lysis buffer (50 mM Tris-HCl pH 8, 100 mM NaCl, 25 mM EDTA, and 1% SDS) was applied directly to the cells. The lysates were incubated at 65° C. overnight with 2 mg of proteinase K (Ambion). The lysate was diluted 2 times with TE buffer before adding 1 mg of RNase A (PureLink) and followed by a 1 hour incubation at 37° C. The NaCl concentration was subsequently adjusted to 200 mM followed by phenol-chloroform extraction at pH 8 and ethanol precipitation. The gDNA pellet was dissolved in TE pH 8 buffer.

Quantitative Real-Time PCR (qRT-PCR)

1 ug of RNA was reverse transcribed using Qscript cDNA Supermix (QuantaBio). The cDNAs were diluted 3 times for expression analysis. qRT-PCR on cDNA or ChIP-DNA were performed on 384 well plates on a QS5 system (Thermo Scientific) with GoTaq qPCR Master Mix (Promega, Madison, Wis.). The fold change or percentage input of the samples was calculated using the QuantStudio™ Design & Analysis Software Version 1.2 (ThermoFisher Scientific) and represented as relative expression (ΔΔCt). All measurements were performed in triplicate. Primers used in this study are listed in Table 1.

5-aza-2′-deoxycytidine (Decitabine) Treatment SNU-398 cells were treated with 2.5 μM of 5-aza-2′-deoxycytidine (Sigma-Aldrich) according to the manufacturer's instructions. Medium and drug were refreshed every 24 h. RNA and genomic DNA were isolated after 3 days (72 h) treatment.

### Deoxvycytidine (Dox) Treatment

In the dCas9 Tet-On SNU-398 cells (inducible CRISPR-DiR system shown in FIG. 23A, 23B), the same targeting strategy shown in FIG. 22A (Region D1+Region D3) was used, and dCas9 expression was induced following treatment with Deoxycytidine (Dox). Dox was freshly added to the culture medium (1 μM) every day for Dox+ sample, while Dox− samples were cultured in normal medium without Dox. For Dox induced 3 days/8 days samples, 1 μM Dox was added to fresh medium for 3 days and 8 days accordingly, then the cells were kept in medium without Dox until Day 32; for Dox induced 32 days samples, 1 μM Dox was added to fresh medium every day for 32 days. All treated cells were cultured and assayed at Day 3, Day 8 and Day 32.

### Transient Transfections

SNU-398 cells were seeded at a density of 3.5×10W cells/well in 6-well plates 24 hours before transfection employing jetPRIME transfection reagent (Polyplus Transfection) as described by the manufacturer. 2 μg mix of sgRNA/MsgDiR and dCas9 μlasmid(s) (sgRNA/MsgDiR: dCas9 molar ratio 1:1) were transfected into each well of cells. The culture medium was changed 12 hours after transfection. Alternatively, the Neon™ Transfection System (Thermo Fisher) was used for cell electroporation according to the manufacturer's instructions. The same plasmid amount and ratios were used in the Neon as in the jetPRIME transfection. The parameters used for the highest SNU-398 transfection efficiency were 0.7 to 1.5 million cells in 100 μl reagent, voltage 1550 V, width 35 ms and 1 pulse. The culture medium was changed 24 hours after transfection. The plasmids used in this study are listed in Table 2.

### Lentivirus Production

pMD2.G, psPAX2, and lentivector (plv-dCas9-mCherry, pcw-dCas9-puro, plv-GN2sgDiR-EGFP, plv-G19sgDiR-EGFP, plv-G36sgDiR-EGFP, plv-G108sgDiR-EGFP, plv-G122sgDiR-EGFP, plv-G110sgDiR-EGFP, plv-G111sgDiR-EGFP) were transfected into 10 million 293T using TransIT-LT1 reagent (Minis), lentivector: psPAX2: pMD2.G 9 μg: 9 μg:1 μg. The medium was changed 18 hours post-transfection, and the virus supeamants were harvested at 48 hr and 72 hr after transfection. The collected virus was filtered through 0.45 μm microfilters and stored at −80° C. The plasmids used in this study are listed in Table 2. To note, few studies have tried to modify the sgRNA scaffold to increase their stability. One option is to remove the putative POL-III terminator (4 consecutive Ts in the beginning of sgRNA scaffold) by replacing the fourth T to G (65). Thus, in the CRISPR-DiR design, the fourth T (in bold, italic, underline below) was substituted with G, to make the structure more stable by enabling efficient transcription, while keeping substantially the same secondary structure and decreasing the minimum free energy (MFE). Accordingly, the corresponding A was substituted with C to preserve base-pairing with the “G”. All the sgRNA, MsgDiRs scaffold sequences are listed in Table 3 and shown in FIG. 31, guide RNA sequences are listed in Table 4 and the locations of each region (Region C, D1, D2, D3, and E) are listed in Table 5.

### Generating CRISPR-DiR and Inducible CRISPR-DiR Stable Cell Lines

Both SNU-398 and U20S cells were seeded in T75 flasks or 10 cm plates 24 hours prior to transduction, and were first transduced with dCas9 or inducible dCas9 virus medium (thawed from −80° C.) together with 4 μg/mL polybrene (Santa Cruz) to make SNU398-dCas9, U20S-dCas9, or inducible SNU398-dCas9 stable lines. Once incubated for 24 hours at 37° C. in a humidified atmosphere of 5% CO2, the medium with virus can be changed to normal culture medium. The dCas9 positive cells were sorted using a mCherry filter setting with a FACS Aria machine (BD Biosciences) at the Cancer Science Institute of Singapore flow cytometry facility, while the inducible dCas9 positive cells were selected by adding puromycin at 2 μg/ml concentration in the culture medium every other day. The cells were further cultured for more than a week to obtain stable cell lines. Once the dCas9 and inducible dCas9 cell lines were generated, sgDiRs virus with different guide RNAs were mixed in equal volume and transduced into dCas9 or inducible dCas9 stable lines with the same method as described above. The sgDiRs used for generating each stable cell line, the location of each sgDiR, as well as the definition of Region D1, Region D2, and Region D3 can be found in Table 4 and Table 5. All sgDiR stable cell lines were sorted using an EGFP filter with a FACS Aria machine (BD Biosciences) at the Cancer Science Institute of Singapore flow cytometry facility, and further assessed in culture by checking the efficiency by microscopy regularly.

Western Blot Analysis Total cell lysates were harvested in RIPA buffer (150 mM NaCl, 1% Nonidet P-40, 50 mM Tris, pH8.0, protease inhibitor cocktail) and protein concentrations were determined by Bradford protein assay (Bio-Rad Laboratories, Inc. Hercules, Calif., USA) and absorbance was measured at 595 nm on the Tecan Infinite* 2000 PRO plate reader (Tecan, Seestrasse, Switzerland). Equal amounts of proteins from each lysate were mixed with 3X loading dye and heated at 95° C. for 10 minutes. The samples were resolved by 12% SDS-PAGE (running buffer: 25 mM Tris, 192 mM Glycine, and 0.1% SDS) and then transferred to PVDF membranes (transfer buffer: 25 mM Tris, 192 mM Glycine, and 20% (v/v) methanol (Fischer Chemical)). Membranes were blocked with TBST buffer containing 5% skim milk one hour at room temperature with gentle shaking. The blocked membranes were further washed three times with TBST buffer and incubated at 4° C. overnight with primary antibodies CDKN2A/p161NK4a (ab108349, Abcam, 1:1000), P-actin (Santa Cruz P-actin (C4) Mouse monoclonal IgG1 #sc-47778, 1:5000), followed by HRP-conjugated secondary antibody incubation at room temperature for one hour. Both the primary and secondary antibodies were diluted in 5% BSA-TBST buffer, and all the incubations were performed in a gentle shaking manner. The immune-reactive proteins were detected using the Luminata Crescendo Western HRP substrate (Millipore).

### Bisulfite Treatment

The methylation profiles of the p16 gene locus or the whole genome were assessed by bisulfite-conversion based assays. For DNA bisulfite conversion, 1.6-1.8 μg of genomic DNA of each sample was converted by the EpiTect Bisulfite Kit (Qiagen) following the manufacturer's instructions.

Methylation-Specific PCR (MSP), Combined Bisulfite Restriction Analysis (COBRA) and bisulfite Sequencing PCR (BSP)

The bisulfite converted DNA samples were further analyzed by three different PCR based methods in different assays for the methylation profiles. For Methylation-Specific PCR (MSP), both methylation specific primers and unmethylation specific primers of p16 were used for the PCR of the same bisulfite converted sample (the transient transfection samples in the sgRNA and MsgDiR1-8 screening assay). The PCR was performed with ZymoTaq PreMix (ZYMO RESEARCH) according to the manufacturer's instructions, with the program: 95° C. 10 min, 35 cycles (95° C. 30s, 56° C. 30s, 72° C. 1 min), 72° C. 7 min, 4° C. hold. Two PCR products of each sample (Methylated and Unmethylated) were obtained and analyzed in 1.5% agarose gels. For Combined Bisulfite Restriction Analysis (COBRA), primers specifically amplify both the methylated and unmethylated DNA (primers annealing to specific locus without any CG site) in each region were used for the PCR of the bisulfite converted samples. The PCR was performed with ZymoTaq PreMix (ZYMO RESEARCH) according to the manufacturer's instructions, with the program: 2 cycles (95° C. 10 min, 55° C. 2 min, 72° C. 2 min), 38 cycles (95° C. 30s, 55° C. 2 min, 72° C. 2 min), 72° C. 7 min, 4° C. hold. The PCR products were therefore loaded in a 1% agarose gel and the bands with predicted amplification size were cut out and gel purified. 400 ng purified PCR fragments were incubated in a 20 μl volume for 2.5h-3h with 1 μl of the restriction enzymes summarized in Table 6. 100 ng of the same PCR fragments were incubated with only the restriction enzyme buffers under the same conditions as uncut control. The uncut and cut DNA were then separated on a 2.5% agarose gel and stained with ethidium the bromide. For bisulfite sequencing PCR, primers specifically amplify both the methylated and unmethylated DNA (primers annealing to the specific locus without any CG site) in Region D were used for the PCR of the bisulfite converted samples. The PCR was performed with ZymoTaq PreMix (ZYMO RESEARCH) according to the manufacturer's instructions, with the program: 2 cycles (95° C. 10 min, 55° C. 2 min, 72° C. 2 min), 38 cycles (95° C. 30s, 55° C. 2 min, 72° C. 2 min), 72° C. 7 min, 4° C. hold. PCR products were gel-purified (Qiagen) from the 1% TAE agarose gel and cloned into the pGEM-T Easy Vector System (Promega) for transformation. The cloned vectors were transformed into Stb13 competent cells and miniprep was performed to extract plasmids for Sanger sequencing with either sequencing primer T7 or SP6. Sequencing results were analyzed using QUMA (Quantification tool for Methylation Analysis).

Samples with conversion rate less than 95% and sequence identity less than 90% as well as clonal variants were excluded from our analysis. The minimum number of clones for each sequenced condition was 8. All the MSP, COBRA, and BSP primers as well as restriction enzymes can be found in Table 6.

Whole Genomic Bisulfite Sequencing (WGBS) 10 cm plates of wild type SNU-398 cells and Decitabine treated SNU-398 cells were washed twice with cold PBS. 2 mL of gDNA lysis buffer (50 mM Tris-HCl pH 8, 100 mM NaCl, 25 mM EDTA, and 1% SDS) was added directly to the cells. The lysates were incubated at 65° C. overnight with 2 mg of proteinase K (Ambion). The lysate was diluted 2 times with TE buffer before adding 1 mg of RNase A (PureLink) and followed by a one-hour incubation at 37° C. The NaCl concentration was subsequently adjusted to 200 mM followed by phenol-chloroform extraction at pH 8 and ethanol precipitation. The gDNA pellet was dissolved in 1 mL TE pH 8 buffer and incubated with RNase A with a concentration of 100 ug/mL (Qiagen) for 1 hour at 37° C. The pure gDNA was recovered by phenol-chloroform pH 8 extraction and ethanol precipitation and dissolved in TE pH 8 buffer. 10 ug of each gDNA samples (wild type and decitabine treated) were sent to BGI (Beijing Genomics Institute) for WGBS library construction and sequencing. The samples were sequenced to approximate 30× human genome coverage (˜90 Gb) on a Hiseq X platform with 2×150 paired end reads.

### Chromatin Immunoprecipitation (ChIP)

ChIP was performed as described previously (66). Briefly, samples of 60 million cells were trypsinized by washing one time with room temperature PBS, then every 50-60 million cells were resuspended in 30 ml room temperature PBS. Cells were fixed with 1% formaldehyde for 8 mins at room temperature with rotation. Excessive formaldehyde was quenched with 0.25M glycine. Fixed cells were washed twice with cold PBS supplemented with 1 mM PMSF. After washing with PBS, cells were lysed with ChIP SDS lysis buffer (100 mM NaCl, 50 mM Tris-Cl pH8.0, 5 mM EDTA, 0.5% SDS, 0.02% NaN₃, and fresh protease inhibitor complete tablet EDTA-free (5056489001, Roche) and then stored at −80° C. until further processing. Nuclei were collected by spinning down at 3000 rpm at 4° C. for 10 mins. The nuclear pellet was resuspended in IP solution (2 volume ChIP SDS lysis buffer plus 1 volume ChIP triton dilution buffer (100 mM Tris-Cl pH8.6, 100 mM NaCl, 5 mM EDTA, 5% Triton X-100), and fresh proteinase inhibitor) with 10 million cells/ml IP buffer concentration (for histone marker ChIP) or 20million cells/ml IP buffer concentration (for CTCF ChIP) for sonication using a Bioruptor (8-10 cycles, 30s on, 30s off, High power) to obtain 200 bp to 500 bp DNA fragments. After spinning down to remove debris, 1.2 ml sonicated chromatin was pre-cleared by adding 50 μl washed dynabeads protein A (Thermo Scientific) and rotated at 4° C. for 2 hrs. Pre-cleared chromatin was incubated with antibody pre-bound dynabeads protein A 30 (Thermo Scientific) overnight at 4 C. For histone marker antibodies, 50 μl of Dynabeads protein A was loaded with 3 sg antibody. For CTCF, 50 μl of Dynabeads protein A was loaded with 20 μl antibody. The next day, magnetic beads were washed through the following steps: buffer 1 (150 mM NaCl, 50 mM Tris-Cl, 1 mM EDTA, 5% sucrose, 0.02% NaN₃, 1% Triton X-100, 0.2% SDS, pH 8.0) two times; buffer 2 (0.1% deoxycholic acid, 1 mM EDTA, 50 mM HEPES, 500 mM NaCl, 1% Triton X-100, 0.02% NaCl, pH 8.0) two times; buffer 3 (0.5% deoxycholic acid, 1 mM EDTA, 250 mM LiCl, 0.5% NP40, 0.02% NaN₃) two times; TE buffer one time. To reverse crosslinks, samples were incubated with 20 μg/ml proteinase K (Ambion) at 65° C. overnight. The samples were then extracted with phenol:chloroform:isoamyl alcohol (25:24:1) followed by chloroform, ethanol precipitated in the presence of glycogen, and re-suspended in 10 mM Tris buffer (pH 8). After reverse crosslinking and purification of DNA, qPCR was performed with the primers listed in Table 7. Briefly, p16 primer detecting the enrichments of all histone markers and CTCF is located in the proximal promoter region within 100 bp around TSS; primers located 50 kb upstream of p16 (Neg 1) and 10 kb downstream of p16 (Neg 2) are the negative control primers located in the regions without enrichment of any of the above proteins. The antibodies used in ChIP assays were: H3K4Me3 (C42D8, #9751, Cell Signaling Technologies), H3K27Ac (ab45173, Abcam), H3K9Me3 (D4W1U, #13969, Cell Signaling Technologies), CTCF (#07-729, Sigma), Rabbit IgG monoclonal (ab172730, Santa Cruz).

### Circularized Chromosome Conformation Capture (4C)-Seq

4C-seq was performed as described previously (67) with modifications (68). In brief, SNU398 cells with stable CRISPR-DiR treatment for 13 days were used for 4C-Seq. 30 million sample a) guided by GN2 non-targeting and 30 million sample b) guided by guides (G19, G36, G110, and G111) targeting region D1+D3 were crosslinked in 1% formaldehyde for 10 mins at RT with rotation. Then formaldehyde was neutralized by adding 2.5 M glycine to a final concentration of 0.25 M and rotating for 5 mins at RT. After washing in cold PBS, cells were resuspended in 9 ml lysis buffer (10 mM Tris-HCl pH8.0, 10 mM NaCl, 5 mM EDTA, 0.5% NP 40, with addition of EDTA-free protease inhibitor (complete tablet, freshly dissolved in nuclease free water to make a 100× stock, 5056489001, Roche) and lysed multiple times with resuspension every 2-3 mins during the 10 mins incubation on ice. After lysis, each lysate was split into two 15 ml falcon tubes for viewpoint 1 (Csp6I) or viewpoint 2 (DpnII), respectively (4.5 ml/tube, 15 million cells). After spinning down at 3,000 rpm for 10 mins at 4° C., each nuclear preparation was washed with 500 μl 1× CutSmart buffer from NEB and spun at 800g for 10 min at 4° C., followed by resuspension into 450 μl nuclease free (NF) H₂O and transferring exactly 450 μl of the sample into a 1.5 mL eppendorf tube. To each tube, 60 μl of 10× restriction enzyme buffer provided together with the corresponding restriction enzyme (viewpoint 1:10× Buffer B (ER0211, Invitrogen); viewpoint 2:10× NEBuffer™ DpnII (R0543M, NEB)) and 15 μl of 10% SDS buffer were added to the 450 μl sample, followed by an incubation at 37° C. for 1 hour with shaking (900 RPM, EppendorfThermomixer), followed by adding 75 μl of 20% Triton X-100 to each tube for 1-hour incubation at 37° C. with shaking (900 RPM). 20 μl samples from each tube were taken out as “undigested” and stored at −20 C. For viewpoint 1, 50 μl Csp6I (ER0211, Invitrogen) was added (500 U per tube) together with 5.6 μl 10× Buffer B (ER0211, Invitrogen) for 18 hours digestion at 37° C. with shaking (700 RPM); for viewpoint 2, 10 μl DpnII (R0543M, NEB) (500 U per tube) together with 8 μl NF H₂O and 2 μl 10× NEBuffer™ DpnII were added for 18 hours digestion at 37° C. with shaking (700 RPM). The next day, after removing 20 μl of the sample for de-crosslinking, confirming a digestion efficiency over 80%, and performing PicoGreen DNA quantification to check the DNA concentration in each reaction, 10 ug of the digested DNA was taken out into a new tube and the volume was adjusted to 600 μl with NF H₂O. The samples were heat inactivated at 65° C. for 20 min. Heat inactivated chromatin was added into 1× ligation buffer (EL0013, Invitrogen) supplemented with 1% Triton X-100, 0.1 mg/ml BSA, and the volume was adjusted with NF H₂O to 10 ml with a final DNA concentration of 1 ng/μl. After adding 660 U T4 DNA ligase (EL0013, Invitrogen 30U/μl), samples were incubated at 16° C. in thermal incubator without shaking. The next day, a final concentration of 0.5% SDS and 0.05 mg/ml proteinase K (Ambion) were added to each sample, followed by 65° C. incubation overnight for de-crosslinking. The next day, after adding 30 μl of RNase A (10 mg/ml, PureLink), samples were incubated at 37° C. for hour, followed by phenol: chloroform DNA purification. The chromatin was extracted with phenol:chloroform:isoamyl alcohol (25:24:1) followed by chloroform, ethanol precipitated (split to 5 ml/tube and topped up with NF H₂O to 15 ml, then adding 100% ethanol to 68% to avoid SDS precipitation) in the presence of glycogen and dissolved in 10 mM Tris buffer (pH8). The ligated chromatin was analyzed by agarose gel electrophoresis and the concentration was determined by QUBIT HS DNA kit. 7 μg of ligated chromatin was digested with 10U specific second cutter NlaIII (R0125S, NEB) in 100 μl system with CutSmart Buffer (NEB), 37° C. overnight without shaking. 5 μl digested chromatin was analyzed by gel electrophoresis prior to heat inactivation. Restriction enzyme was heat-inactivated by incubating the chromatin at 65° C. for 20 mins. 7 μg NlaIII digested chromatin was ligated with T4 DNA ligase (EL0013, Invitrogen, 30U/μl) at 20 U/ml in 1× ligation buffer (EL0013, Invitrogen), incubated at 16° C. overnight. The ligated DNA was recovered by phenol:chloroform:isoamyl alcohol (25:24:1) extraction and ethanol precipitation. 100 ng DNA of each sample was used for 4C library preparation. The library was constructed by inverse PCR and nested PCR with KAPA HiFi HotStart ReadyMix (KK2602). The 1st PCR was performed at 100 ng DNA+1.75 μl 1^{st} PCR primer mix+12.5 μl KAPA HiFi HotStart ReadyMix+H2O to 25 μl. The 1^{st} PCR program was 95° C., 3 min, 15 cycle of (98° C., 20s; 65° C., 15s; 72° C., 1 min), 722° C., 5 min, 42° C. hold. The 1 PCR products were purified by MinElute PCR Purification Kit (28004, Qiagen) and eluted in 13 μl Elution Buffer in the kit. The 2^{nd} PCR was performed at purified 1^{st} PCR product+1.75 μl 2^{nd} PCR primer mix+12.5 μl KAPA HiFi HotStart ReadyMix+H₂O to 25 μl. The 2^{d} PCR program was 95° C., 3 min, 13 cycle of (98° C., 20s; 65° C., 15s; 720° C., 1 min), 720° C., 5 min, 4° C. hold. The 2^{d} PCR products were purified by MinElute PCR Purification Kit (28004, Qiagen) and eluted in 10 μl Elution Buffer in the kit. The primer mix was 5 μl 100M forward primer+5 μl 100M reverse primer+90 μl H₂O. All primer sequences and barcodes are listed in Table 8. The libraries were subjected to size selection (250-600 bp) on a 4-20% TBE PAGE gel (Thermo Scientific). The TBE gel was run at 180V, 55 mins, stained with Sybr Safe and visualized with gel safe, and the libraries were extracted from PAGE using a gel crush protocol. Picogreen quantification, Bioanalyzer, and KAPA library quantification were performed to check the quality, size and amount of the recovered libraries, and NextSeq 500/550 Mid Output kit V2.5 (150 Cycles)(20024904, Illumina) was used for single end Nextseq sequencing.

### Statistical Analysis

Methylation changes of clones analysed by bisulphite sequencing PCR (BSP) were calculated using the online methylation analysis tool QUMA (http://quma.cdb.riken.ip/, and the FIG. 22B was generated by R functions (http://www.r-project.org). For mRNA qRT-PCR and ChIP-qPCR, p values were calculated by t-test in GraphPad Prism Software. Values of P<0.05 were considered statistically significant (*P<0.05; **P<0.01; ***P<0.001). The Mean f SD of triplicates is reported.

### Bioinformatic Analysis

### TF Bindings and Motif Analysis

TF direct binding motifs surrounding p16 transcription start site were searched out using the TFregulomeR package, which is a TF motif analysis tool linking to 1,468 public TF ChIP-seq datasets in human (52). Specifically, the function intersectPeakMatrix from the TFregulomeR package was used to map the occurrences of TF motifs derived from ChIP-seq across the genomic regions of interest. CTCF binding was analyzed in our study using ChIP-Seq data from cell lines analyzed by TFregulomeR (FB8470, GM12891, GM19240, prostate epithelial cells, and H1-derived mesenchymal stem cells).

### Histone Marks ChIP-Seq Analysis

Histone marks (H3K4Me3, H3K27Ac, H3K4MeI) enrichments shown in FIG. 24A were determined by ChIP-seq data cross 7 cell lines (GM12878, H1-hESC, HSMM, HUVEC, K562, NHEK, NHLF) obtained from ENCODE.

### WGBS Analysis

For WGBS analysis, the leading 3 bases and adaptor sequences were trimmed from paired-end reads by TrimGalore. The resulting FASTQ files were analyzed by BISMARK (69). PCR duplicates were removed by SAMtools rmdup (70). Then bismark_methylation_extractor continued the extraction of the DNA methylation status on every cytosine sites. DNA methylation levels were converted into bedGraph and then to bigWig format by bedGraphToBigWig.

### 4C-Seq Analysis

For the 4C-seq analysis, the long-range genomic interaction regions generated by the 4C-Seq experiment were first processed using the CSI portal (71). Briefly, raw fq files were aligned to a masked hg19 reference (masked for the gap, repetitive and ambiguous sequences) using bwa mem (72). Barn files were converted to read coverage files by bedtools genomecov (73). The read coverage was normalized according to the sequencing depth. BedGraph files of the aligned bams were converted to bigWig format by bedGraphToBigWig. Next, the processed alignment files were analyzed using r3CSeq (74) and using the associated masked hg19 genome (BSgenome.Hsapiens.UCSC.hg19.masked) (75), from the R Bioconductor repository. Chromosome 9 was selected as the viewpoint, and Csp6I, DpnII were used as the restriction enzyme to digest the genome. Smoothed bam coverage maps were generate using bamCoverage from the deeptools suite (76) with the flags “—normalizeUsing RPGC—binSize 2000—smoothLength 6000-effectiveGenomeSize 2864785220-outFileFormat bedgraph” and plotted using the Bioconductor package Sushi (77) to get the viewpoint coverage depth maps. BigInteract files for UCSC and bedpe files were manually generated with the “score” values being calculated as −log(interaction_q-value_from_r3CSeq+1*10⁻¹⁰). Sushi was then used to plot the bedpe files to get the 4C looping plots. To identify differential interaction peaks, HOMER's (78) get DifferentialPeaks was used with the flag “−F 1.5” afterwhich the corresponding bigInteract and bedpe files were generated as described.

The WGBS data and 4C-seq data generated by this study can be accessed in Gene Expression Omnibus (with access number GSE153563).

One or more illustrative embodiments have been described by way of example. It will be understood to persons skilled in the art that a number of variations and modifications can be made without departing from the scope of the invention as defined in the claims.

TABLE 1
Primer Sequences used for qRT-PCR

| Primer Name | Sequence (5′ to 3′) | SEQ ID NO: |
|---|---|---|
| p16-F | CAACGCACCGAATAGTTACG | 56 |
| p16-R | AGCACCACCAGCGTGTC | 57 |
| CEBPA-F | TATAGGCTGGGCTTCCCCTT | 58 |
| CEBPA-R | AGCTTTCTGGTGTGACTCGG | 59 |
| p14-F | GCAGGTTCTTGGTGACCCTC | 60 |
| p14-R | CCATCATCATGACCTGGTCTTCTA | 61 |
| p15-F | TAGTGGAGAAGGTGCGACAG | 62 |
| p15-R | GCGCTGCCCATCATCATG | 63 |
| ACTB-F | TGAAGTGTGACGTGGACATC | 64 |
| ACTB-R | GGAGGAGCAATGATCTTGAT | 65 |

TABLE 2
Plasmids used in transient transfection and lentivirus generated stable lines

| Plasmid | Purpose | Description |
|---|---|---|
| pMD2.G | Lentivirus packaging | |
| (Addgene: | | |
| 12259) | | |
| psPAX2 | Lentivirus packaging | |
| (Addgene: | | |
| 12259) | | |
| plv-dCas9- | Lentivirus plasmid for | The Cas9 sequence in FUCas9Cherry (Addgene: 70182) |
| mCherry | generate stable dCas9 cell | was replaced by introducing two point mutations to ger |
| | line | dCas9 sequence. |
| pcw-dCas9- | Lentivirus plasmid for | The Cas9 sequence in pcw-Cas9 (Addgene: 50661) was |
| puro | generate stable inducible | replaced by dCas9 sequence same as plv-dCas9-mCherry. |
| | dCas9 cell line | |
| plv-sgDiR- | The guide-empty | The guide-empty backbone lentivirus plasmid was |
| EGFP | backbone lentivirus | modified from pLV hUbC-dCas9-T2A-GFP (Addgene: |
| | plasmid for generating | 53191). Briefly, the original hUbC-dCas9 sequence was |
| | sgDiR plasmids with all | replaced by U6-sgDiR sequence generated by gBlock |
| | different guide RNA | (IDT), to obtain the guide-empty backbone plasmid with |
| | sequence for stable cell | EGFP selection marker. Once the backbone plasmid was |
| | lines | ready, any guide RNA sequence (IDT) listed in Table 4 |
| | | can be ligated to the BsmBI (NEB #R0580) cut backbone |
| | | plasmid. |
| MLM3636 | Transient transcfection of | The guide-empty backbone plasmid for original sgRNA |
| (Addgene: | sgRNA (original, no DiR) | transient transfection. MLM3636 (Addgene: 43860) was |
| 43860) | | used as the backbone plasmid, with guide GN2 and G2 |
| | | ligated to the plasmid. |
| MLMsgDiR | Transient transcfection of | The guide-empty backbone plasmid for MsgDiR1-8 |
| | MsgDiR1-8 | transient transfection. It was modified from MLM3636 |
| | | (Addgene: 43860), replacing the proginal sgRNA |
| | | sequence with sgDiR1-8 seuqences indicated in Table 3. |
| | | Once the guide-empty backbones were ready, guide RNA |
| | | GN2 and G2 were ligated into the backbones to obtain |
| | | MsgDiR plamids with corresponding guides. |
| pEF_dCas9 | Transient transcfection of | |
| (Addgene: | dCas9 | |
| 68416) | | |

TABLE 3
Sequences of sgRNA and MsgDiR1-8

Legend:
nnnnnnnnnnnnnnnnnnn: 20 bp guide RNA sequence
GAAA: tetra-loop
GAAAA: the sequence within stem-loop 2 which can be replaced by R2 or R5
R2: CCCGGGACGCGGGUCCGGGACAG
R5: CUGAGGCCUUGGCGAGGCUUCUG.
Few studies have tried to modify the sgRNA scaffold to increase their stability. One option may be
to remove the putative POL-III terminator (4 consecutive Us in the beginning of sgRNA scafold) by
replacing the fourth U to G (7). Thus, in the CRISPR-DiR designs, the fourth U (in bold, italic,
underline below) was substituted with G, to make the structure more stable by enabling efficient
transcription, while keeping substantially the same secondary structure and decreasing the minimum
free energy (MFE).
C: Accordingly, the corresponding A was substituted with C (in bold, underline below) to preserve
base-pairing with the “G”.
Original sgRNA (SEQ ID NO: 45)
nnnnnnnnnnnnnnnnnnnGUUUUAGAGCUAGAAAUAGCAAGUUAAAAUAAGGCUAGUCCGUU
AUCAACUUGAAAAAGUGGCACCGAGUCGGUGCUUUUU
Original sgRNA (U to G, present version) (SEQ ID NO: 46)
nnnnnnnnnnnnnnnnnnnGUUUGAGAGCUAGAAAUAGCAAGUUCAAAUAAGGCUAGUCCGUU
AUCAACUUGAAAAAGUGGCACCGAGUCGGUGCUUUUU
sgSAM (sgRNA fused with MS2, for comparison) (SEQ ID NO: 47)
nnnnnnnnnnnnnnnnnnnGUUUUAGAGCUAGGCCAACAUGAGGAUCACCCAUGUCUGCAGGGC
CUAGCAAGUUAAAAUAAGGCUAGUCCGUUAUCAACUUGGCCAACAUGAGGAUCACCCA
UGUCUGCAGGGCCAAGUGGCACCGAGUCGGUGCUUUUU
MsgDiR1 (R2-stemloop2) (SEQ ID NO: 48)
nnnnnnnnnnnnnnnnnnnGUUUGAGAGCUACCCGGGACGCGGGUCCGGGACAGUAGCAAGUUC
AAAUAAGGCUAGUCCGUUAUCAACUUGAAAAAGUGGCACCGAGUCGGUGCUUUUU
MsgDiR2 (R5-stemloop2) (SEQ ID NO: 49)
nnnnnnnnnnnnnnnnnnnGUUUGAGAGCUACUGAGGCCUUGGCGAGGCUUCUUAGCAAGUUC
AAAUA
AGGCUAGUCCGUUAUCAACUUGAAAAAGUGGCACCGAGUCGGUGCUUUUU
MsgDiR3 (tetraloop-R2) (SEQ ID NO: 50)
nnnnnnnnnnnnnnnnnnnGUUUGAGAGCUAGAAAUAGCAAGUUCAAAUAAGGCUAGUCCGUU
AUCAACUUCCCGGGACGCGGGUCCGGGACAGAGUGGCACCGAGUCGGUGCUUUUU
MsgDiR4 (tetraloop-R5) (SEQ ID NO: 51)
nnnnnnnnnnnnnnnnnnnGUUUGAGAGCUAGAAAUAGCAAGUUCAAAUAAGGCUAGUCCGUU
AUCAACUUCUGAGGCCUUGGCGAGGCUUCUAGUGGCACCGAGUCGGUGCUUUUU
MsgDiR5 (R5-R2) (SEQ ID NO: 52)
nnnnnnnnnnnnnnnnnnnGUUUGAGAGCUACUGAGGCCUUGGCGAGGCUUCUUAGCAAGUUC
AAAUAAGGCUAGUCCGUUAUCAACUUCCCGGGACGCGGGUCCGGGACAGAAGUGGCAC
CGAGUCGGUGCUUUUUU
MsgDiR6 (R2-R5) CRISPR-DiR (SEQ ID NO: 53)
nnnnnnnnnnnnnnnnnnnGUUUGAGAGCUACCCGGGACGCGGGUCCGGGACAGUAGCAAGUUC
AAAUAAGGCUAGUCCGUUAUCAACUUCUGAGGCCUUGGCGAGGCUUCUAAGUGGCACC
GAGUCGGUGCUUUUUU
MsgDiR7 (R2-R2) (SEQ ID NO: 54)
nnnnnnnnnnnnnnnnnnnGUUUGAGAGCUACCCGGGACGCGGGUCCGGGACAGUAGCAAGUUC
AAAUAAGGCUAGUCCGUUAUCAACUUCCCGGGACGCGGGUCCGGGACAGAAGUGGCAC
CGAGUCGGUGCUUUUUU
MsgDiR8 (RS-R.5) (SEQ ID NO: 55)
nnnnnnnnnnnnnnnnnnnGUUUGAGAGCUACUGAGGCCUUGGCGAGGCUUCUUAGCAAGUUC
AAAUAAGGCUAGUCCGUUAUCAACUUCUGAGGCCUUGGCGAGGCUUCUAAGUGGCACC
GAGUCGGUGCUUUUUU

TABLE 4
The guide RNA Sequences

| Targeting | Guide | Guide RNA | |
|---|---|---|---|
| Region | RNA Name | sequence (5′ to 3′) | SEQ ID NO. |
| Non-targeting | GN2 | GUUAGGAAUAAAAGCUUUGA | 66 |
| Region D1 | G2 | GCACUCAAACACGCCUUUGC | 67 |
| | G19 | GCUCCCCCGCCUGCCAGCAA | 68 |
| | G36 | GCUAACUGCCAAAUUGAAUCG | 69 |
| Region D2 | G108 | GUGGCCAGCCAGUCAGCCGA | 70 |
| | G122 | GCCGCAGCCGCCGAGCGCACG | 71 |
| Region D3 | G110 | GACCCUCUACCCACCUGGAU | 72 |
| | G111 | GCCCCCAGGGCGUCGCCAGG | 113 |

*For 20 nt guide RNA, the first ″G″ is recognized by RNA Pol III to initiate the transcription of the sgRNA. Therefore, for some of the guides which don't start with ″G″ in their original sequence complementary top16 DNA, we changed the first base pair to ″G″ while keep the entire guide length as 20 nt.

TABLE 5
The location of Region C, Region D1,
Region D2, Region D3 and Region E

| | | Gene Coordinates relative to TSS |
|---|---|---|
| Region | Chromatin Posidon in hg38 | (+1) in hg38 |
| Region C | chr9: 21975404-21975826 | −693 to −271 |
| Region D1 | chr9: 21975134-21975332 | −199 to −1 |
| Region D2 | chr9: 21974678-21975133 | +1 to +456 |
| Region D3 | chr9: 21974471-21974677 | +457 to +663 |
| Region E | chr9: 21973931-21974470 | +664 to +1203 |

TABLE 6
The primers and restriction enzymes for methylation assays
COBRA Primers and Enzymes for Region C, D1, D2, D3, and E:

| | | | SEQ ID | Cutting |
|---|---|---|---|---|
| Region | Primer | Sequence (5′ to 3′) | NO: | Enzyme |
| Region C | p16-BSP-C-F1 | TGGTTTTTGGATTATTGTGTAAT | 73 | TaqaI |
| | | TTT | | |
| | p16-BSP-C-R1 | CTTTCCTAATTATAAAAACCCCA | 74 | |
| | | CC | | |
| Region D1 | p16-BSP-4F | AATTTGGTAGTTAGGAAGGTTG | 75 | BstUI |
| | | TA | | |
| | p16-BSP-4R | TCCCCACCTACCCCCCACA | 76 | |
| Region D2 | p16_BSP_original_ | TTTTTAGAGGATTTGAGGGATA | 77 | BstUI |
| | F | GG | | |
| | Rp16_BSP_original | CTACCTAATTCCAATTCCCCTAC | 78 | |
| | R | A | | |
| Region D3 | Fp16-BSP-D3-F1 | TTTAGGTGGGTAGAGGGTTTGT | 79 | AciI |
| | | AG | | |
| | Rp16-BSP-D3-R1 | AACTCCTCATTCCTCTTCCTTAA | 80 | |
| | | CT | | |
| Region E | p16-E-BSP-F1 | TTAGGTGGGTAGAGGGTTTGTA | 81 | BstUI |
| | | G | | |
| | p16-E-BSP-R1 | CAAACTAAAATAAAATAACTCC | 82 | |
| | | ATCT | | |
| Region D | p16_BS_F | ATTTGGTAGTTAGGAAGGTTGT | 83 | HpyCH4IV |
| (Covering | | A | | |
| Region | p16 _BS_R | CCAAAAAACCTCCCCTTTTTCC | 84 | |
| D1 + D2 + D3) | | | | |

BSP Primers:

| Region | Primer | Sequence (5′ to 3′) | SEQ ID NO: |
|---|---|---|---|
| Region D | p16_BS_F | ATTTGGTAGTTAGGAAGGTTGTA | 85 |
| (Covering | p16_BS_R | CCAAAAAACCTCCCCTTTTTCC | 86 |
| Region | | | |
| D1 + D2 + D3) | | | |

MSP Primers:

| Region | Primer | Sequence (5′ to 3′) | SEQ ID NO: |
|---|---|---|---|
| p16 Exon 1 | UF | TTATTAGAGGGTGGGGTGGATTGT | 87 |
| (Region D2) | UR | CCACCTAAATCAACCTCCAACCA | 88 |
| | MF | TTATTAGAGGGTGGGGCGGAtCGC | 89 |
| | MR | CCACCTAAATCGACCTCCGACCG | 90 |

TABLE 7
Primer Sequences for ChIP-qPCR

| Primer name | Sequence (5′ to 3′) | SEQ ID NO: |
|---|---|---|
| P16-F | GGTGGGGCTCTCACAACT | 91 |
| P16-R | CCTTCCTCCGCGATACAA | 92 |
| P14-F (Positive Control) | AGAAGTCTGCCGCTCCTCTA | 93 |
| P14-R (Positive Control) | ACAGATCAGACGTCAAGCCC | 94 |
| P15-F (Positive Control) | GTGAAGCCCAAGTACTGCCT | 95 |
| P15-F (Positive Control) | TCACTGTGGAGACGTTGGTG | 96 |
| Down10K-1F (Negative Control) | AGGAGCCCATAGCTTGTGGA | 97 |
| Down10K-1R (Negative Control) | GATACTTCCACTAGACATCTTGTCA | 98 |
| Up50K-1F (Negative Control) | ATAAAGCATTGCAGGAGCTTACA | 99 |
| Up50K-1R (Negative Control) | CCTACACATTTTTGTGGCCTGTTT | 100 |

TABLE 8
Primer Sequences for 4C-Seq
1^{st} Round PCR Primers:

| Viewpoint | Primer | Sequence (5′ to 3′) | SEQ ID NO: |
|---|---|---|---|
| Viewpoint 1 | Csp6I-NlaIII-1F | GCCTCCGACCGTAACTATTCG | 101 |
| | Csp6I-NlaIII-1R | AGGACGAAGTTTGCAGGGG | 102 |
| Viewpoint 2 | DpnII-NlaIII-1F | CATTGGAAGGACGGACTCCATT | 103 |
| | DpnII-N1aIII-1R | TGGAAAGATACCGCGGTCC | 104 |

2^{nd} Round PCR Primers:

| Viewpoint | Primer | Sequence (5′ to 3′) | SEQ ID NO: |
|---|---|---|---|
| Viewpoint 1 | Csp6I-NlaIII-C-502 | AATGATACGGCGACCACCGAGATC | 105 |
| | | TACACCTCTCTATTCGTCGGCAGCG | |
| | | TCAGATGTGTATAAGAGACAGAAG | |
| | | CCAAGGAAGAGGAATGAGG | |
| | Csp6I-NlaIII-C-501 | AATGATACGGCGACCACCGAGATC | 106 |
| | | TACACTAGATCGCTCGTCGGCAGCG | |
| | | TCAGATGTGTATAAGAGACAGAAG | |
| | | CCAAGGAAGAGGAATGAGG | |
| | Csp6I-NlaIII-N-703 | CAAGCAGAAGACGGCATACGAGAT | 107 |
| | | TTCTGCCTGTCTCGTGGGCTCGGAG | |
| | | ATGTGTATAAGAGACAGCCAGCCA | |
| | | GTCAGCCGAAG | |
| | Csp6I-NlaIII-N-701 | CAAGCAGAAGACGGCATACGAGAT | 108 |
| | | TCGCCTTAGTCTCGTGGGCTCGGAG | |
| | | ATGTGTATAAGAGACAGCCAGCCA | |
| | | GTCAGCCGAAG | |
| Viewpoint 2 | DpnII-NlaIII-D-501 | AATGATACGGCGACCACCGAGATC | 109 |
| | | TACACTAGATCGCTCGTCGGCAGCG | |
| | | TCAGATGTGTATAAGAGACAGTGC | |
| | | TCAGTGTTCTAGAAGCAGA | |
| | DpnII-NlaIII-D-502 | AATGATACGGCGACCACCGAGATC | 110 |
| | | TACACCTCTCTATTCGTCGGCAGCG | |
| | | TCAGATGTGTATAAGAGACAGTGC | |
| | | TCAGTGTTCTAGAAGCAGA | |
| | DpnII-NlaIII-N-704 | CAAGCAGAAGACGGCATACGAGAT | 111 |
| | | GCTCAGGAGTCTCGTGGGCTCGGA | |
| | | GATGTGTATAAGAGACAGGGAGAG | |
| | | GGGGAGAGCAGG | |
| | DpnII-NlaIII-N-702 | CAAGCAGAAGACGGCATACGAGAT | 112 |
| | | CTAGTACGGTCTCGTGGGCTCGGAG | |
| | | ATGTGTATAAGAGACAGGGAGAGG | |
| | | GGGAGAGCAGG | |

*The italics and bold highlighted sequence within the 2nd round PCR primers are the i5 and i7 barcodes for sequencing purpose. i5 barcodes for the primers in the 1st cut (Csp6I/DpnII0 end, while i7 barcodes for the 2nd cut (NlaIII) end. The underlined part indicating primer sequences specific to each sample.

4C PCR Primer Set:

| | 1st Cut | Samples | | |
|---|---|---|---|---|
| Viewpoint | Enzymes | Primer set | Day 13 GN2 | Day 13 D1 + D3 |
| Viewpoint 1 | Csp6I | 1st Round PCR Primers | Csp6I-NlaIII-1F, | Csp6I-NlaIII-1F, |
| | | | Csp6I-NlaIII-1R | Csp6I-NlaIII-1R |
| | | 2nd Round PCR Primers | Csp6I-NlaIII-C- | Csp6I-NlaIII-C-501, |
| | | | 502, | Csp6I-NlaIII-N-701 |
| | | | Csp6I-NlaIII-N- | |
| | | | 703 | |
| Viewpoint 2 | DpnII | 1st Round PCR Primers | DpnII-NlaIII-1F, | DpnII-NlaIII-1F, |
| | | | DpnII-NlaIII-1R | DpnII-NlaIII-1R |
| | | 2nd Round PCR Primers | DpnII-NlaIII-D- | DpnII-NlaIII-D-502, |
| | | | 501, | DpnII-NlaIII-N-702 |
| | | | DpnII-NlaIII-N- | |
| | | | 704 | |

1. L. S. Qi et al., Repurposing CRISPR as an RNA-guided platform for sequence-specific control of gene expression. Cell 152, 1173 (Feb. 28, 2013).
2. F. S. Howe et al., CRISPRi is not strand-specific at all loci and redefines the transcriptional landscape. eLife 6, e29878 (2017/10/23, 2017).
3. S. Konermann et al., Genome-scale transcriptional activation by an engineered CRISPR-Cas9 complex. Nature 517, 583 (Jan. 29, 2015).
4. H. Nishimasu et al., Crystal structure of Cas9 in complex with guide RNA and target DNA. Cell 156, 935 (Feb. 27, 2014).
5. H. Ma et al., Multiplexed labeling of genomic loci with dCas9 and engineered sgRNAs using CRISPRainbow. Nature biotechnology 34, 528 (May, 2016).
6. B. Chen et al., Dynamic imaging of genomic loci in living human cells by an optimized CRISPR/Cas system. Cell 155, 1479 (Dec. 19, 2013).
7. Y. Dang et al., Optimizing sgRNA structure to improve CRISPR-Cas9 knockout efficiency.

Genome biology 16, 280 (Dec. 15, 2015).
8. S. Kalari, G. P. Pfeifer, in Advances in Genetics, Z. Herceg, T. Ushijima, Eds. (Academic Press, 2010), vol. 70, pp. 277-308.
9. L. Xue et al., Sp1 is involved in the transcriptional activation of p161NK4 by p21Waf1 in HeLa cells. FEBS Letters 564, 199 (2004/04/23/, 2004).
10. Y. Stelzer, C. S. Shivalila, F. Soldner, S. Markoulaki, R. Jaenisch, Tracing Dynamic Changes of DNA Methylation at Single-Cell Resolution. Cell 163, 218 (Sep. 24, 2015).
11. M. Jinek et al., A Programmable Dual-RNA-Guided DNA Endonuclease in Adaptive Bacterial Immunity. Science 337, 816 (2012).
12. L. Cong et al., Multiplex genome engineering using CRISPR/Cas systems. Science (New York, N.Y.) 339, 819 (2013).
13. M. H. Larson et al., CRISPR interference (CRISPRi) for sequence-specific control of gene expression. Nature protocols 8, 2180 (November, 2013).
14. D. W. Ballard et al., The 65-kDa subunit of human NF-kappa B functions as a potent transcriptional activator and a target for v-Rel-mediated repression. Proceedings of the National Academy of Sciences of the United States of America 89, 1875 (1992).
15. M. K. Jensen, Design principles for nuclease-deficient CRISPR-based transcriptional regulators. FEMS yeast research 18, (Jun. 1, 2018).
16. H. Mitsunobu, J. Teramoto, K. Nishida, A. Kondo, Beyond Native Cas9: Manipulating Genomic Information and Function. Trends in biotechnology 35, 983 (October, 2017).
17. L. A. Gilbert et al., CRISPR-mediated modular RNA-guided regulation of transcription in eukaryotes. Cell 154, 442 (Jul. 18, 2013).
18. N. A. Kearns et al., Cas9 effector-mediated regulation of transcription and differentiation in human pluripotent stem cells. Development (Cambridge, England) 141, 219 (2014).
19. M. L. Maeder et al., CRISPR RNA-guided activation of endogenous human genes. Nature methods 10, 977 (2013).
20. J. F. Margolin et al., Kriippel-associated boxes are potent transcriptional repression domains. Proceedings of the National Academy of Sciences of the United States of America 91, 4509 (1994).
21. F. Farzadfard, S. D. Perli, T. K. Lu, Tunable and multifunctional eukaryotic transcription factors based on CRISPR/Cas. ACS synthetic biology 2, 604 (2013).
22. M. Deaner, J. Mejia, H. S. Alper, Enabling Graded and Large-Scale Multiplex of Desired Genes Using a Dual-Mode dCas9 Activator in Saccharomyces cerevisiae. ACS synthetic biology 6, 1931 (2017 Oct. 20, 2017).
23. K. G. Vanegas, B. J. Lehka, U. H. Mortensen, SWITCH: a dynamic CRISPR tool for genome engineering and metabolic pathway control for cell factory construction in Saccharomyces cerevisiae. Microbial cell factories 16, 25 (2017).
24. D I RUSCIO, A., EBRALIDZE, A. K., BENOUKRAF, T., AMABILE, G., GOFF, L. A., TERRAGNI, J., FIGUEROA, M. E., DE FIGUEIREDO PONTES, L. L., ALBERICH-JORDA, M., ZHANG, P., WU, M., D′ALO, F., MELNICK, A., LEONE, G., EBRALIDZE, K. K., PRADHAN, S., RINN, J. L. & TENEN, D. G. 2013. DNMT1-interacting RNAs block gene-specific DNA methylation. Nature, 503, 371-6.
25. GILBERT, L. A., HORLBECK, M. A., ADAMSON, B., VILLALTA, J. E., CHEN, Y., WHITEHEAD, E. H., GUIMARAES, C., PANNING, B., PLOEGH, H. L., BASSIK, M. C., QI, L. S., KAMPMANN, M. & WEISSMAN, J. S. 2014. Genome-Scale CRISPR-Mediated Control of Gene Repression and Activation. Cell, 159, 647-661.
26. GILBERT, L. A., LARSON, M. H., MORSUT, L., LIU, Z., BRAR, G. A., TORRES, S. E., STERN-GINOSSAR, N., BRANDMAN, O., WHITEHEAD, E. H., DOUDNA, J. A., LIM, W. A., WEISSMAN, J. S. & QI, L. S. 2013. CRISPR-mediated modular RNA-guided regulation of transcription in eukaryotes. Cell, 154, 442-451.
27. KONERMANN, S., BRIGHAM, M. D., TREVINO, A. E., JOUNG, J., ABUDAYYEH, O. O., BARCENA, C., HSU, P. D., HABIB, N., GOOTENBERG, J. S., NISHIMASU, H., NUREKI, O. & ZHANG, F. 2015. Genome-scale transcriptional activation by an engineered CRISPR-Cas9 complex.

Nature, 517, 583-8.
28. NISHIMASU, H., RAN, F. A., HSU, P. D., KONERMANN, S., SHEHATA, S. I., DOHMAE, N., ISHITANI, R., ZHANG, F. & NUREKI, O. 2014. Crystal structure of Cas9 in complex with guide RNA and target DNA. Cell, 156, 935-49.
29. P. A. Jones, Functions of DNA methylation: islands, start sites, gene bodies and beyond. Nat Rev Genet 13, 484-492 (2012).
30. J. G. Herman, S. B. Baylin, Gene silencing in cancer in association with promoter hypermethylation. N Engl J Med 349(21), 2042-2054 (2003).
31. P. A. Jones, S. B. Baylin, The fundamental role of epigenetic events in cancer. Nature Reviews Genetics 3, 415-428 (2002).
32. M. Esteller, P. G. Corn, S. B. Baylin, J. G. Herman, A Gene Hypermethylation Profile of Human Cancer. Cancer Research 61, 3225 (2001).
33. R. L. Momparler, V. Bovenzi, DNA methylation and cancer. J Cell Physiol 183(2), 145-154 (2000).
34. S. Saxonov, P. Berg, D. L. Brutlag, A genome-wide analysis of CpG dinucleotides in the human genome distinguishes two distinct classes of promoters. Proceedings of the National Academy of Sciences 103, 1412 (2006).
35. L. A. Gilbert et al., CRISPR-mediated modular RNA-guided regulation of transcription in eukaryotes. Cell 154, 442-451 (2013).
36. S. Konermann et al., Genome-scale transcriptional activation by an engineered CRISPR-Cas9 complex. Nature 517, 583-588 (2015).
37. A. Taghbalout et al., Enhanced CRISPR-based DNA demethylation by Casilio-ME-mediated RNA-guided coupling of methylcytosine oxidation and DNA repair pathways. Nat Commun 10, 4296 (2019).
38. A. Sakamoto et al., DNA Methylation in the Exon 1 Region and Complex Regulation of Twist1 Expression in Gastric Cancer Cells. PLoS One 10, e0145630 (2015).
39. R. Tirado-Magallanes, K. Rebbani, R. Lim, S. Pradhan, T. Benoukraf, Whole genome DNA methylation: beyond genes silencing. Oncotarget 8, 5629-5637 (2017).
40. S. Mani, Z. Herceg, DNA demethylating agents and epigenetic therapy of cancer. Adv Genet 70, 327-340 (2010).
41. M. V. C. Greenberg, D. Bourc'his, The diverse roles of DNA methylation in mammalian development and disease. Nature Reviews Molecular Cell Biology 20, 590-607 (2019).
42. B. Li, M. Carey, J. L. Workman, The role of chromatin during transcription. Cell 128, 707-719 (2007).
43. A. Di Ruscio et al., DNMT1-interacting RNAs block gene-specific DNA methylation. Nature 503, 371-376 (2013).
44. H. Ma et al., Multiplexed labeling of genomic loci with dCas9 and engineered sgRNAs using CRISPRainbow. Nat Biotechnol 34, 528-530 (2016).
45. M. Jinek et al., Structures of Cas9 endonucleases reveal RNA-mediated conformational activation.

Science 343, 1247997 (2014).
46. M. Gonzalez-Zulueta et al., Methylation of the 5′ CpG Island of the p16/CDKN2 Tumor Suppressor Gene in Normal and Transformed Human Tissues Correlates with Gene Silencing. Cancer Research 55, 4531 (1995).
47. J. Shen et al., Genome-wide DNA methylation profiles in hepatocellular carcinoma. Hepatology 55(6), 1799-1808 (2012).
48. Y. H. Shim, H.-J. Yoon Gs Fau-Choi, Y. H. Choi Hj Fau-Chung, E. Chung Yh Fau-Yu, E. Yu, p16 Hypermethylation in the early stage of hepatitis B virus-associated hepatocarcinogenesis. Cancer Lett 190(2), 213-219 (2003).
49. K. Pandiyan et al., Functional DNA demethylation is accompanied by chromatin accessibility. Nucleic Acids Research 41, 3973-3985 (2013).
50. J. Gil, G. Peters, Regulation of the INK4b-ARF-INK4a tumour suppressor locus: all for one or one for all. Nat Rev Mol Cell Biol 7, 667-677 (2006).
51. C. Rodriguez et al., CTCF is a DNA methylation-sensitive positive regulator of the INK/ARF locus. Biochem Biophys Res Commun 392, 129-134 (2010).
52. Q. X. X. Lin, D. Thieffry, S. Jha, T. Benoukraf, TFregulomeR reveals transcription factors' context-specific features and functions. Nucleic acids research 48, e10-e10 (2020).
53. F. Recillas-Targa, E. De La Rosa-Velazquez Ia Fau-Soto-Reyes, L. Soto-Reyes E Fau-Benitez-Bribiesca, L. Benitez-Bribiesca, Epigenetic boundaries of tumour suppressor gene promoters: the CTCF connection and its role in carcinogenesis. J Cell Mol Med 10(3), 554-568 (2006).
54. M. Witcher, B. M. Emerson, Epigenetic silencing of the p16(INK4a) tumor suppressor is associated with loss of CTCF binding and a chromatin boundary. Mol Cell 34, 271-284 (2009).
55. A. T. Hark et al., CTCF mediates methylation-sensitive enhancer-blocking activity at the H19/Igf2 locus. Nature 405(6785), 486-489 (2000).
56. C.-T. Ong, V. G. Corces, CTCF: an architectural protein bridging genome topology and function.

Nature Reviews Genetics 15, 234-246 (2014).
57. A. Visel et al., Targeted deletion of the 9p21 non-coding coronary artery disease risk interval in mice. Nature 464, 409-412 (2010).
58. Q. Li et al., FOXA1 mediates p16(INK4a) activation during cellular senescence. EMBO J 32, 858-873 (2013).
59. Y. T. Liu et al., Identification of De Novo Enhancers Activated by TGFbeta to Drive Expression of CDKN2A and B in HeLa Cells. Mol Cancer Res 17, 1854-1866 (2019).
60. M. L. Maeder et al., CRISPR RNA-guided activation of endogenous human genes. Nat Methods 10, 977-979 (2013).
61. A. R. Karpf, Epigenetic alterations in oncogenesis. (Springer, New York, N.Y., 2013).
62. A. Fuso et al., Early demethylation of non-CpG, CpC-rich, elements in the myogenin 5′-flanking region: a priming effect on the spreading of active demethylation. Cell Cycle 9, 3965-3976 (2010).
63. J. K. e. al, Pseudogene-mediated DNA demethylation leads to oncogene activation. Unpublished, (2020).
64. M. R. Dimitrov et al., Successive DNA extractions improve characterization of soil microbial communities. PeerJ 5, e2915-e2915 (2017).
65. Y. Dang et al., Optimizing sgRNA structure to improve CRISPR-Cas9 knockout efficiency. Genome Biology 16, 280 (2015).
66. D. Gonzalez et al., ZNF143 protein is an important regulator of the myeloid transcription factor C/EBPα. J Biol Chem 292(46), 18924-18936 (2017).
67. M. Matelot, D. Noordermeer, Determination of High-Resolution 3D Chromatin Organization Using Circular Chromosome Conformation Capture (4C-seq). Methods Mol Biol 1480, 223-241 (2016).
68. P. H. L. Krijger, G. Geeven, V. Bianchi, C. R. E. Hilvering, W. de Laat, 4C-seq from beginning to end: A detailed protocol for sample preparation and data analysis. Methods 170, 17-32 (2020).
69. F. Krueger, S. R. Andrews, Bismark: a flexible aligner and methylation caller for Bisulfite-Seq applications. Bioinformatics 27(11), 1571-1572 (2011).
70. H. Li et al., The Sequence Alignment/Map format and SAMtools. Bioinformatics 25, 2078-2079 (2009).
71. O. An et al., CSI NGS Portal: An Online Platform for Automated NGS Data Analysis and Sharing. Int J Mol Sci 21, (2020).
72. H. Li, Rₐ Durbin, Fast and accurate long-read alignment with Burrows-Wheeler transform. Bioinformatics 26, 589-595 (2010).
73. A. Rₐ Quinlan, I. M. Hall, BEDTools: a flexible suite of utilities for comparing genomic features. Bioinformatics 26, 841-842 (2010).
74. S. Thongjuea, F. G. Stadhouders R Fau-Grosveld, E. Grosveld Fg Fau-Soler, B. Soler E Fau-Lenhard, B. Lenhard, r3Cseq: an R/Bioconductor package for the discovery of long-range genomic interactions from chromosome conformation capture and next-generation sequencing data. Nucleic Acids Res 41(13), 132 (2013).
75. T. TBD, BSgenome.Hsapiens.UCSC.hgl9.masked: Full masked genome sequences for Homo sapiens (UCSC version hg19, based on GRCh37.p13). R package version 1.3.993., (2020).
76. F. Ramirez et al., deepTools2: a next generation web server for deep-sequencing data analysis.

Nucleic Acids Res 44(W1), W160-W165 (2016).
77. P. DH, Sushi: Tools for visualizing genomics data. R package version 1.26.0., (2020).
78. S. Heinz et al., Simple combinations of lineage-determining transcription factors prime cis-regulatory elements required for macrophage and B cell identities. Molecular cell 38, 576-589 (2010).
79. A. R. Gruber, R. Lorenz, S. H. Bernhart, R. Neub6ck, I. L. Hofacker, The Vienna RNA websuite. Nucleic acids research 36, W70-W74 (2008).
80. Lu et al., Reprogrammable CRISPR/dCas9-based recruitment of DNMT1 for site-specific DNA demethylation and gene regulation, Cell Discovery, 2019, 5:22 (80).
81. Berg, T., Guo, Y., Abdelkarim, M., Fliegauf, M., & Lubbert, M. (2007). Reversal of p15/INK4b hypermethylation in AML1/ETO-positive and -negative myeloid leukemia cell lines. Leuk Res, 31(4), 497-506. doi:10.1016/j.leukres.2006.08.008
82. Konermann, S., Brigham, M. D., Trevino, A. E., Joung, J., Abudayyeh, O. O., Barcena, C., . . . Zhang, F. (2015). Genome-scale transcriptional activation by an engineered CRISPR-Cas9 complex. Nature, 517(7536), 583-588. doi:10.1038/nature14136
83. Lu, A., Wang, J., Sun, W., Huang, W., Cai, Z., Zhao, G., & Wang, J. (2019). Reprogrammable CRISPR/dCas9-based recruitment of DNMT1 for site-specific DNA demethylation and gene regulation. Cell Discovery, 5(1), 22. doi:10.1038/s41421-019-0090-1.
84. R. Itzykson, P. Fenaux, Epigenetics of myelodysplastic syndromes. Leukemia 28, 497-506 (2014).

All references cited herein an elsewhere in the specification are herein incorporated by reference in their entireties.

1-46. (canceled)

47. An oligonucleotide comprising:
a targeting portion having sequence complementarity and binding affinity with a region of genomic DNA within a gene, near a gene, or both; and
a single guide RNA (sgRNA) scaffold portion, wherein a tetra-loop portion of the sgRNA is modified and comprises an R2 stem loop of DNMT1-interacting RNA (DiR), and wherein a stem loop 2 portion of the sgRNA is modified and comprises an R5 step loop of DiR.

48. The oligonucleotide of claim 47, wherein the oligonucleotide is one or more of the following:
(a) an oligonucleotide wherein the targeting portion has sequence complementarity and binding affinity with a non-template strand of the genomic DNA within the gene, near the gene, or both;
(b) an oligonucleotide wherein the R2 and R5 stem loops of DiR are from extra-coding CEBPA (ecCEBPA);
(c) an oligonucleotide wherein the targeting portion targets a methylated region of the genomic DNA;
(d) an oligonucleotide wherein the oligonucleotide comprises the sequence:
(Rₐ)GUUUR_{b}AGAGCUA(R_{c})UAGCAAGUUR_{d}AAAUAAGGCUAGUCCGUUAUCAACUU(Rₑ)AGU GGCACCGAGUCGGUGC(R_{f}) (Formula I)
wherein
Rₐ comprises the targeting portion, and comprises about 20 to about 21 nucleotides in length;
R_{b} is A, G, or C, and R_{d} is the complementary base pair of R_{b};
R_{c} comprises the R2 stem loop of DiR, comprising sequence CCCGGGACGCGGGUCCGGGACAG (SEQ ID NO: 7);
Rₑ comprises the R5 step loop of DiR, comprising sequence CUGAGGCCUUGGCGAGGCUUCU (SEQ ID NO: 8); and
R_{f} is optionally present, and comprises a poly U transcription termination sequence;
(e) an oligonucleotide comprising the sequence:
(Rₐ)GUUUGAGAGCUACCCGGGACGCGGGUCCGGGACAGUAGCAAGUUCAAAUAAGGCUAG UCCGUUAUCAACUUCUGAGGCCUUGGCGAGGCUUCUAAGTGGCACCGAGUCGGUGCUUUU UU; (Formula II)
wherein Rₐ comprises the targeting portion, and comprises about 20 to about 21 nucleotides in length; and
(f) an oligonucleotide wherein the gene is P16, and R comprises:
(SEQ ID NO: 9)
GCUCCCCCGCCUGCCAGCAA;
(SEQ ID NO: 10)
GCUAACUGCCAAAUUGAAUCG;
(SEQ ID NO: 11)
GACCCUCUACCCACCUGGAU;
or
(SEQ ID NO: 12)
GCCCCCAGGGCGUCGCCAGG.

49. A plasmid or vector encoding the oligonucleotide of claim 47.

50. A composition comprising an oligonucleotide of claim 47 and a dead Cas9 (dCas9).

51. A composition comprising any one or more of:
the oligonucleotide of claim 47; and
a plasmid or vector encoding the oligonucleotide;
wherein the composition further comprises one or more of:
a pharmaceutically acceptable carrier, excipient, diluent, or buffer;
a dead Cas9 (dCas9); or
an oligonucleotide, plasmid, or vector encoding a dead Cas9 (dCas9).

52. The composition of claim 51, wherein the dCas9 comprises D1OA and H840A mutations.

53. A method for targeted demethylation and/or activation of a gene, said method comprising:
introducing a dead Cas9 (dCas9) and one or more oligonucleotides into a cell, the one or more oligonucleotides each comprising:
a targeting portion having sequence complementarity and binding affinity with a region of genomic DNA within the gene, near the gene, or both; and
a single guide RNA (sgRNA) scaffold portion, wherein a tetra-loop portion of the sgRNA is modified and comprises an R2 stem loop of DNMT1-interacting RNA (DiR), and wherein a stem loop 2 portion of the sgRNA is modified and comprises an R5 step loop of DiR;
thereby demethylating and/or activating the gene by inhibiting DNA methyltransferase 1 (DNMT1) activity on the gene.

54. The method of claim 53, wherein the method is one or more of the following:
(a) a method wherein the targeting portion of at least one of the one or more oligonucleotides has sequence complementarity and binding affinity with a non-template strand of the genomic DNA within the gene, near the gene, or both;
(b) a method wherein the step of introducing comprises transfecting, delivering, or expressing the one or more oligonucleotides and the dCas9 in the cell;
(c) a method wherein the one or more oligonucleotides comprise a targeting portion having sequence complementarity and binding affinity with a region of genomic DNA within a gene, near a gene, or both; and a single guide RNA (sgRNA) scaffold portion, wherein a tetra-loop portion of the sgRNA is modified and comprises an R2 stem loop of DNMT1-interacting RNA (DiR), and wherein a stem loop 2 portion of the sgRNA is modified and comprises an R5 step loop of DiR; and
(d) a method wherein the cell is exposed to the dCas9 and the one or more oligonucleotides for a period of at least about 3 days, at least about 4 days, at least about 5 days, at least about 6 days, at least about 7 days, or at least about 8 days, or about 3 days to about a week.

55. A method for targeted demethylation and/or activation of a gene, comprising introducing the oligonucleotide of claim 47 into a cell thereby demethylating and/or activating the gene by inhibiting DNA methyltransferase 1 (DNMT1) activity on the gene.

56. A method for treating a disease or disorder associated with decreased expression of at least one gene due to aberrant DNA methylation in a subject in need thereof, said method comprising:
treating the subject with a dead Cas9 (dCas9) and one or more oligonucleotides, the one or more oligonucleotides each comprising:
a targeting portion having sequence complementarity and binding affinity with a region of genomic DNA within the gene, near the gene, or both; and
a single guide RNA (sgRNA) scaffold portion, wherein a tetra-loop portion of the sgRNA is modified and comprises an R2 stem loop of DNMT1-interacting RNA (DiR), and wherein a stem loop 2 portion of the sgRNA is modified and comprises an R5 step loop of DiR;
thereby demethylating and/or activating the gene by inhibiting DNA methyltransferase 1 (DNMT1) activity on the gene and treating the disease or disorder.

57. The method of claim 56, wherein the method is one or more of the following:
(a) a method wherein targeting portion of at least one of the one or more oligonucleotides has sequence complementarity and binding affinity with a non-template strand of the genomic DNA within the gene, near the gene, or both;
(b) a method wherein the step of treating comprises transfecting, delivering, or expressing the one or more oligonucleotides and the dCas9 in at least one cell of the subject; (c) a method wherein the one or more oligonucleotides comprise: a targeting portion having sequence complementarity and binding affinity with a region of genomic DNA within a gene, near a gene, or both; and a single guide RNA (sgRNA) scaffold portion, wherein a tetra-loop portion of the sgRNA is modified and comprises an R2 stem loop of DNMT1-interacting RNA (DiR), and wherein a stem loop 2 portion of the sgRNA is modified and comprises an R5 step loop of DiR;
(d) a method wherein the subject is exposed to the dCas9 and the one or more oligonucleotides for a period of at least about 3 days, at least about 4 days, at least about 5 days, at least about 6 days, at least about 7 days, or at least about 8 days, or about 3 days to about a week;
(e) a method wherein the promoter region is a CpG-rich region having at least some methylation;
(f) a method wherein the disease or disorder comprises cancer;
(g) a method wherein the gene is a tumor suppressor gene;
(h) a method wherein the targeting portion of at least one of the one or more oligonucleotides targets the promoter-exon1-intron1 region of the P16 gene; and
(i) a method wherein the one or more oligonucleotides comprise one or more of:
G19sgR2R5 (SEQ ID NO: 1):
GCUCCCCCGCCUGCCAGCAAGUUUGAGAGCUACCCGGGACGCGGGUCCG
GGACAGUAGCAAGUUCAAAUAAGGCUAGUCCGUUAUCAACUUCUGAGGC
CUUGGCGAGGCUUCUAAGUGGCACCGAGUCGGUGCUUUUUU;
G36sgR2R5 (SEQ ID NO: 2):
GCUAACUGCCAAAUUGAAUCGGUUUGAGAGCUACCCGGGACGCGGGUCC
GGGACAGUAGCAAGUUCAAAUAAGGCUAGUCCGUUAUCAACUUCUGAGG
CCUUGGCGAGGCUUCUAAGUGGCACCGAGUCGGUGCUUUUUU;
G110sgR2R5 (SEQ ID NO: 3):
GACCCUCUACCCACCUGGAUGUUUGAGAGCUACCCGGGACGCGGGUCCG
GGACAGUAGCAAGUUCAAAUAAGGCUAGUCCGUUAUCAACUUCUGAGGC
CUUGGCGAGGCUUCUAAGUGGCACCGAGUCGGUGCUUUUUU;
G111sgR2R5 (SEQ ID NO: 4):
GCCCCCAGGGCGUCGCCAGGGUUUGAGAGCUACCCGGGACGCGGGUCCG
GGACAGUAGCAAGUUCAAAUAAGGCUAGUCCGUUAUCAACUUCUGAGGC
CUUGGCGAGGCUUCUAAGUGGCACCGAGUCGGUGCUUUUUU;
G108sgR2R5 (SEQ ID NO: 5):
GUGGCCAGCCAGUCAGCCGAGUUUGAGAGCUACCCGGGACGCGGGUCCG
GGACAGUAGCAAGUUCAAAUAAGGCUAGUCCGUUAUCAACUUCUGAGGC
CUUGGCGAGGCUUCUAAGUGGCACCGAGUCGGUGCUUUUUU;
or
G122sgR2R5 (SEQ ID NO: 6):
GCCGCAGCCGCCGAGCGCACGGUUUGAGAGCUACCCGGGACGCGGGUCC
GGGACAGUAGCAAGUUCAAAUAAGGCUAGUCCGUUAUCAACUUCUGAGG
CCUUGGCGAGGCUUCUAAGUGGCACCGAGUCGGUGCUUUUUU;
or any combinations thereof.

58. A method of treating a disease or disorder associated with decreased expression of at least one gene due to aberrant DNA methylation, comprising administering the oligonucleotide of claim 47 in a subject in need thereof.


SEQUENCE LISTING (113 sequences)

SEQ ID NO: 1 - RNA, 139 nt, Artificial Sequence
  G19sgR2R5
gcucccccgccugccagcaaguuugagagcuacccgggacgcggguccgggacaguagcaaguucaaauaaggcuaguccguuaucaacuucugaggccuuggcgaggcuucuaaguggcaccgagucggugcuuuuuu

SEQ ID NO: 2 - RNA, 140 nt, Artificial Sequence
  G36sgR2R5
gcuaacugccaaauugaaucgguuugagagcuacccgggacgcggguccgggacaguagcaaguucaaauaaggcuaguccguuaucaacuucugaggccuuggcgaggcuucuaaguggcaccgagucggugcuuuuuu

SEQ ID NO: 3 - RNA, 139 nt, Artificial Sequence
  G110sgR2R5
gacccucuacccaccuggauguuugagagcuacccgggacgcggguccgggacaguagcaaguucaaauaaggcuaguccguuaucaacuucugaggccuuggcgaggcuucuaaguggcaccgagucggugcuuuuuu

SEQ ID NO: 4 - RNA, 139 nt, Artificial Sequence
  G111sgR2R5
gcccccagggcgucgccaggguuugagagcuacccgggacgcggguccgggacaguagcaaguucaaauaaggcuaguccguuaucaacuucugaggccuuggcgaggcuucuaaguggcaccgagucggugcuuuuuu

SEQ ID NO: 5 - RNA, 139 nt, Artificial Sequence
  G108sgR2R5
guggccagccagucagccgaguuugagagcuacccgggacgcggguccgggacaguagcaaguucaaauaaggcuaguccguuaucaacuucugaggccuuggcgaggcuucuaaguggcaccgagucggugcuuuuuu

SEQ ID NO: 6 - RNA, 140 nt, Artificial Sequence
  G122sgR2R5
gccgcagccgccgagcgcacgguuugagagcuacccgggacgcggguccgggacaguagcaaguucaaauaaggcuaguccguuaucaacuucugaggccuuggcgaggcuucuaaguggcaccgagucggugcuuuuuu

SEQ ID NO: 7 - RNA, 23 nt, Artificial Sequence
  R2 stem loop of DiR
cccgggacgcggguccgggacag

SEQ ID NO: 8 - RNA, 22 nt, Artificial Sequence
  R5 stem loop of DiR
cugaggccuuggcgaggcuucu

SEQ ID NO: 9 - RNA, 20 nt, Artificial Sequence
  P16 targeting portion example
gcucccccgccugccagcaa

SEQ ID NO: 10 - RNA, 21 nt, Artificial Sequence
  P16 targeting portion example
gcuaacugccaaauugaaucg

SEQ ID NO: 11 - RNA, 20 nt, Artificial Sequence
  P16 targeting portion example
gacccucuacccaccuggau

SEQ ID NO: 12 - RNA, 20 nt, Artificial Sequence
  P16 targeting portion example
gcccccagggcgucgccagg

SEQ ID NO: 13 - RNA, 20 nt, Artificial Sequence
  Guide 1.6 sgDiR
gcugcggcugcugcucgccc

SEQ ID NO: 14 - DNA, 5040 bp, Artificial Sequence
  dCas9-mCherry Plasmid
atggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagggcggtgagggcagaggaagtctgctaacatgcggtgacgtcgaggagaatcctggcccaatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 15 - RNA, 139 nt, Artificial Sequence
  MsgRNA
gcacucaaacacgccuuugcguuuuagagcuacccgggacgcggguccgggacaguagcaaguuaaaauaaggcuaguccguuaucaacuucugaggccuuggcgaggcuucuaaguggcaccgagucggugcuuuuuu

SEQ ID NO: 16 - DNA, 20 bp, Artificial Sequence
  P16 Forward Primer
ccccttgcctggaaagatac

SEQ ID NO: 17 - DNA, 20 bp, Artificial Sequence
  P16 Reverse Primer
agcccctcctctttcttcct

SEQ ID NO: 18 - DNA, 20 bp, Artificial Sequence
  P16 Forward Primer
caacgcaccgaatagttacg

SEQ ID NO: 19 - DNA, 17 bp, Artificial Sequence
  P16 Reverse Primer
agcaccaccagcgtgtc

SEQ ID NO: 20 - RNA, 20 nt, Artificial Sequence
  GN2 Guide Sequence
guuaggaauaaaagcuuuga

SEQ ID NO: 21 - RNA, 20 nt, Artificial Sequence
  G100 Guide Sequence
gugaaccgagagagaucgug

SEQ ID NO: 22 - RNA, 21 nt, Artificial Sequence
  G101 Guide Sequence
gcccccauuaagaaccacugu

SEQ ID NO: 23 - RNA, 20 nt, Artificial Sequence
  G102 Guide Sequence
gguugccaggaugggaggga

SEQ ID NO: 24 - RNA, 21 nt, Artificial Sequence
  G103 Guide Sequence
guucuucucaaaaaagaaagu

SEQ ID NO: 25 - RNA, 20 nt, Artificial Sequence
  G25 Guide Sequence
gacaggacaguauuugaagc

SEQ ID NO: 26 - RNA, 20 nt, Artificial Sequence
  G126 Guide Sequence
gguuuauuuaauacggacgg

SEQ ID NO: 27 - RNA, 21 nt, Artificial Sequence
  G127 Guide Sequence
gacagccguuuuacacgcagg

SEQ ID NO: 28 - RNA, 21 nt, Artificial Sequence
  G128 Guide Sequence
gcaggugauuucgauucucgg

SEQ ID NO: 29 - RNA, 20 nt, Artificial Sequence
  G2 Guide Sequence
gcacucaaacacgccuuugc

SEQ ID NO: 30 - RNA, 21 nt, Artificial Sequence
  G82 Guide Sequence
guaucgcggaggaaggaaacg

SEQ ID NO: 31 - RNA, 20 nt, Artificial Sequence
  G107 Guide Sequence
gcauggagccuucggcugac

SEQ ID NO: 32 - RNA, 20 nt, Artificial Sequence
  G108 Guide Se
guggccagccagucagccga

SEQ ID NO: 33 - RNA, 21 nt, Artificial Sequence
  G122 Guide Sequence
gccgcagccgccgagcgcacg

SEQ ID NO: 34 - RNA, 21 nt, Artificial Sequence
  G123 Guide Sequence
gaggggcuggcuggucaccag

SEQ ID NO: 35 - RNA, 21 nt, Artificial Sequence
  G109 Guide Sequence
gcaccgaauaguuacggucgg

SEQ ID NO: 36 - RNA, 20 nt, Artificial Sequence
  G112 Guide Sequence
gaaaaaggggaggcuuccug

SEQ ID NO: 37 - RNA, 20 nt, Artificial Sequence
  G113 Guide Sequence
ggauuaucaguggaaaucug

SEQ ID NO: 38 - RNA, 21 nt, Artificial Sequence
  G114 Guide Sequence
gaaagaaauguaagaugugcu

SEQ ID NO: 39 - RNA, 21 nt, Artificial Sequence
  G115 Guide Sequence
gaagaaagauaagcuccaucc

SEQ ID NO: 40 - RNA, 21 nt, Artificial Sequence
  G116 Guide Sequence
gugaagggauuacaaggcgug

SEQ ID NO: 41 - DNA, 20 bp, Artificial Sequence
  P16 Forward Primer
caacgcaccgaatagttacg

SEQ ID NO: 42 - DNA, 17 bp, Artificial Sequence
  P16 Reverse Primer
agcaccaccagcgtgtc

SEQ ID NO: 43 - RNA, 23 nt, Artificial Sequence
  R2 Sequence
cccgggacgcggguccgggacag

SEQ ID NO: 44 - RNA, 22 nt, Artificial Sequence
  R5 Sequence
cugaggccuuggcgaggcuucu

SEQ ID NO: 45 - RNA, 100 nt, Artificial Sequence
  Original sgRNA Sequence
  misc_feature (1)..(19): n is a, c, g, or u
nnnnnnnnnnnnnnnnnnnguuuuagagcuagaaauagcaaguuaaaauaaggcuaguccguuaucaacuugaaaaaguggcaccgagucggugcuuuuu

SEQ ID NO: 46 - RNA, 100 nt, Artificial Sequence
  sgRNA Sequence Revision
  misc_feature (1)..(19): n is a, c, g, or u
nnnnnnnnnnnnnnnnnnnguuugagagcuagaaauagcaaguucaaauaaggcuaguccguuaucaacuugaaaaaguggcaccgagucggugcuuuuu

SEQ ID NO: 47 - RNA, 160 nt, Artificial Sequence
  sgSAM Comparison Sequence
  misc_feature (1)..(19): n is a, c, g, or u
nnnnnnnnnnnnnnnnnnnguuuuagagcuaggccaacaugaggaucacccaugucugcagggccuagcaaguuaaaauaaggcuaguccguuaucaacuuggccaacaugaggaucacccaugucugcagggccaaguggcaccgagucggugcuuuuu

SEQ ID NO: 48 - RNA, 119 nt, Artificial Sequence
  MsgDiR1 R2-Stemloop2 Sequence
  misc_feature (1)..(19): n is a, c, g, or u
nnnnnnnnnnnnnnnnnnnguuugagagcuacccgggacgcggguccgggacaguagcaaguucaaauaaggcuaguccguuaucaacuugaaaaaguggcaccgagucggugcuuuuu

SEQ ID NO: 49 - RNA, 118 nt, Artificial Sequence
  MsgDiR2 R5-Stemloop2 Sequence
  misc_feature (1)..(19): n is a, c, g, or u
nnnnnnnnnnnnnnnnnnnguuugagagcuacugaggccuuggcgaggcuucuuagcaaguucaaauaaggcuaguccguuaucaacuugaaaaaguggcaccgagucggugcuuuuu

SEQ ID NO: 50 - RNA, 118 nt, Artificial Sequence
  MsgDiR3 Tetraloop-R2 Sequence
  misc_feature (1)..(19): n is a, c, g, or u
nnnnnnnnnnnnnnnnnnnguuugagagcuagaaauagcaaguucaaauaaggcuaguccguuaucaacuucccgggacgcggguccgggacagaguggcaccgagucggugcuuuuu

SEQ ID NO: 51 - RNA, 117 nt, Artificial Sequence
  MsgDiR4 Tetraloop-R5 Sequence
  misc_feature (1)..(19): n is a, c, g, or u
nnnnnnnnnnnnnnnnnnnguuugagagcuagaaauagcaaguucaaauaaggcuaguccguuaucaacuucugaggccuuggcgaggcuucuaguggcaccgagucggugcuuuuu

SEQ ID NO: 52 - RNA, 138 nt, Artificial Sequence
  MsgDiR5 R5-R2 Sequence
  misc_feature (1)..(19): n is a, c, g, or u
nnnnnnnnnnnnnnnnnnnguuugagagcuacugaggccuuggcgaggcuucuuagcaaguucaaauaaggcuaguccguuaucaacuucccgggacgcggguccgggacagaaguggcaccgagucggugcuuuuuu

SEQ ID NO: 53 - RNA, 138 nt, Artificial Sequence
  MsgDiR6 R2-R5 Sequence
  misc_feature (1)..(19): n is a, c, g, or u
nnnnnnnnnnnnnnnnnnnguuugagagcuacccgggacgcggguccgggacaguagcaaguucaaauaaggcuaguccguuaucaacuucugaggccuuggcgaggcuucuaaguggcaccgagucggugcuuuuuu

SEQ ID NO: 54 - RNA, 139 nt, Artificial Sequence
  MsgDiR7 R2-R2 Sequence
  misc_feature (1)..(19): n is a, c, g, or u
nnnnnnnnnnnnnnnnnnnguuugagagcuacccgggacgcggguccgggacaguagcaaguucaaauaaggcuaguccguuaucaacuucccgggacgcggguccgggacagaaguggcaccgagucggugcuuuuuu

SEQ ID NO: 55 - RNA, 137 nt, Artificial Sequence
  MsgDiR8 R5-R5 Sequence
  misc_feature (1)..(19): n is a, c, g, or u
nnnnnnnnnnnnnnnnnnnguuugagagcuacugaggccuuggcgaggcuucuuagcaaguucaaauaaggcuaguccguuaucaacuucugaggccuuggcgaggcuucuaaguggcaccgagucggugcuuuuuu

SEQ ID NO: 56 - DNA, 20 bp, Artificial Sequence
  p16-F primer
caacgcaccgaatagttacg

SEQ ID NO: 57 - DNA, 17 bp, Artificial Sequence
  p16-R primer
agcaccaccagcgtgtc

SEQ ID NO: 58 - DNA, 20 bp, Artificial Sequence
  CEBPA-F primer
tataggctgggcttcccctt

SEQ ID NO: 59 - DNA, 20 bp, Artificial Sequence
  CEBPA-R primer
agctttctggtgtgactcgg

SEQ ID NO: 60 - DNA, 20 bp, Artificial Sequence
  p14-F primer
gcaggttcttggtgaccctc

SEQ ID NO: 61 - DNA, 24 bp, Artificial Sequence
  p14-R primer
ccatcatcatgacctggtcttcta

SEQ ID NO: 62 - DNA, 20 bp, Artificial Sequence
  p15-F primer
tagtggagaaggtgcgacag

SEQ ID NO: 63 - DNA, 18 bp, Artificial Sequence
  p15-R primer
gcgctgcccatcatcatg

SEQ ID NO: 64 - DNA, 20 bp, Artificial Sequence
  ACTB-F primer
tgaagtgtgacgtggacatc

SEQ ID NO: 65 - DNA, 20 bp, Artificial Sequence
  ACTB-R primer
ggaggagcaatgatcttgat

SEQ ID NO: 66 - RNA, 20 nt, Artificial Sequence
  GN2 Guide RNA
guuaggaauaaaagcuuuga

SEQ ID NO: 67 - RNA, 20 nt, Artificial Sequence
  G2 Guide RNA
gcacucaaacacgccuuugc

SEQ ID NO: 68 - RNA, 20 nt, Artificial Sequence
  G19 Guide RNA
gcucccccgccugccagcaa

SEQ ID NO: 69 - RNA, 21 nt, Artificial Sequence
  G36 Guide RNA
gcuaacugccaaauugaaucg

SEQ ID NO: 70 - RNA, 20 nt, Artificial Sequence
  G108 Guide RNA
guggccagccagucagccga

SEQ ID NO: 71 - RNA, 21 nt, Artificial Sequence
  G122 Guide RNA
gccgcagccgccgagcgcacg

SEQ ID NO: 72 - RNA, 20 nt, Artificial Sequence
  G110 Guide RNA
gacccucuacccaccuggau

SEQ ID NO: 73 - DNA, 26 bp, Artificial Sequence
  p16-BSP-C-F1 primer
tggtttttggattattgtgtaatttt

SEQ ID NO: 74 - DNA, 25 bp, Artificial Sequence
  p16-BSP-C-R1 primer
ctttcctaattataaaaaccccacc

SEQ ID NO: 75 - DNA, 24 bp, Artificial Sequence
  p16-BSP-4F primer
aatttggtagttaggaaggttgta

SEQ ID NO: 76 - DNA, 19 bp, Artificial Sequence
  p16-BSP-4R primer
tccccacctaccccccaca

SEQ ID NO: 77 - DNA, 24 bp, Artificial Sequence
  p16_BSP_original_F primer
tttttagaggatttgagggatagg

SEQ ID NO: 78 - DNA, 24 bp, Artificial Sequence
  p16_BSP_original_R primer
ctacctaattccaattcccctaca

SEQ ID NO: 79 - DNA, 24 bp, Artificial Sequence
  Fp16-BSP-D3-F1 primer
tttaggtgggtagagggtttgtag

SEQ ID NO: 80 - DNA, 25 bp, Artificial Sequence
  Rp16-BSP-D3-R1 primer
aactcctcattcctcttccttaact

SEQ ID NO: 81 - DNA, 23 bp, Artificial Sequence
  p16-E-BSP-F1 primer
ttaggtgggtagagggtttgtag

SEQ ID NO: 82 - DNA, 26 bp, Artificial Sequence
  p16-E-BSP-R1 primer
caaactaaaataaaataactccatct

SEQ ID NO: 83 - DNA, 23 bp, Artificial Sequence
  p16_BS_F primer
atttggtagttaggaaggttgta

SEQ ID NO: 84 - DNA, 22 bp, Artificial Sequence
  p16 _BS_R primer
ccaaaaaacctcccctttttcc

SEQ ID NO: 85 - DNA, 23 bp, Artificial Sequence
  p16_BS_F primer
atttggtagttaggaaggttgta

SEQ ID NO: 86 - DNA, 22 bp, Artificial Sequence
  p16_BS_R primer
ccaaaaaacctcccctttttcc

SEQ ID NO: 87 - DNA, 24 bp, Artificial Sequence
  p16 Exon 1 UF primer
ttattagagggtggggtggattgt

SEQ ID NO: 88 - DNA, 23 bp, Artificial Sequence
  p16 Exon 1 UR primer
ccacctaaatcaacctccaacca

SEQ ID NO: 89 - DNA, 24 bp, Artificial Sequence
  p16 Exon 1 MF primer
ttattagagggtggggcggatcgc

SEQ ID NO: 90 - DNA, 23 bp, Artificial Sequence
  p16 Exon 1 MR primer
ccacctaaatcgacctccgaccg

SEQ ID NO: 91 - DNA, 18 bp, Artificial Sequence
  P16-F
ggtggggctctcacaact

SEQ ID NO: 92 - DNA, 18 bp, Artificial Sequence
  P16-R
ccttcctccgcgatacaa

SEQ ID NO: 93 - DNA, 20 bp, Artificial Sequence
  P14-F (Positive Control)
agaagtctgccgctcctcta

SEQ ID NO: 94 - DNA, 20 bp, Artificial Sequence
  P14-R (Positive Control)
acagatcagacgtcaagccc

SEQ ID NO: 95 - DNA, 20 bp, Artificial Sequence
  P15-F (Positive Control)
gtgaagcccaagtactgcct

SEQ ID NO: 96 - DNA, 20 bp, Artificial Sequence
  P15-F (Positive Control)
tcactgtggagacgttggtg

SEQ ID NO: 97 - DNA, 20 bp, Artificial Sequence
  Down10K-1F (Negative Control)
aggagcccatagcttgtgga

SEQ ID NO: 98 - DNA, 25 bp, Artificial Sequence
  Down10K-1R (Negative Control)
gatacttccactagacatcttgtca

SEQ ID NO: 99 - DNA, 23 bp, Artificial Sequence
  Up50K-1F (Negative Control)
ataaagcattgcaggagcttaca

SEQ ID NO: 100 - DNA, 24 bp, Artificial Sequence
  Up50K-1R (Negative Control)
cctacacatttttgtggcctgttt

SEQ ID NO: 101 - DNA, 21 bp, Artificial Sequence
  Csp6I-NlaIII-1F primer
gcctccgaccgtaactattcg

SEQ ID NO: 102 - DNA, 19 bp, Artificial Sequence
  Csp6I-NlaIII-1R primer
aggacgaagtttgcagggg

SEQ ID NO: 103 - DNA, 22 bp, Artificial Sequence
  DpnII-NlaIII-1F primer
cattggaaggacggactccatt

SEQ ID NO: 104 - DNA, 19 bp, Artificial Sequence
  DpnII-NlaIII-1R primer
tggaaagataccgcggtcc

SEQ ID NO: 105 - DNA, 92 bp, Artificial Sequence
  Csp6I-NlaIII-C-502 primer
aatgatacggcgaccaccgagatctacacctctctattcgtcggcagcgtcagatgtgtataagagacagaagccaaggaagaggaatgagg

SEQ ID NO: 106 - DNA, 92 bp, Artificial Sequence
  Csp6I-NlaIII-C-501 primer
aatgatacggcgaccaccgagatctacactagatcgctcgtcggcagcgtcagatgtgtataagagacagaagccaaggaagaggaatgagg

SEQ ID NO: 107 - DNA, 84 bp, Artificial Sequence
  Csp6I-NlaIII-N-703 primer
caagcagaagacggcatacgagatttctgcctgtctcgtgggctcggagatgtgtataagagacagccagccagtcagccgaag

SEQ ID NO: 108 - DNA, 84 bp, Artificial Sequence
  Csp6I-NlaIII-N-701 primer
caagcagaagacggcatacgagattcgccttagtctcgtgggctcggagatgtgtataagagacagccagccagtcagccgaag

SEQ ID NO: 109 - DNA, 92 bp, Artificial Sequence
  DpnII-NlaIII-D-501 primer
aatgatacggcgaccaccgagatctacactagatcgctcgtcggcagcgtcagatgtgtataagagacagtgctcagtgttctagaagcaga

SEQ ID NO: 110 - DNA, 92 bp, Artificial Sequence
  DpnII-NlaIII-D-502 primer
aatgatacggcgaccaccgagatctacacctctctattcgtcggcagcgtcagatgtgtataagagacagtgctcagtgttctagaagcaga

SEQ ID NO: 111 - DNA, 84 bp, Artificial Sequence
  DpnII-NlaIII-N-704 primer
caagcagaagacggcatacgagatgctcaggagtctcgtgggctcggagatgtgtataagagacagggagagggggagagcagg

SEQ ID NO: 112 - DNA, 84 bp, Artificial Sequence
  DpnII-NlaIII-N-702 primer
caagcagaagacggcatacgagatctagtacggtctcgtgggctcggagatgtgtataagagacagggagagggggagagcagg

SEQ ID NO: 113 - RNA, 20 nt, Artificial Sequence
  G111 Guide RNA
gcccccagggcgucgccagg